SEQUENCE LISTING (23 sequences)

SEQ ID NO: 1 - protein, 11 aa, Artificial Sequence
  Synthetic
RASQSVSSAVA

SEQ ID NO: 2 - protein, 7 aa, Artificial Sequence
  Synthetic
SASSLYS

SEQ ID NO: 3 - protein, 9 aa, Artificial Sequence
  Synthetic
QQSSTDPIT

SEQ ID NO: 4 - protein, 9 aa, Artificial Sequence
  Synthetic
FTVSSSSIH

SEQ ID NO: 5 - protein, 17 aa, Artificial Sequence
  Synthetic
YISSSSGYTYYADSVKG

SEQ ID NO: 6 - protein, 15 aa, Artificial Sequence
  Synthetic
YWSYPSWWPYRGMDY

SEQ ID NO: 7 - protein, 124 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSS

SEQ ID NO: 8 - protein, 108 aa, Artificial Sequence
  Synthetic
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKR

SEQ ID NO: 9 - protein, 20 aa, Artificial Sequence
  Synthetic
MYRMQLLSCIALSLALVTNS

SEQ ID NO: 10 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 106 aa, Artificial Sequence
  Synthetic
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 14 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 15 - protein, 214 aa, Artificial Sequence
  Synthetic
DIQMTQSPSSLSASVGDRVTITCRASQSVSSAVAWYQQKPGKAPKLLIYSASSLYSGVPSRFSGSRSGTDFTLTISSLQPEDFATYYCQQSSTDPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 454 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 454 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 19 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 20 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 451 aa, Artificial Sequence
  Synthetic
EVQLVESGGGLVQPGGSLRLSCAASGFTVSSSSIHWVRQAPGKGLEWVAYISSSSGYTYYADSVKGRFTISADTSKNTAYLQMNSLRAEDTAVYYCARYWSYPSWWPYRGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2024/0287195 A1
(43) Pub. Date: Aug. 29, 2024

(54) ANTI-GALECTIN-9 ANTIBODIES AND USES THEREOF IN THE TREATMENT OF OCULAR MELANOMA

(71) Applicant: PURETECH LYT, INC., Boston, MA (US)

(72) Inventors: Aleksandra FILIPOVIC, London (GB); Joseph BOLEN, Boston, MA (US); Eric ELENKO, Boston, MA (US)

(73) Assignee: PURETECH LYT, INC., Boston, MA (US)

(21) Appl. No.: 18/250,165

(22) PCT Filed: Oct. 26, 2021
(86) PCT No.: PCT/US2021/056681

§ 371 Date: Apr. 21, 2023

Related U.S. Application Data
(60) Provisional application No. 63/105,772, filed on Oct. 26, 2020.

(51) Int. Cl. C07K 16/28; A61K 39/00; A61P 35/04; G01N 33/574

(52) U.S. Cl. CPC C07K 16/2851; A61P 35/04; C07K 16/2818; G01N 33/57407; G01N 33/57492; A61K 2039/505; A61K 2039/507; A61K 2039/545; C07K 2317/55; C07K 2317/732; G01N 2333/4724

(57) ABSTRACT

Methods for treating an ocular melanoma in a patient. e.g., uveal melanoma, comprising administering to the patient an effective amount of an antibody that binds galectin-9). Also provided herein are methods for identifying a subject as having the ocular melanoma based on the level of Galectin-9) in a biological sample (e.g., a blood sample) from a subject suspected of having the ocular melanoma.

### CROSS-REFERENCE TO RELATED APPLICATIONS

The present application is a United States National Phase under 35 U.S.C. § 371 of International Application No. PCT/US2021/056681, filed Oct. 26, 2021, which claims the benefit of the filing date of U.S. Provisional Application No. 63/105,772, filed Oct. 26, 2020, the entire contents of each of which are incorporated by reference herein.

### SEQUENCE LISTING

The instant application contains a Sequence Listing which has been filed electronically in TXT format and is hereby incorporated by reference in its entirety. Said TXT copy, created on Apr. 21, 2023, is named 112174-0247-NP001US00_SEQ.txt and is 48,109 bytes in size.

### BACKGROUND OF INVENTION

Galectin-9 is a tandem-repeat lectin consisting of two carbohydrate recognition domains (CRDs) and was discovered and described for the first time in 1997 in patients suffering from Hodgkin's lymphoma (HL) (Tureci et al., J. Biol. Chem. 1997, 272, 6416-6422). Three isoforms exist, and can be located within the cell or extracellularly. Elevated Galectin-9 levels have been in observed a wide range of cancers, including melanoma, Hodgkin's lymphoma, hepatocellular, pancreatic, gastric, colon and clear cell renal cell cancers (Wdowiak et al. Int. J. Mol. Sci. 2018, 19, 210). In renal cancer, patients with high Galectin-9 expression showed more advanced progression of the disease with larger tumor size (Kawashima et al.; BJU Int. 2014; 113:320-332). In melanoma, galectin-9 was expressed in 57% of tumors and was significantly increased in the plasma of patients with advanced melanoma compared to healthy controls (Enninga et al., Melanoma Res. 2016 October; 26(5): 429-441).

Galectin-9 has been described to play an important role in in a number of cellular processes such as adhesion, cancer cell aggregation, apoptosis, and chemotaxis. Recent studies have shown a role for Galectin-9 in immune modulation in support of the tumor, e.g., through negative regulation of Th1 type responses, Th2 polarization and polarization of macrophages to the M2 phenotype. This work also includes studies that have shown that Galectin-9 participates in direct inactivation of T cells through interactions with the T-cell immunoglobulin and mucin protein 3 (TIM-3) receptor (Dardalhon et al., J Immunol., 2010, 185, 1383-1392; Sanchez-Fueyo et al., Nat Immunol., 2003, 4, 1093-1101).

Galectin-9 has also been found to play a role in polarizing T cell differentiation into tumor suppressive phenotypes), as well as promoting tolerogenic macrophage programming and adaptive immune suppression (Daley et al., Nat Med., 2017, 23, 556-567). In mouse models of pancreatic ductal adenocarcinoma (PDA), blockade of the checkpoint interaction between Galectin-9 and the receptor Dectin-1 found on innate immune cells in the tumor microenvironment (TME) has been shown to increase anti-tumor immune responses in the TME and to slow tumor progression (Daley et al., Nat Med., 2017, 23, 556-567). Galectin-9 also has been found to bind to CD206, a surface marker of M2 type macrophages, resulting in a reduced secretion of CVL22 (MDC), a macrophage derived chemokine which has been associated with longer survival and lower recurrence risk in lung cancer (Enninga et al, J Pathol. 2018 August; 245(4):468-477).

Ocular melanoma is a subtype of cancer that develops from melanocytes in the eye. The vast majority arise from uvea (greater than 85%), but a minority arise from conjunctiva, and other sites (Kaliki & C L Shields (2017) Eye 31, pp. 241-257). Even though local disease can be well controlled, up to half of patients with uveal melanoma will develop distant metastasis, with 5- and 10-year cumulative metastasis rates of 25% and 34%, respectively, and the liver is the most common site of metastasis, seen in 90% of patients with metastatic disease (Singh et al.,

Oncology and Therapy 6, p. 87-104(2018), and references therein). The median survival after diagnosis of patients with liver metastases is approximately 4-6 months with a 1 year survival of about 10-15% (Woodman, Cancer J. 2012 March-April; 18(2): 148-152). About 50% of these patients with liver metastasis have extrahepatic involvement. The most common extrahepatic metastasis sites are the lungs (30%), bone (23%), and skin (17%) (Bedikian, Int Ophthalmol Clin, Winter 2006; 46(1): 151-66.). Accordingly, there is an immediate medical need for new and improved treatment strategies for uveal melanoma and ocular melanoma in general.

### SUMMARY OF INVENTION

In some aspects, the present disclosure provides a method for treating a solid tumor (e.g., an ocular melanoma) that expresses galectin-9 in a human subject. The method comprises administering to the human subject in need thereof an effective amount of a pharmaceutical composition comprising an antibody that binds galectin-9 (anti-galectin-9 antibody). In some instances, wherein the ocular melanoma is uveal melanoma. In other instances, the ocular melanoma is metastatic. In some examples, the ocular melanoma is uveal melanoma, which may be located at choroid, ciliary body, or iris.

In some embodiments the anti-galectin-9 antibody may comprise (a) a heavy chain variable region (V_{H}), which comprises a heavy chain complementary determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6 and (b) a light chain variable region (V_{L}), which comprises a light chain CDR1 set forth as SEQ ID NO: 1, a light chain CDR2 set forth as SEQ ID NO: 2, and a light chain CDR3 set forth as SEQ ID NO: 3. In some examples, the anti-galectin-9 antibody may comprise a V_{H} set forth as SEQ ID NO: 7 and/or the V_{L} set forth as SEQ ID NO: 8.

In some embodiments, the anti-galectin-9 antibody may be a full-length antibody, an antigen-binding fragment thereof, or a single chain variable fragment (scFv). In some examples, the anti-galectin-9 antibody can be a full-length antibody, which may be an IgG molecule. In one example, the antibody is an IgG1 molecule. In another example, the antibody is an IgG4 molecule.

In some examples, the anti-galectin-9 antibody disclosed herein may comprise a heavy chain constant region set forth as SEQ ID NO: 14, and/or a light chain constant region set forth as SEQ ID NO: 11. In specific examples, the anti-galectin-9 antibody comprises a heavy chain comprising the amino acid sequence of SEQ ID NO: 19 and a light chain comprising the amino acid sequence of SEQ ID NO: 15. In one example, the antibody is G9.2-17 IgG4.

In some embodiments, the anti-galectin-9 antibody as disclosed herein (e.g., G9.2-17 IgG4) is administered to the human subject at a dose of about 0.2 mg/kg to about 32 mg/kg, for example, at a dose of about 1 mg/kg to about 32 mg/kg. In some examples, the anti-galectin-9 antibody (e.g., G9.2-17 IgG4) is administered to the human subject at a dose of about 0.2 mg/kg to about 16 mg/kg. In some examples, the anti-galectin-9 antibody (e.g., G9.2-17 IgG4) is administered to the human subject at a dose of about 0.2 mg/kg, about 0.63 mg/kg, about 2 mg/kg, about 6.3 mg/kg, about 10 mg/kg, or about 16 mg/kg. In some examples, the anti-galectin-9 antibody (e.g., G9.2-17 IgG4) is administered to the subject at a dose of about 0.5 mg/kg to about 32 mg/kg, or about 2 mg/kg to about 16 mg/kg. Alternatively or in addition, the anti-galectin-9 antibody (e.g., G9.2-17 IgG4) is administered to the human subject once every two weeks. In some instances, the anti-galectin-9 antibody is administered to the subject by intravenous infusion.

In some embodiments, the human subject for treatment by any of the methods disclosed herein is free of other anti-cancer therapy concurrently with the anti-galectin-9 antibody.

Alternatively, any of the methods disclosed herein may further comprise administering to the human subject an effective amount of a checkpoint inhibitor. In some embodiments, the checkpoint inhibitor is an anti-PD-1 or anti-PD-L1 antibody. Examples include, but are not limited to, nivolumab, prembrolizumab, tislelizumab, or cemiplimab, durvalumab, avelumab, and atezolizumab. In other embodiments, the checkpoint inhibitor may be an anti-CTLA-4 antibody. Any of the immune checkpoint inhibitor may be administered by intravenous infusion. In one example, the antibody that binds PD-1 is nivolumab, which may be administered to the subject at a dose of 240 mg once every two weeks. In another example, the antibody that binds PD-1 is tislelizumab, which may be administered at a dose of about 200 mg once every 3 weeks or at a dose of about 400 mg every six weeks (e.g., at a dose of about 300 mg every 4 weeks).

In other embodiments, any of the methods disclosed herein may further comprise administering a chemotherapy to the human patient. In some examples, the chemotherapy comprise an antimetabolite, a microtubule inhibitor, or a combination thereof. For example, the antimetabolite is gemcitabine and/or the microtubule inhibitor is paclitaxel. In some examples, the paclitaxel is a protein-bound paclitaxel, which preferably is a nanoparticle albumin-bound paclitaxel. The paclitaxel may be administered to the subject at 125 mg/m² intraveneously. Alternatively or in addition, the gemcitabine is administered to the subject at 1000 mg/m². In some instances, the method comprises one or more cycles, each having 28 days, and wherein in each cycle the anti-galectin-9 antibody is administered to the human subject on day 1 and day 15 and the gemcitabine and paclitaxel are administered to the human subject on day 1, day 8, and day 15.

Alternatively or in addition, any of the anti-galectin-9 antibodies disclosed herein (e.g., G9.2-17 IgG4) may be co-used with one or more additional therapeutic agents (e.g., chemotherapeutics or biologic agents) for treating ocular melanoma such as uveal melanoma. Examples include, but are not limited to, dacarbazine, temozolomide, cisplatin, treosulfan, fotemustine, selumetinib, trametinib, binimetinib, sotrastaurin, trichostatin, tenocin-6, valproic acid, crizotinib, bevacizumab, ranibizumab, cabozantinib, bortezomib, cetuximab, gefitinbv, tebentafusp, and ruthenium-106 brachytherapy. In some examples, one or more of selumetinib, trametinib, binimetinib, sotrastaurin, trichostatin, tenocin-6, valproic acid, crizotinib, bevacizumab, ranibizumab, cabozantinib, bortezomib, cetuximab, gefitinbv, tebentafusp, and ruthenium-106 brachytherapy may be co-used with the anti-galectin-9 antibody for treating uveal melanoma.

In some embodiments, the human subject has undergone one or more prior anti-cancer therapies. In some examples, the one or more prior anti-cancer therapies comprise chemotherapy, immunotherapy, radiation therapy, a therapy involving a biologic agent, or a combination thereof. In some instances, the human subject has progressed disease through the one or more prior anti-cancer therapies, or is resistant to the one or more prior therapies.

In some embodiments, the human subject may have an elevated level of galectin-9 relative to a control value. For example, the human subject may have an elevated serum or plasma level of galectin-9 relative to the control value. Alternatively or in addition, the human subject may have cancer cells and/or immune cells expressing galectin-9, which may be in tumor organoids derived from the human patient.

In some instances, the human subject is examined for one or more of the following features before, during, and/or after the treatment:
(a) one or more tumor markers in tumor biopsy samples from the subject, optionally wherein the one or more tumor markers comprise CA15-3, CA-125, CEA, CA19-9, S100, alpha fetoprotein, neuron-specific enolase (NSE), and/or cytokeratin-fragment-21 (CYFRA-21);
   (b) cytokine profile, which optionally comprises interferon gamma (IFN-γ), IL-10, IL-12p70, IL-13, IL-1β, IL-2, IL-4, IL-6, IL-8, TNF-α, MIP-1b, monocyte chemoattractant protein 1 (MCP-1), MIP-1a, IL-17a, IL-5, and/or TGF-β; and
   (c) galectin 9 levels, optionally in blood and/or in tumor tissues;
   (d) PD-L1 level, optionally in tissues;
   (e) Mismatch repair status, optionally in tissues, and
   (f) Tumor mutation burden (TMB).

Any of the treatment methods disclosed herein may further comprise monitoring occurrence of one or more adverse effects in the human subject. In some examples, the one or more adverse effects comprise hepatic impairment, hematologic toxicity, neurologic toxicity, cutaneous toxicity, gastrointestinal toxicity, or a combination thereof. In some embodiments, the method disclosed herein may further comprising reducing the dose of the anti-galectin-9 antibody, optionally the dose of the checkpoint inhibitor, the dose of the chemotherapy, or a combination thereof, when an adverse effect is observed in the human subject.

In other aspects, the present disclosure features a method for identifying an ocular melanoma patient and optionally treating the patient, the method comprising:
(i) providing a biological sample of a subject suspected of having an ocular melanoma,
(ii) measuring the level of galectin-9 in the biological sample, and
(iii) identifying the subject as having the ocular melanoma or being at risk for the ocular melanoma or based on the level of galectin-9 in the biological sample, wherein an elevated level of galectin-9 in the biological sample of the subject relative to a predetermined reference level indicates that the subject has the ocular melanoma or is at risk for the ocular melanoma.

Also within the scope of the present disclosure are (b) pharmaceutical compositions comprising any of the anti-galectin-9 antibodies for use in treating ocular melanoma, either taken alone or in combination with a checkpoint inhibitor or a chemotherapy as disclosed herein, and (b) uses of the anti-galectin-9 antibody for manufacturing a medicament for use in treating ocular melanoma, either taken alone or in combination with the checkpoint inhibitor or chemotherapy as disclosed herein.

Further, the present disclosure also features a method for determining tumor burden or metastatic status in a patient having ocular melanoma, and optionally treating the patient, the method comprising:
(i) providing a biological sample of a patient having an ocular melanoma,
(ii) measuring the level of galectin-9 in the biological sample, and
(iii) determining tumor burden of the patient based on the level of galectin-9 in the blood sample, wherein an elevated level of galectin-9 in the biological sample of the subject relative to a predetermined reference level indicates that the subject has a high tumor burden.

The method disclosed above may further comprise: performing one or more additional diagnostic assays to confirm occurrence of the cancer or tumor burden of the cancer.

Moreover, the present disclosure features a method for assessing progression of ocular melanoma in a subject and optionally treating the subject, the method comprising:
(i) providing a first biological sample of a subject having ocular melanoma at a first time point;
(ii) providing a second biological sample of the subject at a second time point, wherein the second time point is after the first time point;
(iii) measuring the levels of galectin-9 in the first and second biological samples; and
(iv) determining the progression of ocular melanoma in the subject based on the levels of galectin-9 in the first and second biological samples, wherein an increased level of galectin-9 in the second biological sample relative to the level of galectin-9 in the first biological sample indicates progression of ocular melanoma in the subject.

Any of the diagnostic/prognostic methods disclosed herein may further comprise performing to the patient a treatment of the ocular melanoma, e.g., based on the diagnostic and/or prognostic results. Any of the treatment methods disclosed herein may be performed to the ocular melanoma patient.

In yet other aspects, provided herein is a method for determining a response to a treatment in a subject having an ocular melanoma, and optionally modifying the treatment, the method comprising:
(i) providing multiple biological samples of a ocular melanoma patient who is under a treatment comprising an anti-cancer therapy, wherein the multiple biological samples comprise at least a first biological sample collected before the first dose of the anti-cancer therapy and a second biological sample collected after the first dose of the anti-cancer therapy;
(ii) measuring the level of galectin-9 in the biological samples; and
(iii) determining the patient's response to the anti-cancer therapy based on the difference between the level of Gal-9 in the first biological sample and the level of Gal-9 in the second biological sample; wherein a reduced level of Gal-9 in the second biological sample relative to the first biological sample indicates a response to the anti-cancer therapy in the subject.

Such a method may comprise multiple doses of the anti-cancer therapy and the multiple biological samples comprises the first biological sample collected before the first dose of the anti-cancer therapy and a plurality of the second biological samples each collected after one dose of the anti-cancer therapy, and wherein a reduced level of galectin-9 in a later collected biological sample relative to the level of galectin-9 in an earlier collected biological sample indicates that the patient is responsive to the treatment.

In some instances, the method may further comprise modifying the anti-cancer therapy based on the patient's response to the anti-cancer therapy determined in step (iii), wherein the modification is selected from the group consisting of: (i) increasing or decreasing the dosage of the anti-cancer therapy, (ii) terminating the anti-cancer therapy, (iii) increasing or decreasing the frequency of the anti-cancer therapy, and (iv) increasing or decreasing the duration of the anti-cancer therapy. Any of the treatment methods disclosed herein may be used as the anti-cancer therapy.

In any of the methods disclosed herein, the biological sample(s) is a blood sample(s), which optionally is a serum sample or a plasma sample. In some embodiments, the level of galectin-9 is measured by an immunoassay.

In addition, provided herein are methods for determining a prognosis of a ocular melanoma cancer patient and optionally treating the patient, the method comprising: (i) obtaining a tumor tissue sample from a human ocular melanoma patient; (ii) measuring the level of galectin-9 in the tumor sample; and (iii) assessing the patient's prognosis based on the level of galectin-9 measured in step (ii) relative to a predetermined reference level, and wherein the cancer is ocular melanoma.

Further provided herein are methods for predicting a survival rate or likelihood of survival of a ocular melanoma patient, the method comprising: (i) obtaining a tumor tissue sample from a human ocular melanoma patient; (ii) measuring the level of galectin-9 in the tumor sample; and (iii) assessing a survival rate of the cancer patient based on the level of galectin-9 measured in step (ii) relative to a predetermined reference level, and wherein the cancer is ocular melanoma.

The details of one or more embodiments of the invention are set forth in the description below. Other features or advantages of the present invention are be apparent from the following drawing and detailed description of several embodiments, and also from the appended claims.

### BRIEF DESCRIPTION OF DRAWINGS

The following drawings form part of the present specification and are included to further demonstrate certain aspects of the present disclosure, which can be better understood by reference to the drawing in combination with the detailed description of specific embodiments presented herein.

FIGS. 1A-1F include bar graphs showing levels of Galectin-9 expression as measured in T cells (CD3⁺), macrophages (CD11b+,) and tumor cells (Epcam+) in S2 and S3 organoid fractions derived from a pancreatic adenocarcinoma biopsy using anti-Galectin-9 G9.2-17 Fab fragment and a commercially available anti-Galectin-9 antibody (9M1-3). S2 fraction: organoids. S3 fraction: single cells. Corresponding isotype for G9.2-17 Fab (“Fab isotype”) and “fluorescence minus one” (FMO) 9M1-3 (“Gal9 FMO”) were used as controls for specificity, background staining and fluorescence bleed through from other channels. FIG. 1A shows levels of Galectin-9 in CD3⁺ cells as measured in the S3 fraction. FIG. 1B shows levels of Galectin-9 in CD11b⁺ cells as measured in the S3 fraction. FIG. 1C shows levels of Galectin-9 in Epcam⁺ cells as measured in the S3 fraction. FIG. 1D shows levels of Galectin-9 in CD3⁺ cells as measured in the S2 fraction. FIG. 1E shows levels of Galectin-9 in CD11b⁺ cells as measured in the S2 fraction. FIG. 1F shows levels of Galectin-9 in Epcam⁺ cells as measured in the S2 fraction.

FIGS. 2A-2F include bar graphs showing levels of Galectin-9 expression as measured in T cells (CD3⁺), macrophages (CD11b+,) and tumor cells (Epcam+) in S2 and S3 organoid fractions derived from a colorectal carcinoma biopsy using anti-Galectin-9 G9.2-17 Fab fragment and a commercially available anti-Galectin-9 antibody (9M1-3). S2 fraction: organoids. S3 fraction: single cells. Corresponding isotype for G9.2-17 Fab (“Fab isotype”) and FMO 9M1-3 (“Gal9 FMO”) were used controls for specificity, background staining and fluorescence bleed through from other channels. FIG. 2A shows levels of Galectin-9 in CD3⁺ cells as measured in the S3 fraction. FIG. 2B shows levels of Galectin-9 in CD11b⁺ cells as measured in the S3 fraction. FIG. 2C shows levels of Galectin-9 in Epcam⁺ cells as measured in the S3 fraction. FIG. 2D shows levels of Galectin-9 in CD3⁺ cells as measured in the S2 fraction. FIG. 2E shows levels of Galectin-9 in CD11b⁺ cells as measured in the S2 fraction. FIG. 2F shows levels of Galectin-9 in Epcam⁺ cells as measured in the S2 fraction.

FIGS. 3A-3F include bar graphs showing levels of Galectin-9 expression as measured in T cells (CD3⁺), macrophages (CD11b+,) and tumor cells (Epcam+) in S2 and S3 organoid fractions derived from a second pancreatic adenocarcinoma biopsy using anti-Galectin-9 G9.2-17 Fab fragment and a commercially available Galectin-9 antibody (9M1-3). S2 fraction: organoids. S3 fraction: single cells. Corresponding isotype for G9.2-17 Fab (“Fab isotype”) and FMO 9M1-3 (“Gal9 FMO”) were used as controls for specificity, background staining and fluorescence bleed through from other channels. FIG. 3A shows levels of Galectin-9 in CD3⁺ cells as measured in the S3 fraction. FIG. 3B shows levels of Galectin-9 in CD11b⁺ cells as measured in the S3 fraction. FIG. 3C shows levels of Galectin-9 in Epcam⁺ cells as measured in the S3 fraction. FIG. 3D shows levels of Galectin-9 in CD3⁺ cells as measured in the S2 fraction. FIG. 3E shows levels of Galectin-9 in CD11b⁺ cells as measured in the S2 fraction. FIG. 3F shows levels of Galectin-9 in Epcam⁺ cells as measured in the S2 fraction.

FIG. 4 is a graph showing measurements of galectin-9 levels in patient sera. Galectin-9 levels in patient sera (sample size in parentheses) was measured using enzyme-linked immunosorbant assay (ELISA). The ELISA was run in sandwich format using the Human Galectin-9 PicoKine ELISA kit (Bolster Biological Technology). Patient serum galectin-9 levels were compared to healthy human serum controls. Statistical analysis performed by unpaired Student's t-test. (*p<0.05; **p<0.01; ***p<0.001; ****p<0.0001). Results show Galectin-9 levels in serum is significantly increased in cancer patients.

FIGS. 5A-5C include photographs of immunohistochemical analysis of various tumors using anti-Galectin-9 antibody 1G3. All magnifications are 200× FIG. 5A shows Chemotherapy treated colorectal cancer with heterogeneous intensity score 2 and 3 (moderate and high) Galectin-9 expression. Galectin-9 staining was observed at the cell membrane in particular; additionally, intraglandular macrophages are moderately positive and stromal reaction in tumor shows multinucleated macrophage giant cells with moderately strong Galectin-9 expression. FIG. 5B shows liver metastasis of colorectal carcinoma with high (intensity score 3) Galectin-9 expression. Staining is located on the membrane and in the cytoplasm. FIG. 5C shows Galectin-9 positive (intensity score 2) entrapped bile ducts and Galectin-9 negative cancer.

FIGS. 6A and 6B depict graphs showing the effect of 9.2-17 in a B16F10 subcutaneous syngeneic model. Tumors were engrafted subcutaneously and treated with G9.2-17 IgG1 mouse mAb, anti-PD1 antibody or a combination of G9.2-17 IgG1 mouse mAb and anti-PD1 antibody. FIG. 6A depicts a graph showing the effect on tumor volume. FIG. 6B depicts a graph showing intratumoral CD8 T cell infiltration. Results show that intra-tumoral presence effector T cells were enhanced in the combination arm.

FIG. 7 includes a graph showing the fraction of annexin V- and propidium iodide (PI)-positive cells plotted as a function of antibody concentration used. MOLM-13 cells were co-incubated with varying concentrations of either G9.2-17 or human IgG4 isotype antibody and recombinant human Galectin-9 for 16 hours. Cells were stained with annexin V and propidium iodide prior to analysis by flow cytometry. Each condition was performed in triplicate. Analysis was performed on FlowJo software.

FIGS. 8A and 8B depict graphs showing the results of ADCC assays performed with the IgG1 form of G9.2-17 (FIG. 8A) and the IgG4 form of G9.2-17 (FIG. 8B). As expected for a human IgG4 mAb, G9.2-17 does not mediate ADCC (FIG. 8B). This was tested against the IgG1 human counterpart of G9.2-17 as a positive control, which mediates ADCC and ADCP, as expected (FIG. 8A).

### DETAILED DESCRIPTION OF INVENTION

Galectin-9, a tandem-repeat lectin, is a beta-galactoside-binding protein, which has been shown to have a role in modulating cell-cell and cell-matrix interactions. The present disclosure is based, at least in part, on the unexpected discovery of galectin-9 (e.g., blood level of galectin-9) as a biomarker that is correlated with occurrence, tumor burden, and/or metastatic status of certain solid tumors. Across multiple cohorts, galectin-9 was significantly increased in blood samples of individuals with primary and metastatic pancreatic cancer, lung tumors, and colorectal carcinoma, compared to healthy individuals. Accordingly, in some embodiments of the disclosure, measurement of galectin-9 levels can be used as a biomarker, for example, to select or identify patients, or to diagnose patients.

Ocular melanoma is a subtype of cancer that develops from melanocytes in the eye. Among ocular melanomas, the vast majority arise from uvea (greater than 85%), but a minority arise from conjunctiva, and from other sites (Kaliki & C L Shields (2017) Eye 31, pp. 241-257, and references therein). Uveal melanoma is a subtype of melanoma occurring in the uveal tract of the eye and is the most common primary intraocular tumor in adults with a mean age-adjusted incidence of 5.1 cases per million per year (Damato and Damato (2012), Ophthalmology, 119(8), 1582-1589). Conjunctival malignant melanoma is an uncommon tumor which comprises about 2% of all eye tumors, 5% of melanomas in the ocular region (Wong et al., Expert Rev Ophthalmol. 2014 June; 9(3): 185-204).

Even though local disease can be well controlled, up to half of patients with uveal melanoma will develop distant metastasis, with 5- and 10-year cumulative metastasis rates of 25% and 34%, respectively, and the liver is the most common site of metastasis, seen in 90% of patients with metastatic disease (Singh et al., Oncology and Therapy 6, p. 87-104(2018), and references therein). The median survival after diagnosis of patients with liver metastases is approximately 4-6 months with a 1 year survival of about 10-15% (Woodman, Cancer J. 2012 March-April; 18(2): 148-152). About 50% of these patients with liver metastasis have extrahepatic involvement. The most common extrahepatic metastasis sites are the lungs (30%), bone (23%), and skin (17%) (Bedikian, Int Ophthalmol Clin, Winter 2006; 46(1):151-66.)

Monosomy of chromosome 3 and amplification of 8q are cytogenetic alterations observed most frequently. These amplifications are conserved in metastases and are associated with poor prognosis (Singh et al., Archives of Pathology & Laboratory Medicine. 2009; 133(8): 1223-1227).

The biology of cutaneous and ocular melanomas seem to differ. For example, in uveal melanoma, the successes seen in cutaneous melanoma with MAP kinase and immune checkpoint inhibitors are not recapitulated. For example, immune checkpoint blockade therapy against PD1 or its ligand PD-L1 induces long-term survival in a large subset of cutaneous melanoma patients, but shows only limited activity in metastatic uveal melanoma patients (Algazi et al., Cancer. 2016 Nov. 15; 122(21): 3344-3353).

Two genes encoding G protein Gα subunits, GNAQ and GNA11 are found in a mutually exclusive manner in over 80% of uveal melanomas, and activate the classical G protein signaling cascade via inositol-3-phosphate, diacylglycerol, and cyclic AMP, resulting in activation of MAP kinases, protein kinase B (Akt) and protein kinase C (PKC), phosphoinositide 3-kinase (PI3K), and mammalian target of rapamycin (mTOR). GNAQ and GNA11 have also been shown to activate the transcription factor complex YAP/TAZ in a HIPPO-independent manner (Amaro et al., Cancer Metastasis Rev. 2017; 36(1): 109-140 and references therein).

Globe-preserving therapies (radiation, laser therapy, surgical resection) or surgery to remove the entire eye (enucleation) are employed for local treatment of ocular melanoma, such as uveal melanoma (Yang et al., (2018) Ther Adv Med Oncol, 10: 1-17). To date, there are no effective treatment options for patients with advanced and metastatic ocular melanoma, and no FDA-approved standard of care is available. Because the biology of ocular melanoma such as uveal melanoma is different from that of cutaneous melanoma, the development of treatments the development of specific treatments and dedicated management is necessary (Yang et al. (2018), Ther Adv Med Oncol.). Several experimental treatments have been assessed, including immunotherapy, systemic chemotherapies, various kinase inhibitor therapies and liver-directed therapies, with low response rates and no overall improvement in survival. Accordingly, there is an immediate medical need for new and improved treatment strategies for uveal melanoma and ocular melanoma in general.

The present disclosure is based, at least in part, on the surprising observation that the serum level of galectin-9 correlates with occurrence, tumor burden, and/or metastatic status of solid cancers, such as melanoma. Thus, galectin-9 can serve as a reliable biomarker for diagnosing such solid cancers and/or assessing tumor burden and/or metastatic status of the cancers Accordingly, provided herein are methods for diagnosing subjects (e.g., human patients) for having a target solid cancer, such as ocular melanoma, using galectin-9 as a biomarker, for example, the blood level of galectin-9 as a biomarker. Also provided herein are methods for assessing tumor burden, metastatic status, or prognostic status of a solid cancer, such as ocular melanoma, in a subject using galectin-9 as a biomarker, for example, the blood level of galectin-9 as a biomarker. Methods of treating ocular melanoma, comprising administering an anti-Galectin-9 antibody are also provided. In some embodiments, the ocular melanoma is metastatic. In some embodiments, the metastatic tumor is in the liver. In some embodiments, the ocular melanoma is uveal melanoma. In some embodiments, the uveal melanoma is located in the choroid, the ciliary body or the iris. In some embodiments, the ocular melanoma is conjunctival melanoma. In some embodiments, the uveal or conjunctival melanoma is metastatic.

### I. Treatment of Ocular Melanoma

In some aspects, provided herein are methods for treating ocular melanoma in a subject (e.g., a human patient) using an anti-galectin-9 antibody. Such a patient may be identified by any of the diagnostic methods disclosed herein, e.g., having the cancer with a high tumor burden, or having the cancer with metastasis or at risk for such).

### A. Anti-Galectin-9 Antibodies

Anti-Galectin-9 antibodies can serve as therapeutic agents for treating diseases associated with Galectin-9 (e.g., those in which a Galectin-9 signaling plays a role). Without being bound by theory, an anti-Galectin-9 antibody may block a signaling pathway mediated by Galectin-9. For example, the antibody may interfere with the interaction between Galectin-9 and its binding partner (e.g., Dectin-1, TIM-3 or CD206), thereby blocking the signaling triggered by the Galectin-9/Ligand interaction. Alternatively, or in addition, an anti-Galectin-9 antibody may also exert its therapeutic effect by inducing blockade and/or cytotoxicity, for example, ADCC, CDC, or ADCP against pathologic cells that express Galectin-9. A pathologic cell refers to a cell that contributes to the initiation and/or development of a disease, either directly or indirectly.

The anti-Galectin-9 antibodies disclosed herein are capable of suppressing the signaling mediated by Galectin-9 (e.g., the signaling pathway mediated by Galectin-9/Dectin-1 or Galectin-9/Tim-3) or eliminating pathologic cells expressing Galectin-9 via, e.g., ADCC. Accordingly, the anti-Galectin-9 antibodies described herein can be used for inhibiting any of the Galectin-9 signaling and/or eliminating Galectin-9 positive pathologic cells, thereby benefiting treatment of diseases associated with Galectin-9, for example, autoimmune diseases, infectious disorders, solid tumors and other cancers, allergic disorders, or hematological disorders such as hematological malignancies.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-Galectin-9 antibody, encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the “Contact” numbering scheme, the IMGT” numbering scheme, the “AHo” numbering scheme, the Chothia definition, the AbM definition, the EU definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917; Al-lazikani et al (1997) J. Molec. Biol. 273:927-948; Edelman et al., Proc Natl Acad Sci USA. 1969 May; 63(1):78-85; Almagro, J. Mol. Recognit. 17:132-143 (2004); MacCallum et al., J. Mol. Biol. 262:732-745 (1996); Lefranc M P et al., Dev Comp Immunol, 2003 January; 27(1):55-77; and Honegger A and Pluckthun A, J Mol Biol, 2001 Jun. 8; 309(3):657-70). See also hgmp.mrc.ac.uk and bioinf.org.uk/abs).

In some embodiments, the anti-Galectin-9 antibody described herein is a full-length antibody, which contains two heavy chains and two light chains, each including a variable domain and a constant domain. Alternatively, the anti-Galectin-9 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

Any of the antibodies described herein, e.g., anti-Galectin-9 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some examples, an anti-Galectin 9 antibody is G9.2-17 or a functional equivalent thereof. Reference antibody G9.2-17 refers to an antibody capable of binding to human Galectin-9 and comprises a heavy chain variable region of SEQ ID NO:7 and a light chain variable domain of SEQ ID NO:8, both of which are provided below. The CDRs provided in Table 1 and identified in the V_{H} and V_{L} sequences are based on the Kabat method.

TABLE 1
Heavy and Light Chain CDRs of G9.2-17

| | | | |
|---|---|---|---|
| G9.2-17 | V_{L} CDR1 | RASQSVSSAVA | SEQ ID NO: 1 |
| | V_{L} CDR2 | SASSLYS | SEQ ID NO: 2 |
| | V_{L} CDR3 | QQSSTDPIT | SEQ ID NO: 3 |
| | V_{H} CDR1 | FTVSSSSIH | SEQ ID NO: 4 |
| | V_{H} CDR2 | YISSSSGYTYYADSVKG | SEQ ID NO: 5 |
| | V_{H} CDR3 | YWSYPSWWPYRGMDY | SEQ ID NO: 6 |

V_{H}: EVQLVESGGGLVQPGGSLRLSCAASGFTVSSSSIHWVRQAPGKGLEWVAYISSSSG
YTYYADSVKGRFTISADTSKNTAYLQMNSLRAEDTAVYYCARYWSYPSWWPYRGMDYWGQ
GTLVTVSS (SEQ ID NO: 7)
V_{L}: DIQMTQSPSSLSASVGDRVTITCRASQSVSSAVAWYQQKPGKAPKLLIYSASSLYS
SGVPSRFSGSRSGTDFTLTISSLQPEDFATYYCQQSTDPITFGQGTKVEIKR(SEQ ID
NO: 8)

In some embodiments, the anti-Galectin-9 antibody for use in the methods disclosed herein is an antibody having the same heavy chain complementary determining regions (CDRs) as reference antibody G9.2-17 and/or the same light chain complementary determining regions as reference antibody G9.2-17. In some embodiments, the anti-Galectin-9 antibody for use in the method disclosed herein can be an antibody having the same heavy chain variable region (V_{H}) and/or the same light chain variable region (V_{L}) as reference antibody G9.2-17. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Exemplary numbering schemes for determining antibody CDRs include the “Kabat” numbering scheme (Kabat et al. (1991), 5th Ed. Public Health Service, National Institutes of Health, Bethesda, Md.), the “Chothia” numbering scheme (Al-Lazikani et al., (1997) JMB 273,927-948), the “Contact” numbering scheme (MacCallum et al., J. Mol. Biol. 262:732-745 (1996)), the “IMGT” numbering scheme (Lefranc M P et al., Dev Comp Immunol, 2003 January; 27(1):55-77), and the “AHo” numbering scheme (Honegger A and Pluckthun A, J Mol Biol, 2001 Jun. 8; 309(3):657-70).

In some instances, the anti-Galectin-9 antibody disclosed herein is a functional variant of reference antibody G9.2-17. A functional variant can be structurally similar as the reference antibody (e.g., comprising the limited number of amino acid residue variations in one or more of the heavy chain and/or light chain CDRs as G9.2-17 as disclosed herein, or the sequence identity relative to the heavy chain and/or light chain CDRs of G9.2-17, or the V_{H} and/or V_{L} of G9.2-17 as disclosed herein) with substantially similar binding affinity (e.g., having a KD value in the same order) to human Galectin-9.

In some embodiments, the anti-Galectin-9 antibody comprises heavy and light chain variable regions, wherein the light chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the light chain variable region CDR1, CDR2, and CDR3 amino acid sequences set forth in SEQ ID NOs: 1, 2, and 3, respectively. In some embodiments, the anti-Galectin-9 antibody comprises heavy and light chain variable regions, wherein the heavy chain variable region CDR1, CDR2, and CDR3 amino acid sequences have at least 80% (e.g., 80%, 85%, 90%, 91%, 92%, 93%, 94%, 95%, 96%, 97%, 98%, or 99% and any increment therein) sequence identity to the heavy chain variable region CDR1, CDR2, and CDR3 amino acid sequences set forth in SEQ ID NO: 4, 5, and 6, respectively.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-Galectin-9 antibody described herein comprises a V_{H} that comprises the HC CDR1, HC CDR2, and HC CDR3, which collectively contain up to 8 amino acid residue variations (8, 7, 6, 5, 4, 3, 2, or 1 variations(s), including additions, deletions, and/or substitutions) relative to the HC CDR1, HC CDR2, and HC CDR3 of reference antibody G9.2-17. Alternatively or in addition, in some embodiments, the anti-Galectin-9 antibody described herein comprises a V_{L} that comprises the LC CDR1, LC CDR2, and LC CDR3, which collectively contain up to 8 amino acid residue variations (8, 7, 6, 5, 4, 3, 2, or 1 variations(s) including additions, deletions, and/or substitutions) relative to the LC CDR1, LC CDR2, and LC CDR3 of reference antibody G9.2-17.

In one example, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Additional Galectin-9 antibodies, e.g., which bind to the CRD1 and/or CRD2 region of Galectin-9 are described U.S. Pat. No. 10,344,091 and WO 2019/084553, the relevant disclosures of each of which are incorporated by reference for the subject matter and purpose referenced herein.

The anti-Gal9 antibody, including the reference antibody G9.2-17, can be in any format as disclosed herein, for example, a full-length antibody or a Fab. In some embodiments, the heavy chain of any of any of the anti-Galectin-9 antibodies as described herein further comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain) of any IgG subfamily as described herein.

In some embodiments, the heavy chain constant region of the antibodies described herein comprises a single domain (e.g., CH1, CH2, or CH3) or a combination of any of the single domains, of a constant region (e.g., SEQ ID NO: 10, 12, 13, 14, 20, and 21). In some embodiments, the light chain constant region of the antibodies described herein comprise a single domain (e.g., CL), of a constant region. Exemplary light and heavy chain sequences are listed below. Exemplary light and heavy chain sequences are listed below. The hIgG1 LALA sequence includes two mutations, L234A and L235A (EU numbering), which suppress FcgR binding as well as a P329G mutation (EU numbering) to abolish complement C1q binding, thus abolishing all immune effector functions. The hIgG4 Fab Arm Exchange Mutant sequence includes a mutation to suppress Fab Arm Exchange (S228P; EU numbering). An IL2 signal sequence (MYRMQLLSCIALSLALVTNS; SEQ ID NO: 9) can be located N-terminally of the variable region. It is used in expression vectors, which is cleaved during secretion and thus not in the mature antibody molecule. The mature protein (after secretion) starts with “EVQ” for the heavy chain and “DIM” for the light chain. Amino acid sequences of exemplary heavy chain constant regions are provided below:

hIgG1 Heavy Chain Constant Region
(SEQ ID NO: 10)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK*
hIgG1 LALA Heavy Chain Constant Region
(SEQ ID NO: 12)
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPEAAGGPSVFLFPPKPKDTLMI
SRTPEVTCVVVDVSHEDPEVKFNWYVDGVEVHNAKTKPREEQYNSTYRVVSVLTVLHQDWLNGKEYKC
KVSNKALGAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK*
hIgG4 Heavy Chain Constant Region
(SEQ ID NO: 13)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSPGK*
hIgG4 Heavy Chain Constant Region
(SEQ ID NO: 20)
ASTKGPSVEPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPSCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGK*
hIgG4 mut Heavy Chain Constant Region
(SEQ ID NO: 14)
ASTKGPSVFPLAPCSRSTSESTAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFLGGPSVFLFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSPGK*
hIgG4 mut Heavy Chain Constant Region
(SEQ ID NO: 21)
ASTKGPSVFPLAPCSRSTSESTAALGCLVKDYFPEPVTVSWNSGALTSGVHTFPAVLOSSGLYSLSSV
VTVPSSSLGTKTYTCNVDHKPSNTKVDKRVESKYGPPCPPCPAPEFLGGPSVELFPPKPKDTLMISRT
PEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLSLSLGK*

The term “G9.2-17(Ig4)” used herein refers to a G9.2-17 antibody which is an IgG4 molecule. Likewise, the term “G9.2-17 (Fab)” refers to a G9.2-17 antibody, which is a Fab molecule. The heavy and light chain CDRs of reference antibody G9.2-17 are provided in Table 1 (determined using the Kabat methodology).

In some embodiments, anti-Galectin antibodies having any of the above heavy chain constant regions are paired with a light chain having the following light chain constant region:

Light Chain Constant Region (SEQ ID NO: 11)
TVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Exemplary full length anti-Galectin-9 antibodies are provided below:
G9.2-17 hIgG1 Heavy Chain (SEQ ID NO: 16)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVEPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVELFPPKPKDTLMISRTPEVTCVVVD
VSHEDPEVKFNWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK*
G9.2-17 hIgG1 LALA Heavy Chain (SEQ ID NO: 17)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVEPLA
PSSKSTSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPEAAGGPSVELFPPKPKDTLMISRTPEVTCVVVD
VSHEDPEVKFNWYVDGVEVHNAKTKPREEQYNSTYRVVSVLTVLHQDWLNGKEYKCKVSNKALGAPIE
KTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK*
G9.2-17 hIgG4 Heavy Chain (SEQ ID NO: 18)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDY FPEPVTVSWNSGALTSGVHT FPAVLQSSGLYSLSSVVTVPSSSLGTKT
YTCNVDHKPSNTKVDKRVESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSE
FLYSRLTVDKSRWQEGNVFSCSVMHEALHNHYTQKSLSLSPGK*
G9.2-17 hIgG4 Heavy Chain (SEQ ID NO: 22)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPSCPAPEFLGGPSVELFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK*
G9.2-17 hIgG4 Fab Arm Exchange mut Heavy Chain (SEQ ID NO: 19)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVFPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVELFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSPGK*
G9.2-17 hIgG4 Fab Arm Exchange mut Heavy Chain (SEQ ID NO: 23)
EVQLVESGGGLVQPGGSLRLSCAASGFTVSSSSIHWVRQAPGKGLEWVAYISSSSGYTYYADSVKGRF
TISADTSKNTAYLQMNSLRAEDTAVYYCARYWSYPSWWPYRGMDYWGQGTLVTVSSASTKGPSVEPLA
PCSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK*
Any of the above heavy chain can be paired with a Light Chain of
(SEQ ID NO: 15) shown below:
DIQMTQSPSSLSASVGDRVTITCRASQSVSSAVAWYQQKPGKAPKLLIYSASSLYSGVPSRFSGSRSG
TDFTLTISSLOPEDFATYYCQQSSTDPITFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSENRGEC*

Any of the anti-Galectin 9 antibodies disclosed herein may be produced by any method known in the art, including but not limited to, recombinant technology. The anti-Galectin-9 antibodies thus prepared can be characterized using methods known in the art, whereby reduction, amelioration, or neutralization of Galectin-9 biological activity is detected and/or measured. For example, in some embodiments, an ELISA-type assay is suitable for qualitative or quantitative measurement of Galectin-9 inhibition of Dectin-1 or TIM-3 signaling.

The bioactivity of an anti-Galectin-9 antibody can verified by incubating a candidate antibody with Galectin-9, and monitoring any one or more of the following characteristics: (a) binding between Dectin-1 and Galectin-9 and inhibition of the signaling transduction mediated by the binding; (b) preventing, ameliorating, or treating any aspect of a solid tumor as those described herein; (c) blocking or decreasing Dectin-1 activation; (d) inhibiting (reducing) synthesis, production or release of Galectin-9. Alternatively, TIM-3 can be used to verify the bioactivity of an anti-Galectin-9 antibody using the protocol described above. Alternatively, CD206 can be used to verify the bioactivity of an anti-Galectin-9 antibody using the protocol described above.

In some embodiments, bioactivity or efficacy is assessed in a subject, e.g., by measuring peripheral and intra-tumoral T cell ratios, T cell activation, or by macrophage phenotyping. Additional assays to determine bioactivity of an anti-Galectin-9 antibody include measurement of CD8+ and CD4+ (conventional) T-cell activation (in an in vitro or in vivo assay, e.g., by measuring inflammatory cytokine levels, e.g., IFNgamma, TNFalpha, CD44, ICOS granzymeB, Perforin, IL-2 (upregulation); CD26L and IL-10 (downregulation)); measurement of reprogramming of macrophages (in vitro or in vivo), e.g., from the M2 to the M1 phenotype (e.g., increased MHCII, reduced CD206, increased TNF-alpha and iNOS), Alternatively, levels of ADCC can be assessed, e.g., in an in vitro assay, as described herein.

### B. Pharmaceutical Compositions

The anti-Galectin-9 antibodies, as well as the encoding nucleic acids or nucleic acid sets, vectors comprising such, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Areiams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Areiams and Wilkins, Ed. K. E. Hoover). Acceptable carriers, excipients, or stabilizers are nontoxic to recipients at the dosages and concentrations used, and comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

In some embodiments, the anti-Galectin-9 antibodies, or the encoding nucleic acid(s), are be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Suitable surface-active agents include, in particular, non-ionic agents, such as polyoxyethylenesorbitans (e.g., Tween™ 20, 40, 60, 80 or 85) and other sorbitans (e.g., Span™M 20, 40, 60, 80 or 85). Compositions with a surface-active agent are conveniently comprise between 0.05 and 5% surface-active agent, and can be between 0.1 and 2.5%. It are be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It are be appreciated that other ingredients may be added, for example glycerol or glucose, to adjust the tonicity of the emulsion. Suitable emulsions are typically contain up to 20% oil, for example, between 5 and 20%. The fat emulsion can comprise fat droplets between 0.1 and 1.0 .im, particularly 0.1 and 0.5 .im, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

C. Treatment of Ocular Melanoma with Anti-Galectin-9 Antibodies

Any of the anti-Galectin 9 antibodies or a pharmaceutical composition comprising such may be used for treating a cancer patient diagnosed by the methods disclosed herein.

To perform the treatment methods disclosed herein, an effective amount of any of the anti-Galectin 9 antibodies as disclosed (e.g., G9.2-17 or a functional equivalent thereof) herein may be administered to the subject. In some embodiments, the anti-Galectin-9 antibody is G9.2-17. In some embodiments, the anti-Galectin-9 antibody is an antibody having the same heavy chain CDR sequences and/or the same light chain CDR sequences as reference antibody G9.2-17. In some embodiments, the anti-Galectin-9 antibody is an antibody having the same V_{H} and V_{L} sequences as reference antibody G9.2-17. In some embodiments, such an antibody is an IgG1 molecule (e.g., having a wild-type IgG1 constant region or a mutant thereof as those disclosed herein). Alternatively, the antibody is an IgG4 molecule (e.g., having a wild-type IgG4 constant region or a mutant thereof as those described herein). In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain variable region comprising SEQ ID NO: 7. In some embodiments, the antibody comprises a light chain variable region comprising SEQ ID NO: 8 and heavy chain comprising SEQ ID NO: 19. In some embodiments, the antibody comprises a light chain comprising SEQ ID NO: 15. In specific examples, the anti-Galectin-9 antibody used herein has a heavy chain of SEQ ID NO: 19 and a light chain of SEQ ID NO: 15. In some embodiments, the antibody is G9.2-17 IgG4.

In some embodiments, the patient to be treated by a method as disclosed herein may have metastatic ocular melanoma. As used herein, metastatic solid tumors/cancer refer to tumors/cancers having tumor/cancer cells from the place where they first form to another part of the body. In metastasis, cancer cells break away from the original tumor, travel through the blood or lymph system, and form a new tumor in other organs or tissues of the body. As used herein, relapsed solid tumors refer to the return of a tumor/cancer or the signs or symptoms of the tumor/cancer after a period of treatment and improvement. As used herein, refractory solid tumors refer to tumors/cancers that do not respond to a treatment, including those that are resistant at the beginning of the treatment or those that are responsive to a treatment but become resistant during the treatment.

As used herein, tumor burden refers to amount of cancer, the size or the volume of the tumor in the body of a subject, accounting for all sites of disease. Tumor burden can be measured using methods known in the art, including but not limited to, FDG positron emission tomography (FDG-PET), magnetic resonance imaging (MRI), and optical imaging, comprising bioluminescence imaging (BLI) and fluorescence imaging (FLI).

A subject having ocular melanoma can be identified by routine medical examination, e.g., laboratory tests, organ functional tests, genetic tests, interventional procedure (biopsy, surgery) any and all relevant imaging modalities. In some embodiments, the subject to be treated by the method described herein is a human cancer patient who has undergone or is subjecting to an anti-cancer therapy, for example, chemotherapy, radiotherapy, immunotherapy, or surgery.

In some embodiments, subjects have received prior immune-modulatory anti-tumor agents. Non-limiting examples of such immune-modulatory agents include, but are not limited to as anti-PD1, anti-PD-L1, anti-CTLA-4, anti-OX40, anti-CD137, etc. In some embodiments, the subject shows disease progression through the treatment. In other embodiments, the subject is resistant to the treatment (either de novo or acquired). In some embodiments, such a subject is demonstrated as having advanced malignancies (e.g., inoperable or metastatic). Alternatively or in addition, in some embodiments, the subject has no standard therapeutic options available or ineligible for standard treatment options, which refer to therapies commonly used in clinical settings for treating the corresponding solid tumor.

In some instances, the subject is a human patient having an elevated level of Galectin-9 as relative to a control level. The level of Galectin-9 can be a plasma or serum level of Galectin-9 in the human patient. In other examples, the level of Galectin-9 can be the level of cell-surface Galectin-9, for example the level of Galectin-9 on cancer cells. In one example, the level of Galectin-9 can be the level of surface Galectin-9 expressed on cancer cells in patient-derived organotypic tumor spheroids (PDOT), which can be prepared by, e.g., the method disclosed in Examples below. A control level may refer to the level of Galectin-9 in a matched sample of a subject of the same species (e.g., human) who are free of the solid tumor. In some examples, the control level represents the level of Galectin-9 in healthy subjects. See also above disclosures

In some embodiments, the anti-Galectin-9 antibody is administered once every 2 weeks for one cycle, once every 2 weeks for two cycles, once every 2 weeks for 3 cycles, once every 2 weeks for 4 cycles, or once every 2 weeks for more than 4 cycles. In some embodiments, the anti-Galectin-9 antibody is administered once every 2 weeks for 4 cycles. In some embodiments, the duration of treatment is 12-24 months or longer. In some embodiments, the cycles extend for a duration of 3 months to 6 months, or 6 months to 12 months or 12 months to 24 months or longer. In some embodiments, the cycle length is modified, e.g., temporarily or permanently to a longer duration, e.g., 3 weeks or 4 weeks. In some embodiments, the use further comprises administering to the subject an immune checkpoint inhibitor, e.g., an anti-PD-1 antibody, as described herein, e.g., administered according to a regimen described herein. In some embodiments, the solid tumor is ocular, e.g., uveal, melanoma. In some embodiments, the tumor is a metastatic tumor.

Given that pro-tumor action of Galectin-9 is mediated through interaction with immune cells (e.g., interactions with lymphoid cells via TIM-3, CD44, and 41BB, and with macrophages via dectin-1 and CD206) and given that Galectin-9 is expressed in a large number of tumors, targeting Galectin-9, e.g., using a Galectin-9 binding antibody to inhibit interaction with its receptors provides a therapeutic approach that can be applied across a variety of different tumor types.

In some embodiments, the disclosure provides a method for treating a solid tumor in a subject, the method comprising administering to a subject in need thereof an effective amount of an anti-Galectin-9 antibody described herein, including but not limited to, G9.2-17 IgG4. In some examples, the method disclosed herein is applied to a human patient having an ocular melanoma. In some instances, the ocular melanoma patient may have a metastatic cancer. In some examples, the method disclosed herein is applied to a human patient having uveal melanoma.

A subject having ocular cancer can be identified by routine ophthalmic examination. Uveal melanoma often causes painless distortion of vision and other non-specific visual symptoms. Diagnosis of UM relies primarily on clinical examination and ocular ultrasonography (Luke et al, Cancer. 2013; 119(20):3687-3695). In some embodiments, the subject to be treated by the method described herein is a human cancer patient who has undergone or is subjecting to an anti-cancer therapy, for example, chemotherapy, radiotherapy, immunotherapy, or surgery. In some embodiments, subjects have received prior immune-modulatory anti-tumor agents. Non-limiting examples of such immune-modulatory agents include, but are not limited to as anti-PD1, anti-PD-L1, anti-CTLA-4, anti-OX40, anti-CD137, etc. In some embodiments, the subject shows disease progression through the treatment. In other embodiments, the subject is resistant to the treatment (either de novo or acquired). In some embodiments, such a subject is demonstrated as having advanced malignancies (e.g., inoperable or metastatic). Alternatively or in addition, in some embodiments, the subject has no standard therapeutic options available or ineligible for standard treatment options, which refer to therapies commonly used in clinical settings for treating the corresponding solid tumor.

In some instances, the subject may be a human patient having a refractory disease, for example, a refractory ocular, e.g., uveal melanoma. As used herein, “refractory” refers to the tumor that does not respond to or becomes resistant to a treatment. In some instances, the subject may be a human patient having a relapsed disease, for example, a relapsed ocular, e.g., uveal, melanoma. As used herein, “relapsed” or “relapses” refers to the tumor that returns or progresses following a period of improvement (e.g., a partial or complete response) with treatment.

In some embodiments, the human patient to be treated by the methods disclosed herein meets one or more of the inclusion and exclusion criteria disclosed in Examples 10 and 17 below. For example, the human patient may be 18 or older; having histologically confirmed unresectable metastatic or inoperable cancer (e.g., without standard therapeutic options), having a life expectancy>3 months, having recent archival tumor sample available for biomarker analysis (e.g., an archival species for Galectin-9 tumor tissue expression levels assessed by IHC); having a measurable disease, according to RECIST v1.1, having Eastern Cooperative Oncology Group (ECOG) performance status 0-1 or Karnofsky score>70; having no available standard of care options, having MSI-H (Microsatellite instability high and MSS (Microsatellite Stable); received at least one line of systemic therapy in the advanced/metastatic setting; having adequate hematologic and end organ function (defined in Example 1 below); having completed treatment for brain metastases if any (see Example 1 below); having no evidence of active infection and no serious infection within the past month; having at least four (4) weeks s or 5 half lives (whichever is shorter) since the last dose of anti-cancer therapy before the first anti-Gal-9 antibody administration; having continued bisphosphonate treatment (zolendronic acid) or denosumab for bone metastases if applicable.

Alternatively or in addition, the subject suitable for the treatment disclosed herein may not have one or more of the following: diagnosed with metastatic cancer of an unknown primary; any active uncontrolled bleeding, and any patients with a bleeding diathesis (e.g., active peptic ulcer disease); receiving any other investigational agents within 4 weeks or 5 half-lives of anti-galectin-9 antibody administration; receiving radiation therapy within 4 weeks of the first dose of the anti-Galectin-9 antibody, except for palliative radiotherapy to a limited field, such as for the treatment of bone pain or a focally painful tumor mass; having fungating tumor masses, having active clinically serious infection>grade 2 NCI-CTCAE version 5.0; having symptomatic or active brain metastases; having >CTCAE grade 3 toxicity (see details and exceptions in Example 1); having history of second malignancy (see exceptions in Example 1); having evidence of severe or uncontrolled systemic diseases, congestive cardiac failure; having serious non-healing wound, active ulcer or untreated bone fracture; having uncontrolled pleural effusion, pericardial effusion, or ascites requiring recurrent drainage procedures; having spinal cord compression not definitively treated with surgery and/or radiation. Leptomeningeal disease, active or previously treated; having significant vascular disease; having active auto-immune disorder (see exceptions in Example 1); require systemic immunosuppressive treatment; having tumor-related pain (>grade 3) unresponsive to broad analgesic interventions (oral and/or patches); having uncontrolled hypercalcemia, despite use of bisphosphonates; having any history of an immune-related Grade 4 adverse event attributed to prior checkpoint inhibitor therapy (CIT); received an organ transplant(s); and/or on undergoing dialysis; having Child-Pugh score ≥7; having metastatic hepatocellular carcinoma that progressed while receiving at least one previous line of systemic therapy; having refuse or not toleratedsorafenib;, or having had standard therapy considered ineffective, intolerable, or inappropriate or for which no effective standard therapy is available.

Alternatively or in addition, patient for treatment by any of the methods disclosed herein may meet one or more patient inclusion and exclusion criteria disclosed in Example 10 and Example 17 below.

In some instances, the subject is a human patient having an elevated level of Galectin-9 as relative to a control level. The level of Galectin-9 can be a plasma or serum level of Galectin-9 in the human patient. In other examples, the level of Galectin-9 can be the level of cell-surface Galectin-9, for example the level of Galectin-9 on cancer cells. In one example, the level of Galectin-9 can be the level of surface Galectin-9 expressed on cancer cells in patient-derived organotypic tumor spheroids (PDOT), which can be prepared by, e.g., the method disclosed in Examples below. A control level may refer to the level of Galectin-9 in a matched sample of a subject of the same species (e.g., human) who is free of the solid tumor. In some examples, the control level represents the level of Galectin-9 in healthy subjects.

To identify such a subject, a suitable biological sample can be obtained from a subject who is suspected of having the solid tumor and the biological sample can be analyzed to determine the level of Galectin-9 contained therein (e.g., free, cell-surface expressed, or total) using conventional methods, e.g., ELISA or FACS. In some embodiments, organoid cultures are prepared, e.g., as described herein, and used to assess Galectin-9 levels in a subject. Single cells derived from certain fractions obtained as part of the organoid preparation process are also suitable for assessment of Galectin-9 levels in a subject. In some instances, an assay for measuring the level of Galectin-9, either in free form or expressed on cell surface, involves the use of an antibody that specifically binds the Galectin-9 (e.g., specifically binds human Galectin-9). Any of the anti-Galectin-9 antibodies known in the art can be tested for suitability in any of the assays described above and then used in such assays in a routine manner. In some embodiments, an antibody described herein (e.g., a G9.2-17 antibody) can be used in such as assay. In some embodiments, an antibody described in U.S. Pat. No. 10,344,091 and WO2019/084553, the relevant disclosures of each of which are incorporated by reference for the purpose and subject matter referenced herein. In some examples, the anti-Galectin-9 antibody is a Fab molecule. Assay methods for determining Galectin-9 levels as disclosed herein are also within the scope of the present disclosure.

An effective amount of the pharmaceutical composition described herein can be administered to a subject (e.g., a human) in need of the treatment via a suitable route, systemically or locally. In some embodiments, the anti-Galectin-9 antibodies are administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In some embodiments, the anti-Galectin-9 antibodies are administered by intraocular administration. In some embodiments, the anti-Galectin-9 antibodies are administered topically to the eye. In some embodiments, the anti-Galectin-9 antibodies are administered by Intravitreal injection. In one embodiment, the anti-Galectin-9 antibody is administered to the subject by intravenous infusion. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

As used herein, “an effective amount” refers to the amount of each active agent required to confer therapeutic effect on the subject, either alone or in combination with one or more other active agents. In some embodiments, the therapeutic effect is reduced Galectin-9 activity and/or amount/expression, reduced Dectin-1 signaling, reduced TIM-3 signaling, reduced CD206 signaling, or increased anti-tumor immune responses in the tumor microenvironment. Non-limiting examples of increased anti-tumor responses include increased activation levels of effector T cells, or switching of the TAMs from the M2 to the M1 phenotype. In some cases, the anti-tumor response includes increased ADCC responses. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally contribute to the determination of the dosage. For example, antibodies that are compatible with the human immune system, such as humanized antibodies or fully human antibodies, are in some instances used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein are determined empirically in individuals who have been given one or more administration(s) of the antibody. Individuals are given incremental dosages of the antagonist. To assess efficacy of the antagonist, an indicator of the disease/disorder can be followed.

In some instances, the anti-Galectin-9 antibody as disclosed herein (e.g., G9.2-17) can be administered to a subject at a dose of about 1 mg/kg to about 3 mg/kg, about 3 mg/kg to about 4 mg/kg, about 4 mg/kg to about 8 mg/kg, about 8 mg/kg to about 12 mg/kg, about 12 mg/kg to about 16 mg/kg, about 16 mg/kg to about 20 mg/kg, about 20 mg/kg to about 24 mg/kg, about 24 mg/kg to about 28 mg/kg, or about 28 mg/kg to about 32 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, or about 32 mg/kg) once every two to four weeks (e.g., every two, three, or four weeks).

In some instances, the anti-Galectin-9 antibody as disclosed herein (e.g., G9.2-17) can be administered to a subject at a suitable dose, for example, about 1 to about 32 mg/kg. Examples include 1 mg/kg to 3 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any incremental doses within these ranges. In some embodiments, the Galectin-9 antibody is administered at 2 mg/kg. In some embodiments, the Galectin-9 antibody is administered at 4 mg/kg. In some embodiments, the Galectin-9 antibody is administered at 8 mg/kg. In some embodiments, the Galectin-9 antibody is administered at 12 mg/kg. In some embodiments, the Galectin-9 antibody is administered at 16 mg/kg. In some instances, multiple doses of the anti-Galectin-9 antibody can be administered to a subject at a suitable interval or cycle, for example, once every two to four weeks (e.g., every two, three, or four weeks). The treatment may last for a suitable period, for example, up to 3 months, up to 6 months, or up to 12 months or up to 24 months.

In specific embodiments, the interval or cycle is 2 weeks. In some embodiments, the regimen is once every 2 weeks for one cycle, once every 2 weeks for two cycles, once every 2 weeks for three cycles, once every 2 weeks for four cycles, or once every 2 weeks for more than four cycles. In some embodiments, the treatment is once every 2 weeks for 1 to 3 months, once every 2 weeks for 3 to 6 months, once every 2 weeks for 6 to 12 months, or once every 2 weeks for 12 to 24 months, or longer.

In specific embodiments, the interval or cycle is 3 weeks. In some embodiments, the regimen is once every 3 weeks for one cycle, once every 3 weeks for two cycles, once every 3 weeks for three cycles, once every 3 weeks for four cycles, or once every 3 weeks for more than four cycles. In some embodiments, the treatment is once every 3 weeks for 1 to 3 months, once every 3 weeks for 3 to 6 months, once every 3 weeks for 6 to 12 months, or once every 3 weeks for 12 to 24 months, or longer.

In specific embodiments, the interval or cycle is 4 or more weeks. In some embodiments, the regimen is once every 4 or more weeks for one cycle, once every 4 or more weeks for two cycles, once every 4 or more weeks for three cycles, once every 4 or more weeks for four cycles, or once every 4 or more weeks for more than four cycles. In some embodiments, the treatment is once every 4 or more weeks for 1 to 3 months, once every 4 or more weeks for 3 to 6 months, once every 4 or more weeks for 6 to 12 months, or once every 4 or more weeks for 12 to 24 months, or longer. In some embodiments, the treatment is a combination of treatment at various time, e.g., a combination or 2 weeks, 3 weeks, 4 or more 4 weeks. In some embodiments, the treatment interval is adjusted in accordance with the patient's response to treatment. In some embodiments, the dosage(s) is adjusted in accordance with the patient's response to treatment. In some embodiments, the dosages are altered between treatment intervals. In some embodiments, the treatment may be temporarily stopped.

In some examples, the anti-Galectin-9 antibody is administered to a human patient having a target solid tumor as disclosed herein (e.g., ocular melanoma, e.g., uveal melanoma) at a dose of about 3 mg/kg once every two weeks via intravenous infusion. In other examples, the anti-Galectin-9 antibody is administered to the human patient having the target solid tumor at a dose of about 15 mg/kg once every two weeks via intravenous infusion. In one embodiment, the anti-Galectin-9 antibody as disclosed herein (e.g., G9.2-17) is administered every 2 weeks. In one embodiment, the anti-Galectin-9 antibody as disclosed herein (e.g., G9.2-17) is administered every 2 weeks intravenously, e.g., for 3 months.

The term “about” or “approximately” means within an acceptable error range for the particular value as determined by one of ordinary skill in the art, which are depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In some instances, the anti-Galectin-9 antibody as disclosed herein (e.g., G9.2-17) can be administered to a subject at a dose of 1 mg/kg to 3 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) once every two to four weeks (e.g., every two, three, or four weeks).

In some instances, the anti-Galectin-9 antibody as disclosed herein (e.g., G9.2-17) can be administered to a subject at a suitable dose, for example, about 1 to about 32 mg/kg. Examples include 1 mg/kg to 3 mg/kg, 3 mg/kg to 4 mg/kg, 4 mg/kg to 8 mg/kg, 8 mg/kg to 12 mg/kg, 12 mg/kg to 16 mg/kg, 16 mg/kg to 20 mg/kg, 20 mg/kg to 24 mg/kg, 24 mg/kg to 28 mg/kg, or 28 mg/kg to 32 mg/kg (e.g., 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, or 32 mg/kg) or any incremental doses within these ranges. In some embodiments, the Galectin-9 antibody is administered at 2 mg/kg. In some embodiments, the Galectin-9 antibody is administered at 4 mg/kg. In some embodiments, the Galectin-9 antibody is administered at 8 mg/kg. In some embodiments, the Galectin-9 antibody is administered at 12 mg/kg. In some embodiments, the Galectin-9 antibody is administered at 16 mg/kg. In some instances, multiple doses of the anti-Galectin-9 antibody can be administered to a subject at a suitable interval or cycle, for example, once every two to four weeks (e.g., every two, three, or four weeks). The treatment may last for a suitable period, for example, up to 3 months, up to 6 months, or up to 12 months or up to 24 months.

In specific embodiments, the interval or cycle is 2 weeks. In some embodiments, the regimen is once every 2 weeks for one cycle, once every 2 weeks for two cycles, once every 2 weeks for three cycles, once every 2 weeks for four cycles, or once every 2 weeks for more than four cycles. In some embodiments, the treatment is once every 2 weeks for 1 to 3 months, once every 2 weeks for 3 to 6 months, once every 2 weeks for 6 to 12 months, or once every 2 weeks for 12 to 24 months, or longer.

In specific embodiments, the interval or cycle is 3 weeks. In some embodiments, the regimen is once every 3 weeks for one cycle, once every 3 weeks for two cycles, once every 3 weeks for three cycles, once every 3 weeks for four cycles, or once every 3 weeks for more than four cycles. In some embodiments, the treatment is once every 3 weeks for 1 to 3 months, once every 3 weeks for 3 to 6 months, once every 3 weeks for 6 to 12 months, or once every 3 weeks for 12 to 24 months, or longer.

In specific embodiments, the interval or cycle is 4 or more weeks. In some embodiments, the regimen is once every 4 or more weeks for one cycle, once every 4 or more weeks for two cycles, once every 4 or more weeks for three cycles, once every 4 or more weeks for four cycles, or once every 4 or more weeks for more than four cycles. In some embodiments, the treatment is once every 4 or more weeks for 1 to 3 months, once every 4 or more weeks for 3 to 6 months, once every 4 or more weeks for 6 to 12 months, or once every 4 or more weeks for 12 to 24 months, or longer. In some embodiments, the treatment is a combination of treatment at various time, e.g., a combination or 2 weeks, 3 weeks, 4 or more 4 weeks. In some embodiments, the treatment interval is adjusted in accordance with the patient's response to treatment. In some embodiments, the dosage(s) is adjusted in accordance with the patient's response to treatment. In some embodiments, the dosages are altered between treatment intervals. In some embodiments, the treatment may be temporarily stopped.

In some examples, the anti-Galectin-9 antibody is administered to a human patient having a target solid tumor as disclosed herein (e.g., ocular melanoma, e.g., uveal melanoma) at a dose of about 3 mg/kg once every two weeks via intravenous infusion. In other examples, the anti-Galectin-9 antibody is administered to the human patient having the target solid tumor at a dose of about 15 mg/kg once every two weeks via intravenous infusion.

In some embodiments, the methods of the present disclosure increase anti-tumor activity (e.g., reduce cell proliferation, tumor growth, tumor volume, and/or tumor burden or load or reduce the number of metastatic lesions over time) by at least about 10%, 20%, 25%, 30%, 40%, 50%, 60%, 70%, 75%, 80%, 85%, 90%, 95%, or more as compared to levels prior to treatment or in a control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more. In some embodiments, before, during, and after the administration of the pharmaceutical composition, cancerous cells and/or biomarkers in a subject are measured in a biological sample, such as blood, serum, plasma, urine, peritoneal fluid, and/or a biopsy from a tissue or organ. In some embodiments, the methods include administration of the compositions of the invention to reduce tumor volume, size, load or burden in a subject to an undetectable size, or to less than about 1%, 2%, 5%, 10%, 20%, 25%, 30%, 40%, 50%, 60%, 70%, 75%, 80%, or 90% of the subject's tumor volume, size, load or burden prior to treatment. In other embodiments, the methods include administration of the compositions of the invention to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment. In other embodiments, the methods include administration of the compositions of the invention to reduce the development of or the number or size of metastatic lesions in a subject to an undetectable rate, or to less than about 1%, 2%, 5%, 10%, 20%, 25%, 30%, 40%, 50%, 60%, 70%, 75%, 80%, or 90% of the rate prior to treatment.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence. Conventional methods, known to those of ordinary skill in the art of medicine, can be used to administer the pharmaceutical composition to the subject, depending upon the type of disease to be treated or the site of the disease. In some embodiments, the anti-Galectin-9 antibody can be administered to a subject by intravenous infusion.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, the anti-Galectin-9 antibodies described herein are be used as a monotherapy for treating the target cancer disclosed herein, i.e., free of other anti-cancer therapy concurrently with the therapy using the anti-Galectin-9 antibody.

In other embodiments, the treatment method further comprises administering to the subject an inhibitor of a checkpoint molecule, for example, PD-1. Examples of PD-1 inhibitors include anti-PD-1 antibodies, such as pembrolizumab, nivolumab, and cemiplimab. Such checkpoint inhibitors can be administered simultaneously or sequentially (in any order) with the anti-Galectin-9 antibody according to the present disclosure. In some embodiments, the checkpoint molecule is PD-L1. Examples of PD-L1 inhibitors include anti-PD-L1 antibodies, such as durvalumab, avelumab, and atezolizumab. In some embodiments, the checkpoint molecule is CTLA-4. An example of a CTLA-4 inhibitor is the anti-CTLA-4 antibody ipilimumab. In some embodiments, the inhibitor targets a checkpoint molecule selected from CD40, GITR, LAG-3, OX40, TIGIT and TIM-3.

In some embodiments, the methods are provided, the anti-Galectin-9 antibody is administered concurrently with a checkpoint inhibitor. In some embodiments, the anti-Galectin-9 antibody is administered before or after a checkpoint inhibitor. In some embodiments, the checkpoint inhibitor is administered systemically. In some embodiments, the checkpoint inhibitor is administered locally. In some embodiments, the checkpoint inhibitor is administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In one embodiment, the checkpoint inhibitor is administered to the subject by intravenous infusion.

A response to treatment, e.g., a treatment of a solid tumor as described herein, can be assessed according to RECIST or the updated RECIST 1.1 criteria, as described in Example 1 below and Eisenhower et al., New response evaluation criteria in solid tumors: Revised RECIST guideline (version 1.1); European Journal Of Cancer 45 (2009) 228-247, the contents of which is herein incorporated by reference in its entirety.

In some embodiments, treating can improve the overall response (e.g., at 3, 6 or 12 months, or a later time), e.g., as compared to a baseline level prior to initiation of treatment or as compared to a control group not receiving the treatment. In some embodiments, treating can result in a complete response, a partial response or stable disease (e.g., as measured at 3 months, 6 months or 12 months, or at a later time). Such a response can be temporary over a certain time period or permanent. In some embodiments, treating can improve the likelihood of a complete response, a partial response or stable disease (e.g., as measured at 3 months, 6 months or 12 months, or at a later time), e.g., as compared to a control group not receiving the treatment. Such a response can be temporary over a certain time period or permanent. In some embodiments, treating can result in reduced or attenuated progressive disease (e.g., as measured at 3 months, 6 months or 12 months, or at a later time), e.g., as compared to a control group not receiving the treatment. Such an attenuation may be temporary or permanent.

A partial response is a decrease in the size of a tumor, or in the extent of cancer in the body, i.e., the tumor burden, in response to treatment as compared to a baseline level before the initiation of the treatment. For example, according to the RECIST response criteria, a partial response is defined as at least a 30% decrease in the sum of diameters of target lesions, taking as reference the baseline sum diameters. Progressive disease is a disease that is growing, spreading, or getting worse. For example, according to the RECIST response criteria, progressive disease includes disease in which at least a 20% increase in the sum of diameters of target lesions is observed, and the sum must also demonstrate an absolute increase of at least 5 mm. Additionally, the appearance of one or more new lesions is also considered progression. A tumor that is neither decreasing nor increasing in extent or severity as compared to a baseline level before initiation of the treatment is considered stable disease. For example, according to the RECIST response criteria, stable disease occurs when there is neither sufficient shrinkage to qualify for partial response nor sufficient increase to qualify for progressive disease, taking as reference the smallest sum diameters while on study.

Accordingly, in some embodiments, treating can result in overall tumor size reduction, maintenance of tumor size, either permanently or over a minimum time period, relative to a baseline tumor size prior to initiation of the treatment (e.g., as measured at 3 months, 6 months or 12 months, or at a later time). In some embodiments, treating can result in a greater likelihood of overall tumor size reduction or maintenance of tumor size, either permanent or over a minimum time period, e.g., as compared to a control group not receiving the treatment (e.g., as measured at 3 months, 6 months or 12 months, or at a later time). Tumor size, e.g., the diameters of tumors, can be measured according to methods known in the art, which include measurements from CT and MRI images in combination with various software tools, according to specific measurement protocols, e.g., as described in Eisenhower et al., referenced above. Accordingly, in some embodiments, tumor size is measured in regularly scheduled restaging scans (e.g., CT with contrast, MRI with contrast, PET-CT (diagnostic CT) and/or X-ray). In some embodiments, tumor size reduction, maintenance of tumor size refers to the size of target lesions. In some embodiments, tumor size reduction, maintenance of tumor size refers to the size of non-target lesions. According to RECIST 1.1, when more than one measurable lesion is present at baseline, all lesions up to a maximum of five lesions total (and a maximum of two lesions per organ) representative of all involved organs should be identified as target lesions. All other lesions (or sites of disease) including pathological lymph nodes should be identified as non-target lesions.

In some embodiments, treating can result in reduction of tumor burden, or maintenance of tumor burden as compared to baseline levels prior to initiation of the treatment (e.g., as measured at 3 months, 6 months or 12 months, or at a later time). The reduction in tumor burden can be temporary over a certain time period or permanent. In some embodiments, treating can result in in a greater likelihood of a reduction of tumor burden, or maintenance of tumor burden, e.g., as compared to a control group not receiving the treatment (e.g., as measured at 3 months, 6 months or 12 months, or at a later time). As used herein, tumor burden refers to amount of cancer, the size or the volume of the tumor in the body of a subject, accounting for all sites of disease. Tumor burden can be measured using methods known in the art, including but not limited to, FDG positron emission tomography (FDG-PET), magnetic resonance imaging (MRI), and optical imaging, comprising bioluminescence imaging (BLI) and fluorescence imaging (FLI).

In some embodiments, treating can result in an increase in the time to disease progression or in progression free survival (e.g., as measured at 3 months, 6 months or 12 months, or at a later time post initiation of treatment) as compared to a control group that does not receive the treatment. Progression free survival can be either permanent or progression free survival over a certain amount of time. In some embodiments, treating can result in a greater likelihood of progression free survival (either permanent progression free survival or progression free survival over a certain amount of time, e.g., 3, 6 or 12 months or e.g., as measured at 3 months, 6 months or 12 months, or at a later time post initiation of treatment) as compared to a control group that does not receive the treatment. Progression-free survival (PFS) is defined as the time from random assignment in a clinical trial, e.g., from initiation of a treatment to disease progression or death from any cause. In some embodiments, treating can result in longer survival or greater likelihood of survival, e.g., at a certain time, e.g., at 6 or 12 months.

A response to treatment, e.g., a treatment of a solid tumor as described herein, can be assessed according to iRECIST criteria, as described in Seymour et al, iRECIST: guidelines for response criteria for use in trials; The Lancet, Vol18, March 2017, the contents of which is herein incorporated by reference in its entirety. iRECIST was developed for the use of modified RECIST1.1 criteria specifically in cancer immunotherapy trials, to ensure consistent design and data collection and can be used as guidelines to a standard approach to solid tumor measurements and definitions for objective change in tumor size for use in trials in which an immunotherapy is used. iRECIST is based on RECIST 1.1. Responses assigned using iRECIST have a prefix of “i” (ie, immune)—e.g., “immune” complete response (iCR) or partial response (iPR), and unconfirmed progressive disease (iUPD) or confirmed progressive disease (iCPD) or stable disease (iSD) to differentiate them from responses assigned using RECIST 1.1, and all of which are defined in Seymour et al.

Accordingly , in some embodiments, treating can result in a “immune” complete response (iCR), a partial response (iPR) or stable disease (iSD) (e.g., as measured at 3 months, 6 months or 12 months, or at a later time), as compared to the baseline level of disease prior to initiation of the treatment. The reduction in the “immune” response, e.g., iCR, iPR, or iSD can be temporary over a certain time period or permanent. In some embodiments, treating can improve the likelihood of a complete response (iCR), a partial response (iPR) or stable disease (iSD) (e.g., as measured at 3 months, 6 months or 12 months, or at a later time), e.g., as compared to a control group not receiving the treatment. In some embodiments, treating can result in overall reduction in unconfirmed progressive disease (iUPD) or confirmed progressive disease (iCPD) (e.g., as measured at 3 months, 6 months or 12 months, or at a later time), e.g., as compared to a baseline prior to initiation of treatment. The reduction in iUPD or iCPD can be temporary over a certain time period or permanent. In some embodiments, treating can result in greater likelihood of overall reduction in unconfirmed progressive disease (iUPD) or confirmed progressive disease (iCPD) (e.g., as measured at 3 months, 6 months or 12 months, or at a later time), e.g., as compared to a control group not receiving the treatment. In some embodiments, treating can result in overall reduced number of new lesions according to iRECIST criteria, as compared to a control group not receiving the treatment or as compared to a baseline prior to initiation of the treatment (e.g., as measured at 3 months, 6 months or 12 months, or at a later time). The reduction in lesions can be temporary over a certain time period or permanent.

Response to treatment can also be characterized by one or more of immunophenotype in blood and tumors, cytokine profile (serum), soluble galectin-9 levels in blood (serum or plasma), galectin-9 tumor tissue expression levels and pattern of expression by immunohistochemistry (tumor, stroma, immune cells), tumor mutational burden (TMB), PDL-1 expression (e.g., by immunohistochemistry), mismatch repair status, or tumor markers relevant for the disease (e.g., as measured at 3 months, 6 months or 12 months, or at a later time). Non-limiting examples of such tumor markers include Ca15-3, CA-125, CEA, CA19-9, S100, alpha fetoprotein. These parameters can either be compared to baseline levels prior to initiation of treatment or can be compared to a control group not receiving the treatment.

In some embodiments, treating can result in changes in levels of immune cells and immune cell markers in the blood or in tumors, e.g., can result in immune activation. Such changes can be measured in patient blood and tissue samples using methods known in the art, such as multiplex flow cytometry and multiplex immunohistochemistry. For example, a panel of phenotypic and functional PBMC immune markers can be assessed at baseline prior to commencement of the treatment and at various time point during treatment. Table 2 lists non-limiting examples of markers useful for these assessment methods. Flow cytometry (FC) is a fast and highly informative method of choice technology to analyze cellular phenotype and function, and has gained prominence in immune phenotype monitoring. It allows for the characterization of many subsets of cells, including rare subsets, in a complex mixture such as blood, and represents a rapid method to obtain large amounts of data. Advantages of FC are high speed, sensitivity, and specificity. Standardized antibody panels and procedures can be used to analyze and classify immune cell subtypes. Multiplex IHC is a powerful investigative tool which provides objective quantitative data describing the tumor immune context in both immune subset number and location and allows for multiple markers to be assessed on a single tissue section. Computer algorithms can be used to quantify IHC-based biomarker content from whole slide images of patient biopsies, combining chromogenic IHC methods and stains with digital pathology approaches.

TABLE 2
PBMC phenotyping markers

| PBMC phenotyping markers | | PBMC phenotyping markers | |
|---|---|---|---|
| CD3 | Total T cells | CD16 | NK cells |
| CD4 | CD4+ T cells | CD11b | Monocytes/macrophages |
| CD8 | CD8+ T cells | CD11c | Monocytes/macrophages, |
| | | | DCs |
| CD25 | Treg activation | CD14 | Monocyte subsets, |
| | | | macrophages |
| CD27 | T cell maturation; B cell | CD33 | Total myeloid cells |
| | naïve/memory | | |
| CD38 | T cell maturation; B cell | Fc3R1 a | Antigen presenting DC cells |
| | naïve/memory | | |
| CD45RA | Naïve/memory cells | CD19 | Total B cells |
| CD45RO | Naïve/memory cells | T-bet | T cells subsets |
| CD56 | NKT/NK cells (T cell subset) | gdTCR | Gamma delta T cells |
| CD127 | T cell subsets | CD274 (PDL-1) | Checkpoint |
| CD152 (CLTA-4) | Checkpoint | Tim-3 | Checkpoint |
| CD279 (PD-1) | Checkpoint | TCRVa24-Ja18 | iNKT cells |
| FoxP3 | Treg cells | Live/dead | General |
| HLA-DR | Activation/Antigen presentation | CD45 | General |

Accordingly, in some embodiments, treating results in modulation of immune activation markers such as those in Table 2, e.g., treating results in one or more of (1) an increase in more CD8 cells in plasma or tumor tissue, (2) a reduction in T regulatory cells (Tregs) in plasma or tumor tissue, (3) an increase in M1 macrophages in plasma or tumor tissue and (4) a decrease in MDSCs in plasma or tumor tissue, and (5) a decrease in M2 macrophages in plasma or tumor tissue (e.g., as measured at 3 months, 6 months or 12 months, or at a later time). In some embodiments, the markers that are assessed using the techniques described above or known in the art are selected from CD4, CD8 CD14, CD11b/c, and CD25. These parameters can either be compared to baseline levels prior to initiation of treatment or can be compared to a control group not receiving the treatment.

In some embodiments, treating as described herein results in changes in proinflammatory and anti-inflammatory cytokines. In some embodiments, treating as described herein results in one or more of (1) increased levels of IFNgamma in plasma or tumor tissue; (2) increased levels of TNFalpha in plasma or tumor tissue; (3) decreased levels of IL-10 in plasma or tumor tissue (e.g., as measured at 3 months, 6 months or 12 months, or at a later time). These parameters can either be compared to baseline levels prior to initiation of treatment or can be compared to a control group not receiving the treatment.

In some embodiments, changes in cytokines or immune cells may be assessed between a pre dose 1 tumor biopsy and repeat biopsy conducted at a feasible time. In some embodiments, changes in cytokines or immune cells may be assessed between 2 repeat biopsies. In some embodiments, treating results in a change one or more of in soluble galectin-9 levels in blood (serum or plasma), or in galectin-9 tumor tissue expression levels and pattern of expression by immunohistochemistry (tumor, stroma, immune cells), (e.g., as measured at 3 months, 6 months or 12 months, or at a later time). In some embodiments, treating results in a decrease of one or more of soluble galectin-9 levels in blood (serum or plasma), or in galectin-9 tumor tissue expression levels and pattern of expression by immunohistochemistry (tumor, stroma, immune cells) decrease. (e.g., as measured at 3 months, 6 months or 12 months, or at a later time). These galectin-9 levels can either be compared to baseline levels prior to initiation of treatment or can be compared to a control group not receiving the treatment.

In some embodiments, treating results in a change in PDL-1 expression, e.g., as assessed by immunohistochemistry. In some embodiments, treatments results in a change in one or more tumor markers (increase or decrease) relevant for the disease (e.g., as measured at 3 months, 6 months or 12 months, or at a later time). Non-limiting examples of such tumor markers include Ca15-3, CA-125, CEA, CA19-9, S100, alpha fetoprotein. These parameters can either be compared to baseline levels prior to initiation of treatment or can be compared to a control group not receiving the treatment.

In some embodiments, treating results in improved quality of life and symptom control as compared to baseline prior to initiation of treatment or as compared to a control group not receiving the treatment (e.g., as measured at 3 months, 6 months or 12 months, or at a later time). In some embodiments, improvements can be measured on the ECOG scale described in Example 1 herein.

In any of the above embodiments, treating may comprise administering an anti-Galectin-9 antibody described herein alone or in combination with a checkpoint inhibitor therapy, e.g., an anti-PD-1 antibody. In some embodiments, the disclosure provides methods for treating a ocular melanoma in a subject, including a human subject, comprising administering to the subject a therapeutically effective amount of an anti-Galectin-9 antibody as disclosed herein. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD-1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma. In some embodiments, the disclosure provides methods for improving an overall response, e.g., according to RECIST 1.1. criteria (e.g., as measured at 3 months, 6 months or 12 months, or at a later time), in a subject, including a human subject, comprising administering to the subject a therapeutically effective amount of an anti-Galectin-9 antibody as disclosed herein. RECIST 1.1. criteria can either be compared to baseline levels prior to initiation of treatment or can be compared to a control group not receiving the treatment. In some embodiments, the disclosure provides methods for achieving a complete response, a partial response or stable disease (e.g., as measured at 3 months, 6 months or 12 months, or at a later time), the methods comprising administering to the subject a therapeutically effective amount of an anti-Galectin-9 antibody as disclosed herein. These responses can be temporary over a certain time period or permanent and can either be compared to baseline levels prior to initiation of treatment or can be compared to a control group not receiving the treatment.

In some embodiments, the methods can improve the likelihood of a complete response, a partial response or stable disease (e.g., as measured at 3 months, 6 months or 12 months, or at a later time; and being either temporary or permanent), e.g., as compared to a control group not receiving the treatment. In some embodiments, the disclosure provides methods for attenuating disease progression or reducing progressive disease (e.g., as measured at 3 months, 6 months or 12 months, or at a later time), e.g., as compared to a control group not receiving the treatment or as compared to baseline prior to initiation of the treatment, the method comprising administering to the subject a therapeutically effective amount of an anti-Galectin-9 antibody as disclosed herein. The attenuation or reduction can be temporary over a certain time period or permanent. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma.

In some embodiments, the disclosure provides methods for reducing or maintaining tumor size in a subject, including a human subject, (e.g., as measured at 3 months, 6 months or 12 months, or at a later time) either permanently or over a minimum time period, relative to a baseline tumor size prior to initiation of the treatment in the subject, the method comprising administering to the subject a therapeutically effective amount of an anti-Galectin-9 antibody as disclosed herein. In some embodiments, the disclosure provides methods for improving the likelihood of reducing or maintaining tumor size in a subject, including a human subject, either permanently or over a minimum time period, (e.g., as measured at 3 months, 6 months or 12 months, or at a later time) e.g., as compared to a control group not receiving the treatment. In some embodiments, the disclosure provides methods for reducing or maintaining a tumor burden, in a subject, including a human subject (e.g., as measured at 3 months, 6 months or 12 months, or at a later time), as compared to baseline levels prior to initiation of the treatment or as compared to a control group not receiving the treatment, the methods comprising administering to the subject a therapeutically effective amount of an anti-Galectin-9 antibody as disclosed herein. In some embodiments, the disclosure provides methods for increasing the likelihood of reducing or maintaining a tumor burden (e.g., as measured at 3 months, 6 months or 12 months, or at a later time), e.g., as compared to a control group not receiving the treatment, the methods comprising administering to the subject a therapeutically effective amount of an anti-Galectin-9 antibody as disclosed herein. Accordingly, in some embodiments, tumor size and/or burden is measured in regularly scheduled restaging scans (e.g., CT with contrast, MRI with contrast, PET-CT (diagnostic CT) and/or X-ray). In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma. In some embodiments, the disclosure provides methods for increasing the time to disease progression or increasing the time of progression free survival (e.g., as measured at 3 months, 6 months or 12 months, or at a later time) in a subject, including a human subject, as compared to a control group that does not receive the treatment, the methods comprising administering to the subject a therapeutically effective amount of an anti-Galectin-9 antibody as disclosed herein. The methods can result in either permanent progression free survival or progression free survival over a certain amount of time. In some embodiments, the disclosure provides methods for increasing the likelihood of progression free survival (either permanent progression free survival or progression free survival over a certain amount of time (e.g., as measured at 3 months, 6 months or 12 months, or at a later time) as compared to a control group that does not receive the treatment. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma.

In some embodiments, the disclosure provides methods for improving an overall response (iOR), e.g., according to iRECIST criteria (e.g., as measured at 3 months, 6 months or 12 months, or at a later time), in a subject, including a human subject, comprising administering to the subject a therapeutically effective amount of an anti-Galectin-9 antibody as disclosed herein. In some embodiments, the disclosure provides methods for achieving a “immune” complete response (iCR), a partial response (iPR) or stable disease (iSD) (e.g., as measured at 3 months, 6 months or 12 months, or at a later time), the methods comprising administering to the subject a therapeutically effective amount of an anti-Galectin-9 antibody as disclosed herein. In some embodiments, the methods can improve the likelihood of a “immune” complete response (iCR), a partial response (iPR) or stable disease (iSD) (e.g., as measured at 3 months, 6 months or 12 months, or at a later time). In some embodiments, the disclosure provides methods for attenuating disease progression or reducing progressive disease, e.g., reducing unconfirmed progressive disease (iUPD) or reducing confirmed progressive disease (iCPD)) (e.g., as measured at 3 months, 6 months or 12 months, or at a later time), the method comprising administering to the subject a therapeutically effective amount of an anti-Galectin-9 antibody as disclosed herein. . Any of these above mentioned iRECIST criteria can either be compared to baseline levels prior to initiation of treatment or can be compared to a control group not receiving the treatment and response can be temporary over a certain time period or permanent. In some embodiments, the disclosure provides methods for increasing the likelihood of overall reduction in unconfirmed progressive disease (iUPD) or confirmed progressive disease (iCPD) (e.g., as measured at 3 months, 6 months or 12 months, or at a later time), in a subject, including a human subject, e.g., as compared to a control group not receiving the treatment, the methods comprising administering to the subject a therapeutically effective amount of an anti-Galectin-9 antibody as disclosed herein. In some embodiments, the disclosure provides methods for reducing the number of new lesions in a subject, including a human subject, according to iRECIST criteria (e.g., as measured at 3 months, 6 months or 12 months, or at a later time), the methods comprising administering to the subject a therapeutically effective amount of an anti-Galectin-9 antibody as disclosed herein. Reduced number of lesions can either be relative to baseline levels prior to initiation of treatment or relative to a control group not receiving the treatment, and the reduction can be temporary over a certain time period or permanent. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma.

In some embodiments, the disclosure provides methods of modulating an immune response in a subject. As used herein, the term “immune response” includes T cell-mediated and/or B cell-mediated immune responses that are influenced by modulation of immune cell activity, for example, T cell activation. In one embodiment of the disclosure, an immune response is T cell mediated. As used herein, the term “modulating” means changing or altering, and embraces both upmodulating and downmodulating. For example “modulating an immune response” means changing or altering the status of one or more immune response parameter(s). Exemplary parameters of a T cell mediated immune response include levels of T cells (e.g., an increase or decrease in effector T cells) and levels of T cell activation (e.g., an increase or decrease in the production of certain cytokines). Exemplary parameters of a B cell mediated immune response include an increase in levels of B cells, B cell activation and B cell mediated antibody production.

When an immune response is modulated, some immune response parameters may decrease and others may increase. For example, in some instances, modulating the immune response causes an increase (or upregulation) in one or more immune response parameters and a decrease (or downregulation) in one or more other immune response parameters, and the result is an overall increase in the immune response, e.g., an overall increase in an inflammatory immune response. In another example, modulating the immune response causes an increase (or upregulation) in one or more immune response parameters and a decrease (or downregulation) in one or more other immune response parameters, and the result is an overall decrease in the immune response, e.g., an overall decrease in an inflammatory response. In some embodiments an increase in an overall immune response, i.e., an increase in an overall inflammatory immune response, is determined by a reduction in tumor weight, tumor size or tumor burden or any RECIST or iRECIST criteria described herein. In some embodiments an increase in an overall immune response is determined by increased level(s) of one or more proinflammatory cytokine(s), e.g., including two or more, three or more, etc or a majority of proinflammatory cytokines (one or more, two or more, etc or a majority of anti-inflammatory and/or immune suppressive cytokines and/or one or more of the most potent anti-inflammatory or immune suppressive cytokines either decrease or remain constant). In some embodiments an increase in an overall immune response is determined by increased levels of one or more of the most potent proinflammatory cytokines (one or more anti-inflammatory and/or immune suppressive cytokines including one or more of the most potent cytokines either decrease or remain constant). In some embodiments an increase in an overall immune response is determined by decreased levels of one or more, including a majority of, immune suppressive and/or anti-inflammatory cytokines (the levels of one or more, or a majority of, proinflammatory cytokines, including e.g., the most potent proinflammatory cytokines, either increase or remain constant). In some embodiments, an increase in an overall immune response is determined by increased levels of one or more of the most potent anti-inflammatory and/or immune suppressive cytokines (one or more, or a majority of, proinflammatory cytokines, including, e.g., the most potent proinflammatory cytokines either increase or remain constant). In some embodiments an increase in an overall immune response is determined by a combination of any of the above. Also, an increase (or upregulation) of one type of immune response parameter can lead to a corresponding decrease (or downregulation) in another type of immune response parameter. For example, an increase in the production of certain proinflammatory cytokines can lead to the downregulation of certain anti-inflammatory and/or immune suppressive cytokines and vice versa.

In some embodiments, the disclosure provides methods for modulating an immune response (e.g., as measured at 3 months, 6 months or 12 months, or at a later time) in a subject, including a human subject, comprising administering to the subject a therapeutically effective amount of an anti-Galectin-9 antibody as disclosed herein. In some embodiments, the disclosure provides methods for modulating levels of immune cells and immune cell markers, including but not limited to those described herein in Table 2, e.g., as compared to baseline levels prior to initiation of treatment, or as compared to a control group not receiving a treatment, in the blood or in tumors of a subject, including a human subject, comprising administering to the subject a therapeutically effective amount of an anti-Galectin-9 antibody as disclosed herein. In some embodiments, the overall result of modulation is upregulation of proinflammatory immune cells and/or down regulation of immune-suppressive immune cells. In some embodiments, the disclosure provides methods for modulating levels of immune cells, wherein the modulating encompasses one or more of (1) increasing CD8 cells in plasma or tumor tissue, (2) reducing Tregs in plasma or tumor tissue, (3) increasing M1 macrophages in plasma or tumor tissue and (4) decreasing MDSC in plasma or tumor tissue, and (5) decreasing in M2 macrophages in plasma or tumor tissue, and wherein the methods comprise administering to the subject a therapeutically effective amount of an anti-Galectin-9 antibody as disclosed herein. In some embodiments, the markers to assess levels of such immune cells include but are not limited to CD4, CD8 CD14, CD11b/c, and CD25. In some embodiments, the disclosure provides methods for modulating levels of proinflammatory and immune suppressive cytokines (e.g., as measured at 3 months, 6 months or 12 months, or at a later time), e.g., as compared to baseline levels prior to initiation of treatment, or as compared to a control group not receiving a treatment, in the blood or in tumors of a subject, including a human subject, comprising administering to the subject a therapeutically effective amount of an anti-Galectin-9 antibody as disclosed herein. In some embodiments, the overall result of modulation is upregulation of proinflammatory cytokines and/or down regulation of immune-suppressive cytokines.

In some embodiments, the disclosure provides methods for modulating levels of cytokines cells, wherein the modulating encompasses one or more of (1) increasing levels of IFNgamma in plasma or tumor tissue; (2) increasing levels of TNFalpha in plasma or tumor tissue; (3) decreasing levels of IL-10 in plasma or tumor tissue. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma.

In some embodiments, the disclosure provides methods for changing one or more of soluble galectin-9 levels in blood (serum or plasma), or in galectin-9 tumor tissue expression levels and pattern of expression by immunohistochemistry (tumor, stroma, immune cells) (e.g., as measured at 2 weeks, 4 weeks, 1 month, 3 months, 6 months or 12 months, or at a later time), comprising administering to the subject a therapeutically effective amount of an anti-Galectin-9 antibody as disclosed herein. In some embodiments of the methods, one or more of soluble galectin-9 levels in blood (serum or plasma), or in galectin-9 tumor tissue expression levels and pattern of expression by immunohistochemistry (tumor, stroma, immune cells) remain unchanged. In some embodiments, the methods provided herein decrease one or more of soluble galectin-9 levels in blood (serum or plasma), or in galectin-9 tumor tissue expression levels and pattern of expression by immunohistochemistry (tumor, stroma, immune cells) (e.g., e.g., as measured at 2 weeks, 4 weeks, 1 month, 3 months, 6 months or 12 months, or at a later time). Galectin-9 levels can either be compared to baseline levels prior to initiation of treatment or can be compared to a control group not receiving the treatment.

In some embodiments, treating results in a change in PDL-1 expression, e.g., by immunohistochemistry. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma.

In some embodiments, the disclosure provides methods for changing PD-L1 expression, e.g., as assessed by immunohistochemistry (e.g., as measured at 2 weeks, 4 weeks, 1 month, 3 months, 6 months or 12 months, or at a later time), comprising administering to the subject a therapeutically effective amount of an anti-Galectin-9 antibody as disclosed herein. In some embodiments of the methods, PD-L1 expression, e.g., as assessed by immunohistochemistry, remains unchanged. PD-L1 levels can either be compared to baseline levels prior to initiation of treatment or can be compared to a control group not receiving the treatment. In some embodiments, the methods provided herein decrease PDL-1 expression, e.g., as assessed by immunohistochemistry. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4.

In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the dose is about 30 mg/kg. In some embodiments, the anti-galectin-9 antibody is administered to the human subject at a dose of 0.2 mg/kg to 32 mg/kg, optionally at a dose of 1 mg/kg to 32 mg/kg. in some embodiments, the anti-galectin-9 antibody is administered to the human subject at a dose of 0.2 mg/kg to16 mg/kg. In some embodiments, the anti-galectin-9 antibody is administered to the human subject at a dose of 0.2 mg/kg, 0.63 mg/kg, 2 mg/kg, 6.3 mg/kg, 10 mg/kg, 16 mg/kg, or 32 mg/kg. In some embodiments, the anti-galectin-9 antibody is administered to the subject at a dose of 0.5 mg/kg to 32 mg/kg, or 2 mg/kg to16 mg/kg. In some embodiments, the antibody is administered once every two weeks, e.g., via intravenous infusion or intra-ocular, e.g., intravitreal injection. In some embodiments, the antibody is administered once every week, e.g., via intravenous infusion or intra-ocular, e.g., intravitreal injection. In some embodiments, the anti-Galectin-9 antibody is administered to the subject at a dose of about 0.1 mg/kg to about 5 mg/kg, e.g., the dose may be selected from 0.1 mg/kg, 0.2 mg/kg, 0.5 mg/kg, 1 mg/kg, 2 mg/kg, and 5 mg/kg, e.g., for intravitreal injection. In some embodiments, the antibody is administered once every month, e.g., via intravenous infusion or intra-ocular, e.g., intravitreal injection. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma.

In some embodiments, the disclosure provides methods for changing one or more tumor markers (increasing or decreasing) relevant for the disease (e.g., as measured at 2 weeks, 4 weeks, 1 month, 3 months, 6 months or 12 months, or at a later time), comprising administering to the subject a therapeutically effective amount of an anti-Galectin-9 antibody as disclosed herein. In some embodiments of the methods, one or more tumor markers (increasing or decreasing) relevant for the disease, remain unchanged. Non-limiting examples of such tumor markers include Ca15-3, CA-125, CEA, CA19-9, S100, alpha fetoprotein. Levels of tumor markers can either be compared to baseline levels prior to initiation of treatment or can be compared to a control group not receiving the treatment. In some embodiments, the methods provided herein decrease the occurrence of one or more tumor markers relevant for the disease. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma.

In some embodiments, the disclosure provides methods for improving quality of life and/or improving symptom control (e.g., as measured at 1 month, 3 months, 6 months or 12 months, or at a later time) in a subject, including a human subject, comprising administering to the subject a therapeutically effective amount of an anti-Galectin-9 antibody as disclosed herein. in improved quality of life and symptom control as compared to baseline prior to initiation of treatment or as compared to a control group not receiving the treatment. The improvements in quality of life can be temporary over a certain time period or permanent. In some embodiments, improvements can be measured on the ECOG scale. In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19.

In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma.

In some embodiments, the antibodies described herein, e.g., G9.2-17, are administered to a subject in need of the treatment at an amount sufficient to inhibit the activity of Galectin-9 (and/or Dectin-1 or TIM-3 or CD206) in immune suppressive immune cells in a tumor by at least 20% (e.g., 30%, 40%, 50%, 60%, 70%, 80%, 90% or greater) in vivo. In other embodiments, the antibodies described herein, e.g, G9.2-17, are administered in an amount effective in reducing the activity level of Galectin-9 (and/or Dectin-1 or TIM-3 or CD206) in immune suppressive immune cells in a tumor by at least 20% (e.g., 30%, 40%, 50%, 60%, 70%, 80%, 90% or greater) (as compared to levels prior to treatment or in a control subject). In some embodiments, the antibodies described herein, e.g., G9.2-17, are administered to a subject in need of the treatment at an amount sufficient to promote M1-like programming in TAMs by at least 20% (e.g., 30%, 40%, 50%, 60%, 70%, 80%, 90% or greater) in vivo (as compared to levels prior to treatment or in a control subject).

Conventional methods, known to those of ordinary skill in the art of medicine, can be used to administer the pharmaceutical composition to the subject, depending upon the type of disease to be treated or the site of the disease. In some embodiments, the anti-Galectin-9 antibody can be administered to a subject by intravenous infusion.

In some embodiments, the anti-Galectin-9 antibodies described herein are be used as a monotherapy for treating the target cancer disclosed herein, i.e., free of other anti-cancer therapy concurrently with the therapy using the anti-Galectin-9 antibody.

### D. Combined Therapy

In other embodiments, the treatment method further comprises administering to the subject an inhibitor of a checkpoint molecule, for example, PD-1. Examples of PD-1 inhibitors include anti-PD-1 antibodies, such as pembrolizumab, nivolumab, tislelizumab and cemiplimab. Such checkpoint inhibitors can be administered simultaneously or sequentially (in any order) with the anti-Galectin-9 antibody according to the present disclosure. In some embodiments, the checkpoint molecule is PD-L1. Examples of PD-L1 inhibitors include anti-PD-L1 antibodies, such as durvalumab, avelumab, and atezolizumab. In some embodiments, the checkpoint molecule is CTLA-4. An example of a CTLA-4 inhibitor is the anti-CTLA-4 antibody ipilimumab. In some embodiments, the inhibitor targets a checkpoint molecule selected from CD40, GITR, LAG-3, OX40, TIGIT and TIM-3.

In some embodiments, the anti-Galectin-9 antibody improves the overall response, e.g., at 3 months, relative to a regimen comprising the inhibitor of the checkpoint molecule (e.g., anti-PD1, for example, nivilumab) alone.

In some embodiments, the anti-PD-1 antibody is PD-1 is nivolumab, and the method described herein comprises administration of nivolumab to the subject at a dose of 240 mg intravenously once every two weeks.

In some embodiments, the antibody that binds PD-1 is administered using a flat dose. In some embodiments, the antibody that binds PD-1 is nivolumab, which is administered to the subject at a dose of 480 mg once every 4 weeks. In some embodiments, the antibody that binds PD-1 is prembrolizumab, which is administered at a dose of 200 mg once every 3 weeks. In some embodiments, the antibody that binds PD-1 is cemiplimab. In some embodiments, the antibody that binds PD-1 is cemiplimab. In some embodiments, the methods described herein comprise administration of cemiplimab to the subject at a dose of 350 mg intravenously once every 3 weeks. In some embodiments, the antibody that binds PD-1 is Tislelizumab. In some embodiments, the methods described herein comprise administration of Tislelizumab to the subject at a dose of 200 mg intravenously once every 3 weeks.

In some embodiments, the antibody that binds PD-L1 is administered using a flat dose. In some embodiments, the antibody that binds PD-L1 is Atezolizumab. In some embodiments, the methods described herein comprise administration of Atezolizumab to the subject at a dose of 1200 mg intravenously once every 3 weeks. In some embodiments, the antibody that binds PD-L1 is Avelumab. In some embodiments, the methods described herein comprise administration of Avelumab to the subject at a dose of 10 mg/kg intravenously every 2 weeks. In some embodiments, the antibody that binds PD-1 is Durvalumab. In some embodiments, the methods described herein comprise administration of Durvalumab to the subject at a dose of 1500 mg intravenously every 4 weeks.

In specific examples, any of the methods disclosed herein comprise (i) administering to a human patient having a target solid tumor as disclosed herein (e.g., ocular melanoma, e.g., uveal melanoma) any of the anti-Galectin-9 antibodies disclosed herein (e.g., G9.2-17 or the antibody having the heavy chain of SEQ ID NO: 19 and the light chain of SEQ ID NO:5) at a dose of about 1 to about 32 mg/kg (e.g., about 3 mg/kg or about 15 mg/kg) once every two weeks; and (ii) administering to the human patient an effective amount of an anti-PD-1 antibody (e.g., nivolumab, prembrolizumab, Tislelizumab, or cemiplimab, durvalumab, avelumab, and atezolizumab). In some embodiments, the antibody comprises a light chain complementarity determining region 1 (CDR1) set forth as SEQ ID NO: 1, a light chain complementary determining region 2 (CDR2) set forth as SEQ ID NO: 2, and a light chain complementary determining region 3 (CDR3) set forth as SEQ ID NO: 3 and/or comprises a heavy chain complementarity determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6. In some embodiments, the antibody comprises a heavy chain variable region comprising SEQ ID NO: 7. In some embodiments, the antibody comprises a light chain variable region comprising SEQ ID NO: 8. In some embodiments, the antibody comprises a heavy chain comprising SEQ ID NO: 19. In some embodiments, the antibody comprises a light chain comprising SEQ ID NO: 15. In some embodiments, the antibody is G9.2-17 IgG4. In some embodiments, the anti-Galectin-9 antibody is administered to the subject at a dose of about 1 mg/kg to about 32 mg/kg, e.g., the dose may be selected from 2 mg/kg, 4 mg/kg, 8 mg/kg, 12 mg/kg, and 16 mg/kg. In some embodiments, the antibody is administered once every two weeks, e.g., via intravenous infusion. In some embodiments, the method further comprises administering to the subject an immune checkpoint inhibitor, e.g., an anti-PD1 antibody. In some embodiments, the cancer is ocular melanoma. In some embodiments, the cancer is metastatic ocular melanoma. In some embodiments, the cancer is uveal melanoma. In some embodiments the cancer is metastatic uveal melanoma.

When nivolumab is used, a suitable dosing schedule can be about 480 mg once every 4 weeks. When prembrolizumab is used, a suitable dosing schedule can be about 200 mg once every 3 weeks. When cemiplimab is used, a suitable dosing schedule can be about 350 mg intravenously once every three weeks. When tislelizumab is used, a suitable dosing schedule can be about 200 mg intravenously once every 3 weeks. Alternatively, tislelizumab may be used at about 400 mg once very 6 weeks. In some instances, tislelizumab may be used at about 300 mg once every 4 weeks. In some embodiments an anti-PD-L1 antibody is used instead of an anti-PD-1 antibody. When Atezolizumab is used, a suitable dosing schedule can be about 1200 mg intravenously once every 3 weeks. When Avelumab is used, a suitable dosing schedule can be about 10 mg/kg intravenously every 2 weeks. When Durvalumab is used, a suitable dosing schedule can be about 1500 mg intravenously every 4 weeks.

Without being bound by theory, it is thought that anti-Galectin-9 antibodies, through their inhibition of Dectin-1, can reprogram immune responses against tumor cells via, e.g., inhibiting the activity of γδ T cells infiltrated into tumor microenvironment, and/or enhancing immune surveillance against tumor cells by, e.g., activating CD4+ and/or CD8+ T cells. Thus, combined use of an anti-Galectin-9 antibody and an immunomodulatory agent such as those described herein would be expected to significantly enhance anti-tumor efficacy.

In some embodiments, the anti-Galectin-9 antibody described herein is administered in combination with another therapy. Examples include, but are not limited to, melanoma include brachytherapy, internal radiation with Yttrium90 microspheres, HDAC inhibitors (e.g., verinostat), tyrosine kinase inhibitors (e.g., entrectinib, selumetinib, sorafenib, sunitinib, crizotinib etc.), PAC-1 (first procaspase activating compound), or intravitral avastin. In some embosiments, the additional therapy comprises a cell based therapy (e.g., an adoptive T cell transfer of TILs, CAR-Ts, or dentritic cells), cancer vaccines, autologous tumor RNA therapy, isolated hepatic perfusion, or ercutaneous hepatic perfusion (PHP).

Any of the anti-Galectin-9 antibodies described herein may be utilized in conjunction with other types of therapy for ocular melanoma, such as chemotherapy, surgery, radiation, gene therapy, or in conjunction with other types of therapy for autoimmune diseases, such as immunosuppressive mediation, hormone replacement therapy, blood transfusions, anti-inflammatory medication, and/or pain medication and so forth. Such therapies can be administered simultaneously or sequentially (in any order) with the immunotherapy according to the present disclosure.

In some embodiments, the methods are provided herein, wherein the anti-Galectin-9 antibody, for example antibody 9.2-17 or 9.1-8mut13, is combined with other immunomodulatory treatments such as, e.g., inhibitors of a checkpoint molecule (e.g., PD-1, PD-L1, PD-L2, CTLA-4, LAG3, TIM3, or A2aR), activators of a co-stimulatory receptor (e.g., DX40, GITR, CD137, CD40, CD27, and ICOS), and/or inhibitors of an innate immune cell target (e.g., KIR, NKG2A, CD96, TLR, and IDO).

In some embodiments, the methods are provided, wherein the anti-Galectin-9 antibody, can also be co-administered with a chemotherapeutic agent, including alkylating agents, anthracyclines, cytoskeletal disruptors (Taxanes), epothilones, histone deacetylase inhibitors, inhibitors of topoisomerase I, inhibitors of topoisomerase II, kinase inhibitors, nucleotide analogs and precursor analogs, peptide antibiotics, platinum-based agents, retinoids, vinca alkaloids and derivatives thereof. Non-limiting examples include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., gemcitabine, Dacarbazine, pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

Other suitable combination therapies include surgery, chemotherapy, radiation therapy, hormonal therapy (e.g., RDs, SERMs, and/or aromatase inhibitors), immunotherapy, complementary and holistic medicine, or a combination thereof. Examples include, but are not limited to, Abemaciclib, Abraxane®, Ado-Trastuzumab Emtansine Afinitor (Everolimus), Afinitor Disperz (Everolimus), Alpelisib, Anastrozole, Aredia (Pamidronate Disodium), Arimidex (Anastrozole), Aromasin (Exemestane), Atezolizumab, Capecitabine, Cyclophosphamide, Docetaxel, Doxorubicin Hydrochloride, Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Lynparza (Olaparib), Megestrol Acetate, Methotrexate, Neratinib Maleate, Nerlynx (Neratinib Maleate), Olaparib, Paclitaxel, Paclitaxel Albumin-stabilized Nanoparticle Formulation, Palbociclib, Pamidronate Disodium, Perjeta (Pertuzumab), Pertuzumab, Piqray (Alpelisib), Ribociclib, Talazoparib Tosylate, Talzenna (Talazoparib Tosylate), Tamoxifen Citrate, Taxol (Paclitaxel), Taxotere (Docetaxel), Tecentriq (Atezolizumab), Thiotepa, Toremifene, Trastuzumab, Trastuzumab and Hyaluronidase-oysk, Trexall (Methotrexate), Tykerb (Lapatinib

Ditosylate), Verzenio (Abemaciclib), Vinblastine Sulfate, Xeloda (Capecitabine), and Zoladex (Goserelin Acetate). In some examples, the anti-cancer therapy involves an anti-Galectin-9 antibody.

In some embodiments, the therapeutic agent to be co-used with the anti-galectin-9 antibody may be one or more receptor tyrosine kinase (RTK) inhibitors. Examples include, but are not limited to, dasatinib, imatinib, nilotinib, ponatinib, and sunitinib. Such RTK inhibitors may be in further combination with additional therapeutic agents, for example, bevacizumab, ipilimumab, or other checkpoint inhibitors known in the art or provided herein. See, e.g., Sabbah et al., Cancers (2021), 13:1685.

In some embodiments, the methods are provided, the anti-Galectin-9 antibody is administered concurrently with a checkpoint inhibitor. In some embodiments, the anti-Galectin-9 antibody is administered before or after a checkpoint inhibitor. In some embodiments, the checkpoint inhibitor is administered systemically. In some embodiments, the checkpoint inhibitor is administered locally. In some embodiments, the checkpoint inhibitor is administered by intravenous administration, e.g., as a bolus or by continuous infusion over a period of time, by intramuscular, intraperitoneal, intracerebrospinal, subcutaneous, intra-arterial, intra-articular, intrasynovial, intrathecal, intratumoral, oral, inhalation or topical routes. In one embodiment, the checkpoint inhibitor is administered to the subject by intravenous infusion.

In any of the method embodiments described herein, the anti-galectin-9 antibody can be administered (alone or in combination with an anti-PD1 antibody) once every 2 weeks for one cycle, once every 2 weeks for two cycles, once every 2 weeks for three cycles, once every 2 weeks for four cycles, or once every 2 weeks for more than four cycles. In some embodiments, the treatment is 1 to 3 months, 3 to 6 months, 6 to 12 months, 12 to 24 months, or longer. In some embodiments, the treatment is once every 2 weeks for 1 to 3 months, once every 2 weeks for 3 to 6 months, once every 2 weeks for 6 to 12 months, or once every 2 weeks for 12 to 24 months, or longer.

A subject being treated by any of the anti-galectin-9 antibodies disclosed herein (e.g., G9.2-17), either alone or in combination with a checkpoint inhibitor (e.g., an anti-PD-1 or anti-PD-L1 antibody) or one or more chemotherapeutics as disclosed herein may be monitored for occurrence of adverse effects (for example, severe adverse effects). Exemplary adverse effects to monitor are provided in Example 1 below. If occurrence of adverse effects is observed, treatment conditions may be changed for that subject. For example, the dose of the anti-galectin-9 antibody may be reduced and/or the dosing interval may be extended. Suitability and extent of reduction may be assessed by a qualified clinician. In one specific example, a reduction level as per clinician's assessment or at least by 30% is implemented. If required, one more dose reduction by 30% of dose level-1 is implemented (dose level-2). Alternatively or in addition, the dose of the checkpoint inhibitor can be reduced and/or the dosing interval of the checkpoint inhibitor may be extended. In some instances (e.g., occurring of life threatening adverse effects), the treatment may be terminated.

### E. Animal Models of Ocular Melanoma

The galectin-9 antibodies described herein may be evaluated in vivo, e.g., in an animal model. Any suitable animal model of an ocular melanoma may be used, e.g., a tumor syngeneic or xenograft mouse models (see, e.g., Richards et al., Pigment Cell Melanoma Res. 2020 March; 33(2): 264-278.). The anti-Galectin-9 antibodies may be administered to the animal systemically or locally, e.g., via oral administration (gavage), intravenous, or subcutaneous injection or via intratumoral injection, and treatment efficacy determined, e.g., by measuring tumor volume.

Implantation of cultured cells derived from various human cancer cell types or a patient's tumor mass into mouse tissue sites has been widely used for generations of cancer mouse models (xenograft modeling). In xenograft modeling, human tumors or cell lines are implanted either subcutaneously or orthotopically into immune-compromised host animals (e.g., nude or SCID mice) to avoid graft rejection. Because the original human tumor microenvironment is not recapitulated in such models, the activity of anti-cancer agents that target immune modulators may not be accurately measured in these models, making mouse models with an intact immune system more desirable. Accordingly, implantation of murine cancer cells in a syngeneic immunocompetent host (allograft) are used to generate mouse models with tumor tissues derived from the same genetic background as a given mouse strain. In syngeneic models, the host immune system is normal, which may more closely represent the real life situation of the tumor's micro-environment. The tumor cells or cancer cell lines are implanted either subcutaneously or orthotopically into the syngeneic immunocompetent host animal (e.g., mouse). Representative murine tumor cell lines, which can be used in syngeneic mouse models for immune checkpoint benchmarking are dexcribed in Richards et al., Pigment Cell Melanoma Res. 2020 March; 33(2): 264-278. Selected cell lines for use in syngeneic mouse models.

Examples of mouse strains that can be used in syngeneic mouse models, depending on the cell line include C57BL/6, FVB/N, Balb/c, C3H, HeJ, C3H/HeJ, NOD/ShiLT, A/J, 129S1/SvlmJ, NOD. Additionally, several further genetically engineered mouse strains have been reported to mimic human tumorigenesis at both molecular and histologic levels. These genetically engineered mouse models also provide excellent tools to the field and additionally, the cancer cell lines derived from the invasive tumors developed in these models are also good resources for cell lines for syngeneic tumor models.

More recently, “humanized” mouse models have been developed, in which immunodeficient mice are reconstituted with a human immune system, and which have helped overcome issues relating to the differences between the mouse and human immune systems, allowing the in vivo study of human immunity. Severely immunodeficient mice which combine the IL2receptor null and the severe combined immune deficiency mutation (scid) (NOD-scid IL2Rgnull mice) lack mature T cells, B cells, or functional NK cells, and are deficient in cytokine signaling. These mice can be engrafted with human hematopoietic stem cells and peripheral-blood mononuclear cells. CD34+hematopoietic stem cells (hu-CD34) are injected into the immune deficient mice, resulting in multi-lineage engraftment of human immune cell populations including very good T cell maturation and function for long-term studies. This model has a research span of 12 months with a functional human immune system displaying T-cell dependent inflammatory responses with no donor cell immune reactivity towards the host. Patient derived xenografts can readily be implanted in these models and the effects of immune modulatory agents studied in an in vivo setting more reflective of the human tumor microenvironment (both immune and non-immune cell-based) (Baia et al., 2015).

Examplary cell lines are listed in Table 3 Exemplary human cell lines for xeongrafts are in Table 4. Table 5 lists selected Patient-derived mouse xenograft models of uveal melanoma. Table 6 lists genetically engineered mouse models of uveal melanoma Tables 3-6 are adopted from Pigment Cell Melanoma Res. 2020 March; 33(2): 264-278), and references can be found therein.

TABLE 3
Syngeneic mouse cutaneous melanoma models for simulating uveal melanoma
(adopted from Pigment Cell Melanoma Res. 2020 March; 33(2): 264-278)

| | | Original | | |
|---|---|---|---|---|
| | | publication | | |
| | | (Laboratory of | | |
| Cell line | Source | origin) | Inoculation method | Metastasis |
| B16LS9 | Mouse cutaneous | Rusciano et al. | Suprachoroidal | Liver, |
| | melanoma | (1994) (Max | | lungs, and |
| | | Burger) | | lymph |
| | | | | nodes |
| | | | Ant. chamber | Not |
| | | | | reported |
| | | | Intravitreal | Liver |
| | | | Intrasplenic | |
| | | | Intrahepatic | Not |
| | | | | reported |
| B16F10 | Mouse cutaneous | Fidler, Gersten, | Suprachoroidal | None |
| | melanoma | and Budmen | Ant. chamber | Not |
| | | (1976) (Marilyn | | reported |
| | | Budmen) | | Lungs |
| | | | Tail vein | Lungs |
| Queens | Mouse cutaneous | Harning et al. | Suprachoroidal | Lungs |
| | melanoma | (1987) (Jeanne | Ant. chamber | None |
| | | Szalay) | | Lungs |
| | | | Tail vein | Lungs |
| HCmel12 | Mouse cutaneous | Kilian et al. | Intravitreal | Lungs and |
| | melanoma | (2016) (Thomas | | lymph |
| | | Tüting) | | nodes |
| Oncogene- | Immortalized | Bennett, Cooper, | Subcutaneous | Not |
| transduced | mouse | and Hart (1987) | | reported |
| melan-A | melanocyte | (Ian Hart) | | Lungs and |
| cells | | | | liver |

TABLE 4
Human uveal melanoma cell lines derived from (a) primary tumors used in mouse
xenograft experiments (b) metastases used in mouse xenograft experiments

| (a) Cell line | | Original publication | Inoculation | |
|---|---|---|---|---|
| (mutations) | Source | (Laboratory of origin) | method | Metastasis |
| Mel92.1 | Primary | De Waard-Siebinga et | Subcutaneous | Not |
| (GNAQ^{Q209L.}, | tumor | al. (1995) (Martine | | reported |
| EIF1AX^{G6D}) | | Jager) | Suprachoroidal | Liver |
| | | | | Not |
| | | | | reported |
| | | | Ant. chamber | Liver |
| | | | Tail vein | Liver and |
| | | | | lungs |
| | | | Intrasplenic | Liver |
| Mel202 | Primary | Ksander, Rubsamen, | Subcutaneous | Not |
| (GNAQ^{Q209L.}, | tumor | Olsen, Cousins, and | | reported |
| SF3B1^{R625G}) | | Streilein (1991) (J. | Ant. chamber | Liver |
| | | Wayne Streilein) | Intravitreal | Not |
| | | | | reported |
| | | | Tail vein | Liver |
| Mel270 | Primary | Verbik, Murray, Tran, | Suprachoroidal | Not |
| (GNAQ^{Q209P}) | tumor | and Ksander (1997) | | reported |
| | | (Bruce Ksander) | Subcutaneous | Liver and |
| | | | | lungs |
| | | | | Lot |
| | | | | reported |
| | | | Intrasplenic | Liver |
| MP41 | PDX from | Amirouchene- | Tail vein | Liver |
| (GNA11^{Q209L}) | a primary | Angelozzi et al. (2014) | | |
| | tumor | (Sergio Roman- | | |
| | | Roman) | | |
| T105 and T142 | Primary | Mouriaux et al. (2016) | Subcutaneous | Not |
| (GNA11^{Q209L}) | tumors | (Sylvain Guérin) | | reported |
| UMT2 | Primary | Suesskind et al. (2013) | Suprachoroidal | None |
| (GNA11^{Q209L}) | tumors | (Sigrid Henke-Fahle) | | |

| | | Original publication | Inoculation | |
|---|---|---|---|---|
| (b) Cell line | Source | (Laboratory of origin) | site | Metastasis |
| OMM1 | Subcutis | Luyten et al. (1996) | Subcutaneous | Not |
| (GNA11Q209L) | metastasis | (Theo Luider) | | reported |
| | | | Ant. | Liver |
| | | | chamber | |
| | | | Tail vein | Liver |
| OMM1.3^{a} = OM | Liver | Verbik et al. (1997) | Subcutaneous | Not |
| M2.3 | metastasis | (Bruce Ksander) | | reported |
| (GNAQ^{Q209P}) | | | Suprachoroidal | Liver |
| | | | Retro-orbital | Liver and |
| | | | | lungs |
| | | | Intrasplenic | Liver |
| TJU-UM001 | Liver | Yoshida et al. (2014) | Intrahepatic | Peritoneum, |
| (GNAQ^{Q209P}) | metastasis | (Takami Sato) | | lymph |
| | | | | nodes |
| | | | Intrasplenic | Liver |
| TJU-UM004 | Orbital | Cheng et al. (2015) | Intrahepatic | None |
| (GNAQ^{Q209P}) | metastasis | (Takami Sato) | | |

TABLE 5
Patient-derived mouse xenograft models of uveal melanoma

| | | Original Publication | Inoculation |
|---|---|---|---|
| PDX model | Source | (Laboratory of origin) | site |
| 6 cases successfully grafted 3 | Liver | Kageyama et al. (2017) | Liver |
| times | metastases | (Takami Sato) | |
| MP34, MP38, MP41, MP42, | Primary | Némati et al. (2010) (Didier | Interscapular |
| MP46, MP47, MP55, MP71, | tumors | Decaudin) | fat pad |
| MP77, and MP80 | | | |
| MM33 | Subcutis | | |
| | metastasis | | |
| MM26, MM28, MM52, MM66, | Liver | | |
| and MM74 | metastases | | |
| ØPI-204 | Primary | Heegaard, Spang-Thomsen, | Subcutaneous |
| | tumor | and Prause (2003) (Jan Ulrik | |
| | | Prause) | |

TABLE 6
Genetically engineered mouse models of uveal melanoma

| | | | Original |
|---|---|---|---|
| | | | Publication |
| | | | (Laboratory |
| Model genotype | Induction | Phenotype | of origin) |
| Dct-rtTA/+; tet- | 5- to 6-week-old | >50% of mice developed | Feng et al. |
| HA- | mice; doxycycline in | cutaneous melanoma; no | (2014) (J. |
| GNAQ^{Q209L}/+; p | food | report of lesions in the uveal | Silvio |
| 16p19^{KO} | | tract | Gutkind) |
| Rosa26-floxed | Embryonic (E15.5) | Skin hyperpigmentation overt | Huang et al. |
| stop- | activation by | uveal melanoma and | (2015) |
| GNAQ^{Q209L}/+; | constitutive Cre | occasional dermal melanoma | (Catherine |
| Mitf-cre/+ | driver | at 3 months in 15/15 mice; | Van |
| | | melanocytic neoplasia of the | Raamsdonk) |
| | | leptomeninges, harderian | |
| | | gland, cochlea, and vestibular | |
| | | system; putative metastases | |
| | | in the lungs at 3 months in | |
| | | 18/19 mice | |
| Rosa26-floxed | 8-week-old mice; | Skin hyperpigmentation; | |
| stop- | daily IP injection of | melanocytic hyperplasia of | |
| GNAQ^{Q209L}/+; Tyrosinase- | tamoxifen and tail | the uveal tract (but not overt | |
| creER/+ | dip in 4-HT for | melanoma) in 3/3 mice | |
| | 5 days | | |
| R26-LSL- | 4-week-old mice; | Skin hyperpigmentation; | Moore et al. |
| GNA11^{Q209L}/+; | single IP injection of | overt uveal and dermal | (2018) (Yu |
| Tyrosinase- | tamoxifen | melanoma at 6 months in | Chen) |
| creER^{T2}/+ | | 50% of mice; melanocytic | |
| | | neoplasia of the | |
| | | leptomeninges, third | |
| | | ventricle, harderian gland, | |
| | | and heart; putative | |
| | | metastases in axillary lymph | |
| | | nodes and lungs at 3-6 | |
| | | months in 100% of mice | |
| R26-LSL- | | Compared to above: | |
| GNA11^{Q209L}/+; | | increased dermal melanoma | |
| BAP1^{lox/lox.}, Tyrosinase- | | burden and proliferative | |
| creER^{T2}/+ | | index, no change in number | |
| | | or size of uveal melanoma | |
| | | tumors or lung lesions | |
| AAV5-CMV-Cre | 2- to 4-month-old | Eye bulging at 2 months and | Li et al. |
| or AAV5-Trp2- | mice; suprachoroidal | uveal melanoma formation at | (2019) |
| GFPCre; | injection of AAV | 6 months in 12/14 and 8/10 | (Junhao |
| Lats1^{f/f}; Lats2^{f/f} | | mice, respectively | Mao) |
| AAV5-Trp2- | | Compared to above: larger | |
| GFPCre; | | uveal melanoma tumors in | |
| Lats1^{f/f}; Lats2^{f/f}; | | 7/7 mice and reduced | |
| LSL-KrasG12D | | survival (<4 months) | |

### II. Methods for Cancer Diagnosis or Prognosis Using Galectin-9 as a Biomarker

In some aspects provided herein, are diagnostic and prognostic methods for ocular melanoma by measurement of Galectin-9 (Gal-9) levels in a biological sample obtained from a subject in need thereof. The Galectin-9 levels can be used as a biomarker, for example, to identify a patient as having the target cancer, to measure tumor burden in the patient, and/or to assess metastatic status of the cancer.

As used herein, “biomarker” refers to a distinctive biological or biologically derived indicator of a process, event or conditions. In certain embodiments, the biomarker is a gene or gene product (i.e., a polypeptide).

As used herein, “predictive biomarker” as used herein refers to a biomarker that can be used in advance of therapy to estimate the likelihood or predictability of response to a given therapeutic agent or class of therapeutic agents. In certain embodiments, the therapeutic agent is an anti-Galectin-9 immunotherapy, for example, an anti-Galectin-9 antibody as disclosed herein. In some examples, the predictive biomarker is serum or plasma Galectin-9 levels. In other examples, the predictive biomarker is Galectin-9 levels in a tumor and/or Galectin-9 levels in organoid cultures derived from a tumor. In some embodiments, more than one predictive biomarker is used. In some embodiments, at least one biomarker is used are used in combination with Galectin-9 levels as predictive biomarker. In some embodiments, PD-L1 levels are used in combination with Galectin-9 levels a predictive biomarker. In some embodiments, the predictive biomarker used in combination with Galectin-9 levels is serum or plasma PD-L1 levels. In some embodiments, the predictive biomarker used in combination with Galectin-9 levels is serum or plasma PD-L1 levels in a tumor and/or PD-L1 levels in organoid cultures derived from a tumor.

### A. Biological Samples for Measurement of Galectin-9

Any of the methods disclosed herein involve one or more biological samples collected from a subject such as a human subject at one or more suitable time points for measurement of Galectin-9 levels. In some examples, the subject may be a human subject suspected of having or at risk for a target cancer, for example, ocular melanoma, e.g., uveal melanoma. In some embodiments, the ocular e.g., uveal, melanoma is metastatic. Such a patient may be free of any prior anti-cancer therapy. Alternatively, the patient may be free of any anti-cancer therapy. In some instances, the patient may have previously undergone an anti-cancer therapy, for example, immunotherapies, checkpoint inhibitor therapies, chemotherapies, radiotherapies, surgery, and combinations thereof.

In some examples, the biological sample can be a blood sample such as a serum sample or a plasma sample. In some embodiments, Galectin-9 levels are measured in choroidal fluid, ascites, pleural effusion, or cerebrospinal fluid.

In some examples, the biological sample can be a blood sample such as a serum sample or a plasma sample. The Galectin-9 levels may refer to the total amount of Galectin-9 in such a sample. Alternatively, the Galectin-9 levels may refer to the amount of circulating Galectin-9 in the sample. In other examples, the Galectin-9 levels may refer to the amount of cell surface Galectin-9 (e.g., Galectin-9 on tumor cells or Galectin-9 on immune cells) in the sample. In other examples, the biological sample can be a tissue sample, for example, a tumor tissue sample. In some specific examples, the tissue sample is a patient derived organoid (PDO) sample. The Galectin-9 level in such sample may refer to the total amount of Galectin-9 in the sample. In some examples, the Galectin-9 level may refer to the amount of Galectin-9 on a specific type of cells, e.g., tumor cells or tumor infiltrated lymphocytes (TILs).

In some embodiments, the level of Galectin-9 refers to the protein level of Galectin-9 in a biological sample. In other embodiments, the level of Galectin-9 refers to the messenger RNA level of Galectin-9 in a biological sample.

In some examples, only levels of Gal-9 are measured in the biological samples disclosed herein. In other examples, levels of PD-L1 are also measured in addition to the levels of Gal-9. In some embodiments, levels of Gal-9 and levels of PD-L1 are measured in the same biological sample. For example, in some embodiments, Gal-9 levels and PD-L1 levels are both measured in a serum sample or a plasma sample. For example, in some embodiments, Gal-9 levels and PD-L1 levels are both measured in a in a tissue sample, e.g., a tumor tissue sample or a patient derived organoid (PDO) sample. In some embodiments, levels of Gal-9 and levels of PD-L1 are measured in the separate biological samples. For example, in some embodiments, Gal-9 levels are measured in a serum sample or a plasma sample and PD-L1 is measured in a tissue sample, e.g., a tumor tissue sample or a patient derived organoid (PDO) sample, or vice versa.

Levels of Galectin-9 in any of the biological samples disclosed herein may be measured by conventional methods. In some instances, levels of Galectin-9 may be measured by an immune assay, which refers to a biochemical assay for determining the presence or concentration of a target molecule through the use of an antibody or an antigen. Examples include, but are not limited to, enzyme-linked immunosorbent assays (ELISAs), Westernblot, radioimmunoassays (RIA), counting immunoassays (CIA), fluoroimmunoassays (FIA), and chemiluminescenceimmunoassays (CLIA). In some instances, flow cytometry may be used for measuring Galectin-9 positive cells in a sample. In some instances, immunohistochemistry may be used for measuring Galectin-9 positive cells in a sample. Other emerging protein analysis techniques which may be used are extensively known in the art (see e.g., Powers and Palecek, Protein analytical assays for diagnosing, monitoring, and choosing treatment for cancer patients J Health Eng. 2012 December; 3(4): 503-534) and include mass spectrometric techniques, such as matrix assisted laser desorption/ionization and surface enhanced laser desorption/ionization mass spectroscopy. Minaturization can be accomplished using techniques known in the art, such as using microfluidics. For example, the combination of microfluidics with traditional immunoassays, including IHC, flow cytometry, and ELISA, reduces antibody consumption by several orders of magnitude and offers the potential for assay automation. Additional assay tools include nanoparticles, e.g., polystyrene beads, quantum dots, gold particles or carbon nanotubes.

Levels of Galectin-9 nucleic acid levels, e.g., mRNA levels may be measured according to methods known in the art, e.g., using PCR-based techniques.

In some embodiments, levels of Galectin-9 expressed on the surface of cells derived from the human patient (e.g., from the blood or tumor of the human patient) are measured and compared to a reference level. In some embodiments, Galectin-9 expressed on the surface of cancer or immune cells derived from corresponding a patient (e.g., macrophages, alpha/beta T cells or gamma/delta T cells), e.g., derived from a biopsy, a patient's tumor culture, including but not limited to, PDOTs, or a blood sample, are measured and compared to a reference level.

### B. Identification and Diagnosis of Ocular Melanoma Patients Cancer Patients

In some embodiments, provided here is a method for diagnosing a patient, for example, determining occurrence, tumor burden, and/or metastatic risk/status of a target cancer (e.g., ocular melanoma, e.g., uveal melanoma. in a subject, such as a human patient. In some embodiments, the ocular e.g., uveal, melanoma is metastatic.

In some instances, the diagnosis is based on levels of Galectin-9 measured in a biological sample (e.g., those disclosed herein) collected from a subject, e.g., a human subject. In some embodiments, galectin-9 levels are measured in serum or plasma of a patient. In some embodiments, galectin-9 levels are measured in a tissue sample from the patient.

Accordingly, in some examples, the method comprises: (i) providing a biological sample such as a blood sample (e.g., a plasma sample or a serum sample) of a subject, e.g., a human subject, in need thereof, (ii) measuring the level of galectin-9 in the blood sample, and (iii) identifying the subject as having a cancer or being at risk for the cancer based on the level of galectin-9 in the blood sample. An elevated level of galectin-9 in the biological sample (e.g., a blood sample such as a serum sample or a plasma sample) of the subject relative to a predetermined reference level may indicate that the subject has the cancer or is at risk for the cancer. An elevated level of galectin-9 in the biological sample of the subject relative to a control may also indicate that the subject is a ocular melanoma patient suitable for an immunotherapy, such as an immunotherapy involving a Gal-9 antagonist, for example, an anti-Gal-9 antibody as those disclosed herein.

In any of the methods disclosed above, the predetermined reference level may represent the level of Galectin-9 in a biological sample (e.g., the same type of biological sample, such as a blood sample) from a control subject of the same species (e.g., a human subject) who is free of the target cancer. Preferably, the control subject is free of any type of cancer. In some embodiments, the predetermined reference level refers to a pre-determined reference range of values representing the level of Galectin-9 in control subjects of the same species (e.g., human subjects) who are free of the target cancer, preferably free of any cancer. The control subjects may have matched physiological features as the subject, for example, age, gender, ethnic background, etc. Accordingly, levels higher than the reference values are indicative of an increased level. If the Galectin-9 level in the biological sample of a candidate subject (e.g., a human subject) is elevated relative to the predetermined reference level, this indicates that the candidate subject has or is at risk for the target cancer as those disclosed herein.

In some embodiments, the predetermined reference level may represent the level of Galectin-9 in a biological sample (e.g., the same type of biological sample, such as a blood sample) from a control patient having the target cancer at a low tumor burden. The control patient may have matched physiological features as the subject, for example, age, gender, ethnic background, etc. Accordingly, levels higher than the reference values are indicative of an increased level. If the Galectin-9 level in the biological sample of a candidate subject (e.g., a human subject) is elevated relative to the predetermined reference level, this indicates that the candidate subject has the target cancer as those disclosed herein at a high tumor burden. As used herein, tumor burden refers to amount of cancer, the size or the volume of the tumor in the body of a subject, accounting for all sites of disease. When a subject is identified as having a high tumor burden by a method disclosed herein, the tumor burden in the subject can be confirmed using methods known in the art, including but not limited to, FDG positron emission tomography (FDG-PET), magnetic resonance imaging (MRI), and optical imaging, comprising bioluminescence imaging (BLI) and fluorescence imaging (FLI).

In some embodiments, the predetermined reference level may represent the level of Galectin-9 in a biological sample (e.g., the same type of biological sample, such as a blood sample) from a control patient having the target cancer without metastasis. The control patient may have matched physiological features as the subject, for example, age, gender, ethnic background, etc. Accordingly, levels higher than the reference values are indicative of an increased level. If the Galectin-9 level in the biological sample of a candidate subject (e.g., a human subject) is elevated relative to the predetermined reference level, this indicates that the candidate subject has the target cancer in metastatic status. As used herein, metastatic solid tumors/cancer refer to tumors/cancers having tumor/cancer cells from the place where they first form to another part of the body. In metastasis, cancer cells break away from the original tumor, travel through the blood or lymph system, and form a new tumor in other organs or tissues of the body.

In some embodiments, a Galectin-9 level may be deemed elevated as compared with a predetermined reference level when the Galectin-9 level is at least 20% higher, 30% higher, 40% higher, for example, at least 50% higher, at least 80% higher (including any numerical increment between the listed percentages), or at least 2-fold higher, than the predetermined reference level. In some embodiments, a Galectin-9 level may be deemed elevated as compared with a predetermined reference level when the Galectin-9 level is at least about 20% higher, 30% higher, 40% higher, for example, at least about 50% higher, at least about 80% higher (including any numerical increment between the listed percentages), or at least about 2-fold higher, than the predetermined reference level. In some embodiments, a Galectin-9 level may be deemed elevated as compared with a predetermined reference level when the Galectin-9 level is at least 2-fold higher, at least 3-fold higher, at least 4-fold higher, at least 5-fold higher, at least 6-fold higher, at least 7-fold higher, at least 8-fold higher, at least 9-fold higher, at least 10-fold higher, at least 10-fold to 15-fold higher, at least 15-fold to 20-fold higher, at least 20-fold to 25-fold higher, or at least 25-fold to 30-fold higher (including any numerical increment between the listed values). In some embodiments, a Galectin-9 level may be deemed elevated as compared with a predetermined reference level when the Galectin-9 level is at least about 2-fold higher, at least about 3-fold higher, at least about 4-fold higher, at least about 5-fold higher, at least about 6-fold higher, at least about 7-fold higher, at least about 8-fold higher, at least about 9-fold higher, at least about 10-fold higher, at least about 10-fold to 15-fold higher, at least about 15-fold to 20-fold higher, at least about 20-fold to 25-fold higher, or at least about 25-fold to 30-fold higher (including any numerical increment between the listed values).

In some embodiments, the methods of diagnosing a subject and optionally treating the subject described herein comprise a step (iii), wherein a Galectin-9 level is deemed elevated as compared with a predetermined reference level when the Galectin-9 level is at least 2-fold higher or at least about 2-fold higher. In some embodiments, the methods of diagnosing a subject and optionally treating the subject described herein comprise a step (iii), wherein a Galectin-9 level is deemed elevated as compared with a predetermined reference level when the Galectin-9 level is at least 2.5-fold higher or about 2.5-fold higher. In some embodiments, the methods of diagnosing a subject and optionally treating the subject described herein comprise a step (iii), wherein a Galectin-9 level is deemed elevated as compared with a predetermined reference level when the Galectin-9 level is at least 4-fold higher or at least about 4-fold higher. In some embodiments, the methods of diagnosing a subject and optionally treating the subject described herein comprise a step (iii), wherein a Galectin-9 level is deemed elevated as compared with a predetermined reference level when the Galectin-9 level is at least 20-fold higher or at least about 20-fold higher.

In some embodiments, the methods of identifying a cancer patient and optionally treating the cancer patient described herein comprise a step (iii), wherein a Galectin-9 level is deemed elevated as compared with a predetermined reference level when the Galectin-9 level is at least 2-fold higher or at least about 2-fold higher. In some embodiments, the methods of identifying a cancer patient and optionally treating the cancer patient described herein comprise a step (iii), wherein a Galectin-9 level is deemed elevated as compared with a predetermined reference level when the Galectin-9 level is at least 2.5-fold higher or about 2.5-fold higher. In some embodiments, the methods of identifying a cancer patient and optionally treating the cancer patient described herein comprise a step (iii), wherein a Galectin-9 level is deemed elevated as compared with a predetermined reference level when the Galectin-9 level is at least 4-fold higher or at least about 4-fold higher. In some embodiments, the methods of identifying a cancer patient and optionally treating the cancer patient described herein comprise a step (iii), wherein a Galectin-9 level is deemed elevated as compared with a predetermined reference level when the Galectin-9 level is at least 20-fold higher or at least about 20-fold higher.

In some examples, the methods disclosed herein may comprise (i) providing a biopsy sample of a subject, e.g., a human subject, in need thereof, (ii) measuring the level of galectin-9 in the biopsy sample from a subject or in a sample derived from a biological sample of a subject, e.g., patient-derived organotypic tumor spheroids (PDOTs), e.g., prepared as described herein, and (iii) identifying the subject as having a cancer or being at risk for the cancer based on the level of galectin-9 in the sample. In some embodiments, the elevated level of galectin-9 in the sample of the subject is relative to a control and indicates that the subject has the cancer or is at risk for the cancer. In some embodiments, the control is healthy organ tissue or a part of the organ which is not affected by the cancer from the same subject. In some embodiments, the control is a reference value or range of values. In some embodiments, the control is derived from a healthy subject. In some embodiments, the measuring involves determining levels of Galectin-9 protein or gene expression, e.g., mRNA levels.

In any of the methods disclosed above, the control may represent the level of Gal-9 in a biological sample (e.g., the same type of biological sample, such as a blood sample) from a control subject of the same species (e.g., a human subject) who is free of ocular melanoma. Preferably, the control subject is free of any type of cancer. In some embodiments, the control comprises a pre-determined reference value or range of values representing the level of Gal-9 in control subjects of the same species (e.g., human subjects) who are free of ocular melanoma, preferably free of any cancer. The control subjects may have matched physiological features as the subject, for example, age, gender, ethnic background, etc. Accordingly, levels higher than the reference values be indicative of an increased level.

In some examples, one or more other diagnostic tests known in the art can be used in conjunction with the method to confirm cancer occurrence and/or risk. When a subject is identified as having ocular melanoma or at risk for ocular melanoma, a suitable anti-cancer therapy can be applied to such a patient. Details of anti-cancer therapies for treating ocular melanoma are provided below.

### C. Determination of Tumor Burden and Tumor Status

In other embodiments, provided herein are methods for determining tumor burden or tumor status in a ocular melanoma patient. Such a method may comprise: (i) providing a biological sample (e.g., a blood sample such as a plasma or serum sample, a biopsy or biopsy derived samples, e.g., PDOTs or other tissue sample) of a subject having ocular melanoma (e.g., a human ocular melanoma patient), (ii) measuring the level of galectin-9 in the sample, and (iii) determining tumor burden or tumor status of the subject based on the level of galectin-9 in the sample. For example, an elevated level of galectin-9 in the biological sample of the subject relative to a control indicates that the subject has a high tumor burden. In some examples, the level of Gal-9 on TILs, the level of Gal-9 on tumor associated macrophages (TAMs), the level of Gal-9 on tumor cells, or both, are measured. A higher level of Gal-9 on TILs and/or on tumor cells relative to a control may indicate high tumor grade and/or high mitotic index of tumor cells, which is indicative of tumor cell proliferation.

In some examples, the methods for determining tumor burden in a ocular melanoma patient may comprise: (i) providing a biological sample e.g., blood plasma or serum, biopsy or biopsy derived samples, e.g., PDOTs or other tissue sample, of a subject having a cancer, (ii) using an anti-galectin-9 antibody to measure the level of galectin-9 in the biological sample, and (iii) determining tumor burden of the subject based on the level of galectin-9 in the biological sample, wherein an elevated level of galectin-9 in the biological sample of the subject relative to a control indicates that the subject has a high tumor burden. In some embodiments, the level of gal-9 on TILs and/or on tumor cells is measured.

In any of the methods for determining tumor burden or tumor status disclosed above, the control may represent the level of Gal-9 in a biological sample (e.g., the same type of biological sample, such as a blood sample) from a control subject or a group of control subjects of the same species (e.g., human subjects). The control subjects may have matched physiological features as the subject, for example, age, gender, ethnic background, etc. In some instances, the control subjects are free of ocular melanoma, preferably free of any cancer. In other instances, the control subjects may be ocular melanoma patients having a low tumor burden as determined by routine medical practice. In other examples, the control may comprise a pre-determined reference value or range of values representing the level of Gal-9 in any of the control subjects disclosed above.

In other examples, the method comprises: (i) providing a biological sample such as a blood sample (e.g., a plasma sample or a serum sample) of a subject, e.g., a human subject, in need thereof, (ii) measuring the level of galectin-9 in the blood sample, and (iii) assessing metastatic status of the subject based on the level of galectin-9 in the blood sample. An elevated level of galectin-9 in the biological sample (e.g., a blood sample such as a serum sample or a plasma sample) of the subject relative to a predetermined reference level may indicate that the subject has a metastatic ocular melanoma, e.g., a metastatic uveal melanoma.

In some embodiments, the methods for determining tumor burden or metastatic status in a cancer patient and optionally treating the cancer patient described herein comprise a step (iii), wherein a Galectin-9 level is deemed elevated as compared with a predetermined reference level when the Galectin-9 level is at least 3-fold higher or at least about 3-fold higher. In some embodiments, the methods for determining tumor burden or metastatic status in a cancer patient and optionally treating the cancer patient described herein comprise a step (iii), wherein a Galectin-9 level is deemed elevated as compared with a predetermined reference level when the Galectin-9 level is at least 4-fold higher or about 4-fold higher. In some embodiments, the methods for determining tumor burden or metastatic status in a cancer patient and optionally treating the cancer patient described herein comprise a step (iii), wherein a Galectin-9 level is deemed elevated as compared with a predetermined reference level when the Galectin-9 level is at least 5-fold higher or at least about 5-fold higher. In some embodiments, the methods for determining tumor burden or metastatic status in a cancer patient and optionally treating the cancer patient described herein comprise a step (iii), wherein a Galectin-9 level is deemed elevated as compared with a predetermined reference level when the Galectin-9 level is at least 6-fold higher or at least about 6-fold higher.

In other examples, the method comprises: (i) providing a biological sample such as a blood sample (e.g., a plasma sample or a serum sample) of a subject, e.g., a human subject, in need thereof, (ii) measuring the level of galectin-9 in the blood sample, and (iii) assessing tumor burden of the subject based on the level of galectin-9 in the blood sample. An elevated level of galectin-9 in the biological sample (e.g., a blood sample such as a serum sample or a plasma sample) of the subject relative to a predetermined reference level may indicate that the subject has a high tumor burden.

In some embodiments, the methods of treating a subject having cancer described herein comprise a step (iii), wherein a Galectin-9 level is deemed elevated as compared with a predetermined reference level when the Galectin-9 level is at least 2-fold higher or at least about 2-fold higher. In some embodiments, the methods of treating a subject having cancer described herein comprise a step (iii), wherein a Galectin-9 level is deemed elevated as compared with a predetermined reference level when the Galectin-9 level is at least 2.5-fold higher or about 2.5-fold higher. In some embodiments, the methods of treating a subject having cancer described herein comprise a step (iii), wherein a Galectin-9 level is deemed elevated as compared with a predetermined reference level when the Galectin-9 level is at least 4-fold higher or at least about 4-fold higher. In some embodiments, the methods of treating a subject having cancer described herein comprise a step (iii), wherein a Galectin-9 level is deemed elevated as compared with a predetermined reference level when the Galectin-9 level is at least 20-fold higher or at least about 20-fold higher.

In some examples, the level of Gal-9 in a tumor tissue sample may be used as a biomarker for determining the tumor grade of a ocular melanoma patient. In some instances, the tumor grade can be represented by the mitotic index, which is an important factor indicating the proliferative level of cancer cells. A high level of Gal-9 observed in a tumor tissue sample relative to a control level can be indicative of high tumor grade and/or high mitotic index. In some examples, the level of Gal-9 on tumor infiltrating lymphocytes (TILs) in a tumor tissue sample can be used as the biomarker for determining tumor grade or mitotic index for a ocular melanoma patient. A high level of Gal-9 observed on TILs relative to a control level can be indicative of high tumor grade and/or high mitotic index. A control level may be a pre-determined value or a range of values representing the level of Gal-9 in ocular melanoma patients having a determined tumor grade or mitotic index level. In some instances, the control level may be a cutoff value or a range of values distinguishing the levels of Gal-9 in patients having high grade tumor or high mitotic index relative to patients having low grade tumor or low mitotic index. The levels of high or low tumor grade and mitotic index are based on standard in medical practice as known to those medical practitioners.

When needed, one or more additional diagnostic assays as known in the art can be performed on the subject in conjunction with the methods disclosed herein to confirm tumor burden and/or tumor status of the ocular melanoma patient.
A suitable anti-cancer therapy (e.g., comprising the use of a Gal-9 antagonist such as an anti-Gal-9 antibody) can be selected based on the tumor burden and/or status thus determined and applied to the ocular melanoma patient.

In some examples, Gal-9 is used as a sole biomarker in the methods disclosed herein. In other examples, Gal-9 is used in conjunction with PD-L1.

In some examples, one or more other diagnostic tests known in the art can be used in conjunction with the method to confirm cancer occurrence, risk, tumor burden, and/or metastatic status. When a subject is identified as having the target cancer or at risk for the target cancer, a suitable anti-cancer therapy can be applied to such a patient. When a subject is identified as having the target cancer with a high tumor burden and/or with metastasis (or at risk for it), a suitable anti-cancer therapy can be selected based on the assessment. Details of anti-cancer therapies for treating any of the target cancers are provided below.

Melanocytes within the uveal tract (iris, ciliary body, and choroid) of the eye can give rise to uveal melanoma. Tumors that develop in the iris are deemed anterior uveal melanomas versus tumors that develop in the ciliary body and/or choroid are termed posterior uveal melanomas.

Accordingly, in some embodiments, the cancer is ocular melanoma. In some embodiments, the cancer is metastatic ocular melanoma. In some embodiments, the cancer is uveal melanoma. In some embodiments the cancer is metastatic uveal melanoma. In some embodiments, the cancer is anterior uveal melanoma or metastatic anterior uveal melanoma. In some embodiments, the cancer is posterior uveal melanoma or metastatic anterior uveal melanoma. In some embodiments, the primary tumor is in the uveal tract. In some embodiments, the primary tumor is in the iris, ciliary body or the choroid. In some embodiments, the metastatic tumor is in the liver.

### D. Assessment of Responsiveness to an Anti-Cancer Therapy

In other embodiments, provided herein are methods for predicting the responsiveness of a ocular melanoma patient to an anti-cancer therapy such as an anti-galectin-9 immunotherapy. Such a method, in some examples, may comprise (i) determining the level of Galectin-9 in multiple biological samples (two or more) from a ocular melanoma patient who is on an anti-cancer therapy at multiple time points, at least one of which is before the treatment and at least one of which is during or after the treatment; (ii) comparing the level of Gal-9 in a later collected biological sample with that in an earlier collected biological sample. If levels of Galectin-9 decreases after the treatment or over the course of the treatment, it indicates that the patient is responsive to the anti-cancer therapy. In some instances, the multiple biological samples are blood samples such as plasma samples or serum samples. In other instances, the multiple biological samples are biopsy or biopsy derived samples, e.g., PDOTs or other tissue samples such as tumor tissue samples.

In some examples, the multiple biological samples comprise one or more first biological samples collected before the first dose of the anti-cancer therapy (e.g., comprising an anti-galectin-9 antibody such as those disclosed herein) and one or more second biological samples collected after the first dose of the anti-cancer therapy. At least one of the first biological samples may be collected within 48 hours before the first dose of the anti-cancer therapy (e.g., within 36 hours, within 24 hours, within 18 hours, within 12 hours, or within 6 hours). Alternatively or in addition, the one or more second biological samples can be collected within a suitable period after the first dose (e.g., within 6 hours, 12 hours, 24 hours, 48 hours, or 72 hours after the first dose) and at suitable time points afterwards with suitable intervals (e.g., every 48 hours, every 72 hours, every 7 days, every 14 days, every 2 weeks, or every one month). In some embodiments, the intervals are once every 3 months, once every 6 months, or once a year. In some examples, multiple doses of the anti-cancer therapy such as anti-galectin 9 antibody may be given to the patient. In that case, a biological sample (e.g., a blood sample) may be collected from the patient after each dose. Selection of the suitable time points for collecting biological samples from the patients for assessing treatment responsiveness is within the knowledge of a medical practitioner.

The levels of Gal-9 in each of the biological samples can be measured using conventional methods or methods disclosed herein, e.g., using an assay comprising an anti-galectin 9 antibody. A decrease of Gal-9 levels in samples collected after the treatment as compared with the Gal-9 level in samples collected before the treatment is indicative of responsiveness to the treatment. Alternatively or in addition, a trend of decrease in Gal-9 levels over the course of treatment is also indicative of responsiveness to the treatment. In some embodiments, other diagnostic tests known in the art can be used in conjunction with the methods disclosed herein to confirm therapeutic effectiveness of the anti-cancer therapy.

The results regarding responsiveness to the anti-cancer therapy such as anti-galectin 9 antibody therapy can be relied on to determine treatment approaches to the patients. For example, if the patient is responsive to the anti-cancer therapy, such a therapy can be continued. Thus, any of the methods for assessing drug responsiveness may further comprise continuing the anti-cancer therapy, for example, administering an effective amount of an anti-galectin 9 antibody (e.g., those disclosed herein) to the subject. In some examples, doses and/or frequencies of the treatment may be decreased when a trend of Gal-9 decrease is observed.

On the other hand, if the patient is not responsive to the anti-cancer therapy, then the methods may further comprise adjusting or modifying treatment regimens applied to the patient. For example, doses and/or frequencies of the treatment may be increased, additional therapy may be applied, or the current anti-cancer therapy may be switched to a different one.

In specific examples, a method for measuring a response to a treatment in a cancer patient may comprise: (i) providing a biological sample e.g., blood plasma, serum, biopsy or biopsy derived samples, e.g., PDOTs or other tissue sample, of a subject having a cancer, (ii) measuring the level of galectin-9 in the biological sample, and (iii) determining the response to treatment in a subject based on the level of galectin-9 in the biological sample, wherein a reduced level of galectin-9 in the biological sample of the subject relative to a control (e.g., gal-9 level in a biological sample collected from the same patient before the treatment) indicates that the subject has responded to the treatment. In some embodiments, the control comprises a biological sample previously collected from the subject. Accordingly, in some embodiments, the method comprises (i) providing a first biological sample of a subject having a cancer, (ii) measuring the level of galectin-9 in the first biological sample e.g., blood plasma, serum, biopsy or biopsy derived samples, e.g., PDOTs or other tissue sample, (iii) administering one or more anti-cancer therapy(ies) over an amount of time (iv) providing a second biological sample, biological sample e.g., blood plasma, serum, biopsy or biopsy derived samples, e.g., PDOTs or other tissue, of the subject having a cancer, (v) measuring the level of galectin-9 in the second biological sample, and (vi) determining the response to treatment in a subject based on the difference in the level of galectin-9 between the first biological sample and the second biological sample, wherein a reduced level of galectin-9 in the second biological sample of the subject relative to the first biological sample indicates that the subject has responded to the treatment.

In other examples, provided herein are methods for preparing a treatment regimen in a cancer patient. In some embodiments, the methods comprise (i) providing a biological sample e.g., blood plasma, serum, biopsy or biopsy derived samples, e.g., PDOTs or other tissue sample, of a subject having a cancer, (ii) measuring the level of galectin-9 in the biological sample, and (iii) preparing a treatment regimen in a subject based on the level of galectin-9 in the biological sample, wherein the aggressiveness of the treatment regimen, e.g., dosage and/or frequency of dosage, correlates with the level of galectin-9 in the biological sample of the subject relative to a control, such that the higher the galectin-9 levels in the biological sample relative to control, the greater the aggressiveness of the treatment regimen.

In yet other examples, provided herein are methods for predicting the degree of success in the treatment of a cancer patient. In some embodiments, provided herein are methods for predicting the degree of success in the treatment of a cancer patient to anti-galectin-9 immunotherapy. Accordingly, in some embodiments, the methods comprise (i) determining the level of Galectin-9 in a biological sample, e.g., blood plasma, serum, biopsy or biopsy derived samples, e.g., PDOTs or other tissue, from the patient; (ii) comparing the level of Gal-9 in the biological sample of the cancer patient with a control level, wherein an elevated level of galectin-9 in the biological sample of the cancer patient relative to a control predicts a lower degree of success in the treatment of the cancer patient. In some embodiments, the level of gal-9 on TILs is measured. In some embodiments, the method further comprises (iii) treating the cancer patient. In some embodiments, the treatment is an anti-Galectin-9 immunotherapy. In some embodiments, the anti-galectin-9 antibody is any of the anti-galectin-9 antibodies provided herein. In some embodiments, the control level corresponds to the levels observed in blood or tissue from a healthy subject. In some embodiments, the control level corresponds to the levels observed in tumor adjacent healthy tissue of the subject having cancer. In some embodiments, other diagnostic tests known in the art are used in conjunction with the method.

In addition, provided herein are methods for predicting the degree of success of treatment of a cancer patient. In some embodiments, provided herein are methods for predicting the degree of success in the treatment of a cancer patient to anti-galectin-9 immunotherapy. Accordingly, in some embodiments, the methods comprise (i) using an anti-galectin-9 antibody to determine the level of Galectin-9 in a biological sample, e.g., blood plasma, serum, biopsy or biopsy derived samples, e.g., PDOTs or tissue, from the patient; (ii) comparing the level of Gal-9 in the biological sample of the cancer patient with a control level, wherein an elevated level of galectin-9 in the biological sample of the cancer patient relative to a control predicts a lower degree of success in the treatment of the cancer patient. In some embodiments, the level of gal-9 on TILs is measured. In some embodiments, the method further comprises (iii) treating the cancer patient. In some embodiments, the treatment is an anti-Galectin-9 immunotherapy. In some embodiments, the anti-galectin-9 antibody is any of the anti-galectin-9 antibodies provided herein. In some embodiments, the control level corresponds to the levels observed in blood or tissue from a healthy subject. In some embodiments, the control level corresponds to the levels observed in tumor adjacent healthy tissue of the subject having cancer. In some embodiments, other diagnostic tests known in the art are used in conjunction with the method.

In some examples, Gal-9 is used as a sole biomarker in the methods disclosed herein. In other examples, Gal-9 is used in conjunction with PD-L1.

### E. Monitoring Disease Progression

In some embodiments, the present disclosure provides methods for monitoring disease progression in a ocular melanoma patient (e.g., a human patient) using Gal-9 as a biomarker. Such a method may comprise collecting multiple biological samples at multiple time points from the patient and measuring levels of Gal-9 in the biological samples. As used herein, multiple means at least two. Any of the biological samples disclosed herein can be collected from the patient. Examples include blood samples such as serum samples or plasma samples, or biopsy and biopsy-derived samples such as tumor tissue samples or PDOT samples. The levels of Gal-9 can be measured by conventional methods or those disclosed herein, for example, an immunoassay. Disease progression can be determined based on changes of the Gal-9 levels as measured over time. For example, an increase of Gal-9 levels over time may be indicative of disease progression, while a decrease of Gal-9 levels over time may be indicative of improvement.

Based on the status of disease progression as determined by any of the methods disclosed herein, a suitable treatment regimen may be selected, which is within the knowledge of a medical practitioner. Thus, in some embodiments, the methods may further comprise selecting an anti-cancer therapy based on the progression of ocular melanoma in the subject as determined, and applying the anti-cancer therapy to the subject for treating the cancer. In some examples, the anti-cancer therapy may comprise an anti-galectin 9 antibody such as those disclosed herein. When needed, one or more additional diagnostic assays may be performed in any of the methods disclosed herein to confirm status of the cancer in the patient.

In some examples, Gal-9 is used as a sole biomarker in the methods disclosed herein. In other examples, Gal-9 is used in conjunction with PD-L1.

### F. Determination of Survival Rate

In another aspect, provided herein are methods for predicting the survival rate and/or the length of cancer-free survival time of a cancer patient. Accordingly, in some embodiments, the methods comprise (i) determining the level of Galectin-9 in a biological sample, e.g., blood plasma, serum, biopsy or biopsy derived samples, e.g., PDOTs or other tissue, from the cancer patient; (ii) comparing the level of Gal-9 in the biological sample of the cancer patient with a control level, wherein an elevated level of galectin-9 in the biological sample of the cancer patient relative to a control predicts a lower survival rate and/or a shorter length of cancer-free survival time of the cancer patient. In some embodiments, the level of gal-9 on TILs is measured. In some embodiments, the method further comprises (iii) treating the cancer patient. In some embodiments, the treatment is an anti-Galectin-9 immunotherapy. In some embodiments, the anti-galectin-9 antibody is any of the anti-galectin-9 antibodies provided herein. In some embodiments, the cancer patient in step (i) has cancer. In some embodiments, the cancer patient in step (i) has cancer and is being treated for said cancer. In some embodiments, the cancer patient in step (i) is cancer free at the time of determining the level of Gal-9. In some embodiments, the control level corresponds to the levels observed in blood or tissue from a healthy subject. In some embodiments, the control level corresponds to the levels observed in tumor adjacent healthy tissue of the subject having cancer. In some embodiments, other diagnostic tests known in the art are used in conjunction with the method.

In another aspect, provided herein are methods for predicting the survival rate or the length of cancer-free survival time of a cancer patient. Accordingly, in some embodiments, the methods comprise (i) using an anti-galectin-9 antibody to determine the level of Galectin-9 in a biological sample, e.g., blood plasma, serum, biopsy or biopsy derived samples, e.g., PDOTs or tissue, from the patient; (ii) comparing the level of Gal-9 in the biological sample of the cancer patient with a control level, wherein an elevated level of galectin-9 in the biological sample of the cancer patient relative to a control predicts a lower survival rate and/or a shorter length of cancer-free survival time of the cancer patient. In some embodiments, the level of gal-9 on TILs is measured. In some embodiments, the method further comprises (iii) treating the cancer patient. In some embodiments, the treatment is an anti-Galectin-9 immunotherapy. In some embodiments, the anti-galectin-9 antibody is any of the anti-galectin-9 antibodies provided herein. In some embodiments, the cancer patient in step (i) has cancer. In some embodiments, the cancer patient in step (i) has cancer and is being treated for said cancer. In some embodiments, the cancer patient in step (i) is cancer free at the time of determining the level of Gal-9.

In some embodiments, the control level corresponds to the levels observed in blood or tissue from a healthy subject. In some embodiments, the control level corresponds to the levels observed in tumor adjacent healthy tissue of the subject having cancer. In some embodiments, other diagnostic tests known in the art are used in conjunction with the method.

In some embodiments, the present disclosure features a method for assessing survival rate of a cancer patient (e.g., a ocular melanoma patient), the method comprising: (i) obtaining a tumor tissue sample (e.g., a ocular tumor sample) from a human cancer patient; (ii) determining PD-L1 presence in the tumor sample; (iii) measuring the level of galectin-9 in the tumor sample; and (iv) assessing survival rate of the cancer patient based on the PD-L1 presence and the level of galectin-9 in the tumor sample.

A survival rate refers to the likelihood of a cancer patient (e.g., an ocular melanoma patient) who would alive for a given period of time after diagnosis (e.g., within 5 years). Survival rate can be used as yardstick for the assessment of standards of therapy. The survival rate disclosed herein can be disease-free survival rate at five years (DFS_5), ocular melanoma specific survival rate at five years (OCSS_5), or distant metastases free survival at 5 years (DMFS_5).

DFS_5 rate refers to the likelihood of staying free of cancer 5 years after a particular treatment. A poor DFS_5 rate indicates a low likelihood that a ocular melanoma patient could stay free of cancer within 5 years after diagnosis or after a treatment. On the other hand, a patient having a good DFS_5 rate indicates that the patient is more likely to stay free of cancer within five years after diagnosis or after a treatment.

OCSS 5 rate refers to the likelihood of survival from ocular melanoma 5 years after diagnosis or after a treatment. A poor OCOCSS_5 rate indicates a low likelihood that a ocular melanoma patient could survive from ocular melanoma within 5 years after diagnosis or after a treatment. On the other hand, a patient having a good OCSS_5 rate indicates that the patient is more likely to survive from ocular melanoma within five years after diagnosis or after a treatment.

DMFS 5 rate refers to the likelihood of free of cancer that spreads from the original tumor to a distant organ or distant lymph node (distant metastases) in five years after diagnosis or after a treatment. A poor DMFS_5 rate indicates a low likelihood that a ocular melanoma patient could be free of distant metastases within 5 years after diagnosis or after a treatment. On the other hand, a patient having a good DMFS_5 rate indicates that the patient is more likely to be free of distant metastases within five years after diagnosis or after a treatment. In some instances, the survival rate is ocular melanoma specific survival at five years (OCSS_5).

A control level used in assessing a survival rate may be a pre-determined value or a range of values representing the level of Gal-9 in ocular melanoma patients having a determined survival rate. In some instances, the control level may be a cutoff value or a range of values distinguishing the levels of Gal-9 in patients having a poor survival rate relative to patients having a good survival rate. Good or poor survival rates can be determined based on standards in medical practices as known to those medical practitioners.

Assessment of potential survival rate of a ocular melanoma patient would help determine proper treatment of the patient. For example, when a ocular melanoma patient is determined to have a bad survival rate, a more aggressive treatment (e.g., involving multiple therapeutic agents or multiple types of treatment, or high dose of therapeutic agents) may be selected for treating that patient.

### III. Kit for Cancer Treatment or Diagnosis

The present disclosure also provides kits for use in any of the treatment, diagnostic and/or prognostic methods disclosed herein.

### Diagnostic Prognostic Kits

In some embodiments, the kits disclosed herein comprise one or more agents for measuring levels of galectin-9 in biological samples. Such agents can be antibodies specific to galectin-9. Alternatively, the agents may be nucleic acids for measuring mRNA levels of galectin-9 in a biological sample. The kit may further comprise reagents or devices for collecting and processing biological samples, and optionally containers for placing the biological samples. In some examples, the kit may further comprise one or more therapeutic agents for treating ocular melanoma, for example, any of the anti-galectin 9 antibodies as disclosed herein.

Any of the kits disclosed herein may comprise one or more containers for placing the one or more detection agents and optionally reagents and/or therapeutic agents. In some embodiments, the kit can comprise instructions for use in accordance with any of the methods described herein. The included instructions can comprise a description of collecting biological samples, processing such, and measuring Gal-9 levels in such biological samples. In addition, the included instructions may further comprise descriptions for identifying ocular melanoma patients, determining their tumor burden and/or tumor status, disease progression levels, responsiveness to a currently treatment, and/or potential survival rates according to any of the methods disclosed herein. Further, the instructions may comprise descriptions of selecting suitable treatment and how to apply such a treatment to the patient. Instructions supplied in the kits disclosed herein are typically written instructions on a label or package insert (e.g., a paper sheet included in the kit), but machine-readable instructions (e.g., instructions carried on a magnetic or optical storage disk) are also acceptable.

The kits disclosed herein are in suitable packaging. Suitable packaging includes, but is not limited to, vials, bottles, jars, flexible packaging (e.g., sealed Mylar or plastic bags), and the like. Any of the kits may optionally provide additional components such as buffers and interpretive information. Normally, the kit comprises a container and a label or package insert(s) on or associated with the container.

### Kits for Treating Ocular Melanoma

In other embodiments, the kit disclosed herein may include one or more containers comprising an anti-Galectin-9 antibody, e.g., any of those described herein, and optionally a second therapeutic agent (e.g., a checkpoint inhibitor such as an anti-PD-1 antibody as disclosed herein) or the chemotherapeutics to be co-used with the anti-Galectin-9 antibody, which is also described herein.

In some embodiments, the kit can comprise instructions for use in accordance with any of the methods described herein. The included instructions can comprise a description of administration of the anti-Galectin-9 antibody, and optionally the second therapeutic agent, to treat, delay the onset, or alleviate a target disease as those described herein. In some embodiments, the kit further comprises a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-Galectin-9 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the target tumor disclosed herein. In some embodiments, instructions are provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. In some embodiments, a kit has a sterile access port (for example the container may be an intravenous solution bag or a vial having a stopper pierceable by a hypodermic injection needle). In some embodiments, the container also has a sterile access port (for example the container is an intravenous solution bag or a vial having a stopper pierceable by a hypodermic injection needle). At least one active agent in the composition is an anti-Galectin-9 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention are employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Assessment of Galectin-9 Expression in Tumor Biopsy-Derived Organoid Fractions

Biopsy derived organoids can be used as a proxy to assess levels of Galectin-9 in the original tumor. Accordingly, the ability to assess Galectin-9 levels in single cell or organoid fractions was tested.

Biopsies were received from representative pancreatic adenocarcinoma and colorectal cancers and processed as follows. Human surgically resected tumor specimens were received fresh in DMEM media on ice and minced in 10 cm dishes. Minced tumors were resuspended in DMEM+10% FBS with 100 U/mL collagenase type IV to obtain spheroids. Partially digested samples were pelleted and then re-suspended in fresh DMEM+10% FBS and strained over both 100 mm and 40 mm filters to generate S1 (>100 mm), S2 (40-100 mm), and S3 (<40 mm) spheroid fractions, which were subsequently maintained in ultra-low-attachment tissue culture plates.

S2 fractions were digested by trypsin for 15 mins to generate into single cells. For flow cytometry preparation, cell pellets from S2 and S3 fractions were re-suspended and cell labeling was performed after Fc receptor blocking ( #422301; BioLegend, San Diego, CA) by incubating cells with fluorescently conjugated mAbs directed against human CD45 (HI30), CD3 (UCHT1), CD11b (M1/70), Epcam (9C4) and Gal9 (9M1-3; all Biolegend) or Gal9 Fab of G9.2-17 or Fab isotype. Dead cells were excluded from analysis using zombie yellow (BioLegend). Flow cytometry was carried out on the Attune NxT flow cytometer (Thermo Scientific). Data were analyzed using FlowJo v.10.1 (Treestar, Ashland, OR).

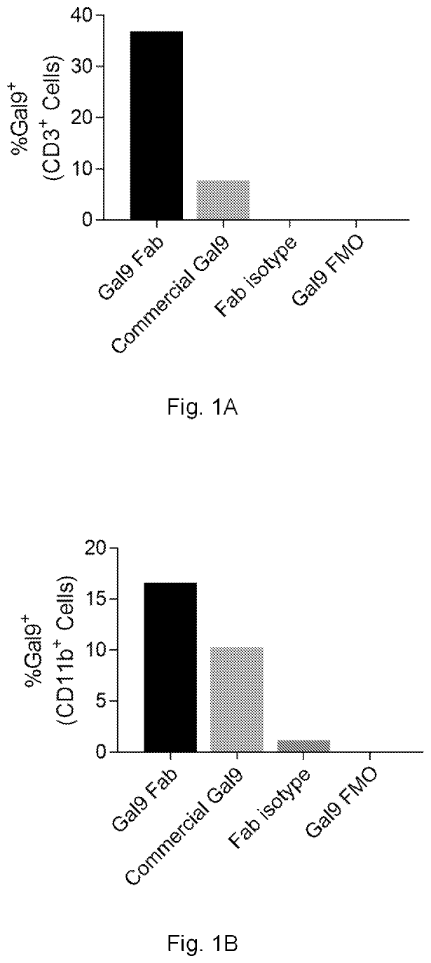

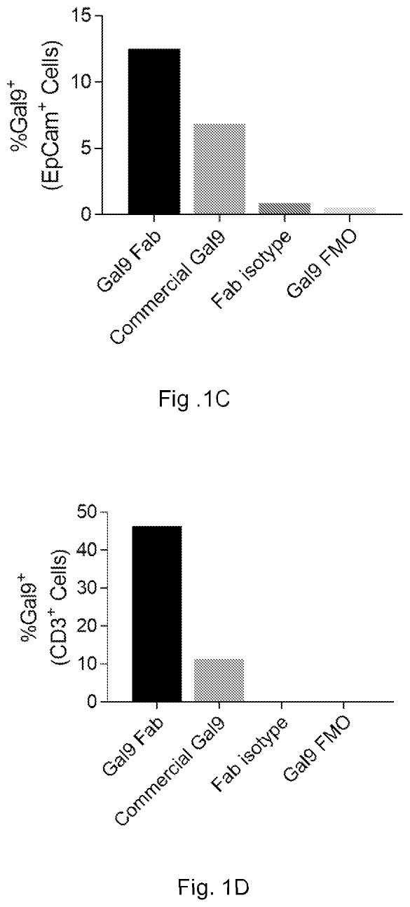

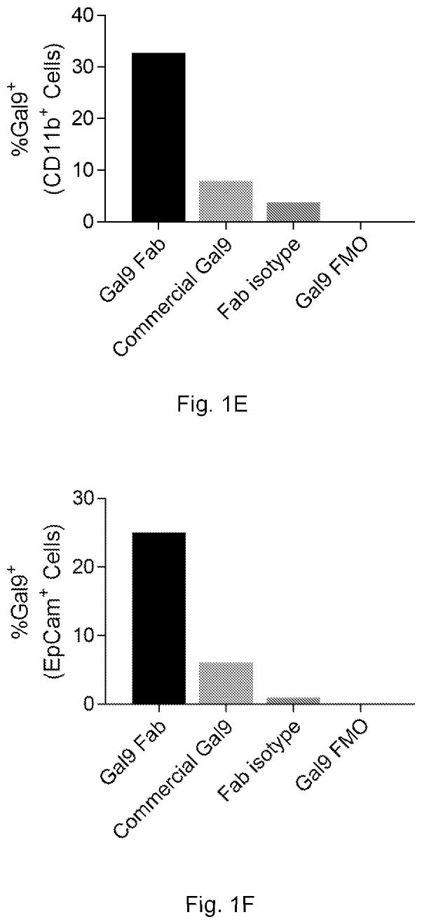

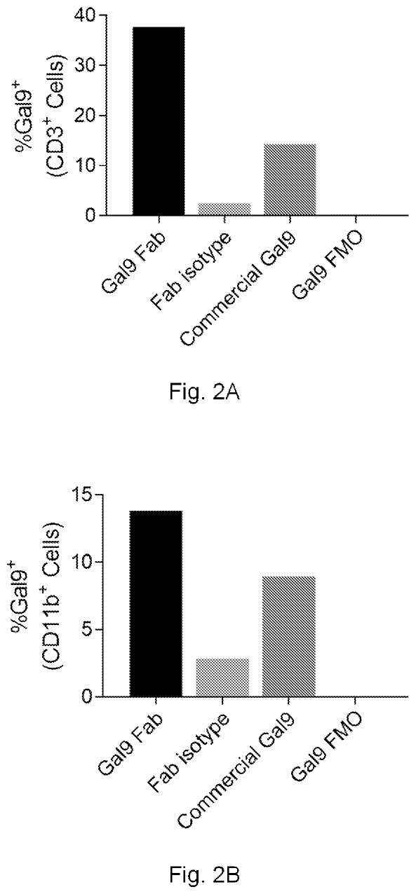

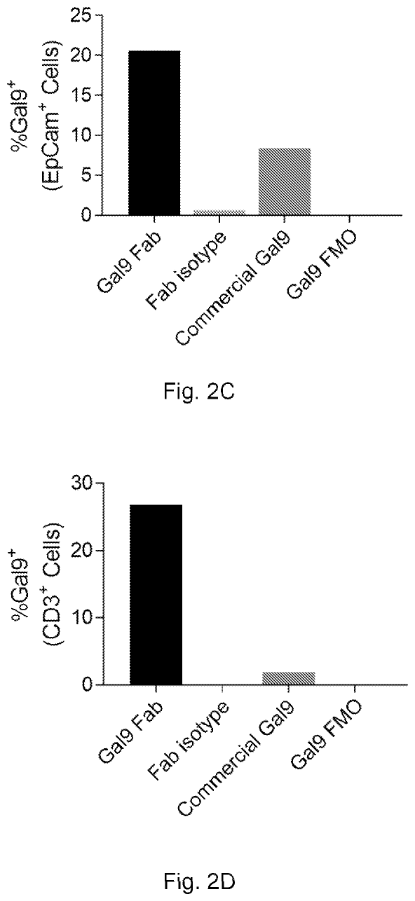

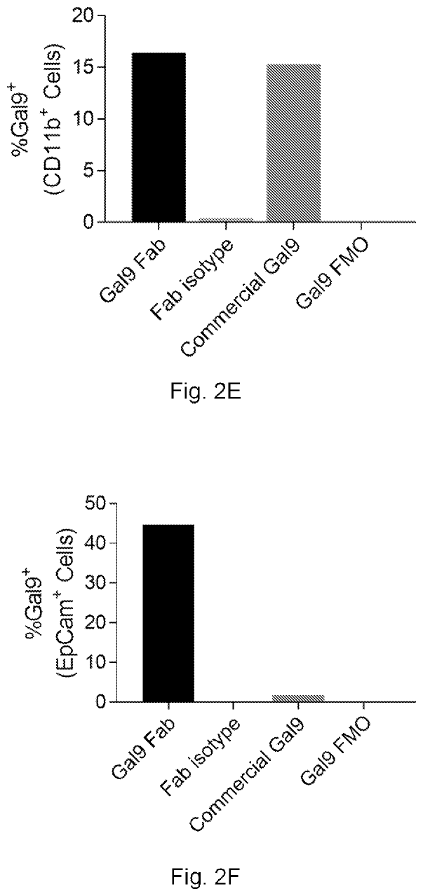

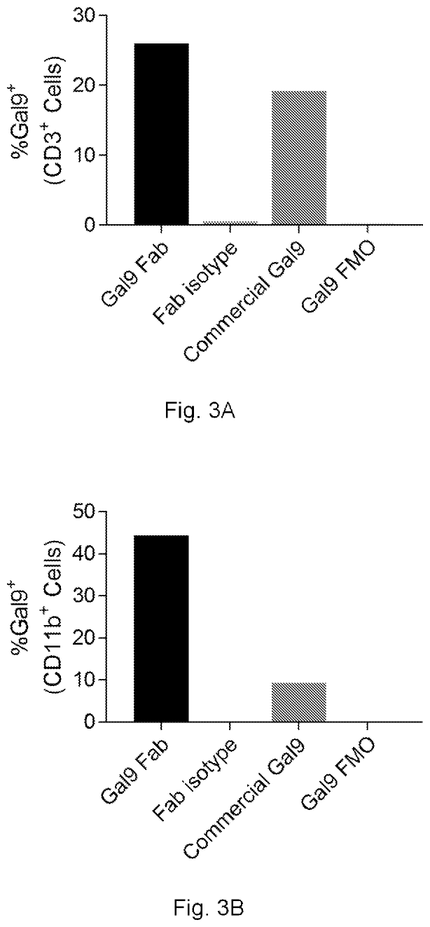

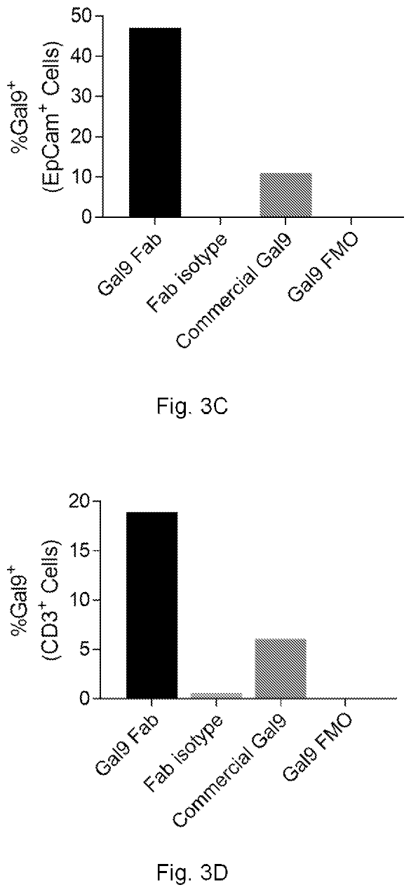

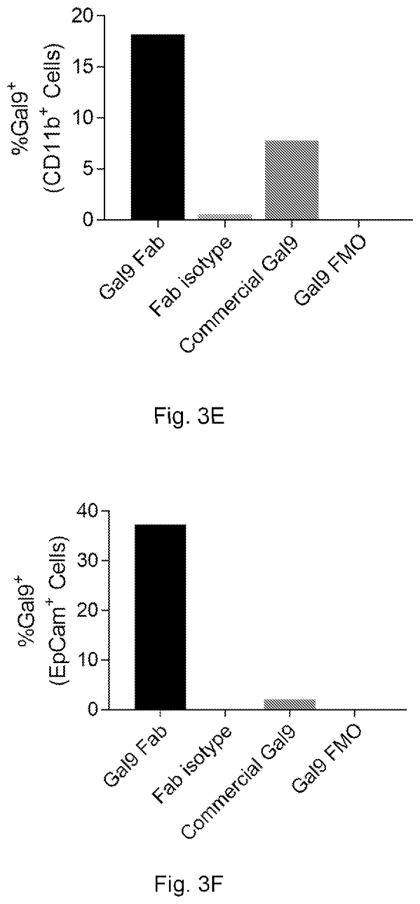

Results are shown in FIGS. 1A-1F, 2A-2F and 3A-3F and indicate that levels of Galectin-9 detected by the Gal9 G9.2-17 Fab in S2 single cell and S3 organoid fractions correlate. Accordingly, both S2 single cells and S3 organoids can be used for assessment of Galectin-9 levels in organoids derived from tumor biopsies.

Additionally, pancreatic adenocarcinoma (PDA), colorectal cancer (CRC), and hepatocellular carcinoma (HCC) tumors were processed as described above. The table below indicates that both S2 single cells and S3 organoids can be used for assessment of galectin-9 levels in organoids derived from tumor biopsies.

TABLE 7
Gal9 expression on EPCAM+ tumor cells

| | S2 Single Cell Fractions | | S3 Organoid Fractions | |
|---|---|---|---|---|
| | % Gal9 | % Gal9 | % Gal9 | % Gal9 |
| | expression based | expression based | expression based | expression based |
| | on Gal9 Fab of | on Gal9 Fab | on Gal9 Fab of | on Gal9 Fab |
| Tumor type | G9.2-17 staining | isotype staining | G9.2-17 staining | isotype staining |
| PDA | 12.5 | 0.88 | 25 | 0.94 |
| CRC liver | 33.5 | 0.15 | 42.2 | 0.06 |
| metastasis | | | | |
| Liver NET | 13.8 | 0.45 | 64.3 | 0.02 |
| CRC | 20.6 | 0.64 | 44.7 | 0.21 |
| PDA | 47 | 0.15 | 37.2 | 0.17 |
| Pancreas NET | 15.8 | 2.41 | 12.3 | 4.19 |
| CRC | 9.15 | 3.97 | 2.9 | 2.27 |
| CRC | | | 4.58 | 0.55 |
| HCC | | | 49.8 | 5.84 |

Galectin-9 acts as a potent mediator of cancer-associated immunosuppression and is expressed on tumor-associated macrophages, as well as intra-tumoral immunosuppressive gamma delta T cells. Table 7 shows galectin-9 expression on macrophages as seen in both S2 single cells and S3 organoids and Table 8 shows galectin-9 expression on T cells as seen in both S2 single cells and S3 organoids. Table 9 shows expression of the delta 1 chain of a T cell receptor in S2 single cells and S3 organoids as detected by a delta 1 Fab and Fab isotype.

TABLE 8
Galectin-9 expression on CD11b+ myeloid cells

| | S2 Single Cell Fractions | | S3 Organoid Fractions | |
|---|---|---|---|---|
| | % Gal9 | % Gal9 | % Gal9 | % Gal9 |
| | expression based | expression based | expression based | expression based |
| | on Gal9 Fab of | on Gal9 Fab | on Gal9 Fab of | on Gal9 Fab |
| Tumor type | G9.2-17 staining | isotype staining | G9.2-17 staining | isotype staining |
| PDA | 16.6 | 1.19 | 32.8 | 3.83 |
| CRC liver | 45.9 | 1.2 | 49.8 | 1.5 |
| metastasis | | | | |
| Liver NET | | | | |
| CRC | 13.8 | 2.86 | 16.4 | 0.38 |
| PDA | 44.4 | 0 | 18.2 | 0.54 |
| Pancreas NET | 45.2 | 16.2 | 34.3 | 1.87 |
| CRC | 15 | 5.84 | 15.6 | 11.8 |
| CRC | | | 6.16 | 2.4 |
| HCC | | | 26.2 | 1.72 |

TABLE 9
Galectin-9 expression on CD3+ T cells

| | S2 Single Cell Fractions | | S3 Organoid Fractions | |
|---|---|---|---|---|
| | % Gal9 | % Gal9 | % Gal9 | % Gal9 |
| | expression based | expression based | expression based | expression based |
| | on Gal9 Fab of | on Gal9 Fab | on Gal9 Fab of | on Gal9 Fab |
| Tumor type | G9.2-17 staining | isotype staining | G9.2-17 staining | isotype staining |
| PDA | 36.9 | 0 | 46.3 | 0 |
| CRC liver | 31.1 | 0.56 | 34.2 | 0 |
| metastasis | | | | |
| Liver NET | | | | |
| CRC | 37.7 | 2.5 | 26.8 | 0 |
| PDA | 26 | 0.52 | 18.9 | 0.59 |
| Pancreas NET | 63.7 | 32.2 | 27.5 | 4.96 |
| CRC | 12.5 | 1.59 | 25 | 15.3 |
| CRC | | | 3.74 | 0.87 |
| HCC | | | 12.3 | 1.14 |
| Liver NET | 3.32 | 0.27 | 50.4 | 25.3 |
| CRC | 19.9 | 0.93 | 41.3 | 1.61 |
| PDA | 41.3 | 55 | 46.9 | 1.49 |
| PDA | 24.7 | 4.49 | 11.1 | 3.21 |
| Pancreas NET | 23 | 39.1 | 5.77 | 2.61 |
| CRC | 36.5 | 5.08 | 13.1 | 3.54 |
| CRC | | | 17.5 | 0.85 |
| CRC | | | 55.2 | 0.33 |
| CRC liver | | | 22.8 | 0.6 |
| metastasis | | | | |
| CRC | | | 33 | 0 |
| HCC | | | 35.1 | 0.3 |

The above results show that high levels of Galectin-9 were detected in patient blood/tissue samples compared with healthy controls. Analyses of PDOTs show high levels of Galectin-9 on tumor, myeloid and T cells, suggesting Galectin-9 as a promising disease specific target. Additionally, measurement of Galectin-9 levels in a tumor may constitute a means for determining a population of cancer patients that are likely to respond to anti-Galectin-9 therapy.

### Example 2. Preparation of Patient-Derived Organotypic Tumor Spheroids (PDOTs) for Cellular Analysis

Biopsy-derived organoids can be a useful measure to assess the ability of a therapeutic to stimulate an immune response. Accordingly, S2 fractions described in the previous Example 1 above used for ex vivo culture were treated with anti-Galectin-9 antibody G9.2-17and prepared for immune profiling.

An aliquot of the S2 fraction was pelleted and resuspended in type I rat tail collagen (Corning) at a concentration of 2.5 mg/mL following the addition of 10×PBS with phenol red with pH adjusted using NaOH. pH 7.0-7.5 is confirmed using PANPEHA Whatman paper (Sigma-Aldrich). The spheroid-collagen mixture is then injected into the center gel region of a 3-D microfluidic culture device as described in Jenkins et al., Cancer Discov. 2018 February;8(2): 196-215; Ex Vivo Profiling of PD-1 Blockade Using Organotypic Tumor Spheroids, the contents of which is herein incorporated by reference in its entirety. Collagen hydrogels containing patient-derived organotypic tumor spheroids (PDOTS) were hydrated with media with or without anti-Galectin-9 monoclonal antibody G9.2-17 after 30 minutes at 37° C. The PDOTS were then incubated at 37° C. for 3 days.

Cell pellets were re-suspended in the FACS buffer and 1×10⁶ cells were first stained with zombie yellow (BioLegend) to exclude dead cells. After viability staining, cells were incubated with an anti-CD16/CD32 mAb (eBiosciences, San Diego, CA) for blocking FcγRIII/II followed by antibody staining with 1 μg of fluorescently conjugated extracellular mAbs. Intracellular staining for cytokines and transcription factors was performed using the Fixation/Permeabilization Solution Kit (eBiosciences). Useful human flow cytometry antibodies included CD45 (HI30), CD3 (UCHT1), CD4 (A161A1), CD8 (HIT8a), CD44 (BJ18), TNFα (MAb11), IFNγ (4S.B3), and Epcam (9C4); all Biolegend. Flow cytometry was carried out on the LSR-II flow cytometer (BD Biosciences). Data were analyzed using FlowJo v.10.1 (Treestar, Ashland, OR).

### Example 3. Assessment of Galectin-9 Levels in Plasma and Serum of Cancer Patients

Plasma and serum Galectin-9 levels were assessed in patient samples and compared to healthy volunteers. Blood (10 ml) was drawn from peripheral venous access from 10 healthy controls and 10 inoperable cancer patients. Serum and plasma were extracted from each sample of blood. Blood was collected in standard EDTA tubes PicoKine™ ELISA; Catalog number: EK1113 was used essentially according to manufacturer's instructions. Results of individual values are tabulated in Table 10 and Table 11.

TABLE 10
Patient Samples

| | | Serum | Plasma |
|---|---|---|---|
| Cancer Type | Patient No. | (pg/ml) | (pg/ml) |
| Breast cancer with metastases in liver and bones | Patient 1 | 11362.29 | 12107.56 |
| Melanoma brain and lung metastases braf+ | Patient 2 | 978.97 | 1106.79 |
| Melanoma lung metastases braf− | Patient 3 | 838.83 | 695.08 |
| Rectal cancer with liver metastases | Patient 4 | 579.42 | 725.62 |
| Locally advanced gastric cancer | Patient 5 | 666.67 | 645.2 |
| Gastric cancer with liver, spleen and adrenal | Patient 6 | 674.3 | 877.69 |
| metastases | | | |
| Stage III ovarian cancer | Patient 7 | 1439.61 | 1341.6 |
| Metastatic cancer of unknown primary | Patient 8 | 1432.39 | 1671.8 |
| Testicular cancer | Patient 9 | 1352.56 | 1696.11 |
| Sarcoma | Patient 10 | 968.18 | 1073.57 |
| | Average | 2029.322 | 2194.102 |

TABLE 11
Healthy Volunteer Samples

| Sample Number | Serum | Plasma |
|---|---|---|
| Control 1 | 536.4 | 611.97 |
| Control 2 | 476.43 | 592.58 |
| Control 3 | 612.66 | 651.43 |
| Control 4 | 269.75 | 414.41 |
| Control 5 | 460.26 | 602.28 |
| Control 6 | 206.66 | 405.8 |
| Control 7 | 385.88 | 439.85 |
| Control 8 | 525.283 | 654.2 |
| Control 9 | 711.047 | 718.68 |
| Control 10 | 296.85 | 349.09 |
| Average | 448.122 | 544.029 |

The above results show that high levels of Galectin-9 were detected in the plasma and serum samples of cancer patients (e.g., breast cancer patient, melanoma patient, ovarian cancer patient, testicular cancer patient, and sarcoma patient) as compared with healthy controls.

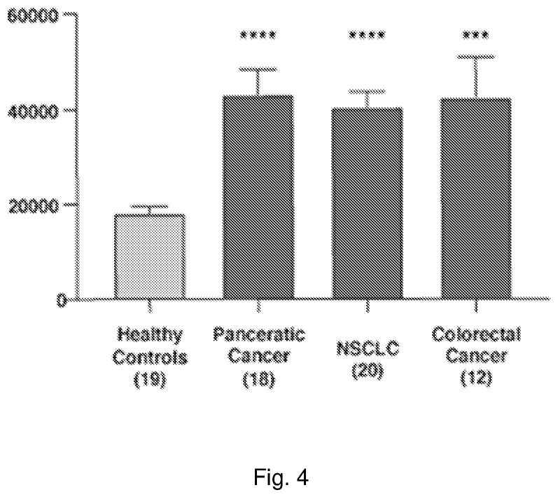

Follow on studies using the same procedures described above for serum measurements for in a larger number of healthy controls (19 controls) and subjects (18 subjects with pancreatic cancer, 20 subjects with non-small cell lung cancer (NSCLC), and 12 subjects with colorectal cancer) are shown in FIG. 4. Briefly, Galectin-9 levels in patient sera (sample size in parentheses) was measured using enzyme-linked immunosorbant assay (ELISA). The ELISA was run in sandwich format using the Human Galectin-9 PicoKine ELISA kit (Bolster Biological Technology). Patient serum galectin-9 levels were compared to healthy human serum controls. Statistical analysis performed by unpaired Student's t-test. (*p<0.05; **p<0.01; ***p<0.001; ****p<0.0001) Results show Galectin-9 levels in serum is significantly increased in cancer patients. Table 12 provides a values for the individual patient samples and Table 13 provides the average values.

TABLE 12
Blood Levels of Galectin-9 in Healthy
Controls and Cancer Patients

| Healthy Controls | Panceratic Cancer | NSCLC | Colorectal Cancer |
|---|---|---|---|
| (Galectin-9 | (Galectin-9 | (Galectin-9 | (Galectin-9 |
| levels in | levels in | levels in | levels in |
| pg/ml) | pg/ml) | pg/ml) | pg/ml) |
| 22018 | 67781 | 63257 | 18146 |
| 15702 | 38064 | 19113 | 24952 |
| 13319 | 60664 | 21476 | 123670 |
| 13727 | 35114 | 45978 | 36430 |
| 15359 | 38212 | 51598 | 30573 |
| 14152 | 74782 | 24745 | 20868 |
| 10884 | 34932 | 40816 | 58977 |
| 15343 | 35196 | 37675 | 36385 |
| 8812 | 39558 | 40486 | 43970 |
| 17463 | 20916 | 78336 | 60134 |
| 27907 | 22917 | 48317 | 32573 |
| 15990 | 35654 | 30918 | 23227 |
| 14500 | 109239 | 25709 | |
| 32615 | 22223 | 42351 | |
| 21935 | 37523 | 22501 | |
| 18822 | 25310 | 42600 | |
| 20685 | 36620 | 43266 | |
| 25845 | 41730 | 41877 | |
| 19727 | | 33186 | |
| | | 54602 | |

TABLE 13
Average Blood Levels of Galectin-9 in Cancer
Patients Relative to Healthy Controls

| | Healthy | Pancreatic | | Colorectal |
|---|---|---|---|---|
| | Control | Cancer | NSCLC | Cancer |
| Average (pg/ml) | 18148 | 43135 | 40440 | 42492.08333 |
| Standard Deviation | 5968.8331 | 22139.526 | 14767.5495 | 28964.95828 |
| SEM | 1369.3442 | 5218.336262 | 3302.124452 | 8361.46323 |
| P-value | | 3.46372E−05 | | 0.001238666 |

Galectin-9 serum levels in patients with pancreatic cancer having various tumor burden are provided in Table 14 below:

TABLE 14
Correlation Between Galectin-9 Serum Levels and Tumor Burden

| | Galectin-9 | |
|---|---|---|
| Patient | serum levels | |
| Number | (pg/ml) | Tumor burden |
| 1 | 19774 | low tumor burden, only locally advanced |
| 2 | 67781 | high tumor burden, multiple liver mets |
| 3 | 60664 | high tumor burden, multiple liver mets |
| 4 | 74782 | low tumor burden, only locally advanced |
| 5 | 20916 | low tumor burden, only locally advanced |
| 6 | 109239 | high tumor burden, multiple liver mets and |
| | | lymphnode mets |
| 7 | 22223 | low tumor burden, only locally advanced |

### Example 4. Assessment of Galectin-9 Levels in Plasma and Serum of Cancer Patients

Plasma and serum Galectin-9 levels are assessed in metastatic uveal melanoma patient samples and compared to healthy volunteers. Blood (1ml-10 ml) is drawn from peripheral venous access from 10 healthy controls and 10 metastatic uveal melanoma cancer patients. Serum and plasma are extracted from each sample of blood. Blood is collected in standard EDTA tubes PicoKine™ ELISA; Catalog number: EK1113 was is essentially according to manufacturer's instructions.

### Example 5 Assessment of Galectin-9 Expression and Localization Using Immunohistochemical Analysis

The ability to use immunohistochemical analysis to determine Galectin-9 expression levels in tumors was assessed using paraffin-embedded biopsy-derived tumor samples.

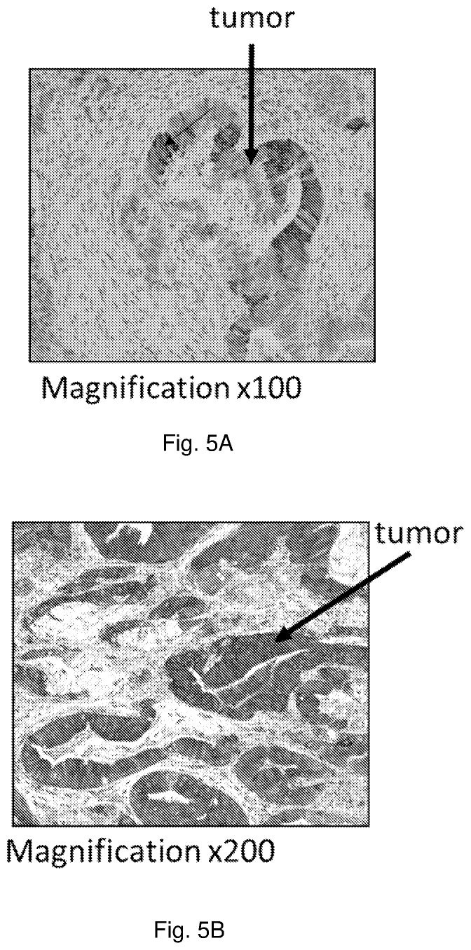

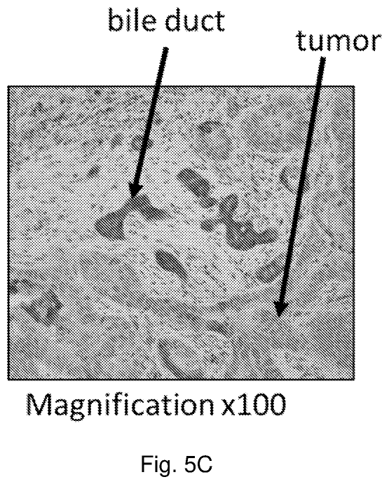

In brief, slides were deparaffinized (xylene: 2×3 min; absolute alcohol: 2×3 min., methanol: 1×3 min) and rinsed in cold tap water. For antigen retrieval, citrate buffer (pH 6) was preheated to 100 C in a water bath and slides were incubated in citrate buffer for 5 minutes. Slides were left to cool for about 10 min at room temperature and put in running water. Slides were washed in PBS, a pap pen circle was drawn around the section, and sections were incubated in blocking buffer (DAKO-Peroxidase blocking solution-S2023) for 5 minutes. Serum free blocker was added (Novocastra serum free Protein Blocker), and then rinsed off with PBS. Primary antibody (Sigma, anti-Galectin-9 clone 1G3) was used at 1:2000 dilution in DAKO-S2022 diluent and sections were incubated over night at 4C. Slides were washed with PBS and then incubated with the secondary antibody (anti-mouse) for 45 minutes at room temperature. Slides were washed and stained with ABC VECTOR STAIN (45 mins), washed with PBS, stained with DAB (1 ml stable DAB buffer+1 drop DAB)) for 5 minutes and washed in running water. Haematoxylin was added for 1 minute and 70% ETOH+1% HCL was applied to avoid over staining. Slides were left in running water for 2-3 min, then dipped in water, then absolute alcohol, and then xylene, 2 times for 30 seconds each. Cover slip and images were captured. Galectin-9 staining in a chemotherapy treated colorectal cancer and a liver metastasis of colorectal carcinoma are shown in FIGS. 5A and 5B. Results from Galectin-9 negative cholangiocarcinoma is shown in FIG. 5C.

### Example 6: Assessment of Galectin-9 Expression and Localization in Metastatic Uveal Melanoma Using Immunohistochemical Analysis

Samples from uveal melanoma liver metastases are prepared as described above and slides are assessed for galectin-9 levels.

### Example 7: Evaluation of Gal-9 Antibody in a B16F10 Melanoma Syngeneic Tumor Model in Immunocompetent Mice

Gal-9 antibody G9.2-17 was evaluated in the B16F10 syngeneic mouse model of melanoma immunocompetent mice. Pre-study animals (female C57BL/6, 6-8 weeks of age (Charles River Labs)) were unilaterally implanted subcutaneously on the left flank with 5e5 B16.F10 in 100 μl PBS. Pre-study tumor volumes were recorded for each experiment beginning 2-3 days after implantation. When tumors reached an average tumor volume of 50-100 mm³ (preferably 50-75 mm³) animals were matched by tumor volume into treatment or control groups (n=8) to be used for dosing and dosing was initiated on Day 0. Animals were dosed on day 0 and day 4 i.v. The study design for testing of Anti-Gal9 G9.2-17 IgG1 and Anti-Gal9 G9.2-17 IgG2 is summarized in Table 15.

TABLE 15
Anti-Gal9 IgG2

| | | | | | Route of | | |
|---|---|---|---|---|---|---|---|
| | | | | | Admin- | | Total |
| | | | Dose | Dose | istration | | Number |
| Group | -n- | Test Agent | (ug/mouse) | Volume | (ROA) | Schedule | of Doses |
| 1 | 10 | Control Untreated | — | — | — | — | — |
| 2 | 10 | Control mIgG2 | 200 ug | 200 ul | IV | Q4D × 6 | 6 |
| 3 | 10 | Control mIgG2 | 200 ug | 200 ul | IP | BIW × 4 | 8 |
| 4 | 10 | Anti-Gal9 mIgG2 | 200 ug | 200 ul | IV | Q4D × 6 | 6 |
| | | (G9.2-17) | | | | | |
| 5 | 10 | Anti-Gal9 mIgG2 + | 200 ug/ | 200 ul | IV | Q4D × 6 | 6 8 |
| | | mAnti-PD1 | 200 ug | 200 ul | IP | BIW × 4 | |
| 6 | 10 | mAnti-PD1 | 200 ug | 200 ul | IP | BIW × 4 | 8 |

Tumor volumes were taken and animals were weighed three times weekly. The study endpoint was set when the mean tumor volume of the control group (uncensored) reached 1500 mm³. A final tumor volume was taken on the day the study reached endpoint. A final weight was taken on the day the study reached end point (day 10). Tumor volume is shown in Table 30 and FIG. 31.

### Example 8: Evaluation of Gal-9 Antibody in aSyngeneic Models of Melanoma in Immunocompetent Mice

Gal-9 antibodies G9.2-17 and G9.1-8m13 are evaluated a syngeneic model melanoma in immunocompetent mice.

### Experimental Design

Pre-study animals (female C57BL/6, 6-8 weeks of age (Charles River Labs) are acclimatized for 3 days and then are unilaterally implanted subcutaneously on the left flank with 5e5 B16.F10 (melanoma cell line) resuspended in 100 μl PBS. Pre-study tumor volumes are recorded for each experiment beginning 2-3 days after implantation. When tumors reach an average tumor volume of 50-100 mm³ (preferably 50-75 mm³) animals are matched by tumor volume into treatment or control groups to be used for dosing and dosing initiated on Day 0. The study design for testing of Anti-Gal9 IgG1 is summarized in Table 16.

TABLE 16
Anti-Gal9 IgG1 (B16F10) Testing Conditions

| | | | | | Route of | | |
|---|---|---|---|---|---|---|---|
| | | | | | Admin- | | Total |
| | | | Dose | Dose | istration | | Number |
| Group | -n- | Test Agent | (ug/mouse) | Volume | (ROA) | Schedule | of Doses |
| 1 | 8 | Control Untreated | — | — | — | — | — |
| 2 | 8 | Control mIgG1 | 200 ug | 200 ul | IV | Q4D × 6 | 6 |
| 3 | 8 | Control mIgG1 | 400 ug | 200 ul | IV | Q4D × 6 | 6 |
| 4 | 8 | Control mIgG2 | 200 ug | 200 ul | IP | BIW × 4 | 8 |
| 5 | 8 | Anti-Gal9 mIgG1 | 200 ug | 200 ul | IV | Q4D × 6 | 6 |
| 6 | 8 | Anti-Gal9 mIgG1 | 400 ug | 200 ul | IV | Q4D × 6 | 6 |
| 9 | 8 | Anti-Gal9 mIgG1 + | 200 ug | 200 ul | IV IP | Q4D × 6 | 6 8 |
| | | mAnti-PD1 | 200 ug | 200 ul | | BIW × 4 | |
| 10 | 8 | Anti-Gal9 mIgG1 + | 400 ug | 200 ul | IV IP | Q4D × 6 | 6 8 |
| | | mAnti-PD1 | 200 ug | 200 ul | | BIW × 4 | |
| 13 | 8 | mAnti-PD1 | 200 ug | 200 ul | IP | BIW × 4 | 8 |

Tumor volumes are taken three times weekly. A final tumor volume is taken on the day the study reaches endpoint. A final tumor volume is taken if an animal is found moribund. Animals are weighed three times weekly. A final weight is taken on the day the study reaches end point or if animal is found moribund. Animals exhibiting ≥10% weight loss when compared to Day 0 are provided DietGel® ad libitum. Any animal exhibiting >20% net weight loss for a period lasting 7 days or if mice display >30% net weight loss when compared to Day 0 is considered moribund and is euthanized. The study endpoint is set when the mean tumor volume of the control group (uncensored) reaches 1500 mm3. If this occurs before Day 28, treatment groups and individual mice are dosed and measured up to Day 28. If the mean tumor volume of the control group (uncensored) does not reach 1500 mm3 by Day 28, then the endpoint for all animals is the day when the mean tumor volume of the control group (uncensored) reaches 1500 mm3 up to a maximum of Day 60. Blood is collected from all animals from each group. For blood collection, as much blood as possible is collected via a cardiac puncture into K₂EDTA tubes (400 μl) and serum separator tubes (remaining) under deep anesthesia induced by isoflurane inhalation. The blood collected into K₂EDTA tubes is placed on wet ice until used for performing immune panel flow as shown in Table 17.

TABLE 17
Flow Cytometry Panel 1

| Antibody Description | Conjugate | Clone | Supplier |
|---|---|---|---|
| mCD3 | FITC | 17A2 | BioLegend |
| mCD4 | APC-Fire 750 | RM4-4 | BioLegend |
| mGamma Delta | BV605 | GL3 | BioLegend |
| mCD8 | APC-R700 | 53-6.7 | BioLegend |
| mCD44 | BV786 | IM7 | BioLegend |
| mCD11b | APC | M1/70 | BioLegend |
| mCD45 | BV510 | 30-F11 | BioLegend |
| Live Dead | 7AAD | — | BioLegend |
| mCD62L | PE-Cy7 | MEL-14 | BioLegend |
| mPD-1 | BV711 | 29F.1A12 | BioLegend |
| mCTLA4 | PE | UC10-4B9 | BioLegend |
| mCD27 | BV421 | LG.3A10 | BioLegend |

Blood collected into serum separator tubes is allowed to clot at room temperature for at least 15 minutes. Samples are centrifuged at 3500 for 10 minutes at room temperature. The resultant serum is separated, transferred to uniquely labeled clear polypropylene tubes, and frozen immediately over dry ice or in a freezer set to maintain-80° C. until shipment for the bridging ADA assay (shipped within one week).

Tumors from all animals are collected as follows. Tumors less than 400 mm³ in size are snap frozen, placed on dry ice, and stored at −80 C until used for RT-qPCR analysis. For tumors of 400-500 mm³ in size, whole tumors are collected into MACS media for use in the Flow Panel (shown in Table 18 below). For tumors greater than 500 mm³ in size, a small piece (about 50 mm³) is snap frozen placed on dry ice, and stored at −80 C for RT-qPCR, and the remaining tumor is collected in MACS media for flow cytometry (as shown in Table 18). For flow cytometry, tumors are placed in MACS media and stored on wet ice until processed. A summary of the flow cytometry analysis performed is shown in Table 19.

TABLE 18
Flow cytometry Panel 2

| Antibody | | | |
|---|---|---|---|
| Description | Conjugate | Clone | Supplier |
| mCD3 | FITC | 17A2 | BioLegend |
| mCD4 | APC-Fire 750 | RM4-4 | BioLegend |
| mGamma | BV605 | GL3 | BioLegend |
| Delta | | | |
| mCD8 | APC-R700 | 53-6.7 | BioLegend |
| mCD69 | BV421 | H1.2F3 | BioLegend |
| mCD11b | APC | M1/70 | BioLegend |
| mCD45 | BV510 | 30-F11 | BioLegend |
| Live Dead | 7AAD | — | BioLegend |
| mCD62L | PE-Cy7 | MEL-14 | BioLegend |
| mPD-1 | BV711 | 29F.1A12 | BioLegend |
| mCTLA4 | PE | UC10-4B9 | BioLegend |
| mNk1.1 | BV786 | PK136 | BioLegend |

Spleen, liver, colon, lungs, heart, and kidneys from all animals are retained in 10% neutral buffered formalin (NBF) for 18-24 hours, transferred to 70% ethanol and stored at room temperature. Formalin fixed samples are paraffin embedded.

TABLE 20
Anti-Gal9 IgG2 Testing Conditions

| | | | | | Route of | | |
|---|---|---|---|---|---|---|---|
| | | | | | Admin- | | Total |
| | | | Dose | Dose | istration | | Number |
| Group | -n- | Test Agent | (ug/mouse) | Volume | (ROA) | Schedule | of Doses |
| 1 | 10 | Control Untreated | — | — | — | — | — |
| 2 | 10 | Control mIgG2 | 200 ug | 200 ul | IV | Q4D × 6 | 6 |
| 3 | 10 | Control mIgG2 | 200 ug | 200 ul | IP | BIW × 4 | 8 |
| 4 | 10 | Anti-Gal9 mIgG2 | 200 ug | 200 ul | IV | Q4D × 6 | 6 |
| | | (G9.2-17) | | | | | |
| 5 | 10 | Anti-Gal9 mIgG2 + | 200 ug/ | 200 ul | IV | Q4D × 6 | 6 8 |
| | | mAnti-PD1 | 200 ug | 200 ul | IP | BIW × 4 | |
| 6 | 10 | mAnti-PD1 | 200 ug | 200 ul | IP | BIW × 4 | 8 |

Tumor volumes are taken via CT scan and animals are weighed three times weekly. The study endpoint is set when the mean tumor volume of the control group (uncensored) reaches 1,500 mm3. A final tumor volume is taken on the day the study reached endpoint. A final weight was taken on the day the study reached end point.

CT scanning is performed to monitor and assess tumor volume. Micro-CT scan (Inveon Micro-CT, Siemens, Germany) is performed 4 h after injection of contrast agent. The contrast agent (ExiTron nano 12000, Miltenyi Biotec, Germany) is an alkaline earth-based nanoparticulate contrast agent for mouse liver CT imaging. Upon intravenous injection, the agent is taken up by cells of the reticuloendothelial system, including macrophages within the liver, termed Kupffer cells. Mice are injected with 100 μl of agent (per mouse, 25-30 g body weight) via a lateral tail vein.

PDX derived tumor masses are collected from the mice at the scheduled time of sacrifice. The tumor volume is calculated using the following formula: Volume=(W2×L)/2, with W being the shortest diameter and L being the longest diameter. Resected tumors are washed with sterile PBS. A portion of the tumor is cut into 1 mm cubes for implantation into the liver of recipient mice. The remaining tumor was used for analyses of tumor characteristics and cryopreservation.

Flow cytometry for inflammatory markers is conducted as described in the previous example.

### Example 9: Evaluation of Gal-9 Antibody in a Xenograft Uveal Melanoma Tumor Model in Immunocompetent Mice

Gal-9 antibody G9.2-17 is evaluated in the an orthotopic hepatic patient derived xenograft (PDX) model of metastatic uveal melanoma in immunocompetent mice, as described in Kageyama et al. (J Transl Med. 2017; 15: 145).

In brief, tumor specimens are obtained after surgery or biopsy from liver metastatic uveal melanoma patients. Tumor masses are washed with sterile PBS and are cut into 1 mm cubes for implantation into the mouse liver. Procurement of tumor specimens and tumor implantation are performed within 2 h.

Eight-week-old male and female NOD.Cg-Prkdcscid Il2rgtmlWjl/SzJ (NSG) mice (e.g., available from Jackson Laboratory, Bar Harbor, ME, USA) are used for tumor injection or implantation into the liver. For the tumor injection or implantation, each mouse is anesthetized with 3% Isoflurane for induction and 2% for maintenance.

For laparotomy, mice are placed on a heating pad in the supine position. A 1 cm skin incision is made in the left subcostal area, followed by a 1 cm incision in the peritoneum to expose the liver. Using a cotton swab, the left lobe of the liver is moved outside the body and placed on a nonwoven absorbent fabric sheet for implantation. The liver is incised using a No. 11 sharp scalpel (AD Surgical, Sunnyvale, CA, USA) horizontally in parallel with the surface of the liver to form a pocket in the parenchyma without cutting any major vessels. A tumor piece is implanted into the pocket. The incision site is then sealed with absorbable hemostatic material (SURGICEL, Johnson and Johnson, New Brunswick, NJ, USA) to curtail bleeding. After the surgical implantation, the liver is returned within the body, and the abdominal incision is closed in 2 layers with 5-0 polydioxanone absorbable thread (AD Surgical, Sunnyvale, CA, USA).

Pre-study NSG mice 3, 4 or 8 weeks post tumor implantation or when tumors reached an average tumor volume, for example of 50-100 mm³ as measured by CT scan, are matched by tumor volume into treatment or control groups (n=10) to be used for dosing and dosing is initiated on Day 0. Animals are dosed on day 0 and day 4 i.v. The study design for testing of

Anti-Gal9 G9.2-17 IgG1 is summarized in Table 21.

TABLE 21
Anti-Gal9 IgG2 Testing Conditions

| | | | | | Route of | | |
|---|---|---|---|---|---|---|---|
| | | | | | Admin- | | Total |
| | | | Dose | Dose | istration | | Number |
| Group | -n- | Test Agent | (ug/mouse) | Volume | (ROA) | Schedule | of Doses |
| 1 | 10 | Control Untreated | — | — | — | — | — |
| 2 | 10 | Control mIgG2 | 200 ug | 200 ul | IV | Q4D × 6 | 6 |
| 3 | 10 | Control mIgG2 | 200 ug | 200 ul | IP | BIW × 4 | 8 |
| 4 | 10 | Anti-Gal9 mIgG2 | 200 ug | 200 ul | IV | Q4D × 6 | 6 |
| | | (G9.2-17) | | | | | |
| 5 | 10 | Anti-Gal9 mIgG2 + | 200 ug/ | 200 ul | IV | Q4D × 6 | 6 8 |
| | | mAnti-PD1 | 200 ug | 200 ul | IP | BIW × 4 | |
| 6 | 10 | mAnti-PD1 | 200 ug | 200 ul | IP | BIW × 4 | 8 |

Tumor volumes are taken via CT scan and animals are weighed three times weekly. The study endpoint is set when the mean tumor volume of the control group (uncensored) reaches 1,500 mm3. A final tumor volume is taken on the day the study reached endpoint. A final weight was taken on the day the study reached end point.

CT scanning is performed to monitor and assess tumor volume. Micro-CT scan (Inveon Micro-CT, Siemens, Germany) is performed 4 h after injection of contrast agent. The contrast agent (ExiTron nano 12000, Miltenyi Biotec, Germany) is an alkaline earth-based nanoparticulate contrast agent for mouse liver CT imaging. Upon intravenous injection, the agent is taken up by cells of the reticuloendothelial system, including macrophages within the liver, termed Kupffer cells. Mice are injected with 100 μl of agent (per mouse, 25-30 g body weight) via a lateral tail vein.

PDX derived tumor masses are collected from the mice at the scheduled time of sacrifice. The tumor volume is calculated using the following formula: Volume=(W2×L)/2, with W being the shortest diameter and L being the longest diameter. Resected tumors are washed with sterile PBS. A portion of the tumor is cut into 1 mm cubes for implantation into the liver of recipient mice. The remaining tumor was used for analyses of tumor characteristics and cryopreservation.

Flow cytometry for inflammatory markers is conducted as described in the previous example.

### Example 10. A Study Using Anti-Galectin-9 Monoclonal Antibody Alone or in Combination with an Anti-PD1 Antibody in Patients with Ocular Melanoma

Galectin-9 is a molecule overexpressed by many solid tumors, including those in pancreatic cancer, colorectal cancer, and hepatocellular carcinoma. Moreover, Galectin-9 is expressed on tumor-associated macrophages, as well as intra-tumoral immunosuppressive gamma delta T cells, thereby acting as a potent mediator of cancer-associated immunosuppression. As described herein, monoclonal antibodies targeting Galectin-9 (e.g., G9.2-17, IgG4) have been developed. Data have demonstrated that the G9.2-17 halts pancreatic tumor growth by 50% in orthotopic KPC models and extended the survival of KPC animals by more than double. Without wishing to be bound by theory, it is thought that the anti-Galectin-9 antibodies reverse the M2 to M1 phenotype, facilitating intra-tumoral CD8 T cell activation. In additional, antibody G9.2-17 (IgG4) (having a heavy chain of SEQ ID NO: 19 and a light chain of SEQ ID NO: 15) has been found to synergize with anti-PD1.

Further, preclinical proof of concept data demonstrate that G9.2-17 (IgG4) (a.k.a., G9.2-17 IgG4) reduces pancreatic tumor growth by up to 50% in orthotopic ((LSL-Kras(G12D/±); LSL-Trp53(R172H/+); Pdx-1-Cre)-pancreatic ductal adenocarcinoma) KPC models and B16F10 melanoma, subcutaneous model, as a single agent. Blocking galectin-9 also extends survival of KPC animals. Mechanistically, targeting galectin-9 facilitates intra-tumoral effector T cell activation. There is an indication of synergy between G9.2-17antibody and anti-PD-1 in vivo. Namely, in the B16F10 melanoma model, a significantly greater increase in intratumoral CD8+ T cells in groups treated with anti-galectin-9 antibody and anti-PD-1 was observed, as relative to groups treated with either single agent alone. In non GLP toxicity studies, G9.2-17 (IgG4) is safe in rodents and cynomolgus monkeys at doses up to and inclusive of 100 mg/kg in rodents and 300 mg/kg in monkeys.

The purpose of this study is to determine the safety, tolerability, maximum tolerated or maximum administered dose (MTD), and objective tumor response after three months of treatment in subjects having an ocular melanoma, such as metastatic ocular melanoma. The study also examines progression-free survival (PFS), the duration of response (by RESIST), disease stabilization, the proportion of subjects alive at 3, 6, and 12 months, as well as pharmacokinetic (PK) and pharmacodynamics (PD) parameters. Subjects undergo pre- and post-treatment biopsies, as well as PET-CT imaging pre-study and once every 8 weeks for the duration of the study. In addition, immunological endpoints, such as peripheral and intra-tumoral T cell ratios, T cell activation, macrophage phenotyping, and galectin-9 serum levels are examined. The study is performed under a master study protocol, and the study lasts for 12-24 months.

This study also aims at evaluating safety and tolerability of the maximum tolerated dose (or maximum administered dose), PK, PD, immunogenicity, efficacy response outcome, patient survival, and other exploratory parameters.

Primary objectives include safety, tolerability, maximum tolerated dose (MTD), objective tumor response (ORR) at 3 months. Secondary objectives include progression free survival (PFS), duration of response by RECIST 1.1, disease stabilization, proportion alive at 3, 6 and 12 months as well as pharmacokinetic (PK) and pharmacodynamic parameters (PD).

Subject, disease, and all clinical and safety data are presented descriptively as means, medians, or proportions, with appropriate measures of variance (e.g., 95% confidence interval range). Waterfall and Swimmers plots are used to graphically present the ORR and duration of responses for subjects for each study arm, within each disease site, as described below. Exploratory correlations analysis are also undertaken to identify potential biomarkers that may be associated with ORR. All statistical analyses are performed using SAS, version 9.2 (SAS, Cary, NC).

This study includes both monotherapy of G9.2-17 (IgG4) and combination of G9.2-17 and a checkpoint inhibitor such as an approved anti-PD-1/PD-L1 antibody (e.g., nivolumab) or an investigational anti-PD-1/PD-L1 antibody. See examples below. In some examples, doses of G9.2-17 may range from about 0.2 mg/kg to 16 mg/kg or higher dose level once every two weeks. In other examples, doses of G9.2-17 may range from about 3 mg/kg to 15 mg/kg once every two weeks. The antibody is administered by intravenous infusion.

Study Objectives, Duration and Study Population are summarized in Table 22.

TABLE 22
Study Overview

| | |
|---|---|
| Study | Part 1 (Phase 1a) |
| Objectives | Primary Objective(s) |
| | Safety, tolerability, optimal biological dose (OBD), or maximum |
| | administered dose (MTD), recommended Phase 2 dose (RP2D) |
| | Secondary Objective(s) |
| | Pharmacokinetic (PK), pharmacodynamic (PD) parameters, immunogenicity |
| | Exploratory Objective(s) |
| | Exploratory end points for part 1, in addition to exploratory end points listed |
| | below: Objective Response Rate (ORR), disease control rate (DCR), |
| | progression free survival (PFS), patient survival at 6 and 12 months. |
| | Part 2 in Ocular Melanoma |
| | Primary Objective(s) |
| | Objective Response Rate (ORR) or Progression free survival (PFS) at 6 |
| | months |
| | Secondary Objective(s) |
| | Objective Response Rate (ORR), Progression free survival (PFS), disease |
| | control rate (DCR) (e.g., at 3, 6, and 12 months), duration and depth of |
| | response by RECIST 1.1, patient survival at 6 and 12 months, time to |
| | response, safety and tolerability |
| | Exploratory Endpoints for All Study Parts: iRECIST criteria, |
| | immunophenotyping from blood and tumors, cytokine profile (serum), |
| | soluble galectin-9 levels in blood (serum or plasma), galectin-9 tumor tissue |
| | expression levels and pattern of expression by immunohistochemistry |
| | (tumor, stroma, immune cells), tumor mutational burden (TMB), PDL-1 |
| | expression by immunohistochemistry, mismatch repair status, tumor markers |
| | relevant for the disease, ctDNA - and correlation of these parameters with |
| | response. Time to response (TTR). Quality of life and symptom control. |
| | Part 3: Before proceeding to Part 3, the trial is revised to specify the Part 3 |
| | objectives, trial population, treatment, and statistical analysis plan. |
| Duration of | Study duration: 12-24 months |
| Treatment/ | Study drug administration continues until progression of disease, |
| Study | unacceptable toxicity or withdrawal from the study. Patients who discontinue |
| Participation | study drug prior to progressive disease are followed on study until the time |
| | of disease progression. |
| Study | Part 1: Patients with relapsed/refractory metastatic cancers, irrespective of |
| Population | ocular melanoma type, are eligible for the dose-finding study using the |
| | continual reassessment method (CRM) as described by O'Quigley (1990; |
| | Continual reassessment method: a practical design for phase 1 clinical trials |
| | in cancer; Biometrics. 1990 March; 46(1): 33-48.). |
| | Part 2: Expansion is envisaged in tumor types where mode of action and/or |
| | an early efficacy signal are captured in Part 1. |
| | Combination with an approved anti-PD-1 agent is implemented for patients |
| | who have failed at least one prior line of treatment in the metastatic setting |
| | and are otherwise eligible for the study |
| Other Drugs | An approved Anti-PD-1 mAb; Anti-PD-1 mAb dose is determined |
| | depending on approved drug dose or is determined by initial IND; Mode of |
| | Administration: Intravenous infusion |
| Statistical | Subject, disease and all clinical safety data are presented descriptively as |
| Method | means, medians, or proportions, with appropriate measures of variance (i.e. |
| | 95% CI, range). Waterfall and Swimmers plots are used to graphically |
| | present the RR and duration of responses for patients for each study arm, |
| | within each disease site. |
| | All efficacy analyses are based on the Intention-to-treat (ITT) population |
| | unless otherwise specified. Survival curves for progression-free survival |
| | (PFS) and overall survival (OS) are generated by the method of Kaplan- |
| | Meier. Exploratory correlations analysis is also undertaken to identify |
| | potential biomarkers and other predictors that are potentially associated with |
| | ORR, PFS and OS. |
| Study | Incidence and severity of adverse evernts (AEs)/Common Terminology |
| Procedures and | Criteria adverse event (CTAEs) and Serious adverse events (SAEs), including |
| Evaluations | clinically significant changes in laboratory parameters, vital signs, |
| | performance status and electrocardiogram (ECG). |
| | Incidence of DLTs, PK and PD |
| | Plasma PK parameters (e.g., AUC0-24 h, Cmax, Tmax, estimated half- |
| | life); serum concentration vs. time profiles |
| | Objective response rate (complete response and partial response) (ORR) |
| | and clinical benefit rate (objective response and stable disease 3 months or |
| | longer); progression-free survival (PFS), overall survival (OS), disease |
| | control rate (DCR) |
| | Blood and tumor immunophenotyping, galectin-9 serum or plasma levels, |
| | and tissue expression levels and expression pattern within the tumor, stroma, |
| | immune cells compartments, time to response (TTR), and other biomarker |
| | analysis, CT (PET-CT)imaging, and other clinically relevant imaging. |
| Safety | In Part 1, the dose-escalation phase, dose escalation to the next cohort |
| Monitoring | proceeds following review of Cycle 1 of each cohort. Safety and available |
| | PK data is used to assess for a dose-limiting toxicity (DLT) in all patients of |
| | each cohort. As a safety precaution during dose escalation, new patients are |
| | entered and treated only after the first patient of each cohort has been treated |
| | with G9.2-17 IgG4 and at a minimum 7 days post-treatment has elapsed. |
| | Select DLT safety analysis for each patient will be performed following |
| | completion of Cycle 1. During the expansion phase, toxicities are monitored |
| | to review aggregate toxicity rate prior to each dose escalation. |

### Study Design

Patient population: Metastatic all comers in the 3+3 dose escalation Stage 1 (disclosed below) then expansion in ocular melanoma, for example, tumor types where mode of action and/or an early efficacy signal are captured in Stage 1.

### Stage 1

A dose-finding study is to be conducted using a continuous reassessment method (CRM)—O'Quigley et al. (1990), a model-based design that informs how the dosage of anti-Gal9 antibody should be adapted for the next patient cohort based on past trial data. Stage 1 of the study is a 3+3 dose finding and safety when the anti-Galectin-9 antibody is administered as a single agent.

A one parameter power model is to be used to describe the relationship between the dose of G9.2-17(IgG4) and the probability of observing a dose limiting toxicity (DLT). DLT is defined as a clinically significant non-hematologic adverse event or abnormal laboratory value assessed as unrelated to metastatic tumor disease progression, intercurrent illness, or concomitant medications and is related to the study drug and occurring during the first cycle on study that meets any of the following criteria:
1. All Grade 4 non-hematologic toxicities of any duration
2. All Grade 3 non-hematologic toxicities. Exceptions are as follows:
   Grade 3 nausea, vomiting and diarrhea that does not require hospitalization or TPN support and can be managed with supportive care to <grade 2 within 48 hours.
   Grade 3 electrolyte abnormalities that are corrected to <grade 2 within 24 hours.

DLT Period=One (1) cycle, i.e., two doses of the anti-Gal9 antibody on days 1 and 15 of each cycle.
Incidence and severity of AEs/CTAEs and SAEs, including clinically significant changes in laboratory parameters, vital signs & ECGs
Incidence of DLTs, PK and PD
Plasma PK parameters (e.g., AUC₀₋₂₄ₕ, Cₘₐₓ, Tₘₐₓ, estimated half-life); serum concentration vs. time profiles
Objective response rate (complete response and partial response) and clinical benefit rate (objective response and stable disease 3 months or longer); progression free survival (PFS), overall survival (OS), disease control rate (DCR)

Blood and tumor immunophenotyping, galectin-9 serum, plasma levels, and tissue expression levels and expression pattern of tumor, stroma, immune cells), time to response (TTR), and other biomarker analysis, CT (PET) imaging/other clinically indicated imaging modality.

The OBD is the largest dose that has an estimated probability of a DLT less than or equal to a target toxicity level (TTL) of 25%. Two patients at a time are to be dosed, with a maximum available sample size of 24. As a safety precaution, at each dose escalation, new patients will be entered and treated only after the first patient of each cohort has been treated with the anti-Gal9 antibody and at a minimum 7 days post-treatment has elapsed.

The dose range is shown in Table 23 below and the antibody is administered once every two weeks (Q2W) intravenously.

TABLE 23
Dosing by Cohort

| | | NO. OF | DOSE | | ADMINISTRATION |
|---|---|---|---|---|---|
| STAGE 1 | COHORT | SUBJECTS* | INCREASE^{A} | GALACTIN-9^{B} | AND DURATION** |
| Galactin-9 | COHORT | 2 | | Galectin-9^{B} | 1 administration |
| (single | 1 | | | 0.2 mg/kg | intravenously, every 2 |
| agent, | COHORT | 2 | 100% | Galectin-9^{B}, | weeks for 3 months |
| N = 15-30) | 2 | | | 0.63 mg/kg | |
| | COHORT | 2 | 67% | Galectin-9^{B} | |
| | 3 | | | 2.0 mg/kg | |
| | COHORT | 2 | 50% | Galectin-9^{B} | |
| | 4 | | | 6.3 mg/kg | |
| | COHORT | 2 | 40% | Galectin-9^{B} | |
| | 5 | | | 10 mg/kg | |
| | COHORT | 2 | 30% | Galectin-9^{B} | |
| | 6 | | | 16 mg/kg or | |
| | | | | higher dose level | |

^{A}If none of the first 3 patients experiences a dose limiting toxicity (DLT), another 3 patients are treated at the next higher dose level. However, if one of the three patients has a DLT, another 3 patients are treated at the same dose level. Dose escalation continues until at least 2 patients among the cohort of 3 to 6 patients experiences the DLT. The dose for stage II is the dose just below the level exhibiting the toxicity.
^{B}The respective trial arm is terminated when ≤1 patients respond

Dose escalation follow a modified Fibonacci sequence where the dose is increased by 100% of the preceding first dose, then followed by increases of 67%, 50%, 40%, and 30% of the preceding doses. If none of the first three patients experience a dose limiting toxicity (DLT), then another three subjects are treated at the next highest dose level. Alternatively, if one of the three subjects has a DLT, then another three subjects are treated at the same dose level. Dose escalation continues until at least two patients among the cohort of three to six patients experience a DLT.

In an alternative design, Stage 1 is to be completed when six consecutive patients have received the same dose and that dose will be identified as the OBD. A total of 5 dosage levels are to be evaluated within the CRM design.
1. Dose level 1=2 mg/kg
2. Dose level 2=4 mg/kg
3. Dose level 3=8 mg/kg
4. Dose level 4=12 mg/kg
5. Dose level 5=16 mg/kg
6. Dosing regimen: Q2W
7. Route of administration: Intravenous (IV)

Route of administration: Intravenous (IV)

### Stage 2

Stage 2 of the study is a Simon's two-stage optimal design. The study investigates the use of the anti-Galectin-9 antibody alone (single agent arms of the study) and in conjunction with nivolumab (a 240 mg flat dose administered once every two weeks; IO combination arms of the study). The dose of the anti-Galectin-9 antibody used is below the level found to exhibit toxicity in the Phase I stage.

In some instances, the anti-Gal9 antibody is to be tested as single agent. Alternatively, the anti-Gal9 antibody is to be tested in combination with an approved anti-PD-1 mAb (e.g., nivolumab, pembrolizumab, cemiplimab, or tisleizumab).

The optimal two-stage design is used to test the null hypothesis that the ORR≤5% versus the alternative that the ORR≥15% within the single agent arms. After testing the drug on 23 patients in the first stage, the respective trial arm is terminated if ≤1 patients respond. If the trial goes on to the second part of Simon's optimal design, a total of 56 patients are enrolled into each of the single agent arms. If the total number responding patients is ≤5, the drug within that arm is rejected. If >6 patients have an ORR at 3 months, the expansion cohort for that arm is activated. The above approach is applied to the single agent arms of the study.

For the IO combination arms, the starting dose of G9.2-17 IgG4 is one dose lower than the RP2D dose level (RP2D-1), identified in Part 1. To ensure patient safety, the Sponsor plans a safety run-in whereby the first 8 patients is dosed with the combination and that arm will be continued only if ≤2 patients develop a DLT, which is below the TTL of 25%. If 3 or more patients develop a DLT, the dose of G9.2-17 IgG4 will be reduced by a reduction level as per clinician's assessment or at least by 30% (dose level-1). If required, one more dose reduction by 30% of dose level-1 is allowed (dose level-2). No further dose reductions is allowed. Dose reduction to dose level-1 and -2 is allowed only if the investigator assesses that clinical benefit is being derived and may continue to be derived under dose reduced conditions.

For the anti-PD-1 mAb combination arms, the optimal two-stage design is also used to test the null hypothesis that the ORR≤10% versus the alternative that the ORR≥25%. After testing the combination on 18 patients in the first stage, the respective trial arm is terminated if ≤2 patients respond. If the trial goes on to the second part of Simon optimal design, a total of 43 patients is enrolled into each of the combination arms. If the total number of responding patients is ≤7, the combination within that arm is rejected. If >8 patients have an ORR at 3 months, the expansion cohort for that arm is activated.

### Stage 3

Stage 3 includes expansion of cohorts where early efficacy signal has been detected. If a promising efficacy signal is identified within one of the six trial arms that is attributable to the tumor type, an expansion cohort is launched to confirm the finding. The sample size for each of the expansion arms is determined based on the point estimates determined in Stage 2, in combination with predetermined level of precision for the 95% confidence interval (95% CI) around the ORR.

Pre- and post-treatment biopsy samples are analyzed in this study, e.g., imaging PET-CT pre-study and Q6/8W, as clinically indicated. PK, PD, immunological end points include peripheral and intra-tumoral T cell ratios, T cell activation, macrophage phenotyping, Galectin-9 serum levels, and Galectin-9 tissue expression levels.

### Dosing And Administration

G9.2-17 IgG4 is administered via intravenous (IV) infusion every two weeks (Q2W) until progression of disease, unacceptable toxicity, or withdrawal of consent in Part 1 and Part 2. Subjects who experience a dose-limiting toxicity may resume G9.2-17 IgG4 administration if the patient is experiencing clinical benefit, as per investigator's judgement and after a discussion with the Study Medical Monitor. Dose reduction of 30% or 50% may be performed, or as per the clinical discretion of the investigator and with agreement of the Medical Monitor and the Sponsor. Dose reduction by 30% will considered dose level-1. The next dose reduction of 30% or 50% of the previous dose level will be considered dose level-2. No more than two such dose reductions are allowed.

Part 1: Subjects receive G9.2-17 IgG4alone in accordance with the CRM design.

Part 2: Subjects receive the RP2D of G9.2-17 IgG4 as a single agent or G9.2-17 IgG4 in combination with anti-PD-lusing the RP2D identified within Part 1. However, in the case of the combination arms, the first 8 patients are dosed and that arm is continued on if ≤2 patients develop a DLT, which is below the target toxicity level (TTL) of 30%. If more than 3 patients develop a DLT determined to be G9.2-17 IgG4 related and not related to the drug/regimen used in combination, then G9.2-17 IgG4 will be dose reduced to RP2D-1 dose level (30% dose reduction of G9.2-17 IgG4 or as per clinician's assessment).

### Study Objectives and Study Populations

See Table 22 above for study objectives and study populations.

### Patient Inclusion Criteria:

1. Written Informed Consent (mentally competent, ability to understand and willingness to sign the informed consent form)
2. Aged>18 years male or non-pregnant female
3. Histologically confirmed unresectable metastatic cancer. Patients with resectable disease are excluded.
4. Able to comply with the study protocol, in the investigator's judgment
5. Life expectancy>3 months
6. Eastern Cooperative Oncology Group (ECOG) performance status 0-1 (Table 24)
7. Recent archival tumor sample available for biomarker analysis. Information must be available of therapies received since the biopsy specimen was obtained. Investigator's judgement will be used to determine whether the archival specimen as such is acceptable.
8. Galectin-9 tumor tissue expression levels assessed by IHC on an archival specimen(s), in accordance with inclusion criteria in point 5, is to be recorded if available.
9. Patient able and willing to undergo pre- and on/post-treatment biopsies. According to the investigator's judgment, the planned biopsies should not expose the patient to substantially increased risk of complications. Every effort is made that the same lesion is biopsied on repeat biopsies.
10. Measurable disease, according to RECIST v1.1. Note that lesions intended to be biopsied should not be target lesions.
11. Expected survival according to investigator's judgement>3 months
12. For Part 1: No available standard of care options, or patient has declined available and indicated standard of care therapy, or are not eligible for available and indicated standard of care therapy For Part 2: expansion cohort—received at least one line of systemic therapy in the metastatic cancer setting and for, patients who are either gemcitabine containing regimen naïve or at least 6 months out of having been treated using a gemcitabine containing regimen. CCR and CCA expansion cohorts—received at least one prior line of therapy in the metastatic setting.
13. Vaccination for COVID-19 is allowed before or during the study period. Information on timing and type of vaccine must be recorded.
14. Eastern Cooperative Oncology Group (ECOG) performance status 0-1/Karnofsky score>70 (please record both whenever possible)
15. MSI-H and MSS patients are to be allowed in Part 1 (Stage 1) of the study
16. Adequate hematologic and end organ function, defined by the following laboratory results obtained within 28 days prior to first dose of study drug treatment and within 72 hours before any consecutive dose of the study drug: neutrophil count≥1×10⁹/L, platelet count≥100×10⁹/L, for HCC in Part 1≥50×10⁹/L. hemoglobin>9 g/dL without transfusion in the previous week, Creatinine≤1.5 ULN, Creatinine clearance≥30 mL/min, AST (SGOT)≤3×ULN (≤5×ULN when HCC or hepatic metastases are present), ALT (SGPT)≤3×ULN, (≤5×ULN when HCC or hepatic metastases present) Bilirubin≤1.5×ULN (patients with known Gilbert's disease may have a bilirubin≤3.0×ULN), Albumin≥3.0 g/dLINR and PTT≤1.5×ULN; amylase and lipase≤1.5×ULN.
   If with previously diagnosed brain metastases, must have completed treatment for brain disease, either surgery or radiation therapy, 4 weeks or longer prior to screening, or have stable brain disease for at least 3 months before study start. Brain MRI is required in such cases to demonstrates no current evidence of progressive brain metastases and no new disease in the brain and/or leptomeningeal disease,
   Patients must have discontinued steroids given for the management of brain metastases at least 28 days before study start.
17. No evidence of active infection and no serious infection within 4 weeks before study start
18. Women of child-bearing potential must have a negative pregnancy test within 72 hours prior to start of treatment. For women of childbearing potential: agreement to remain abstinent (refrain from heterosexual intercourse) or use of contraceptive methods that result in a failure rate of <1% per year during the treatment period and for at least 180 days after the last study treatment. A woman is of childbearing potential if she is post-menarchae, has not reached a postmenopausal state (>12 continuous months of amenorrhea with no identified cause other than menopause), and has not undergone surgical sterilization (removal of ovaries and/or uterus). Examples of contraceptive methods with a failure rate of <1% per year include bilateral tubal ligation, male sterilization, hormonal contraceptives that inhibit ovulation, hormone-releasing intrauterine devices and copper intrauterine devices. The reliability of sexual abstinence should be evaluated in relation to the duration of the clinical trial and the preferred and usual lifestyle of the patient. Periodic abstinence (e.g., calendar, ovulation, symptom-thermal, or post ovulation methods) and withdrawal are not acceptable methods of contraception. Fertile men must practice effective contraceptive methods during the study, unless documentation of infertility exists.
19. Four (4) weeks or 5 half lives (whichever is shorter) since the last dose of anti-cancer therapy before the first G9.2-17 IgG4 administration
20. Continuation of bisphosphonate treatment (zolendronic acid) or denosumab for bone metastases which have been stable for at least 6 months before C1D1 is allowed.
21. Biliary or gastric outlet obstruction allowed, provided it is effectively drained by endoscopic, operative, or interventional means
22. Pancreatic, biliary, or enteric fistulae allowed, provided they are controlled with an appropriate non-infected and patent drain (if any drains or stents are in situ, patency needs to be confirmed before study start)
   Additionally, for Part 1 only:
23. Patients:
   a) who have already received at least one prior line of systemic therapy for metastatic disease, or
   b) who have a tumor type for which there are no available standard of care options.
      Additionally, for Part 2 only:
24. Expansion cohort: 1st line metastatic patients who are either gemcitabine-containing regimen naïve or at least 3 months out of having been treated using a gemcitabine-containing regimen previously in a neoadjuvant or adjuvant/locally advanced setting.
25. Expansion cohorts—patients who have received at least one prior line of therapy in the metastatic setting.

TABLE 24
ECOG PERFORMANCE STATUS*

| Grade | ECOG |
|---|---|
| 0 | Fully active, able to carry on all pre-disease performance without restriction |
| 1 | Restricted in physically strenuous activity but ambulatory and able to carry |
| | out work of a light or sedentary nature, e.g., light house work, office work |
| 2 | Ambulatory and capable of all self-care but unable to carry out any work |
| | activities. Up and about more than 50% of waking hours |
| 3 | Capable of only limited self-care, confined to bed or chair more than 50% of |
| | waking hours |
| 4 | Completely disabled. Cannot carry on any self-care. Totally confined to bed |
| | or chair |
| 5 | Dead |

As published in Am J Clin Oncol: Oken M M, Creech R H, Tormey D C, et al. Toxicity and response criteria of the Eastern Cooperative Oncology Group. Am J Clin Oncol. 1982; 5: 649-655.

### Patient Exclusion Criteria:

1. Patient diagnosed with metastatic cancer of an unknown primary
2. Unwilling or unable to follow protocol requirements
3. Prior or current illicit drug addiction (medical and recreational marijuana/cannabidiol (CBD)/Tetrahydrocannabinol (THC) would not be considered “illicit”)
4. Clinically significant, active uncontrolled bleeding, and any patients with a bleeding diathesis (e.g., active peptic ulcer disease). prophylactic or therapeutic use of anticoagulants is allowed.
5 Pregnant or Lactating females
6. Receiving any other investigational agents or participating in any other clinical trial involving another investigational agent for treatment of solid tumors within 4 weeks or 5 half-lives of the administered drug (whichever is shorter) prior to cycle 1, day 1 of the study or other investigational therapy or major surgery within 4 weeks from the date of consent, or planned surgery within 4 weeks from envisaged study start (this includes dental surgery).
7 Radiation therapy within 4 weeks of the first dose of study drug, except for palliative radiotherapy to a limited field, such as for the treatment of bone pain or a focally painful tumor mass, and which does not jeopardize required measurable lesions for response assessment (RECIST v1.1).
8. Patients with fungating tumor masses
9. Patients with locally advanced ocular melanoma without distant organ metastatic deposits.
10. ≥CTCAE grade 3 toxicity (except alopecia and vitiligo) due to prior cancer therapy. Grade 4 immune-mediated toxicities with a prior checkpoint inhibitor. Grade 2 or Grade 3 pneumonitis or any other Grade 3 checkpoint inhibitor-related toxicity that led to immunotherapy treatment discontinuation. Low-grade (<Grade 3) toxicities, such as neuropathy from prior treatments, manageable electrolyte abnormalities and lymphopenia, alopecia and vitiligo are allowed.
11. History of second malignancy except those treated with curative intent more than five years previously without relapse or low likelihood of recurrence (for example non-melanotic skin cancer, cervical carcinoma in situ, early (or localized) prostate cancer or superficial bladder cancer)
12. Active brain or leptomeningeal metastases. Patients with brain metastases are eligible provided they have shown clinically and radiographically stable disease (SD) for at least 4 weeks after definitive therapy and have not used steroids (>10 mg/day of prednisone or equivalent) for at least 4 weeks prior to the first dose of study drug
13. Evidence of severe or uncontrolled systemic diseases, congestive cardiac failure>New York Heart Association (NYHA) class 2 , Myocardial Infarction (MI) within 6 months or laboratory finding that in the view of the Investigator makes it undesirable for the patient to participate in the trial
14. Any medical condition that the Investigator considers significant to compromise the safety of the patient or that impairs the interpretation of G9.2-17 IgG4 toxicity assessment
15. Serious non-healing wound, active ulcer or untreated bone fracture
16. Uncontrolled pleural effusion, pericardial effusion, or ascites requiring recurrent drainage procedures. For the purposes of this study, “recurrent” is defined as ≥3 drains in the previous 30 days.
17. History of severe allergic, anaphylactic, or other hypersensitivity reactions to chimeric or humanized antibodies or fusion proteins
18. Significant vascular disease (e.g., aortic aneurysm requiring surgical repair or recent arterial thrombosis) within 6 months of Cycle 1 Day 1
19. History of pulmonary embolism, stroke or transient ischemic attack within 2 or 3 months prior to Cycle 1 Day
20. History of abdominal fistula or gastrointestinal perforation within 6 months prior to Cycle 1 Day 1
21. Active auto-immune disorder (except type I diabetes, hypothyroidism requiring only hormone replacement, vitiligo, psoriasis, or alopecia)
22. Requires systemic immunosuppressive treatment including, but not limited to (cyclophosphamide, azathioprine, methotrexate, thalidomide, and anti-tumor necrosis factor [anti-TNF] agents). Patients who have received or are receiving acute, low dose, systemic immunosuppressant medications (e.g., dexamethasone or prednisolone) may be enrolled. Replacement therapy (e.g., thyroxine, insulin, physiologic corticosteroid replacement therapy ( (e.g., ≤10 mg/day of prednisone equivalents)for adrenal or pituitary insufficiency) is not considered a form of systemic treatment. The use of inhaled corticosteroids and mineralocorticoids (e.g., fludrocortisone), topical steroids, intra-nasal steroids, intra-articular, and ophthalmic steroids is allowed
23. Severe tumor-related pain (>Grade 3, per the Common Terminology Criteria for Adverse Events, (CTCAE) v.5.0) unresponsive to broad analgesic interventions (oral and/or patches)
24. Uncontrolled hypercalcemia, despite use of bisphosphanates
25. Any other diseases, metabolic dysfunction, physical examination finding, or clinical laboratory finding giving reasonable suspicion of a disease or condition that contraindicates the use of an investigational drug or that may affect the interpretation of the results or render the patient at high risk from treatment complications
26. Received an organ transplant(s)
27 Undergoing dialysis
28. For patients enrolled into nivolumab combination cohorts, no prior exposure to any anti-PD-1 or anti-PD-L1 agent in any prior lines of therapy. Additionally, patients diagnosed as dMMR/MSI-H are excluded.
29. For Part 1, hormonal androgen deprivation therapy is allowed to continue for patients with metastatic castration-resistant prostate cancer.

### Study Assessments

The schedule of assessments is divided into 2-week cycles after the pre-dose screening, which may take place up to 4 weeks prior to commencement of treatment. Study assessments include medical and physical examinations performed by a qualified physician, practitioner, or physician assistant. Medical history taken includes oncology history, radiation therapy history, surgical history, current and past medication. Assessments include restaging scan (CT with contrast, MRI with contrast, PET-CT (diagnostic CT) and/or X-ray).

Assessments also include Tumor biopsies (starting pre dose 1 and repeat biopsy as feasible)—depending upon scan(s). Alternatively, archival tissue may be used pre-dose.

Relevant tumor markers per tumor type—e.g., Ca15-3, CA-125, CEA, CA19-9, S100, alpha fetoprotein, etc., are assessed every cycle pre-dose (which may be decreased to every 3 cycles after 6 months of treatment, following the same schedule as restaging scans), as appropriate. Assessments further include vital signs, ECOG, adverse events, blood count, blood chemistry, blood coagulation (prothrombin time (PT) and partial thromboplastin time (PTT), activated partial thromboplastin time (APTT)), blood and tumor biomarker analysis (immune phenotyping, cytokine measurement) and urine analysis (specific gravity, protein, white blood cell-esterase, glucose, ketones, urobilinogen, nitrite, WBC, RBC, and pH). Serum chemistry includes glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, HgbAlc, blood urea nitrogen, CPK, TSH, fT4, lipase, amylase, PTH, testosterone, estradiol. prolactin, FSH, LH, CRP.

CT with contrast is the preferred modality for restaging Scans-(MRI, PET-CT and/or other imaging modalities instead of or in addition to the CT scan if CT is not feasible or appropriate, given location of the disease). Assessments are done every 6 to 8 weeks+/−1 week and at the End of Treatment if not assessed within the last 4 to 6 weeks.

Patients' blood samples are collected for routine clinical laboratory testing, and include hematology and serum chemistry.

Blood chemistry includes the following: glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK, TSH, fT4, PTH, Estradiol, prolactin, testosterone, FSH, LH, gamma glutamyl transferase (gamma GT), hemoglobin Alc (HgbAlc) (only if history of Type 1 or Type 2 diabetes mellitus), lipase, amylase, free cortisol additionally at specified visits.

### (A) Assessments by Visit

The table of Schedule of Assessments (Table 25) below provides a list of assessments to be performed during the screening period (up to 28 days), the treatment period (presented as 28-day cycles), the End of Treatment/Early Termination period, IMAR follow-up and the long term follow-up period.

TABLE 25
Schedule of Assessments

| | | | | | | | | | | | | | | | | Post Treatment | | |
|---|---|---|---|---|---|---|---|---|---|---|---|---|---|---|---|---|---|---|
| | | | | | | | | | | | | | | | | End of | | Long |
| | | | | | | | | | | | | | | | | Treat- | | Term |
| | | | | | | | | | | | | | | | | ment | | Follow- |
| | | Treatment Phase | | | | | | | | | | | | | | or Early | IMAR | Up U |
| | Pre-Dose | Cycle 1 | | | | Cycle 2 | | | Cycle 3 | | | | Cycle 4 | | | Termi- | Follow- | Every |
| | Screening | (28 days) | | | | (28 days) | | | (28 days) | | | | (28 days) and beyond | | | nation | UpT | 3 mons |
| | Study Day | | | | | | | | | | | | | | | | | |
| | | | | | | | | | | | | | | | | 30 days | 90 days | |
| | | | | | | | | | | | | | | | | after | after | |
| | −28 to −1 | 1 | 2 | 8 | 15 | 1 | 8 | 15 | 1 | 2 | 8 | 15 | 1 | 8 | 15 | last dose | last dose | |
| | Cycle Day | | | | | | | | | | | | | | | | | |
| | | C1 | C1 | C1 | C1 | C2 | C2 | C2 | C3 | C3 | C3 | C3 | CX | CX | CX | | | |
| | | D1 | D2 | D8 | D15 | D1 | D8 | D15 | D1 | D2 | D8 | D15 | D1 | D8 | D15 | — | — | — |
| | +/− days allowable | | | | | | | | | | | | | | | | | |
| | | | 1 | 1 | 1 | 2 | 1 | 2 | 2 | 1 | 1 | 2 | 2 | 1 | 2 | 3 | 7 | n/a |
| Study drug | | X | | X | X | X | X | X | X | | X | X | X | X | X | | | |
| administration^{A} | | | | | | | | | | | | | | | | | | |

Study Procedures & Examinations

| | | | | | | | | | | | | | | | | |
|---|---|---|---|---|---|---|---|---|---|---|---|---|---|---|---|---|
| Eligibility | X | | | | | | | | | | | | | | | |
| assessment and | | | | | | | | | | | | | | | | |
| written informed | | | | | | | | | | | | | | | | |
| consent | | | | | | | | | | | | | | | | |
| Demographics^{B} | X | | | | | | | | | | | | | | | |
| Medical history^{C} | X | | | | | | | | | | | | | | | |
| Previous and | X | X | X | X | X | X | X | X | X | X | X | X | X | X | | |
| concomitant | | | | | | | | | | | | | | | | |
| medications^{D} | | | | | | | | | | | | | | | | |
| Adverse events^{E} | | X | X | X | X | X | X | X | X | X | X | X | X | X | X | |
| ECHO/MUGA^{F} | X | | | | | | | | | | | X | | | | |
| 12-lead ECG | X | X | | | | X | | X | | | | X | | | | |
| (QTcF) | | | | | | | | | | | | | | | | |
| Physical exam^{G} | X | X | | | X | X | X | X | | | X | X | X | X | X | |
| ECOG | X | X | | | X | X | X | X | | | X | X | X | X | | |
| Vital Signs_{H} | X | X | | | X | X | X | X | | | X | X | X | X | | |
| Tumor imaging | X | | | | | | | X | | | | | | X | | X U |
| assessment | | | | | | | | | | | | | | repeat | | |
| (CT with contrast | | | | | | | | | | | | | | if end | | |
| preferred)^{N} | | | | | | | | | | | | | | of study | | |
| | | | | | | | | | | | | | | is >8 | | |
| | | | | | | | | | | | | | | weeks | | |
| | | | | | | | | | | | | | | after | | |
| | | | | | | | | | | | | | | last scan | | |

Clinical Labs

| | | | | | | | | | | | |
|---|---|---|---|---|---|---|---|---|---|---|---|
| Pregnancy tes^{I} | X | | | X | | X | | X | | X | |
| Hematology^{J} | X | X | X | X | X | X | X | X | X | X | X |
| Serum | X | X | X | X | X | X | X | X | X | X | X |
| Chemistry^{K} | | | | | | | | | | | |
| TSH, Free T4, | X | X | | X | | X | | X | | X | X |
| serum lipase, | | | | | | | | | | | |
| amylase, PTH, | | | | | | | | | | | |
| FSH, LH, free | | | | | | | | | | | |
| cortisol | | | | | | | | | | | |
| Blood | X | X | X | X | X | X | X | X | X | X | X |
| Coagulation^{L} | | | | | | | | | | | |
| Urinalysis^{M} | X | X | X | X | X | X | X | X | X | X | X |

Pharmacodynamics and Pharmacokinetics

| | | | | | | | | | | | | | | | |
|---|---|---|---|---|---|---|---|---|---|---|---|---|---|---|---|
| Tumor biopsy^{O} | X | | | | | | | | | | X ± | | | | |
| | | | | | | | | | | | 7 d | | | | |
| Tumor type- | X | X | | | X | X | X | X | | | X | X | X | X | |
| relevant | | | | | | | | | | | | | | | |
| biomarkers^{P} | | | | | | | | | | | | | | | |
| Blood sampling | | X | X | X | X | X | | X | X | X | X | X | | X | |
| for biomarker PD | | | | | | | | | | | | | | | |
| analysis^{Q} | | | | | | | | | | | | | | | |
| PK blood | | X | X | X | X | X | | X | X | X | X | X | | | |
| sampling^{R} | | | | | | | | | | | | | | | |
| Immunogenicity | | X | | | | X | | X | | | | X | | X | |
| (ADA)^{S} | | | | | | | | | | | | | | | |
| Survival Follow- | | | | | | | | | | | | | | | X U |
| up | | | | | | | | | | | | | | | |

ADA: anti-drug antibodies;
AE: adverse event;
ALT: alanine aminotransferase;
APTT: activated partial thromboplastin time;
AST: aspartate aminotransferase;
C: cycle;
CPK: creatine phosphokinase;
COVID19: Coronavirus SARS-COV-2;
CRP: C-reactive protein;
CT: computed tomography;
D or d: day(s);
ECG: electrocardiogram;
ECOG: Eastern Cooperative Oncology Group;
ECHO: echocardiography/cardiac ultrasound;
FSH: follicle-stimulating hormone;
IMAR: immune-mediated adverse reaction;
INR: international normalized ratio;
LDH: lactate dehydrogenase;
LH: luteinizing hormone; min: minute(s);
MUGA: multigated acquisition scan;
PD: pharmacodynamics;
PK: pharmacokinetics;
PT: prothrombin time;
PTH: parathyroid hormone;
PTT: partial thromboplastin time;
QTcF: QT interval, Fridericia's Correction Formula;
RBC: red blood cell count;
SGOT: serum glutamic-oxaloacetic transaminase;
SGPT: serum glutamic pyruvic transaminase;
TSH: thyroid stimulating hormone;
WBC: white blood cell count.
^{A}Study drug administration: G9.2-17 IgG4 treatment will be administered, on C1D1 and C1D15 on every cycle.
^{B}Demographics: Data include age, gender, race, and ethnicity.
^{C}Medical history: In addition to general medical history, data collection also includes oncology history, surgical/transplant and radiation therapy history and COVID-19 history and testing.
^{D}Previous and concomitant medications (including vaccines and complementary treatments/supplements): Data to include name, indication, dose, route, start and end dates for each. Allergies and intolerances, dose modifications while on study, schedule of dosing changes and reasons for them should also be obtained.
^{E}Adverse events: Any AEs starting or worsening after study drug administration will be recorded. AEs should be followed until resolved to one of the following: baseline, stabilized, or deemed irreversible. All SAEs are to be collected until 30 days after last dose of study medication. All study-procedure-related SAEs must be collected from the date of patient's written consent.
^{F}ECHO/MUGA: This assessment of heart function is conducted at Screening and repeated on Day 1 of Cycle 4; the assessment window is +/−5 days. It should be conducted more frequently when clinically indicated and once every 3 months.
^{G}Physical exam: Include height at screening for determination of body surface area. Include weight at all scheduled exam times. A Neurological exam will be conducted only on patients who have stable and/or pre-treated brain metastases.
^{H}Vital Signs: temperature, heart rate, blood pressure, respiratory rate.
^{I}Pregnancy test (blood or urine): Only for women of childbearing potential with uterus in situ. Test results must be available before scheduled dosing.
^{J}Hematology: Analysis includes complete blood count, differential, platelets, hemoglobin. Collect blood samples pre-dose.
^{K}Serum chemistry: Analysis includes albumin, alkaline phosphatase, bilirubin (total, direct), blood urea nitrogen, calcium, CPK, creatinine, electrolytes (sodium, potassium, chloride, magnesium, phosphorus), gamma glutamyl transferase (gamma GT), glucose, hemoglobin Alc (HgbAlc) (only if history of Type 1 or Type 2 diabetes mellitus), LDH, SGPT (ALT) or SGOT (AST), total protein. Fasting glucose to be assessed only if clinically indicated. Collect blood samples pre-dose.
^{L}Blood Coagulation: Collect blood samples pre-dose. Analysis includes APTT, PT, PTT, and INR (if on allowable anti-coagulants), CRP, and troponin.
^{M}Urinalysis: Analysis includes color, appearance, dipstick for specific gravity, protein, white blood cell-esterase, glucose, ketones, urobilinogen, nitrite, WBC, RBC, pH. (Urine culture and sensitivity to be run only if patient is clinically symptomatic.)
^{N}Tumor imaging assessment: For screening, the assessment must be performed within the 28-day screening period. On study, assessments are done every 8 weeks + 7 days (ie, C3D1, C5D1, C7D1, C9D1, etc.) and at the End of Treatment if not assessed within the previous 4-6 weeks. Assessments may be performed more frequently if clinically indicated. If an objective response is seen on a scan, a confirmation scan will be done 4 weeks (+7 d) later. After this confirmatory scan, the scheduled scans are to be resumed at a frequency of every 8 weeks + 7 days from the date of the confirmatory scan.
^{O}Tumor biopsies: If patient MMR/MSI status is unknown at screening, the test should be run at the local laboratory. In Part 2, TMB tissue analysis will be performed. The on-study biopsy is scheduled for C3D15 + 7 days, and should occur only after the tumor imaging scan in Cycle 3. It is recognized that a variety of clinical factors may make it difficult to obtain adequate specimens. Decisions not to perform biopsy on-treatment should be discussed with the Medical Monitor.
^{P}Tumor type-relevant biomarkers: Blood samples are to be collected at screening and every cycle pre-dose administration as appropriate for the tumor type. Blood sampling may be decreased to every 3rd cycle after 6 months of treatment.
^{Q}PD blood sampling: Blood samples will be collected pre-dose administration on dosing days. May be decreased to every 3rd cycle after 6 months of treatment.
^{R}PK blood sampling: Cycle 1 and Cycle 3 Day 1: blood samples will be collected pre-dose and at end of study drug infusion (EOI), 2 and 4 h (+30 min) post-study drug administration. Cycle 1 and Cycle 3 Day 15, blood samples will be collected pre-dose and at EOI only. Cycle 1 and Cycle 3 Day 2 and 8 (non-dosing days), PK blood samples will be collected at only one time point. Cycle 2 and Cycle 4: blood samples will be collected Day 1 only and should occur pre-dose and at EOI. Blood samples for PK will be collected every 2 cycles thereafter (ie, C6D1, C8D1, etc.) pre-dose and at EOI.
^{S}ADA blood sampling: Blood samples will be collected Day 1 of Cycles 1-4, pre-dose. Thereafter, it will be collected every 2 cycles, Day 1, pre-dose (ie, C6D1, C8D1, etc.).
^{T}All patients treated with G9.2-17 IgG4 + nivolumab must return 90-days +/− 7 days after last dose of study drug for an assessment of potential immune-mediated adverse reactions (IMARs).
^{U}Long-Term Follow-up: Tumor imaging should continue, where possible, for patients discontinuing treatment due to reasons other than progression of disease and not receiving additional systemic anticancer treatments. Survival data will be collected at a minimum every 3 months. It can be collected more frequently to support data cleaning or regulatory submission efforts. Follow-up can be conducted by telephone, electronic messaging or chart review and will continue for up to 2 years after the patient has the End of Treatment/Early Termination visit.

During the COVID-19 pandemic many governments require citizens to practice social distancing, and more vulnerable populations are advised to self-isolate. These types of constraints may affect the ability to run this clinical study as originally intended. Planned site visits can be adapted so that the study can safely continue during the pandemic. Possible modifications as approved may include:
visit and/or study procedure postponement
replacement with telephone/video call(s)
replacement with home visits
visits performed at an alternative clinical location
visits performed by a health-care provider outside the study team
visit and/or study procedure completely cancelled.

### (B) Screening Period (Between Day-28 and Day-1)

The following procedures must be conducted within 4 weeks of initiating treatment:

### Study Procedures & Examinations

Written informed consent
Verify inclusion and exclusion criteria for patient eligibility
Patient demographics
Medical history
Previous and concomitant medications
ECHO/multigated acquisition scan (MUGA)
12-lead ECG (QT interval corrected using Fridericia's formula [QTcF])
Physical examination-in patients with stable and pre-treated brain metastases, perform a neurological exam
ECOG performance status
Vital signs
Tumor imaging assessment (computed tomography [CT] or magnetic resonance imaging (MRI), with or without contrast; or positron emission tomography (PET)-CT; CT with contrast is preferred)

### Clinical Labs

Pregnancy test for women of childbearing potential (WOCBP)
Hematology
Serum chemistry
Thyroid stimulating hormone (TSH), free T4 or thyroxine (fT4), serum lipase, amylase, parathyroid hormone (PTH), follicle-stimulating hormone (FSH), luteinizing hormone (LH), free cortisol
Blood coagulation
Urinalysis

### Pharmacodynamics and Pharmacokinetics

Tumor biopsy
   Biopsy can be omitted if it is deemed that the procedure a risk to the patient.
   If a biopsy is unavailable, site will make every effort to obtain an archival tumor tissue sample available as a formalin-fixed paraffin-embedded (FFPE) block. Acceptable archival samples include those obtained via a core needle biopsy or excisional surgery within the last five years.
dMMR-MSI-H status (if the MMR and MSI status of the patients has not been previously determined, the test must be run at the local laboratory)
TMB in tissue for Part 2 G9.2-17 IgG4 +anti-PD1 antibody combo arm only
Tumor type-relevant biomarkers

### (C) Treatment Period

Each treatment cycle has a duration of 28 days. See the Schedule of Assessment Table. For COVID-19 infection diagnosed while on treatment,

### Treatment Procedures for Day 1 of Each Cycle (CXD1; ±2 Days Beginning Cycle 2)

The following procedures are performed on Day 1 of each treatment cycle.

### Study Procedures and Examinations

Concomitant medications
AEs
12-lead ECG (QTcF)
Physical examination
ECOG performance status
Vital signs

### Clinical Labs

Pregnancy test for WOCBP
Hematology
Serum chemistry
TSH, fT4, lipase, amylase, PTH, FSH, LH, free cortisol
Blood coagulation
Urinalysis

### PK/PD Assessments

PD blood sampling
PK blood sampling
ADA blood sampling
Tumor type-relevant biomarkers

### Study Drug Administration

Administered only after all pre-dose assessments and procedures are completed.

Additionally, beginning on Day 1 of Cycle 3, the following assessment will be performed every 8 weeks:
Tumor imaging assessment (CT or MRI, with or without contrast; or PET-CT; CT with contrast is preferred)
   Furthermore, beginning on Day 1 of Cycle 4, the following assessment will be performed every 3 months:
ECHO/MUGA

### Treatment Procedures for Days 2 and 8 of Cycle 1 and Cycle 3 (CXD2 ±1 Day and CXD8 ±1 Day)

### Study Procedures and Examinations

Concomitant medications
AEs

### PK/PD Assessments

PD blood sampling
PK blood sampling

### Treatment Procedures for Day 15 of each Cycle (CXD15 ±1 Day for Cycle 1 and ±2 Days for Beginning Cycle 2)

The following procedures will be performed on Day 15 of each treatment cycle.

### Study Procedures and Examinations

Concomitant medications
AEs
Physical examination
ECOG performance status
Vital signs

### Clinical Labs

Hematology
Serum chemistry
Blood coagulation
Urinalysis

### PK/PD Assessments

PD blood sampling on CID15 and C3D15 only
PK blood sampling on CID15 and C3D15 only
Tumor type-relevant biomarkers
Tumor biopsy on C3D15 +7 days (Cycle 3 ONLY; may be eliminated if deemed it too risky for the patient)

### Study Drug Administration

Administered only after all pre-dose assessments and procedures are completed.

### Additional Treatment beyond Cycle 4

Treatment cycles beyond Cycle 4 can be repeated as indicated in the Schedule of Assessment table. If the patient is experiencing clinical benefit, even in the event of radiological progression, the patient can continue on treatment.

### End of Treatment or Early Termination Procedures

The following procedures are done 30 days (+3 days) after the last dose, including patients who have discontinued treatment early.

### Study Procedures and Examinations

Concomitant medications
AEs
Physical examination
ECOG
Vital signs
Tumor imaging assessment: Confirmatory scan if end of study is >8 weeks after previous scan.

### Clinical Labs

Pregnancy test for WOCBP
Hematology
Serum chemistry
TSH, fT4, lipase, amylase, PTH, FSH, LH, free cortisol
Blood coagulation
Urinalysis

### PD Assessments

PD blood sampling
ADA blood sampling
Tumor type-relevant biomarkers

### IMAR 90-Day Follow-Up

All patients on the combination treatment with nivolumab or tislelizumab in Part 2 must return on Day 90±7 for a safety follow-up in order to evaluate any possible delayed IMARs. The visit will include the following:

### Study Procedures and Examinations

AEs
Physical examination
Vital signs

### Clinical Labs

Hematology
Serum chemistry
TSH, fT4, lipase, amylase, PTH, FSH, LH, free cortisol
Blood coagulation
Urinalysis

### Long-Term Follow-Up

Once a patient has completed the treatment period, follow-up is performed every 3 months for up to 2 years after the patient has the End of Treatment/Early Termination visit for patients discontinuing treatment due to reasons other than progression of disease and not receiving additional systemic anticancer treatments. Tumor imaging assessment will continue, where possible, for patients discontinuing treatment due to reasons other than progression of disease and not receiving additional systemic anticancer treatments.

Survival data as well as information on any new anticancer therapy initiated after disease progression will be collected at a minimum every 3 months. It can be collected more frequently to support data cleaning or regulatory submission efforts.

### (D) Efficacy Assessments

Planned time points for all efficacy assessments are provided in the table of Schedule of Assessment.

### Investigational Product, Dose, And Administration

### Study Intervention Administration

All patients will receive G9.2-17 IgG4 via IV infusion, every 2 weeks, until progression of disease, unacceptable toxicity, or withdrawal of consent.

In Part 1, patients will receive G9.2-17 IgG4 alone at sequentially increasing doses starting at 0.2 mg/kg, in accordance with the CRM design.

In Part 2, patients will receive the RP2D of G9.2-17 IgG4 (as determined in Part 1) as a single agent or the G9.2-17 IgG4 RP2D-1 in combination with a checkpoint inhibitor such as nivolumab or tislelizumab based on each ocular melanoma type

See Table 26 below for a summary description of each study intervention. Patients who experience a DLT in Part 1 will not resume treatment. Patients who experience a DLT in Part 2 will have their treatment interrupted. Their treatment may resume at the same or reduced dose of G9.2-17 IgG4 if they are experiencing a clinical benefit.

TABLE 26
Summary Characteristics of Study Interventions

| Arm Name | G9.2-17 IGG4 | G9.2-17 IGG4 + nivolumab |
|---|---|---|
| Intervention Name | G9.2-17 IGG4 | nivolumab |
| Intervention Type | Biologic | Biologic |
| Dose Formulation | Vial | Vial |
| Unit Dose Strength(s) | 30 mg/mL | 40 mg/4 mL vial |
| | | and |
| | | 100 mg/10 mL vial |
| Dosage Level(s) | Part 1: increasing doses starting | Part 2 only: |
| | at 0.2 mg/kg with a maximum | Nivolumab: 240 mg |
| | dose of 16 mg/kg | |
| | Part 2: RP2D for single agent | |
| | G9.2-17 IGG4 or RP2D-1 for | |
| | G9.2-17 IGG4 in combination | |
| | with nivolumab or | |
| | chemotherapy | |
| Frequency of | Days 1 and 15 of each 28-day | Days 1 and 15 of each 28-day |
| Administration | cycle | cycle |
| Route of | IV infusion | IV infusion |
| Administration | | |
| Use | experimental | experimental |
| IMP and non-IMP | IMP | IMP |
| Packaging and | Study Intervention will be | Study Intervention will be |
| Labeling | provided in appropriate | provided in appropriate |
| | configurations; each will be | configurations; each will be |
| | labeled as required per country | labeled as required per |
| | requirement | country requirement |

IMP: investigational medicinal product,
IV: intravenous,
mAb: monoclonal antibody,
RP2D: recommended Phase 2 dose;
RP2D-1: dose level in cohort immediately preceding the RP2D

### Dose Modification

The decision to proceed to the next dose level of G9.2-17 IgG4 will be made based on safety, tolerability, and preliminary PK data obtained in at least 2 patients at the prior dose level.

The dosing schedule may also be adjusted based on PK data obtained.

Detailed dose modification instructions are available as follows (disclosed herein): Management of Immune-Mediated Adverse Reactions (IMARs) caused by G9.2-17 IgG4 Management of Immune-Mediated Adverse Reactions (IMARs) caused by G9.2-17 IGG4+Nivolumab or Tislelizumab Combination Treatment

Recommended Dose Modifications for OPDIVO® (Nivolumab) for AEs (other than IMARs)

Recommended Dose Modifications for G9.2-17 IGG4 (for AEs outside the DLT window and other than IMARs)

### Dose Administration

If an infusion-related reaction is encountered, interrupt the infusion and if clinically indicated, administer relevant medication(s) (eg, anti-histamine, anti-emetic, steroids, anti-pyretics, beta-blocker(s) etc.). If it is deemed appropriate to resume the infusion, resume at a slower infusion rate.

For subsequent cycles for the same patient, apply the appropriate pre-medications (anti-histamine, anti-emetic, steroids, anti-pyretics, beta-blocker(s) etc., as clinically indicated needed) and consider utilizing a slower infusion rate.

### Dose Delay

If any clinically meaningful AE>Grade 3 possibly related or related to one or more study drugs occurs, it will be discussed with the Medical Monitor before continuing with dosing. A dose delay may be necessary for >Grade 3 AE.

### Dose De-escalation

### Part 1

The CRM design will guide both dose escalations and de-escalations. A total of 6 dosage levels will be evaluated within the CRM design:
Dose Escalation Cohort 1=0.2 mg/kg
Dose Escalation Cohort 2=0.63 mg/kg
Dose Escalation Cohort 3=2 mg/kg
Dose Escalation Cohort 4=6.3 mg/kg
Dose Escalation Cohort 5=10 mg/kg
Dose Escalation Cohort 6=16 mg/kg

After each cohort of 2 patients, the CRM model will make a recommendation to increase to the next dose level for the next cohort of 2 patients, remain at the same dose level or reduce to the previous dose level.

### Dose Reduction

No dose reductions will be allowed for any patient that is being evaluated for DLTs (within the 28 day DLT window). In case a dose reduction is necessary, the study intervention will be administered as follows:

For Parts 1 and 2 G9.2-17 IGG4 alone patients: Dose reduction will be allowed only if it is assessed that clinical benefit is being derived and may continue to be derived under dose reduced conditions, see table below for recommend dose modifications and also IMAR or other AE management. See Table 27 and 28 below.

TABLE 27
Recommended Dose Modifications for G9.2-17 IgG4
(AEs outside the DLT window and other than IMARs)

| Adverse Reaction | Severity | G9.2-17 IGG4 Dose Modification |
|---|---|---|
| Non-hematologic | ≥Grade 4 | Permanently discontinue treatment |
| and hematologic | | with G9.2-17 IGG4. |
| toxicities | | |
| Hematologic | Grade 3 | Withhold treatment until toxicity |
| toxicities | | resolves to ≤Grade 2. |
| | | Resume G9.2-17 IGG4 at 50% |
| | | reduction from previous dose. |
| | | If recurs again at ≥Grade 3 severity, |
| | | hold until resolved to ≤grade 2 and |
| | | implement a further 50% dose |
| | | reduction may be performed. |
| | | No further dose reductions are allowed. |
| Non-hematologic | Grade 3 | Withhold treatment until toxicity |
| toxicities | | resolves to ≤Grade 2. |
| (except those listed | | Resume G9.2-17 IGG4 at 50% |
| in Appendix 4, | | reduction from previous dose. |
| Management of | | If recurs again at ≥Grade 3 severity, |
| Immune-Mediated | | hold until resolved to ≤grade 2 and |
| Adverse Reactions | | implement a further 50% dose |
| (IMARs) Caused by | | reduction may be performed. |
| G9.2-17 IGG4) | | No further dose reductions are allowed. |
| Gastrointestinal | Grade 3 | Withhold G9.2-17 IGG4 until toxicity |
| (Nausea, Vomiting) | Not requiring hospitalization | reverses to ≤Grade 2 |
| | or parenteral nutrition | Resume G9.2-17 IGG4 with no dose |
| | support; managed by | reduction |
| | supportive care | If recurrent, withhold G9.2-17 IGG4 |
| | | until toxicity reverses to ≤Grade 2 |
| | | Then resume G9.2-17 IGG4 at a 50% |
| | | reduction from the previous dose |
| | | If recurrent, withhold G9.2-17 IGG4 |
| | | until toxicity reverses to ≤Grade 2 |
| | | Resume G9.2-17 IGG4 at an additional |
| | | 50% dose reduction |
| | | No further dose reductions are allowed |

TABLE 28
Management of Immune-Mediated Adverse Reactions (IMARs) Caused by G9.2-17 IgG4

| IMAR | Guidance for Management |
|---|---|
| Immune-mediated pneumonitis | Monitor patients for signs with radiographic imaging and |
| | for clinical symptoms of pneumonitis. |
| | Withhold G9.2-17 IGG4 for moderate and permanently |
| | discontinue for severe or life-threatening pneumonitis. |
| | Administer corticosteroids at a dose of 1 to 2 mg/kg/day |
| | prednisone equivalents for moderate (Grade 2) or more |
| | severe (Grade 3-4) pneumonitis, followed by |
| | corticosteroid taper. |
| | Withhold G9.2-17 IGG4 until resolution to <Grade 2. |
| | For moderate (Grade 2) pneumonitis. Resume treatment |
| | at a 50% dose reduction. |
| | Upon recurrence of Grade 2 pneumonitis at the reduced |
| | dose of G9.2-17 IGG4, permanently discontinue G9.2-17 |
| | IGG4. |
| Immune-mediated colitis | Monitor patients for signs and symptoms of colitis. |
| | Administer corticosteroids at a dose of 0.5 to 1 |
| | mg/kg/day prednisone equivalents followed by |
| | corticosteroid taper for moderate (Grade 2) colitis of |
| | more than 5 days duration. |
| | If worsening or no improvement occurs despite initiation |
| | of corticosteroids, increase dose to 1 to 2 mg/kg/day |
| | prednisone equivalents. |
| | When initial Grade 2 colitis resolves to <Grade 2, |
| | resume G9.2-17 IGG4 at a 50% dose reduction. |
| | If Grade ≥2 colitis recurs, withhold G9.2-17 IGG4 until |
| | colitis resolves to <Grade 2. Resume G9.2-17 IGG4 at an |
| | additional 50% dose reduction. No further dose |
| | reductions for G9.2-17 IGG4 are allowed. |
| | Administer corticosteroids at a dose of 1 to 2 mg/kg/day |
| | prednisone equivalents followed by corticosteroid taper |
| | for severe (Grade 3) or life threatening (Grade 4) colitis. |
| | When Grade 3 colitis resolves to ≤Grade 2, resume |
| | G9.2-17 IGG4 at a 50% dose reduction. |
| | Permanently discontinue G9.2-17 IGG4 for life- |
| | threatening (Grade 4) or for recurrent colitis of ≥Grade 2 |
| | upon re-initiation of G9.2-17 IGG4. |
| Immune-mediated hepatitis | Monitor for changes in liver function. |
| | Administer corticosteroids at a dose of 0.5 to 1 |
| | mg/kg/day prednisone equivalents for moderate (Grade 2) |
| | transaminase elevations. |
| | Withhold G9.2-17 IGG4 for moderate (Grade 2) immune- |
| | mediated hepatitis. When resolved to <Grade 2 resume |
| | G9.2-17 IGG4 at a 50% dose reduction. |
| | If ≥Grade 2 hepatitis recurs, permanently discontinue |
| | G9.2-17 IGG4. |
| | Administer corticosteroids at a dose of 1 to 2 mg/kg/day |
| | prednisone equivalents followed by corticosteroid taper |
| | for severe (Grade 3) or life-threatening (Grade 4) |
| | transaminase elevations, with or without concomitant |
| | elevation in total bilirubin. |
| | Permanently discontinue G9.2-17 IGG4 for severe (Grade |
| | 3) or life-threatening (Grade 4) immune-mediated |
| | hepatitis. |
| Immune-mediated | Administer hormone replacement as clinically indicated |
| endocrinopathies: | and corticosteroids at a dose of 1 mg/kg/day prednisone |
| Hypophysitis | equivalents followed by corticosteroid taper for moderate |
| | (Grade 2) or greater hypophysitis. |
| | Withhold G9.2-17 IGG4 for moderate (Grade 2) or |
| | severe (Grade 3) and when resolved to <Grade 2, resume |
| | G9.2-17 IGG4 at a 50% dose reduction. |
| | If Grade ≥2 toxicity recurs, permanently discontinue |
| | G9.2-17 IGG4. |
| | Permanently discontinue G9.2-17 IGG4 for life- |
| | threatening (Grade 4) hypophysitis. |
| Adrenal insufficiency | Monitor patients for signs and symptoms of adrenal |
| | insufficiency. |
| | Withhold G9.2-17 IGG4 for moderate (Grade 2) and |
| | once resolved to <Grade 2, resume G9.2-17 IGG4 at |
| | a 50% dose reduction. |
| | If Grade ≥2 toxicity recurs, permanently discontinue |
| | G9.2-17 IGG4. |
| | Administer corticosteroids at a dose of 1 to 2 |
| | mg/kg/day prednisone equivalents followed by a |
| | corticosteroid taper for severe (Grade 3) or life- |
| | threatening (Grade 4) adrenal insufficiency. |
| | Permanently discontinue G9.2-17 IGG4 for severe |
| | (Grade 3) or life-threatening (Grade 4) adrenal |
| | insufficiency, or for recurrent colitis of ≥Grade 2 |
| | upon re-initiation of G9.2-17 IGG4. |
| Hypothyroidism and | Monitor thyroid function prior to and periodically during |
| Hyperthyroidism | nivolumab treatment. |
| | Administer hormone-replacement therapy for |
| | hypothyroidism. |
| | Initiate medical management for control of |
| | hyperthyroidism. |
| | There are no recommended dose adjustments of G9.2-17 |
| | IGG4 for <Grade 3 hypothyroidism or hyperthyroidism |
| | Discontinue G9.2-17 IGG4 for ≥Grade 3 hypothyroidism |
| | or hyperthyroidism |
| Type 1 Diabetes Mellitus | Withhold G9.2-17 IGG4 in cases of severe (Grade 3) |
| | hyperglycemia until metabolic control is achieved. |
| | Upon resolution to ≤Grade 2, resume G9.2-17 IGG4 at a |
| | 50% dose reduction. |
| | If Grade ≥2 toxicity recurs, permanently discontinue |
| | G9.2-17 IGG4. |
| | Permanently discontinue G9.2-17 IGG4 for life- |
| | threatening (Grade 4) hyperglycemia. |
| Immune-mediated nephritis and | Monitor patients for elevated serum creatinine prior to |
| renal dysfunction | and periodically during treatment. |
| (Defined as renal dysfunction | Administer corticosteroids at a dose of 0.5 to 1 |
| or ≥Grade 2 increased creatinine, | mg/kg/day prednisone equivalents for moderate (Grade 2) |
| requirement for corticosteroids, | or severe (Grade 3) increased serum creatinine, if |
| and no clear alternate etiology) | worsening or no improvement occurs, increase dose of |
| | corticosteroids to 1 to 2 mg/kg/day prednisone |
| | equivalents. |
| | Withhold G9.2-17 IGG4 for moderate (Grade 2) or severe |
| | (Grade 3) increased serum creatinine. When resolved to <Grade |
| | 2, resume G9.2-17 IGG4 at a 50% dose reduction. |
| | If ≥□Grade 2 toxicity recurs, permanently discontinue |
| | G9.2-17 IGG4. |
| | Administer corticosteroids at a dose of 1 to 2 mg/kg/day |
| | prednisone equivalents followed by corticosteroid taper |
| | for life-threatening (Grade 4) increased serum creatinine. |
| | Permanently discontinue G9.2-17 IGG4 for life- |
| | threatening (Grade 4) increased serum creatinine. |
| Immune-mediated skin adverse | Withhold for severe and permanently discontinue for life- |
| reactions | threatening rash. |
| (Immune-mediated rash, including | For symptoms or signs of SJS or TEN, withhold G9.2-17 |
| Stevens-Johnson syndrome (SJS) | IGG4 and refer the patient for specialized care for |
| and toxic epidermal necrolysis | assessment and treatment. |
| (TEN)) | If SJS or TEN is confirmed, permanently discontinue |
| | G9.2-17 IGG4. |
| | For immune-mediated rash ≥Grade 2, administer |
| | corticosteroids at a dose of 1 to 2 mg/kg/day prednisone |
| | equivalents followed by a corticosteroid taper for severe |
| | (Grade 3) or life-threatening (Grade 4) rash. |
| | Withhold G9.2-17 IGG4 for moderate (Grade 2) and |
| | severe (Grade 3) rash and when resolved to <Grade 2, |
| | resume G9.2-17 IGG4 at a 50% dose reduction. |
| | If ≥Grade 2 rash recurs, withhold G9.2-17 IGG4 until |
| | resolved to <Grade 2 and resume G9.2-17 IGG4 at an |
| | additional 50% dose reduction. No further dose |
| | reductions are allowed. |
| | Permanently discontinue G9.2-17 IGG4 for life |
| | threatening (Grade 4) rash. |
| Immune-mediated encephalitis | Monitor for changes in neurologic function. |
| | Evaluation of patients with neurologic symptoms may |
| | include, but not be limited to, consultation with a |
| | neurologist, brain MRI, and lumbar puncture. |
| | Withhold G9.2-17 IGG4 in patients with new-onset |
| | moderate to severe neurologic signs or symptoms and |
| | evaluate to rule out infectious or other causes of moderate |
| | to severe neurologic deterioration. |
| | If other etiologies are ruled out, administer |
| | corticosteroids at a dose of 1 to 2 mg/kg/day prednisone |
| | equivalents for patients with immune-mediated |
| | encephalitis, followed by corticosteroid taper. |
| | Permanently discontinue G9.2-17 IGG4 for immune- |
| | mediated encephalitis. |
| Other Immune-Mediated Adverse | If uveitis occurs in combination with other immune- |
| Reactions | mediated adverse reactions, consider a Vogt-Koyanagi- |
| (May include: myocarditis, | Harada-like syndrome, and may require treatment with |
| rhabdomyolysis, myositis, uveitis, | systemic steroids to reduce the risk of permanent vision |
| iritis, pancreatitis, facial and | loss. |
| abducens nerve paresis, | For any suspected immune-mediated adverse reactions, |
| demyelination, polymyalgia | exclude other causes. |
| rheumatica, autoimmune | Based on the severity (Grade) of the adverse reaction, |
| neuropathy, Guillain-Barré | permanently discontinue or withhold G9.2-17 IGG4, |
| syndrome, hypopituitarism, | administer high-dose corticosteroids, and if appropriate, |
| systemic inflammatory response | initiate hormone-replacement therapy. |
| syndrome, gastritis, duodenitis, | Upon improvement to Grade 1 or less, initiate |
| sarcoidosis, histiocytic necrotizing | corticosteroid taper and continue to taper over at least 1 |
| lymphadenitis (Kikuchi | month. |
| lymphadenitis), motor dysfunction, | Consider restarting G9.2-17 IGG4 after completion of |
| vasculitis, aplastic anemia, | corticosteroid taper based on the severity of the event, |
| pericarditis, and myasthenic | resuming at a 50% dose reduction. |
| syndrome.) | |

### Nivolumab

Nivolumab (OPDIVO®) is a programmed death receptor-1 (PD-1) blocking antibody indicated for the treatment of multiple tumor types. It is administered as an intravenous infusion over 30 minutes (unless guided otherwise) at 240 mg every 2 weeks, in a 28-day cycle. As per the FDA label, there are no contraindications for administrations of nivolumab. Nivolumab AEs are presented in the Tables 33-34 below according to their frequency of occurrence.

### Dose Modifications for Specific AEs Related to Administration of Nivolumab

Recommendations for nivolumab modifications based on specific AEs are provided below. There are no recommended dose modifications of nivolumab for hypothyroidism or hyperthyroidism.

Recommended dose modification for Nivolumab for AEs other than IMAR is provided in Table 29 below:

TABLE 29
Recommended Dose Modification for Nivolumab

| Adverse Reaction | Severity* | Dose Modification |
|---|---|---|
| OAEs | Grade 3 adverse events | Withhold dose^{a} |
| | First occurrence | Permanently discontinue |
| | Recurrence of same Grade 3 adverse | |
| | reaction | |
| | Life-threatening or Grade 4 adverse reaction | Permanently discontinue |
| | Grade 3 myocarditis | |
| | Requirement for ≥10 mg/day prednisone or | |
| | equivalent for >12 weeks | |
| | Persistent Grade 2 or 3 adverse reactions | |
| | lasting ≥12 weeks | |

*Toxicity was graded per NCI CTCAE V4.
^{a}Resume treatment when adverse reaction improves to Grade 0 or 1Source: OPDIVO Highlights of Prescribing Information, Revised April 2019.

### Dose Modification for IMARs

If an IMAR occurs, see guidance provided herein on dose management of G9.2-17 IgG4, nivolumab.

TABLE 30
Adverse Events (non-IMAR-Related) Reported
for Nivolumab According to Frequency
Common: AEs occurring in >30% of patients

| | | | |
|---|---|---|---|
| Fatigue | Lymphocytopenia | Hyponatremia | Decreased Appetite |
| Myalgia | Decreased Appetite | C | |

Less Common: AE's occurring in 10-29% of patients

| | | | |
|---|---|---|---|
| Nausea | Vomiting | Constipation | Weakness |
| Hyperkalemia | Hypokalemia | Hypercalcemia | Hypocalcemia |
| Hypomagnesemia | Swelling | Fever | Abdominal pain |
| Weight loss | Pain | Thrombocytopenia | Pneumonia |

Rare (but severe) AEs occurring in <10% of patients

| | |
|---|---|
| Stomatitis | Peripheral neuropathy |
| Bronchitis | Upper respiratory tract infection |

Source: Section 6.1, OPDIVO Package Insert

TABLE 31
Adverse Events Reported for Nivolumab to be Treated as IMARs*
Common: AEs occurring in >30% of patients

| | |
|---|---|
| Cough | Dyspnea |

Less Common: AE's occurring in 10-29% of patients

| | | | |
|---|---|---|---|
| Increased serum | Increased ALT | Increased ALP | Increased serum |
| AST | | | creatinine |
| Chest pain | | Rash | Itching |
| Diarrhea | Colitis | Joint pain | |

Rare (but severe) AEs occurring in <10% of patients

| Immune-mediated pneumonitis | Immune-mediated hypo- and hyperthyroidism |
|---|---|
| Immune-mediated hepatitis | Immune mediated nephritis and renal dysfunction |
| Immune-mediated colitis | |

Guidance for managing IMARs caused by the combination of G9.2-17 IgG4 and Nivolumab is provided in Table 35 below:

TABLE 32
Management of Immune-Mediated Adverse Reactions (IMARs)
Caused by G9.2-17 IgG4 + Nivolumab Combination Treatment

| IMAR | Guidance for Management |
|---|---|
| Immune-mediated pneumonitis | Monitor patients for signs with radiographic imaging |
| | and for symptoms of pneumonitis. |
| | Withhold nivolumab and G9.2-17 IGG4 for moderate |
| | (Grade 2) and permanently discontinue for severe |
| | (Grade 3) or life-threatening (Grade 4) pneumonitis. |
| | Administer corticosteroids at a dose of 1 to 2 |
| | mg/kg/day prednisone equivalents for moderate (Grade |
| | 2) or more severe (Grade 3-4) pneumonitis, followed |
| | by corticosteroid taper. |
| | Withhold nivolumab and G9.2-17 IGG4 until resolution |
| | to <Grade 2 for moderate (Grade 2) pneumonitis. |
| | Resume G9.2-17 IGG4 at a 50% dose reduction. Any |
| | dose modification for nivolumab may be performed. |
| | Upon recurrence of ≥Grade 2 pneumonitis, |
| | permanently discontinue treatment with both the study |
| | medication and nivolumab. |
| Immune-mediated colitis | Monitor patients for signs and symptoms of colitis. |
| | Administer corticosteroids at a dose of 0.5 to 1 |
| | mg/kg/day prednisone equivalents followed by |
| | corticosteroid taper for moderate (Grade 2) colitis of |
| | more than 5 days duration. |
| | If worsening or no improvement occurs despite |
| | initiation of corticosteroids, increase dose to 1 to 2 |
| | mg/kg/day prednisone equivalents. |
| | Administer corticosteroids at a dose of 1 to 2 |
| | mg/kg/day prednisone equivalents followed by |
| | corticosteroid taper for severe (Grade 3) or life |
| | threatening (Grade 4) colitis. |
| | Withhold nivolumab and G9.2-17 IGG4 for moderate |
| | or severe (Grade 2 or 3) colitis until resolution |
| | to <Grade 2. Resume G9.2-17 IGG4 at a 50% dose |
| | reduction. Any dose modifications for nivolumab may |
| | be performed. |
| | If Grade ≥3 recurs, then permanently discontinue |
| | treatment with both the study medication and |
| | nivolumab. |
| | If Grade ≥2 colitis recurs, withhold G9.2-17 IGG4 and |
| | nivolumab until colitis resolves to <Grade 2. Resume |
| | G9.2-17 IGG4 at an additional 50% dose reduction. |
| | Any dose modification for nivolumab may be |
| | performed. No further dose reductions for G9.2-17 |
| | IGG4 are allowed. |
| | If Grade ≥2 colitis recurs again, permanently |
| | discontinue treatment with both study medications. |
| | Permanently discontinue G9.2-17 IGG4 and nivolumab |
| | for life-threatening (Grade 4). Withhold nivolumab for |
| | moderate or severe and permanently discontinue for |
| | life-threatening colitis. |
| Immune-mediated hepatitis | Monitor for changes in liver function. |
| | Administer corticosteroids at a dose of 0.5 to 1 |
| | mg/kg/day prednisone equivalents for moderate (Grade |
| | 2) transaminase elevations. |
| | Withhold nivolumab and G9.2-17 IGG4 for moderate |
| | (Grade 2) immune-mediated hepatitis. When resolved |
| | to <Grade 2, resume G9.2-17 IGG4 at a 50% dose |
| | reduction. Any dose modification for nivolumab may |
| | be performed. |
| | If ≥Grade 2 hepatitis recurs, permanently discontinue |
| | treatment with both G9.2-17 IGG4 and nivolumab. |
| | Administer corticosteroids at a dose of 1 to 2 |
| | mg/kg/day prednisone equivalents followed by |
| | corticosteroid taper for severe (Grade 3) or life- |
| | threatening (Grade 4) transaminase elevations, with or |
| | without concomitant elevation in total bilirubin. |
| | Permanently discontinue treatment with both G9.2-17 |
| | IGG4 and nivolumab for severe (Grade 3) or life- |
| | threatening (grade 4) immune-mediated hepatitis. |
| Immune-mediated | Administer hormone replacement as clinically indicated |
| endocrinopathies: | and corticosteroids at a dose of 1 mg/kg/day prednisone |
| Hypophysitis | equivalents followed by corticosteroid taper for |
| | moderate (Grade 2) or greater hypophysitis. |
| | Withhold G9.2-17 IGG4 and nivolumab for moderate |
| | (Grade 2) or severe (Grade 3). Resume G9.2-17 IGG4 |
| | at a 50% dose reduction. Any dose modification for |
| | nivolumab may be performed. |
| | If Grade ≥2 toxicity recurs, permanently discontinue |
| | treatment with both G9.2-17 IGG4 and nivolumab. |
| | Permanently discontinue treatment G9.2-17 IGG4 and |
| | nivolumab for life-threatening (Grade 4) hypophysitis. |
| Adrenal insufficiency | Monitor patients for signs and symptoms of adrenal |
| | insufficiency. |
| | Administer corticosteroids at a dose of 1 to 2 |
| | mg/kg/day prednisone equivalents followed by a |
| | corticosteroid taper for severe (Grade 3) or life- |
| | threatening (Grade 4) adrenal insufficiency. |
| | Withhold G9.2-17 IGG4 and nivolumab for moderate |
| | (Grade 2), and when resolved to <Grade 2, resume |
| | G9.2-17 IGG4 at a 50% dose reduction. Dose reduction |
| | for nivolumab may be performed. |
| | If Grade ≥2 recurs, permanently discontinue treatment |
| | with both study medications. |
| | Permanently discontinue treatment with both study |
| | medications for severe (Grade 3) or life-threatening |
| | (Grade 4) adrenal insufficiency. |
| Hypothyroidism and | Monitor thyroid function prior to and periodically |
| Hyperthyroidism | during nivolumab treatment. |
| | Administer hormone-replacement therapy for |
| | hypothyroidism. |
| | Initiate medical management for control of |
| | hyperthyroidism. |
| | There are no recommended dose adjustments of G9.2- |
| | 17 IGG4 or nivolumab for <Grade 3 hypothyroidism |
| | or hyperthyroidism |
| | Discontinue G9.2-17 IGG4 and nivolumab for ≥Grade |
| | 3 hypothyroidism or hyperthyroidism |
| Type 1 Diabetes Mellitus | Withhold G9.2-17 IGG4 and nivolumab in cases of |
| | severe (Grade 3) hyperglycemia until metabolic control |
| | is achieved. |
| | Follow G9.2-17 IGG4 IMAR guidance for Type 1 |
| | Diabetes Mellitus when resolved to ≤Grade 2. Any |
| | dose modification for nivolumab may be performed. |
| | Permanently discontinue treatment with both study |
| | medications for life-threatening (Grade 4) |
| | hyperglycemia. |
| Immune-mediated nephritis and | Monitor patients for elevated serum creatinine prior to |
| renal dysfunction | and periodically during treatment. |
| (Defined as renal dysfunction | Administer corticosteroids at a dose of 0.5 to 1 |
| or ≥Grade 2 increased creatinine, | mg/kg/day prednisone equivalents for moderate (Grade |
| requirement for corticosteroids, | 2) or severe (Grade 3) increased serum creatinine, if |
| and no clear alternate etiology) | worsening or no improvement occurs, increase dose of |
| | corticosteroids to 1 to 2 mg/kg/day prednisone |
| | equivalents. |
| | Withhold G9.2-17 IGG4 and nivolumab for moderate |
| | (Grade 2) or severe (Grade 3) increased serum |
| | creatinine. When resolved to <Grade 2, resume G9.2-17 |
| | IGG4 at a 50% dose reduction. Any dose modification |
| | for nivolumab may be performed. |
| | If ≥Grade 2 toxicity recurs, permanently discontinue |
| | treatment with both G9.2-17 IGG4 and nivolumab. |
| | Administer corticosteroids at a dose of 1 to 2 |
| | mg/kg/day prednisone equivalents followed by |
| | corticosteroid taper for life-threatening (Grade 4) |
| | increased serum creatinine. |
| | Permanently discontinue treatment with both G9.2-17 |
| | IGG4 and nivolumab for life-threatening (Grade 4) |
| | increased serum creatinine. |
| Immune-mediated skin adverse | Withhold both medications for severe (Grade 3) and |
| reactions | permanently discontinue for life-threatening (Grade 4) |
| (Nivolumab can cause immune- | rash. |
| mediated rash, including Stevens- | For symptoms or signs of SJS or TEN, withhold |
| Johnson syndrome (SJS) and toxic | nivolumab and G9.2-17 IGG4 and refer the patient for |
| epidermal necrolysis (TEN), some | specialized care for assessment and treatment. |
| cases with fatal outcome.) | If SJS or TEN is confirmed, permanently discontinue |
| | treatment with both G9.2-17 IGG4 and nivolumab. |
| | For immune-mediated rash, administer corticosteroids |
| | at a dose of 1 to 2 mg/kg/day prednisone equivalents |
| | followed by a corticosteroid taper for severe (Grade 3) |
| | or life-threatening (Grade 4) rash. |
| | Withhold G9.2-17 IGG4 and nivolumab for moderate |
| | (Grade 2) and severe (Grade 3) rash and when resolved |
| | to <Grade 2, resume G9.2-17 IGG4 at a 50% dose |
| | reduction. Any dose modification for nivolumab may |
| | be performed. |
| | If ≥Grade 2 rash recurs, withhold G9.2-17 IGG4 and |
| | nivolumab until resolved to <Grade 2 and resume G9.2- |
| | 17 IGG4 at an additional 50% dose reduction. No |
| | further dose reductions are allowed. Any dose |
| | modification for nivolumab may be performed. |
| Immune-mediated encephalitis | Monitor for changes in neurologic function. |
| | Evaluation of patients with neurologic symptoms may |
| | include, but not be limited to, consultation with a |
| | neurologist, brain MRI, and lumbar puncture. |
| | Withhold G9.2-17 IGG4 and nivolumab in patients |
| | with new-onset moderate to severe neurologic signs or |
| | symptoms and evaluate to rule out infectious or other |
| | causes of moderate to severe neurologic deterioration. |
| | If other etiologies are ruled out, administer |
| | corticosteroids at a dose of 1 to 2 mg/kg/day prednisone |
| | equivalents for patients with immune-mediated |
| | encephalitis, followed by corticosteroid taper. |
| | Permanently discontinue treatment with G9.2-17 IGG4 |
| | and nivolumab for immune-mediated encephalitis. |
| Other Immune-Mediated Adverse | If uveitis occurs in combination with other immune- |
| Reactions | mediated adverse reactions, consider a Vogt-Koyanagi- |
| (The following clinically | Harada-like syndrome, which has been observed in |
| significant immune-mediated | patients receiving nivolumab, and may require |
| adverse reactions, some with fatal | treatment with systemic steroids to reduce the risk of |
| outcome, occurred in less than | permanent vision loss. |
| 1.0% of patients receiving | For any suspected immune-mediated adverse reactions, |
| nivolumab: myocarditis, | exclude other causes. |
| rhabdomyolysis, myositis, uveitis, | Based on the severity of the adverse reaction, |
| iritis, pancreatitis, facial and | permanently discontinue or withhold G9.2-17 IGG4 |
| abducens nerve paresis, | and nivolumab, administer high-dose corticosteroids, |
| demyelination, polymyalgia | and if appropriate, initiate hormone-replacement |
| rheumatica, autoimmune | therapy. |
| neuropathy, Guillain-Barré | Upon improvement to Grade 1 or less, initiate |
| syndrome, hypopituitarism, | corticosteroid taper and continue to taper over at least 1 |
| systemic inflammatory response | month. |
| syndrome, gastritis, duodenitis, | Consider restarting G9.2-17 IGG4 and nivolumab after |
| sarcoidosis, histiocytic necrotizing | completion of corticosteroid taper based on the severity |
| lymphadenitis (Kikuchi | of the event. Resume G9.2-17 IGG4 at a 50% dose |
| lymphadenitis), motor | reduction. Any dose modification for nivolumab may |
| dysfunction, vasculitis, aplastic | be performed. |
| anemia, pericarditis, and | Immune-mediated adverse reactions may occur after |
| myasthenic syndrome.) | discontinuation of nivolumab therapy. |
| Infusion reactions | Discontinue nivolumab in patients with severe or life- |
| (Reported in less than 1.0% of | threatening infusion reactions. |
| patients in clinical trials.) | Interrupt or slow the rate of infusion in patients with |
| | mild or moderate infusion reactions. |
| | Discontinue nivolumab for severe and life-threatening |
| | infusion reactions. |
| | Interrupt or slow the rate of infusion in patients with |
| | mild or moderate infusion reactions. |
| Complications of Allogeneic | Transplant-related complications include hyperacute |
| Hematopoietic Stem Cell | graft-versus-host-disease (GVHD), acute GVHD, |
| Transplantation (HSCT) | chronic GVHD, hepatic veno-occlusive disease (VOD) |
| (Fatal and other serious | after reduced intensity conditioning, and steroid- |
| complications can occur in | requiring febrile syndrome (without an identified |
| patients who receive allogeneic | infectious cause). |
| HSCT before or after being treated | These complications may occur despite intervening |
| with a PD-1 receptor blocking | therapy between PD-1 blockade and allogeneic HSCT. |
| antibody.) | Follow patients closely for evidence of transplant- |
| | related complications and intervene promptly. |
| | Consider the benefit versus risks of treatment with a |
| | PD-1 receptor blocking antibody prior to or after an |
| | allogeneic HSCT. |
| Embryo-Fetal Toxicity | In animal reproduction studies, administration of |
| (Based on its mechanism of action | nivolumab to cynomolgus monkeys from the onset of |
| and data from animal studies, | organogenesis through delivery resulted in increased |
| NIVOLUMAB can cause fetal | abortion and premature infant death. |
| harm when administered to a | Advise pregnant women of the potential risk to a fetus. |
| pregnant woman.) | Advise females of reproductive potential to use |
| | effective contraception during treatment with a |
| | nivolumab-containing regimen and for at least 5 |
| | months after the last dose of nivolumab. |

Source: Nivolumab (OPDIVO ®) Highlights of Prescribing Information, Revised April 2019.

### Tislelizumab

Tislelizumab is a PD-1 inhibiting mAb drug being developed for the treatment of cancer. Tislelizumab is formulated for IV injection in a single-use glass vial (20R glass, USP type I) with a rubber stopper containing a total of 100 mg of tislelizumab mAb in 10 mL of buffered isotonic solution. Tislelizumab is administered as an intravenous infusion over approximately 30 minutes (unless guided otherwise) at 300 mg every 4 weeks, in a 28-day cycle.

The active ingredient of tislelizumab is a humanized IgG4 variant mAb against PD-1, binding to the ECD of human PD-1 with high specificity and affinity (KD=0.15 nM). The excipients of tislelizumab include: sodium citrate dihydrate, citric acid monohydrate, L-histidine hydrochloride monohydrate, L-histidine, trehalose dihydrate, polysorbate-20, and WFI. Tislelizumab competitively blocks the binding of both PD-L1 and PD-L2, inhibiting PD-1-mediated negative signaling and enhancing the functional activity in T cells in in vitro cell-based assays. In addition, tislelizumab demonstrated antitumor activity in several human cancer allogeneic xenograft models and a human PD-1 transgenic mouse model.

The IgG4 variant antibody has very low binding affinity to Fcγ RIIIA and Clq by in vitro assays, suggesting a low or no ADCC and CDC effect in humans. Unlike natural IgG4 antibody, tislelizumab has no observable Fab-arm exchange activity by the in vitro assay, predicting the antibody would be stable in vivo, unlikely forming bispecific antibodies.

Exposure-response (E-R) relationships between tislelizumab exposure and efficacy across a variety of advanced solid tumors support the 300 mg Q4W regimens. 300 mg Q4W regimen is not expected to be clinically different from the 200 mg Q3W in terms of safety or efficacy outcomes.

The safety profile of tislelizumab is consistent with the therapeutic class of the drug with a relatively low rate of treatment-related Grade 3 or above toxicity.

Tislelizumab AEs are presented below in Table 33 according to their frequency of occurrence. Reported AEs that may be IMAR-related are summarized in Table 34. Refer to Table 35 for dose modifications and management of IMARs related to Tislelizumab when given in combination with G9.2-17 IgG4. Table 36 refers tor management of non-IMAR-related AEs.

TABLE 33
Adverse Events (non-IMAR-Related) Reported
for Tislelizumab According to Frequency

| ORGAN SITE | ADVERSE EVENT |
|---|---|
| blood and lymphatic | anemia (8-21%, severe 1-8%) |
| system/febrile | leukopenia (5-12%, severe 2%) |
| neutropenia | lymphopenia (<1%) |
| | neutropenia (3-9%, severe 1-3%) |
| | thrombocytopenia (1-9%, severe 1%) |
| gastrointestinal | emetogenic potential: low³ |
| | constipation (13%, severe <1%) |
| | diarrhea (5-13%, severe 1%) |
| | nausea (4-13%, severe <1%) |
| | stomatitis, aphthous ulcer (3%, severe <1%) |
| | vomiting (10%, severe 1%) |
| general disorders | extravasation hazard: none⁴ |
| andadministration | chills (1%, severe 0%) |
| site conditions | fatigue, asthenia (1-23%, severe 2%) |
| | pyrexia (5-33%, severe 1%) |
| infections | pneumonia (3%) |
| and | |
| infestations | |
| injury, poisoning, | infusion-related reactions (2-29%, severe <2%) |
| and procedural | |
| complications | |
| investigations | alkaline phosphatase increase (6%, severe 1%) |
| | ALT increase (1-16%, severe 2%) |
| | AST increase (1-17%, severe 3%) |
| | bilirubin increase (5-12%, severe 2%) |
| | creatine phosphokinase increase (1-6%, severe 1%) |
| | hyperglycemia (2-5%, severe 1%) |
| | hyponatremia (1%, severe 1%) |
| | lipase increase (1%, severe 1%) |
| | proteinuria (1-6%) |
| | thyroid hormone (free triiodothyronine and thyroxine) decrease (1%) |
| | thyroid stimulating hormone (TSH) increase (1-7%) |
| metabolism and | appetite decrease (1-15%, severe <1%) |
| nutrition | diabetes mellitus, type 1 (1%, severe 1%) |
| | diabetic ketoacidosis (<1%) |
| musculoskeletal | arthralgia (2-6%) |
| andconnective | |
| tissue | |
| respiratory, thoracic | cough (13%) |
| andmediastinal | dyspnea (6%, severe 1%) |
| | pruritus (8-14%, severe <1%) |
| | rash (6-18%, severe 1%) |
| | skin reaction, undefined (1%, severe <1%) |

Clinically important side effects are in bold, italics

By disrupting PD-1-mediated signaling, tislelizumab acts to restore antitumor immunity and halt progression of tumor growth. This restoration of immune system activity may result in immune related adverse reactions involving 1 or more body systems, which can be life threatening or fatal in rare cases. While these events usually become manifest during treatment with tislelizumab, they can also occur after discontinuation of tislelizumab therapy.

TABLE 34
Adverse Events Reported for Tislelizumab to be Treated as IMARS*

| ORGAN SITE | ADVERSE REACTION |
|---|---|
| cardiac | immune-mediated myocarditis (1%, severe <1%) |
| endocrine | immune-mediated adrenal insufficiency (<1%) |
| | immune-mediated hyperthyroidism (3-4%, severe <1%) |
| | immune-mediated hypothyroidism (6-21%, severe 1%) |
| | immune-mediated thyroiditis (1%) |
| eye | immune-mediated uveitis (<1%) |
| gastrointestinal | immune-mediated colitis (1%, severe 1%) |
| | immune-mediated pancreatitis (1%, severe 1%) |
| | immune-mediated hepatitis (1-3%, severe 1-2%) |
| musculoskeletal | arthralgia (2-6%) |
| andconnective | immune-mediated myositis/rhabdomyolysis (1%, severe 1%) |
| tissue | immune-mediated nephritis, renal dysfunction (1%, severe 1%) |
| immune system | immune-mediated adverse events (21-22%, severe 5-11%) |
| respiratory, thoracic and | cough (13%) |
| mediastinal | dyspnea (6%, severe 1%) |
| | immune-mediated pneumonitis, interstitial lung disease (2-7%, severe 1-6%) |
| skin and subcutaneous | immune-mediated dermatitis (1%, severe <1%) |
| tissue | immune-mediated pruritus (2-4%) |
| | immune-mediated rash (3%, severe <1%) |
| | immune-mediated vitiligo (1%) |

### Dose Modification

The decision to proceed to the next dose level of G9.2-17 IgG4 in Part 1 will be made based on safety, tolerability, and preliminary PK data obtained in at least 2 patients at the 5 prior dose level.

The dosing schedule may also be adjusted based on PK data obtained. Detailed dose modification instructions are provided in Tables 35-36 below. See also Tables 27 and 28 above for management of IMARs caused by G9.2-17 IgG4 and recommended dose modification.

TABLE 35
Management of Immune-Mediated Adverse Reactions (IMARs) Caused
by G9.2-17 IgG4 + Tislelizumab Combination Treatment

| IMAR | Guidance for Management |
|---|---|
| Immune-mediated pneumonitis | Monitor patients for signs with radiographic imaging |
| | and for symptoms of pneumonitis. |
| | Grade 1, consider holding tislelizumab until there is |
| | significant improvement in patient's signs and |
| | symptoms |
| | Withhold tislelizumab and G9.2-17 IgG4 for |
| | moderate (Grade 2) and permanently discontinue for |
| | severe (Grade 3) or life-threatening (Grade 4) |
| | pneumonitis. Administer corticosteroids at a dose of |
| | 1 to 2 mg/kg/day prednisone equivalents for |
| | moderate (Grade 2). For more severe (Grade 3-4) |
| | pneumonitis, permanently discontinue G9.2-17 IgG4 |
| | and tislelizumab and treatment in a hospital setting |
| | with IV methylprednisolone 2 to 4 mg/kg/day is |
| | recommended, followed by corticosteroid taper. |
| | Withhold tislelizumab and G9.2-17 IgG4 until |
| | resolution to < Grade 2 for moderate (Grade 2) |
| | pneumonitis. Resume G9.2-17 IgG4 at a 50% dose |
| | reduction. |
| | Tislelizumab may be restarted only if symptoms |
| | resolve completely or are controlled on prednisolone ≤ 10 |
| | mg/day. Withhold tislelizumab for Grade 2 |
| | pneumonitis, and only restart if symptoms resolve |
| | completely or are controlled on prednisolone ≤ 10 |
| | mg/day. Permanently discontinue treatment with |
| | tislelizumab if symptoms persist with the use of |
| | corticosteroids. |
| | Upon recurrence of > Grade 2 pneumonitis, |
| | permanently discontinue treatment with both the |
| | study medication and tislelizumab |
| Immune-mediated colitis and | Monitor patients for signs and symptoms of colitis. |
| diarrhea | Withhold treatment with tislelizumab and G9.2-17 |
| | IgG4 for Grade 2 diarrhea or colitis and start oral |
| | prednisolone 0.5 mg/kg/day (nonenteric coated). If |
| | worsening or no improvement occurs despite |
| | initiation of corticosteroids, increase dose to 1 to 2 |
| | mg/kg/day prednisone equivalents. |
| | Taper steroids over 2 to 4 weeks with resolving |
| | signs and symptoms. Resume treatment with |
| | tislelizumab when signs and symptoms improve or |
| | resolve to baseline. Administer prednisolone 1 to 2 |
| | mg/kg/day for colitis Grade 3 or Grade 4, converting |
| | to oral prednisolone and tapering this dose over at |
| | least 4 weeks when improvement is evident. |
| | Permanently discontinue tislelizumab and G9.2-17 |
| | IgG4 for recurrent severe (Grade 3) or life- |
| | threatening Grade 4 diarrhea or colitis. |
| | Resume G9.2-17 IgG4 at a 50% dose reduction |
| | when signs and symptoms improve or resolve to |
| | baseline. Any dose modifications for tislelizumab |
| | are at the Investigator's discretion. |
| | If ≥ Grade 3 recurs, then permanently discontinue |
| | treatment with both the study medication and |
| | tislelizumab. |
| | If ≥ Grade 2 colitis recurs, withhold G9.2-17 IgG4 |
| | and tislelizumab until colitis resolves to baseline. |
| | Apply treatment regimen outlined above. Resume |
| | G9.2-17 IgG4 at an additional 50% dose reduction. |
| | No further dose reductions for G9.2-17 IgG4 are |
| | allowed. |
| | If ≥ Grade 2 colitis recurs, permanently discontinue |
| | treatment with both study medications. |
| | Permanently discontinue G9.2-17 IgG4 and |
| | tislelizumab for life-threatening (Grade 4). |
| Immune-mediated hepatitis | Monitor for changes in liver function. |
| | Administer corticosteroids at a dose of 0.5 to 1 |
| | mg/kg/day prednisone equivalents for moderate |
| | (Grade 2) transaminase elevations. |
| | Withhold tislelizumab and G9.2-17 IgG4 for |
| | moderate (Grade 2) immune-mediated hepatitis. |
| | When resolved to <Grade 2, and prednisolone |
| | tapered to ≤ 10 mg, resume tislelizumab and G9.2- |
| | 17 IgG4. G9.2-17 IgG4 is to be resumed at a 50% |
| | dose reduction. |
| | If ≥ Grade 2 hepatitis recurs, permanently |
| | discontinue treatment with both G9.2-17 IgG4 and |
| | tislelizumab. |
| | Administer corticosteroids at a dose of 0.5 to 2 |
| | mg/kg/day prednisone equivalents followed by |
| | corticosteroid taper for moderate (grade 2), severe |
| | (Grade 3) or life-threatening (Grade 4) transaminase |
| | elevations, with or without concomitant elevation in |
| | total bilirubin. |
| | Permanently discontinue treatment with both G9.2- |
| | 17 IgG4 and tislelizumab for severe (Grade 3) or |
| | life-threatening (grade 4) immune-mediated |
| | hepatitis. |
| Immune-mediated | Administer hormone replacement as clinically |
| endocrinopathies: | indicated and corticosteroids. Withhold G9.2-17 |
| Hypophysitis | IgG4 and tislelizumab for moderate (Grade 2) |
| | hypophysitis. First administer oral prednisolone 0.5 |
| | to 1 mg/kg/day and add hormone replacement as |
| | clinically indicated until resolved/improved to ≤ |
| | Grade 2. Taper corticosteroids over at least 1 month. |
| | Resume G9.2-17 IgG4 at a 50% dose reduction |
| | when resuming. Discontinuation is usually not |
| | necessary for asymptomatic and manageable patients |
| | who are treated by an endocrinologist. |
| | If ≥ Grade 2 toxicity recurs, permanently |
| | discontinue treatment with both G9.2-17 IgG4 and |
| | tislelizumab. |
| | Permanently discontinue treatment G9.2-17 IgG4 |
| | and tislelizumab for severe (grade 3) and life- |
| | threatening (Grade 4) hypophysitis. |
| Adrenal insufficiency | Monitor patients for signs and symptoms of adrenal |
| | insufficiency. |
| | Withhold G9.2-17 IgG4 and tislelizumab for |
| | moderate (Grade 2) adrenal insufficiency. First |
| | administer oral prednisolone 0.5 to 1 mg/kg/day as |
| | clinically indicated until resolved/improved to ≤ |
| | Grade 2. Taper corticosteroids over at least 1 month. |
| | Resume G9.2-17 IgG4 at a 50% dose reduction |
| | when resuming. Discontinuation is usually not |
| | necessary for asymptomatic and manageable patients |
| | who are treated by an endocrinologist. |
| | If ≥ Grade 2 toxicity recurs, permanently |
| | discontinue treatment with both G9.2-17 IgG4 |
| | and tislelizumab. |
| | Permanently discontinue treatment G9.2-17 |
| | IgG4 and tislelizumab for severe (grade 3) and |
| | life-threatening (Grade 4) adrenal insufficiency. |
| Hypothyroidism and | Monitor thyroid function prior to and periodically |
| Hyperthyroidism | during tislelizumab treatment. |
| | Administer hormone-replacement therapy for |
| | hypothyroidism. |
| | Initiate medical management for control of |
| | hyperthyroidism. Administer oral prednisolone 0.5 |
| | mg/kg/day in patients with thyroid pain. |
| | Thyrotoxic patients should be treated with a beta |
| | blocker and may require additional treatment with a |
| | carbimazole. |
| | Hold tislelizumab for thyroiditis Grade 3 or 4 until |
| | this condition resolves to a Grade 0-1, then consider |
| | resuming treatment. |
| | Discontinue G9.2-17 IgG4 and tislelizumab for ≥ |
| | Grade 3 and 4 hypothyroidism or hyperthyroidism |
| Type 1 Diabetes | Monitor patients for hyperglycemia or other signs |
| Mellitus | and symptoms of diabetes. |
| | Withhold G9.2-17 IgG4 and tislelizumab in cases of |
| | severe (Grade 3) hyperglycemia until metabolic |
| | control is achieved, until blood glucose has been |
| | stabilized to baseline or Grade 0 to 1 and the patient |
| | is hyperglycemia symptom-free. Administer insulin |
| | as clinically indicated for type 1 diabetes. |
| | Follow G9.2-17 IgG4 IMAR guidance for Type 1 |
| | Diabetes Mellitus when resolved to ≤ Grade 2. |
| | Permanently discontinue treatment with both study |
| | medications for life-threatening (Grade 4) |
| | hyperglycemia. |
| Immune-mediated nephritis and | Monitor patients for renal function prior to and |
| renal dysfunction | periodically during treatment. In the event of |
| (Defined as renal dysfunction | immune-mediated nephritis, the frequency of renal |
| or ≥ Grade 2 increased creatinine, | function tests should be increased. |
| requirement for corticosteroids, | Withhold G9.2-17 IgG4 and tislelizumab for |
| and no clear alternate etiology) | moderate (Grade 2) or severe (Grade 3) increased |
| | nephritis. When resolved to < Grade 2, resume G9.2- |
| | 17 IgG4 at a 50% dose reduction. If ≥ Grade 2 |
| | toxicity recurs, permanently discontinue treatment |
| | with both G9.2-17 IgG4 and tislelizumab. |
| | Ensure hydration and administer corticosteroids at a |
| | dose of 0.5 to 1 mg/kg/day prednisone equivalents |
| | for moderate (Grade 2) or 1 to 2 mg/kg/day |
| | prednisone equivalents for severe (Grade 3) |
| | nephritis. |
| | Permanently discontinue treatment with both G9.2- |
| | 17 IgG4 and tislelizumab for life-threatening (Grade |
| | 4) nephritis and recurrent Grade 3 nephritis as well. |
| Immune-mediated skin adverse | For ≤ Grade 2 rashes, treatment that can be |
| reactions | controlled or resolved with moderate strength topical |
| (Tislelizumab can cause | or oral steroids while continuing tislelizumab and |
| immune-mediated rash, | G9.2-17 IgG4. |
| including Stevens-Johnson | Monitor patients for suspected severe skin reactions |
| syndrome (SJS) and toxic | and exclude other causes. |
| epidermal necrolysis (TEN), | Withhold both medications for severe (Grade 3) |
| some cases with fatal outcome.) | until signs and symptoms become mild, and |
| | permanently discontinued for moderate (Grade 2) to |
| | severe (Grade 3) rashes that do not improve or |
| | worsen while treated with oral or IV prednisolone, |
| | and permanently discontinue for life-threatening |
| | (Grade 4) rash. |
| | For symptoms or signs of SJS or TEN, permanently |
| | discontinue tislelizumab and G9.2-17 IgG4 and refer |
| | the patient for specialized care for assessment and |
| | treatment. |
| | The recommended dose of oral prednisolone is 0.5 |
| | to 1 mg/kg/day for 3 days with tapering over 2 to 4 |
| | weeks (moderate rashes) and using methyl |
| | prednisolone 0.5 to 1 mg/kg/day with converting to |
| | oral prednisolone and tapering over at least 4 weeks |
| | (severe rashes). |
| | When resolved to < Grade 2, resume G9.2-17 IgG4 |
| | at a 50% dose reduction. |
| Immune-mediated encephalitis | Monitor for changes in neurologic function. |
| | Evaluation of patients with neurologic symptoms |
| | may include, but not be limited to, consultation with |
| | a neurologist, brain MRI, and lumbar puncture. |
| | Withhold G9.2-17 IgG4 and tislelizumab in patients |
| | with new-onset moderate to severe neurologic signs |
| | or symptoms and evaluate to rule out infectious or |
| | other causes of moderate to severe neurologic |
| | deterioration. |
| | If other etiologies are ruled out, administer |
| | corticosteroids at a dose of 1 to 2 mg/kg/day |
| | prednisone equivalents for patients with immune- |
| | mediated encephalitis, followed by corticosteroid |
| | taper. |
| | Permanently discontinue treatment with G9.2-17 |
| | IgG4 and tislelizumab for immune-mediated |
| | encephalitis. |
| Other Immune-Mediated | For any suspected immune-mediated adverse |
| Adverse Reactions | reactions, exclude other causes. |
| The following immune- | Based on the severity of the adverse reaction, |
| mediated adverse reactions | permanently discontinue or withhold G9.2-17 IgG4 |
| occurred in less than 1% of | and tislelizumab, administer high-dose |
| patients treated with | corticosteroids, and if appropriate, initiate hormone- |
| tislelizumab: arthritis, | replacement therapy. |
| encephalitis, rhabdomyolysis, | Upon improvement to Grade 1 or less, initiate |
| myositis, myocarditis, | corticosteroid taper and continue to taper over at |
| pancreatitis, and uveitis. | least 1 month. |
| | Consider restarting G9.2-17 IgG4 and tislelizumab |
| | after completion of corticosteroid taper based on the |
| | severity of the event. Resume G9.2-17 IgG4 at a |
| | 50% dose reduction. Immune-mediated adverse |
| | reactions may occur after discontinuation |
| | of tislelizumab therapy. |
| Infusion reactions | The symptoms of infusion-related reactions that may |
| (Reported in less than 1.0% of | be observed with tislelizumab include fever, |
| patients in clinical trials.) | chills/rigor, nausea, pruritus, angioedema, |
| | hypotension, headache, bronchospasm, urticaria, |
| | rash, vomiting, myalgia, dizziness, or hypertension. |
| | Patients should be closely monitored for these signs |
| | and symptoms during the infusion. |
| | For Grade 1 reactions, decrease infusion rate by |
| | 50%; for Grade 2 reactions, stop infusion and only |
| | resume with caution at 50% of previous rate if |
| | infusion-related reaction has resolved or decreased |
| | to Grade 1 in severity. For Grade 3 or Grade 4 |
| | infusion-related reactions, stop infusion and |
| | permanently discontinue tislelizumab. Subsequent |
| | infusions should be given after premedication and at |
| | the reduced infusion rate. |
| Complications of Allogeneic | Transplant-related complications include hyperacute |
| Hematopoietic Stem Cell | graft-versus-host-disease (GVHD), acute GVHD, |
| Transplantation (HSCT) | chronic GVHD, hepatic veno-occlusive disease |
| (Fatal and other serious | (VOD) after reduced intensity conditioning, and |
| complications can occur in | steroid-requiring febrile syndrome (without an |
| patients who receive allogeneic | identified infectious cause). |
| HSCT before or after being | These complications may occur despite intervening |
| treated with a PD-1 receptor | therapy between PD-1 blockade and allogeneic |
| blocking antibody.) | HSCT. |
| | Follow patients closely for evidence of transplant- |
| | related complications and intervene promptly. |
| | Consider the benefit versus risks of treatment with a |
| | PD-1 receptor blocking antibody prior to or after an |
| | allogeneic HSCT. |
| Embryo-Fetal Toxicity | Advise pregnant women of the potential risk to a |
| (Based on its mechanism of | fetus. |
| action and data from animal | Advise females of reproductive potential to use |
| studies, tislelizumab can cause | effective contraception during treatment with a |
| fetal harm when administered to | tislelizumab-containing regimen and for at least 5 |
| a pregnant woman.) | months after the last dose of tislelizumab. |

For non-IMAR, hematological and non-hematological AEs that occur in combination arms, upon assessment of causality:
If G9.2-17 IgG4 related, follow AE management instructions for G9.2-17 IgG4
If combination agent related (tislelizumab), follow management instructions in Table 36 for tislelizumab.

TABLE 36
Recommended Dose Modifications for Tislelizumab
for AEs (other than IMARS)

| Adverse | | Dose |
|---|---|---|
| Reaction | Severity* | Modification |
| AEs | Probably related Grade 3 adverse events | Permanently |
| | Life-threatening or Grade 4 adverse | discontinue |
| | reaction | |

*Toxicity was graded per NCI CTCAE V5.

### Concomitant Therapy

Any medication or vaccine (including over-the-counter or prescription medicines, recreational drugs, vitamins, and/or herbal supplements) that the participant is receiving at the time of enrollment or receives during the study must be recorded along with:
Reason for use
Dates of administration including start and end dates
Dosage information including dose and frequency

### Permitted Medications

The following concomitant medications are allowed:
Any standard of care pre-medication for patients on a combination treatment regimen.
Continuation of bisphosphonate treatment (eg, zoledronic acid) or denosumab for bone metastases, which have been stable for at least 6 months before treatment (C1D1),
The use of inhaled corticosteroids and mineralocorticoids (eg, fludrocortisone), topical steroids, intranasal steroids, intra-articular, and ophthalmic steroids
Prophylactic or therapeutic use of anticoagulants
Vaccination for COVID-19, common flu and/or other common clinically required indications (eg tetanus, pneumococcus, HBV, etc.) is allowed before or during the study period. The timing and type of vaccine must be recorded.

### Prohibited Medications

Following medications are not allowed while on this study:
Concomitant administration of other investigational agents, other than G9.2-17 IGG4, for any indication
Systemic immunosuppressive treatment, including, but not limited to cyclophosphamide, azathioprine, methotrexate, thalidomide, and anti-TNF agents. However, patients are allowed to take acute, low dose systemic immunosuppressant medications (eg, ≤10 mg/day of prednisone or equivalent).
Replacement therapy (eg, thyroxine, insulin, physiologic corticosteroid replacement therapy [eg, ≤10 mg/day of prednisone equivalent] for adrenal or pituitary insufficiency) is not considered a form of systemic treatment.

### Study Procedures and Evaluations

Incidence and severity of AEs/CTAEs and SAEs, including clinically significant changes in laboratory parameters, vital signs & ECGs
Incidence of DLTs, PK and PD
Plasma PK parameters (e.g., AUC0-24h, Gm., Tmax, estimated half-life); serum concentration vs. time profiles
Objective response rate (complete response and partial response) (ORR) and clinical benefit rate (objective response and stable disease 3 months or longer); progression free survival (PFS), overall survival (OS), disease control rate (DCR)
Blood and tumor immunophenotyping, galectin-9 serum, plasma levels, and tissue expression levels and expression pattern of tumor, stroma, immune cells), time to response (TTR), and other biomarker analysis, CT (PET) imaging, other clinically relevant imaging.

### Safety Monitoring

Routine safety monitoring is performed by the Medical Monitor. Safety monitoring, including analysis of PK, will be performed by a Safety Monitoring Committee (SMC), consisting of the Principal Investigators (and co-investigators as needed) and sponsor representatives and the study-specific Medical Monitor. Additional investigators and study team members will participate in reviews as needed. An Independent Data Monitoring Board is not be utilized for this open-label study.

In Stage 1, the dose-escalation phase, dose escalation to the next cohort proceeds following review of Cycle 1 of each cohort. Safety and available PK data are used to assess for a dose-limiting toxicity (DLT) in all patients of each cohort by the SMC. As a safety precaution, during dose escalation, new patients are entered and treated only after the first patient of each cohort has been treated with the anti-Gal9 antibody and at a minimum 7 days post-treatment has elapsed. Select DLT safety analysis for each patient is performed following completion of Cycle 1.

Dose-limiting toxicity (DLT) is defined as a clinically significant hematologic or non-hematologic adverse event or abnormal laboratory value assessed as unrelated to metastatic tumor disease progression, intercurrent illness, or concomitant medications and is related to the study drug and occurring during the first cycle on study that meets any of the following criteria:
1. All Grade 4 hematologic and non-hematologic toxicities of any duration
2. All Grade 3 hematologic and non-hematologic toxicities. Exceptions are as follow:
   a. Grade 3 nausea, vomiting and diarrhea that does not require hospitalization or TPN support and can be managed with supportive care to ≤grade 2 within 48 hours.
   b. Grade 3 electrolyte abnormalities that are corrected to ≤grade 2 within 24 hours.

Other grade 3 asymptomatic laboratory abnormalities DLT Period=One (1) cycle, i.e. two doses of G9.2-17 IgG4 on days 1 and 15 of each cycle.

Patients should ordinarily be maintained on study treatment until confirmed radiographic progression. If the patient has radiographic progression but no unequivocal clinical progression and alternate treatment is not initiated, the patient may continue on study treatment, at the investigator's discretion. However, if patients have unequivocal clinical progression without radiographic progression, study treatment is stopped and patients advised regarding available treatment options.

Both the approved checkpoint inhibitor and G9.2-17 IgG4 is withheld in the event of a serious or life-threatening immune related adverse reaction(IMAR) or one that prompts initiation of systemic steroids, although specific exceptions (e.g., for certain endocrinopathies in clinically stable patients) may be described in the approved product labeling.

If the protocol proposes continuation of an experimental agent in the setting of either (a) withholding the approved checkpoint inhibitor, or (b) initiation of systemic steroids for an IMAR, provide sufficient justification supporting the safety of such an approach.

In the event where dose-reduction is used for AE management, two dose reductions are allowed. By 30% of the baseline dose at each dose reduction. Dose reductions are to be pursued only if the investigator assesses that clinical benefit is being and may continue to be derived.

Treatment emergent adverse events (TEAEs) will be defined as events that occur on or after the first dose of study medication. The Medical Dictionary for Regulatory Activities (MedDRA) coding dictionary will be used for the coding of AEs. TEAEs, serious or CTC grade 3 or 4 TEAEs, and TEAEs related to therapy will be summarized overall and by system organ class and preferred term by treatment group. These will summarize the number of events and the number and percent of patients with a given event. In addition, the number and percent of patients with TEAEs will be provided by maximum severity. A summary of all TEAEs by system organ class and preferred term occurring in at least 5 percent of patients in either treatment group will be provided.

Any AE>Grade 3 possible, probably, or definitely related to one or more study drugs will be discussed with the Medical Monitor before continuing with dosing, with the following exceptions, for which no discussion with the Medical Monitor will be required:
Local injection site reactions lasting <72 hours including pain, redness, swelling, induration, or pruritus
Systemic injection reactions lasting <72 hours of fever, myalgia, headache, or fatigue

Where judged appropriate by the Investigator (after discussion with the Medical Monitor) a dose delay may be necessary for >Grade 3 adverse events until resolution of the toxicity (to Grade 1 or less).

In Part 2 of the protocol, if one or more patients develop a DLT, the dose of G9.2-17 IgG4 will be reduced to 1 dose below the recommended Phase 2 dose (RP2D).

Once a patient has completed the treatment period, overall survival follow-up is performed every 3 months for up to 2 years. Radiological assessment continues, where possible, for patients withdrawing due to clinical progression.

The following procedures will be done on Day 59 or thirty days after the last dose, including patients who have discontinued treatment early.
Restaging scan (CT with contrast, MRI, PET-CT or X-ray)—repeat if end of study is >6 to 8 weeks after last cycle and in shorter intervals it investigator's discretion
Relevant tumor marker—e.g., Ca15-3, CA-125, CEA, CA19-9, S100, alpha fetoprotein, etc., will be assessed every cycle pre-dose (which may be decreased to every 3 cycles after 6 months of treatment, following the same schedule as restaging scans), as appropriate
12-lead ECG
Physical examination
ECOG
Vital signs (temperature, HR, BP, RR, including weight) post-supine for 5 minutes
Concomitant medications (name, indication, dose, route, start and end dates)
Adverse events
Pregnancy test, if female
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK, TSH, fT4, PTH, Estradiol, prolactin, testosterone, FSH, LH, gamma glutamyl transferase (gamma GT), hemoglobin Alc (HgbAlc) (only if history of Type 1 or Type 2 diabetes mellitus), lipase, amylase, free cortisol additionally at specified visits
Hematology—complete blood count, differential, platelets, hemoglobin
Blood coagulation (PT, PTT, APTT, INR, CRP, and troponin)
Urinalysis
PD blood—biomarker analysis
PK blood samples

### Pharmacokinetics Assessments

The following serum PK parameters will be calculated for G9.2-17 IgG4, if possible:
AUC₀₋₃₃₆ₕ
Cₘₐₓ
Tₘₐₓ
t_{1/2}
serum concentration vs. time profiles

Blood samples of approximately 5 mL will be collected and processed to serum at each timepoint as specified in the table of Schedule of Assessment.

### Cycle 1 and Cycle 3 Day 1

Pre-dose
At the End of Infusion (EOI)
2 h (±30 min) after EOI
4 h (±30 min) after EOI

### Cycle 1 and Cycle 3 Day 15

Pre-dose
At EOI

### Cycle 1 and Cycle 3 Day 2 and 8 (Non-Dosing Days)

Any point during the visit

### Cycle 2 and Cycle 4 Day 1

Pre-dose
At EOI

### Every 2 Cycles Beyond Cycle 4 on Day 1 (i.e., C6DI, C8D1 etc.):

Pre-dose
At EOI

If the dose of study drug should be interrupted, additional PK and safety assessments will be collected upon resumption of dosing; additional PK assessments may be performed during the interruption. If the dose of study drug is reduced, additional PK assessments will be collected pre-administration of the reduced dose (within 2 h pre-dosing), and 2 to 4 h after starting the reduced study drug dose. Additional PK, and other blood assessments may be taken if clinically indicated.

Concentrations will be determined using validated assays. A minimum of two 50 μL aliquots of serum are needed to determine total G9.2-17 IgG4 concentrations. A minimum of two 100 μL aliquots of serum are needed to determine free and partially free G9.2-17 IgG4 concentrations and residual serum in a third aliquot. Samples collected for analyses of G9.2-17 IgG4 plasma concentration may also be used to evaluate safety or efficacy aspects related to concerns arising during or after the study.

Genetic analyses will not be performed on these blood samples. At visits during which blood samples for the determination of PD, ADA, safety lab of G9.2-17 IgG4 will be taken, one sample of sufficient volume can be used. PD biomarkers are evaluated in this study and are described herein.

### Pharmacodynamic Biomarkers

Planned time points for biomarker assessments are provided in the table of Schedule of Assessment. Sampling may be decreased to every 3rd cycle after 6 months of treatment. Collection of biological samples for other biomarker research is also part of this study. The following samples for biomarker research are required and will be collected from all participants in this study as specified in the table of Schedule of Assessment.
Blood samples, to be collected prior to study drug administration (approximately 15 mL pre-dose)
Tumor biopsy (tissue sample)

Samples will be tested for PD biomarkers (by flow cytometry, ELISA, IHC, or multiplex phenotyping) to evaluate their association with the observed clinical responses to G9.2-17 IgG4 using validated assays.

The following biomarkers are planned to be assessed for this study:
Tumor markers (blood): CA15-3, CA-125, carcinoembryonic antigen (CEA), CA19-9, S100, alpha fetoprotein, neuron-specific enolase (NSE), cytokeratin-fragment-21 (CYFRA-21) to be assessed every cycle pre-dose administration as needed per tumor type. This may be decreased to every 3 cycles after 6 months of treatment, following the same schedule as tumor imaging assessments, as appropriate.
PBMC phenotype (blood): eg, CD3, CD4, CD8, CD45RO, forkhead-box-protein P3 (FOXP3), CD11B, CD14, CD15, CD16, CD33, CD68, human leukocyte antigen (HLA) DR, CD163, arginase 1, granzyme B, KI67, PD-1, PD-L1, pan cytokeratin (PAN-CK)
Cytokines (blood): eg, interferon gamma (IFN-γ), IL-10, IL-12p70, IL-13, IL-1β, IL-2, IL-4, IL-6, IL-8, TNF-α, MIP-1b, monocyte chemoattractant protein 1 (MCP-1), MIP-1a, IL-17a, IL-5, TGF-β
Gal-9 in blood and tumor tissue
PD-L1 (tissue)
Mismatch repair status (tissue)
Tumor Mutational Burden (TMB)

### Immunogenicity Assessments

Blood samples (approximately 3 mL) will be collected from all participants according to the table of Schedule of Assessment and processed to serum. Additionally, serum samples should also be collected at the end of treatment/early termination visit from patients who discontinued study intervention or were withdrawn from the study.

### Cycle 1 to Cycle 4 Day 1

Pre-dose

Every 2 cycles beyond Cycle 4 on Day 1 (i.e., C6D1, C8D1 etc.):
Pre-dose

A minimum of two aliquots of 500 μL serum each, with residual serum in a third tube will be obtained. Serum samples will be screened for antibodies binding to G9.2-17 IgG4

(ADA) and the titer of confirmed positive samples will be reported. Other analyses may be performed to verify the stability of antibodies to G9.2-17 IgG4 and/or further characterize the immunogenicity of G9.2-17 IgG4.

The detection and characterization of antibodies to G9.2-17 IgG4 will be performed using a validated assay method. All samples collected for detection of antibodies to study intervention will also be evaluated for G9.2-17 IgG4 serum concentration to enable interpretation of the antibody data. Antibodies may be further characterized and/or evaluated for their ability to neutralize the activity of the study intervention.

### Other Assessments

### Demographics

At Screening, patient demographic data will be collected. These include age, gender, race, and ethnicity.

### Medical History

The medical history will include oncology history, surgical/transplant history radiation therapy history, and COVID-19 history and testing.
Personal medical history, including prior treatments/surgeries, record of any implants in situ or past implants, prior and/or current use of medical devices, concomitant medications (name, indication, dose, route, start and end dates dose modifications if any and reason), pre-existing symptoms, and AEs), hereditary diseases at risk of based on family history and complete family history to the best knowledge of the patient
Record of any dental work performed in the past 12 months
For patients with previously resected tumor, record where the primary tumor was localized.
Bowel habits/typical frequency and consistency
Record any dietary requirements or preferences (for example, practice of a particular diet regimen: intermittent fasting, keto diet etc.)
Record and allergies past and present (allergen, severity)

### Prior and Concomitant Medications

Prior and concomitant medications, including vaccines and complementary treatments/supplements, will be documented for each patient at each scheduled visit.

### Tumor Imaging Assessments

Tumor assessments will be performed using CT or MRI with or without contrast; a PET-CT will be performed. CT with contrast is the preferred modality (MRI, PET-CT, or other imaging modalities instead of, or in addition to, the CT scan, if CT is not feasible or appropriate, given location of the disease). Assessment should include the chest/abdomen/pelvis at a minimum and should include other anatomic regions as indicated, based on the patient's tumor type and/or disease history. Imaging scans must be de-identified and archived in their native format as part of the patient study file.

On study, assessments are done every 8 weeks±7 days according to the SoA (ie, C3D1, C5D1, C7D1, C9D1, etc.) and at the End of Treatment if not assessed within the last 4-6 weeks. Assessments may be performed more frequently if clinically indicated. For Part 2 only, if an objective response is seen on a scan, a confirmation scan will be done 4 weeks (+7 days) later. After a confirmatory scan, the scheduled scans are to be resumed at a frequency of every 8 weeks (±7 days) from the date of the confirmatory scan.

### Tumor Biopsies

Pre- and on-treatment biopsies are collected. A pre-treatment biopsy is to be collected during screening. If a pre-treatment biopsy is unobtainable as per the reasons outlined in the inclusion criteria, and the patient is enrolled in the study, an archival tumor tissue specimen from that patient will be collected from a primary tumor and/or a metastatic deposit. Excisional or core biopsy (FFPE tissue block(s) OR fresh tissue in formalin) obtained currently or within 5 years before study start from the primary tumor lesion or a metastatic deposit. If both primary and metastatic tissues are available, use of metastatic deposit tissue will be prioritized. If information of treatment(s) received before and after tissue acquisition are available, this will be collected as well.

The on-treatment biopsy is scheduled for C3D15 +7 days and should occur only after the tumor imaging scan in Cycle 3. In instances where the procedure cannot be performed within the protocol-specified timeframe, alternatives may be permitted but must be discussed with the Study Director/Medical Monitor. It is recognized that a variety of clinical factors may make it difficult to obtain adequate specimens. Decisions not to complete biopsy on-treatment should be discussed with the Medical Monitor.

### ECHO/MUGA

ECHO and/or MUGA will be obtained at the timepoints indicated in the Schedule of Assessment table. If clinically indicated, the assessment is to be repeated once every 3 months.

### RECIST Criteria for Tumor Assessment

At the baseline tumor assessment, tumor lesions/lymph nodes are categorized as measurable or non-measurable with measurable tumor lesions recorded according to the longest diameter in the plane of measurement (except for pathological lymph nodes, which are measured in the shortest axis). When more than one measurable lesion is present at baseline all lesions up to a maximum of five lesions total (and a maximum of two lesions per organ) representative of all involved organs should be identified as target lesions. Target lesions are selected on the basis of their size (lesions with the longest diameter). A sum of the diameters for all target lesions is calculated and reported as the baseline sum diameters.

All other lesions (or sites of disease) including pathological lymph nodes is identified as non-target lesions and are also be recorded at baseline. Measurements are not required and these lesions are followed as ‘present’, ‘absent’, or ‘unequivocal progression’.

Disease response (complete response (CR), partial response (PR), stable disease (SD), and progressive disease (PD)) is be assessed as outlined below.

The disease response measures allow for the calculation of the overall disease control rate

(DCR), which includes CR, PR, and SD, the objective response rate (ORR), which includes CR and PR, progression-free survival (PFS), and time to progression (TTP).

### The Response Evaluation Criteria in Solid Tumors (RECIST) Guidelines

The overall response according to RECIST 1.1 is derived from time-point response assessments based on tumor burden as follows below.

### Evaluation of Target Lesions

Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD): Neither sufficient shrinkage to qualify for PR nor sufficient increase to qualify for PD, taking as reference the smallest sum diameters while on study.

### Evaluation of Non-Target Lesions

Complete Response (CR): Disappearance of all non-target lesions and normalization of tumor marker level. All lymph nodes must be non-pathological in size (<10 mm short axis).
Non-CR/Non-PD: Persistence of one or more non-target lesion(s) and/or maintenance of tumor marker level above the normal limits.
Progressive Disease (PD): Unequivocal progression of existing non-target lesions. (Note: the appearance of one or more new lesions is also considered progression).

The disease response measures at different timepoints will allow for the calculation of the following:
Disease control rate (DCR), defined as percentage of patients who have achieved CR, PR and SD.
Objective response rate (ORR), defined as the proportion of patients with tumor size reduction of a predefined amount (tumor shrinkage of ≥30%).
Progression-free survival (PFS), defined as the time from study drug treatment initiation to disease progression (tumor growth by ≥30%).
Duration of response (DoR), defined as the length of time that a tumor continues to respond to treatment without the cancer growing or spreading.
Overall survival (OS) defined as defined as the time from study drug treatment initiation to death from any cause.

TABLE 37
Evaluation of Overall Timepoint Response for
Patients with Measurable Disease at Baseline

| Target | Non-target | New | Overall |
|---|---|---|---|
| Lesions | Lesions | Lesions | Response |
| CR | CR | No | CR |
| CR | Non-CR/Non-PD | No | PR |
| CR | NE | No | PR |
| PR | Non-PD or NE | No | PR |
| SD | Non-PD or NE | No | SD |
| Not all | Non-PD | No | NE |
| evaluated | | | |
| Progressive | Any | Yes or No | Progressive |
| disease | | | disease |
| Any | Progressive | Yes or No | Progressive |
| | disease | | disease |
| Any | Any | Yes | Progressive |
| | | | disease |

CR: Complete Response,
Non-PD: Non-progressive Disease,
PR: Partial Response,
SD: Stable Disease,
NE: Non-evaluable
*When target lesions show SD/PR and some subset of non-target lesions is non-evaluable, a careful decision must be made whether to call the overall response at this timepoint SD/PR or NE. This is based on whether the non-evaluable lesions, if they showed growth, could cause an overall response of progressive disease in the context of the other lesion responses seen. If the non-evaluable non-target lesions comprise a significant proportion of the overall disease burden, the appropriate timepoint response is NE.

### Study Drug And Other Intervention

Study intervention(s) is/are defined as any investigational agent(s), marketed product(s), placebo, or medical device(s) intended to be administered/used to/in a study participant according to the study protocol.

### Adverse Events

### Definitions

### Adverse Events (AE)

An AE is defined in the ICH Guideline for GCP as “any untoward medical occurrence in a patient or clinical investigation patient administered a pharmaceutical product and that does not necessarily have a causal relationship with this treatment.” This definition of AEs is broadened in this study to include any such occurrence (e.g., sign, symptom, or diagnosis) or worsening of a pre-existing medical condition from the time that a patient has signed informed consent to the time of initiation of the investigational drug. Worsening indicates that the pre-existing medical condition (e.g., diabetes, migraine headaches, gout, hypertension, etc.) has increased in severity, frequency, or duration of the condition or an association with significantly worse outcomes.

### Serious Adverse Events (SAE)

A SAE is defined as an AE that:
Results in death;
Is life threatening (places the patient at immediate risk of death);
Requires in-patient hospitalization or prolongation of existing hospitalization;

A hospitalization meeting the definition for “serious” is any inpatient hospital admission that includes a minimum of an overnight stay in a health care facility. Inpatient admission does not include rehabilitation facilities, hospice facilities, skilled nursing facilities, nursing homes, routine emergency room admissions, same day surgeries (as outpatient/same day/ambulatory procedures), or social admission (eg, patient has no place to sleep).
Results in persistent or significant disability/incapacity; or
Is a congenital anomaly/birth defect
Important medical events that may not result in death, be life threatening, or require hospitalization may be considered an SAE when, based upon appropriate medical judgment, they may jeopardize the patient and may require medical or surgical intervention to prevent one of the outcomes listed in this definition. Examples of such medical events include anaphylaxis and allergic bronchospasm requiring intensive treatment in an emergency room or at home, blood dyscrasias or convulsions that do not result in inpatient hospitalization.

### Relatedness

For all AEs, enough information should be obtained to determine the causality of the AE (eg, study drug or other illness). The relationship of the AE to the study treatment will be assessed following the definitions below:
Unrelated: any event that does not follow a reasonable temporal sequence from administration of study drug AND that is likely to have been produced by the patient's clinical state or other modes of therapy administered to the patient.
Unlikely Related: any event that does not follow a reasonable temporal sequence from administration of study drug OR that is likely to have been produced by the patient's clinical state or other modes of therapy administered to the patient.
Possibly Related: any reaction that follows a reasonable temporal sequence from administration of study drug OR that follows a known response pattern to the suspected drug AND that could not be reasonably explained by the known characteristics of the patient's clinical state or other modes of therapy administered to the patient.
Related: any reaction that follows a reasonable temporal sequence from administration of study drug AND that follows a known response pattern to the suspected drug AND that recurs with re-challenge, AND/OR is improved by stopping the drug or reducing the dose.

### Adverse Event Management

AEs will not be recorded prior to the administration of the first dose of study medication. AEs that start, or symptoms related to medical history that worsen after study drug administration will be recorded. AEs should be followed until they are either resolved, have returned to baseline, or are determined to be a stable or chronic condition. All SAEs are to be collected until 30 days after the last dose of study medication.

### Immune-Mediated Adverse Reactions

Immune-mediated adverse reactions (IMARs) are identified for nivolumab in the Warnings and Precautions section of the package insert. The specific IMARs noted are:
Immune-mediated pneumonitis
Immune-mediated colitis
Immune-mediated endocrinopathies
Immune-mediated nephritis and renal dysfunctions
Immune-mediated Skin adverse reactions
Immune-mediated Encephalitis
Infusion reactions
Complications of allogeneic HSCT
Embryo-fetal toxicity

The monitoring plan is intended to limit the severity and duration of IMARs that occur during combination drug development, and encompass: scheduled visits for a physical exam, vital signs, safety laboratory assessments including blood hematology, biochemistry, assessing endocrine functions each Day 1 of a new dosing cycle (pre-dose), assessing coagulation status and urine analyses. The Schedule of Assessments also encompasses assessing the ejection fraction once every three months and conducting regular ECGs.

Instructions for the management of these IMARs are provided below (for G9.2-17 IgG4 alone (Table C), and for the G9.2-17 IgG4+nivolumab combination treatment (Table A2)).

### Dose-Reduction Procedure for Adverse Event Management

In the event where dose-reduction is used for AE management in Part 2 of the study, two dose reductions of 50% each are allowed.

### Clinical Laboratory Abnormalities and Other Abnormal Assessments as AEs and SAEs

Abnormal laboratory findings (e.g., clinical chemistry, hematology, and urinalysis) or other abnormal assessments (e.g., ECGs or vital signs) that are judged as clinically significant will be recorded as AEs and SAEs if they meet the definition of an AE or SAE. Clinically significant abnormal laboratory findings or other abnormal assessments that are detected during the study or are present at screening and significantly worsen following the start of the study will be reported as AEs or SAEs. However, clinically significant abnormal laboratory findings or other abnormal assessments that are associated with the disease being studied, unless judged as more severe than expected for the patient's condition, or that are present or detected at the start of the study and do not worsen, will not be reported as AEs or SAEs.

Laboratory measurements that deviate clinically significantly from previous measurements may be repeated. If warranted, additional or more frequent testing than is specified in the protocol should be done to provide adequate documentation of AEs and the resolution of AEs.

### Statistical Considerations

The study will be completed when the last patient has had their last visit. The database will be locked for the primary analysis after the last patient has had their primary endpoint event. A final study analysis will be performed after study completion.

### Statistical Hypotheses

The current study is designed to identify the MTD of G9.2-17 IGG4 (Part 1) by assessing DLTs, followed by an assessment of drug activity (alone or in combination) in the three disease types using Simon's two-stage optimal design. Study hypotheses for Part 2 are detailed below.

### G9.2-17 IGG4 Single Agent Treatment Arms

Null hypothesis: ORR-3 is ≤5%
Alternative hypothesis: ORR-3 is ≥15%.

### G9.2-17 IGG4+Nivolumab Combination Treatment Arms

Null hypothesis: ORR-3 is ≤10%
Alternative hypothesis: ORR-3 is ≥25%.

### Sample Size Determination

### Part 1

Part 1 of this study (dose escalation phase) will establish DLTs, the OBD/MTD, and the RP2D for G9.2-17 IgG4 using a CRM (O'Quigley et al., 1990), a model-based design that informs how the dosage of G9.2-17 IgG4 should be adapted for the next patient cohort based on past trial data.

Trial simulation analyses with 1000 iterations suggested an average of approximately 20 patients will be needed to identify the OBD/MTD, which is the largest dose that has an estimated probability of a DLT less than or equal to a TTL of 25%. Once the OBD/MTD has been identified, an additional 6 patients may be enrolled to firmly establish the safety profile of G9.2-17 IgG4 in patients with gal-9 positive tumors.

Therefore, a total sample size of 26 patients is anticipated for Part 1 of the study. In cases of screening failures and drop-outs, patient replacement will occur until the MTD has been identified.

### Part 2

Part 2 of this study (cohort expansion phase) will adopt a Simon's two-stage optimal design to establish safety and efficacy for G9.2-17 IgG4 in patients with ocular melanoma. Approximately 223 patients will be treated in Part 2, of whom about 93 will be treated in Simon's Stage I and about 130 in Simon's Stage II.

### Single Agent Treatment Arms

It is estimated that approximately 23 patients are needed per single agent treatment arm in Simon's Stage I to reject the null hypothesis that ORR-3 is ≤5% and to continue to Simon's Stage II.

If the trial goes on to Stage II of Simon's optimal design, approximately 33 patients will be treated additionally in each of the single agent arms to test the alternative hypothesis that ORR-3 is ≥15%. If the alternative hypothesis is not met, the respective arm will be stopped.

### Anti-PD1 Combination Treatment Arms

It is estimated that approximately 18 patients are needed per combination treatment arm in Simon's Stage I to reject the null hypothesis that ORR-3 is ≤10% and to continue to Simon's Stage II.

If the trial goes on to Stage II of Simon's optimal design, approximately 25 patients will be treated additionally in each of the combination arms to test the alternative hypothesis that ORR-3 is >25%. If the alternative hypothesis is not met, the respective arm will be stopped.

For all patients receiving a combination treatment, the treatments can be administered on the same day. The anti-PD-1 antibody should be administered prior to the anti-Galectin-9 antibody. If for any reason same-day administration cannot be accomplished, the anti-PD-1 regimen should be administered on the first day of dosing, and the anti-Galectin-9 antibody on the subsequent day.

### Analysis Sets

The intent-to-treat (ITT) population will be defined as those patients who received at least one dose of the study drug, unless otherwise specified. The primary efficacy analyses will be performed for the ITT. Patient disposition will be performed for the ITT.

The Efficacy Population will be defined as all patients in the ITT and having at least one measurable ORR-3 or PFS-6 assessment. This population will be used for a sensitivity analysis.

The per-protocol (PP) Population will be defined as any patient who received at least one full cycle of G9.2-17 IgG4 and without major protocol deviations.

The safety population (SAF) will be defined as all patients who receive at least one dose of the study drug. The safety analyses will be performed for the SAF.

The PK/PD population will be defined as those patients who have received at least one full cycle of G9.2-17 IgG4.

### Statistical Analyses

The statistical analysis plan (SAP) will be finalized prior to data base lock and it will include a more technical and detailed description of the statistical analyses described in this section. This section is a summary of the planned statistical analyses of the most important endpoints including primary and key secondary endpoints.

### General Considerations

Unless otherwise specified, all continuous endpoints will be summarized using descriptive statistics, which will include the number of patients (n), mean, standard deviation, median, minimum, and maximum. All categorical endpoints will be summarized using frequencies and percentages, with 95% CI. The screening measurement will be the last value on or before the date of first study treatment. Survival curves for, PFS and OS will be generated by the method of Kaplan-Meier from the day treatment was initiated, but there will be no comparative analysis between study arms in any part of the trial. Waterfall and Swimmers plots will be used to graphically present the ORR and DoR for all patients in each study arm, within each tumor type evaluated in Part 2. All statistical analyses will be performed using SAS®, version 9.2. (SAS, Cary, NC).

### Primary Endpoint(s)

### Safety Analysis—Part 1 and Part 2

All safety analyses will be made on the SAF unless otherwise specified.

### Adverse Events

Treatment-emergent adverse events (TEAEs) will be defined as events that occur on or after the first dose of study medication. The MedDRA coding dictionary will be used for the coding of AEs. TEAEs, serious or CTCAE Grade 3 or Grade 4 TEAEs, and TEAEs related to treatment will be summarized overall and by system organ class and preferred term by treatment group. These will summarize the number of events and the number and percent of patients with a given event. In addition, the number and percent of patients with TEAEs will be provided by maximum severity. A summary of all TEAEs by system organ class and preferred term occurring in >5% of patients in either treatment group will be provided. DLTs, the OBD/MTD and the RP2D will be summarized.

### Laboratory Assessments

All laboratory-based data will be presented as listings of all values as well as of abnormal results judged to be clinically significant, which will also be reported as AEs. Numeric summaries of all observed findings and changes from baseline screening laboratory evaluations will be provided by visit and treatment group, including chemistry, hematology, and urinalysis results. No inferential comparisons are planned.

### Vital Signs

Numeric summaries of all observed findings and changes from baseline screening vital signs will be provided by time point and treatment group, including blood pressure, heart rate, respiratory rate, and temperature. No inferential analyses are planned for vital signs.

### ECGs, ECHO/MUGA, and Physical Examination

Physical examination data and changes will be presented as listings. ECG results will be presented as listings and summarized by treatment group and visit, based on incidence of clinically significant abnormalities. No inferential comparisons across treatment groups are planned.

### Primary Efficacy Analysis—Part 2

Disease response is assessed according to RECIST v1.1 and will be summarized descriptively for the ITT, PP, and Efficacy Populations. The primary efficacy endpoints are:
ORR-3
PFS-6

### Secondary Endpoint(s)

### Pharmacokinetics, Pharmacodynamics, and Immunogenicity

PK, PD, and immunogenicity will be summarized descriptively for the PK/PD population in both Part 1 and Part 2.

### Secondary Efficacy Analysis-Part 2

Disease response (ORR, PFS, DCR, DoR, and OS) is assessed according to RECIST v1.1 and will be summarized descriptively for the ITT, PP, and Efficacy Populations.

### Exploratory Endpoints

Analysis of exploratory endpoints will be detailed in the SAP.

### Other Analysis

Other collected data not specifically mentioned will be presented in patient listings.

### Disposition, Demographics, Baseline Characteristics, and Medical History

Disposition information will be summarized including the number of enrolled patients, screening failures, treated patients, and the number of patients withdrawn by reason. Demographics, baseline characteristics, and medical history will be summarized by treatment group and overall using descriptive statistics for the ITT and PP.

### Prior and Concomitant Medications

Number and percentage of patients taking prior and concomitant medications will be summarized by treatment group and overall for the ITT and PP.

### Example 11. Anti-Galectin-9 Antibody Protects T cells from Galectin-9 Mediated Apoptosis

To investigate actions of anti-Galectin-9 antibody G9.2-17, an apoptosis assay was performed to determine if T cells are dying by the process of apoptosis or by other mechanisms.

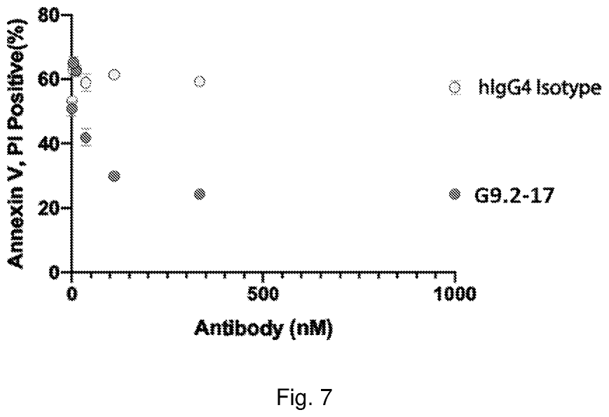

In brief, MOLM-13 (human leukemia) cells were cultured in RPMI media supplemented with 10% FBS, 2 mM L-glutamine, 10 mM HEPES, 1 mM sodium pyruvate, 4.5 g/L glucose and 1.5 g/L sodium bicarbonate at 37° C. in 5% CO2. Cells were then transferred into serum-free RPMI media and suspended at a concentration of 2.5e6 cells/mL in serum-free media. Cells were seeded into the wells of a tissue culture grade 96-well plate at a density of 2e5 cells/well (80 μL of cell suspension per well). Monoclonal anti-Galectin-9 antibody or matched isotype was added to each well and incubated at 37° C., 5% CO2 for 30 min. Following this incubation, recombinant, full length human Galectin-9 (R&D Systems 2045-GA, diluted in PBS) was added to a final concentration of 200 nM. Cells were incubated at 37° C., 5% CO₂ for 16 hours. Cells were then stained with Annexin V-488 and propidium iodide (PI) prior to analysis by flow cytometry. Each condition was performed in triplicate. PI is impermeant to live cells and apoptotic cells, but stains dead cells with red fluorescence, binding tightly to the nucleic acids in the cell. After staining a cell population with Alexa Fluor® 488 annexin V and PI in buffer, apoptotic cells showed green fluorescence, dead cells showed red and green fluorescence, and live cells showed little or no fluorescence. The cells were distinguished using a flow cytometer with the 488 nm line of an argon-ion laser for excitation. Analysis was then performed on FlowJo software. The fraction of annexin V- and propidium iodide (PI)-positive cells is plotted as a function of antibody concentration used in FIG. 7. As shown in FIG. 7, the level of apoptotic T cells treated with the anti-Gal9 antibody was much lower than T cells treated with a human IgG4 isotype control antibody, indicating that the anti-Galectin-9 antibody G9.2-17 protects T cells against galectin-9 mediated cell apoptosis.

### Example 12: In Vitro and In Vivo Characterization of Anti-Gal9 Antibody G9.2-17

In vivo and in vitro pharmacodynamics and pharmacology studies and safety pharmacology were conducted as disclosed below. In vivo studies were conducted with an IgG1 version of anti-galectin-9 mAb G9.2-17 for mouse studies based on the fact that this antibody was developed to have the exact same V_{H} and V_{L} chains and thus the exact same binding epitope as G9.2-17 and the same cross reactivity profile as well as binding affinities across species and same functional profile like G9.2-17.

### In Vitro Studies

G9.2-17 has multi-species cross-reactivity (human, mouse, rat, cynomolgus monkey), with equivalent<1 nmol binding affinities, as assessed in vitro. See, e.g., PCT/US2020/024767, the relevant disclosures of which are incorporated by reference for the subject matter and purpose as referenced herein. G9.2-17 does not cross react with the CRDI domain of galectin-9 protein. It has excellent stability and purification characteristics, and no cross-reactivity to any of the other galectin proteins that exist in primates.

Table 38 below summarizes results from in vitro pharmacology studies.

TABLE 38
In Vitro Primary Pharmacodynamics

| Objective | Assays | Key Results |
|---|---|---|
| Bead based | Binding of G9.2-17 to | Bead based measurements of G9.2-17 binding to the |
| binding - human | CRD1 and CRD2 | human galectin-9 CRD1 and CRD2 domains show |
| | domain of human | that G9.2-17 is specific to only the human CRD2 |
| | galectin-9 | domain of galectin-9. The mouse IgG1 version of |
| | | G9.2-17 show similar specificity to only the CRD2 |
| | | domain of galectin-9. KD Values (nM): G9.2-17 = |
| | | 0.15 ± 0.02, G9.2-17 mIgG1 = 0.18 ± 0.02. |
| Bead based | Binding of G9.2-17 to | Bead based measurements of G9.2-17 binding to the |
| binding - mouse | CRD2 domain of mouse | mouse galectin-9 CRD2 domain show that G9.2-17 |
| | galectin-9 | binds with <1 nMol to the mouse CRD2 domain. |
| | | The mouse IgG1 version of G9.2-17 show similar |
| | | affinity to the CRD2 domain of mouse galectin-9. |
| | | KD Values (nM): G9.2-17 = 0.30 ± 0.03; G9.2-17 |
| | | mIgG1 = 0.30 ± 0.1. |
| Bead based | Binding of G9.2-17 to | Bead based measurements of G9.2-17 binding to the |
| binding - rat | CRD2 domain of rat | rat galectin-9 CRD2 domain show that G9.2-17 binds |
| | galectin-9 | with <1 nMol to the rat CRD2 domain. The mouse |
| | | IgG1 version of G9.2-17 show similar affinity to the |
| | | CRD2 domain of rat galectin-9. KD Values (nM): |
| | | KD Values (nM): G9.2-17 = 0.31 ± 0.06; G9.2-17 |
| | | mIgG1 = 0.35 ± 0.06. |
| Bead based | Binding of G9.2-17 to | Bead based measurements of G9.2-17 binding to the |
| binding - | CRD2 domain of | cynomolgus galectin-9 CRD2 domain show that |
| cynomolgus | cynomolgus monkey | G9.2-17 binds with <1 nMol to the cynomolgus |
| monkey | galectin-9 | CRD2 domain. The mouse IgG1 version of G9.2-17 |
| | | show similar affinity to the CRD2 domain of |
| | | cynomolgus galectin-9. KD Values (nM): G9.2-17 = |
| | | 0.31 ± 0.03; G9.2-17 mIgG1 = 0.30 ± 0.10. |
| Binding - ELISA | ELISA based binding | G9.2-17 binding to human Galectin-9 CRD2 was |
| | assessment of G9.2-17 | assessed in ELISA format over a concentration |
| | to human CRD2 domain | range. G9.2-17 was titrated over immobilized |
| | of galectin-9 | Galectin-9 CRD2 and the resultant saturation curve |
| | | indicates that G9.2-17 has <1 nMol to the CRD2 |
| | | domain of galectin-9. |
| | | The mouse IgG1 version of G9.2-17 show similar |
| | | affinity to the CRD2 domain of galectin-9 when |
| | | assayed in this format. |
| | | KD Values (nM): G9.2-17 = 0.42 ± 0.07; G9.2-17 |
| | | mIgG1 = 0.45 ± 0.04. |
| Binding -SPR | SPR based binding | SPR measurements using the OneStep method on a |
| human | assessment of G9.2-17 | Pioneer SPR showed high binding of G9.2-17 to |
| | to human CRD2 domain | human galectin-9 CRD2. The resultant binding |
| | of galectin-9 | between the antibody and immobilized human |
| | | galectin-9 CRD2 had no measurable off rate even |
| | | after continued dissociation for over 30 minutes. |
| | | This suggests that G9.2-17 has a KD below the |
| | | measurable limit of assay. The mouse IgG1 version |
| | | of G9.2-17 showed similar behavior, with no |
| | | measurable off rate even over an extended |
| | | dissociation time. KD Values (nM): G9.2-17 = below |
| | | limit of detection; G9.2-17 mIgG1 = below limit of |
| | | detection. |
| Binding - SPR | SPR based binding | SPR measurements using the OneStep method on a |
| mouse | assessment of G9.2-17 | Pioneer SPR showed high binding of G9.2-17 to |
| | to mouse CRD2 domain | mouse galectin-9 CRD2. Binding of G9.2-17 to |
| | of galectin-9 | mouse galectin-9 CRD2 had a KD value of 1.8 ± 0.4 |
| | | nM. The mouse IgG1 version of G9.2-17 showed |
| | | similar behavior, with a KD- value of 3.05 ± 0.03 nM. |
| | | KD Values (nM): G9.2-17 = 1.8 ± 0.4; G9.2-17 |
| | | mIgG1 = 3.05 ± 0.03. |
| Binding - Cell- | Assessment of cell | An assessment of G9.2-17 binding to galectin-9 on |
| based | surface based (CRL- | the cell surface was performed using the galectin-9 |
| | 2134 cell line) binding | positive CRL-2134 cell line. First, staining of |
| | of G9.2-17 | CRL2134 with G9.2-17 showed increased signal |
| | | compared to staining of the galectin-9 negative HEK- |
| | | 293 cell line. A saturation curve was then generated |
| | | by titrating G9.2-17 for surface staining of CRL- |
| | | 2134 cells. The curve was generated based on the |
| | | fraction of the cell population that were positive for |
| | | galectin-9 as compared unstained cells. Using the |
| | | generated saturation curve, a cell based KD of |
| | | 0.41 ± 0.07 nM was calculated. This assay was also |
| | | performed with the mouse IgG1 variant of G9.2-17 |
| | | with a resulting cell-based KD of 2.9 ± 0.7 nM. |
| Cell-based | G9.2-17 potency | MOLM-13 cells are sensitive to high concentrations |
| potency | assessment using | of human galectin-9. Incubation of MOLM-13 cells |
| T-cell apoptosis | MOLM-13 T cell-based | for 16 h in the presence of 200 nM galectin-9 results |
| | apoptosis assay | in significant cell death. The addition of G9.2-17 |
| | | protects MOLM-13 from galectin-9 mediated cell |
| | | death in a dose dependent manner, significantly |
| | | reducing the population of necrotic cells. This effect |
| | | is specific for G9.2-17 as well as the mouse IgG1 |
| | | variant of G9.2-17 while the matched human IgG4 |
| | | and mouse IgG1 isotypes show no protection against |
| | | galectin-9 mediated cell death. |
| Non-cell based | G9.2-17 potency | The receptor-ligand interaction between CD206 and |
| potency | assessment using non- | galectin-9 was assayed in ELISA format. Full length |
| T-cell apoptosis | cell based, competition | galectin-9 was immobilized and recombinant, His- |
| | ELISA CD206 binding | tagged CD206 was titrated to confirm CD206 does |
| | assay | bind to galectin-9. In order to determine whether or |
| | | not G9.2-17 blocked the binding between galectin-9 |
| | | and its native receptor CD206, a competitive ELISA |
| | | assay was utilized. Blockade of the galectin-9- |
| | | CD206 interaction resulted in reduced ELISA signal |
| | | compared to the unblocked condition in a dose |
| | | dependent manner. |
| Functional assay: | Bead based | G9.2-17 does not mediate ADCC or ADCP activity. |
| non-cell based | ADCC/ADCP assay | |
| ADCC/ADCP | | |
| assay | | |
| Protein array - | Protein Array - Cross | HuProt ™ array was used for the High-Spec ® |
| cross reactivity | reactivity | antibody cross-reactivity assay. Arrays contained |
| | | native and not denatured proteins. G9.2-17 |
| | | recognized galectin-9 (CDI clone or the positive |
| | | control antigen) as the top hit with high affinity. |
| Expression | Assessing cell surface | Dose dependent effect was observed in detection of |
| | and intra-cellular | cell surface galectin-9 on KPC cells, peaking at 20% |
| | galectin-9 levels by flow | using 60 nM G9.2-17 Fab. Intracellular galectin-9 |
| | cytometry on | expression was uniformly detected in 10% of the |
| | permeabilized and non | cells at 15 nM, 30 nM and 60 nM of G9.2-17 Fab. |
| | permeabilized mouse | |
| | pancreatic cancer (KPC) | |
| | cells | |
| Expression | Assessing cell surface | 27.6% of B16F10 express galectin-9 on their surface |
| | and intra-cellular | and 98.8% intracellularly. 6.9% of MC38 express |
| | galectin-9 levels by flow | galectin-9 on their surface and 41.5% intracellularly. |
| | cytometry on | |
| | permeabilized and non | |
| | permeabilized mouse | |
| | melanoma (B16F10) and | |
| | colorectal cancer | |
| | (MC38) cells | |
| Mechanism of | Patient derived tumor | Activation of T cells measured through IFNg, TNFα |
| Action | cultures ex vivo | and CD44. n = 20 tumors processed. T cell |
| | (organoids) treated with | reactivation from baseline observed in n = 12 out of |
| | G9.2-17 | 20 (60%) of tumors processed. |
| Expression | Patient derived tumor | T cells galectin-9 expression (12.5-63.7% |
| | cultures ex vivo | CD3 + CD45 + intra PTOD T cells). Myeloid cell |
| | (organoids) profiling for | galectin-9 expression (15-45.9% CD45 + CD11b + |
| | galectin-9 expression on | intra PDOT myelod cells). Tumor cell galectin-9 |
| | T cells, tumor cells and | expression (9.15-33.5% CD45-EpCAM + intra PDOT |
| | macrophages | tumor cells) n = 6 PDOTs |
| Expression | Measuring galectin-9 | Sera from healthy controls (n = 16) and cancer |
| | levels in serum of | patients (n = 22; n = 10 primary and n = 12 |
| | healthy controls and | metastatic) with gastrointestinal malignancies. |
| | cancer patients | Galectin-9 serum levels are significantly increased in |
| | | cancer patients vs controls (p = 0.001) |
| Expression | Measuring galectin-9 | Sera and plasma from healthy controls (n = 10) and |
| | levels in serum and | cancer patients (n = 10) with metastatic tumors of |
| | plasma of healthy | diverse site of origin was tested for galectin-9 |
| | controls and cancer | expression. |
| | patients | |

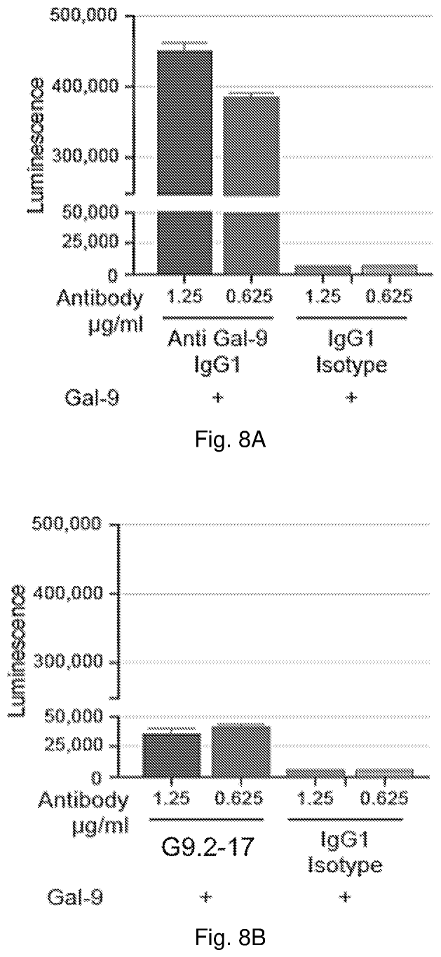

Studies to understand the mechanism of action included ADCC/ADCP (antibody dependent cell mediated cytotoxicity/antibody-dependent cellular phagocytosis) and blocking function assessment. As expected for a human IgG4 mAb, G9.2-17 does not mediate ADCC or ADCP (FIG. 8A). This was tested against the IgG1 human counterpart of G9.2-17 as a positive control, which mediates ADCC and ADCP, as expected (FIG. 8B).

Furthermore, blocking function of G9.2-17 was evaluated in a competition binding ELISA assay. G9.2-17 potently blocks binding of galectin-9 CRD2 domain to its binding partner CD206 human recombinant protein, confirming the intended mode of action for G9.2-17, which is to block galectin-9 activity. Moreover, we optimized a MOLM-13 T cell apoptosis assay where G9.2-17 proficiently rescues the cells from apoptosis caused by galectin-9 protein treatment (˜50% apoptosis with galectin-9 treatment and ˜10% apoptosis with galectin-9+G9.2-17 treatment).

Further extensive in vitro characterization has been done to compare binding and functional characteristics of G9.2-17 to the mouse IgG1 G9.2-17 mAb, which contains exactly the same CDR domains as G9.2-17, hence has the same binding epitope, i.e., CRD2 galectin-9 domain. mIgG1 G9.2-17 was developed for use in mouse syngeneic pharmacology efficacy studies, to avoid any potential development of immunogenicity with G9.2-17 itself. mIgG1 G9.2-17 has equivalent<1 nmol affinity across species, as well as the same cell based binding affinity to human cancer cell line, CRL-2134. mIgG1 G9.2-17 produces equivalent data in the MOLM-13 T cell apoptosis assay, as G9.2-17 itself.

### In Vivo Pharmacology

In vivo assays include syngeneic mouse models conducted using a mouse mAb-G9.2-17 binding epitope cloned into an IgG1 mouse backbone (G9.2-17 surrogate mAb for animal efficacy studies), which shares the cross reactivity and binding affinity characteristics of G9.2-17.

Syngeneic mouse models tested were:
Orthotopic pancreatic adenocarcinoma (KPC) mouse model (single agent and in combination with anti-PD-1): tumor volume assessment and flow cytometry;
Subcutaneous melanoma B16F10 model (single agent and in combination with anti-PD-1): tumor volume assessment and flow cytometry.
Subcutaneous MC38 model (single agent and in combination with anti-PD-1): tumor volume assessment

Further, patient-derived tumor cultures ex vivo (organoids) treated with G9.2-17 are to be used for exploring mechanism of action of G9.2-17.

Mechanistically, G9.2-17 was found to have blocking activity and not ADCC/ADCP activity. Blocking of galectin-9 interactions with its binding receptors, such as CD206 on immunosuppressive macrophages, is observed. Functionally, in vivo studies demonstrated reduction of tumor growth in multiple syngeneic models treated with G9.2-17 mIgG1 surrogate antibody (orthotopic pancreatic KPC tumor growth and s.c. melanoma B16F10 model). In mouse tumors treated with single agent anti-galectin-9 mAb and in combination with anti-PD-1, G9.2-17 reactivates effector T cells and reduces levels of immunosuppressive cytokines. Combination studies with an anti-PD-1 mAb suggest higher intra-tumoral presence of effector T cells, supporting clinical testing of the combinatorial approach. Importantly, mechanistic effects of G9.2-17 have been investigated and demonstrated in patient derived tumor cultures (Jenkins et al., 2018) (tumor excisions from primary and metastatic site, where G9.2-17 induces reproducible and robust T cell reactivation, indicating reversal of galectin-9 imposed intra-tumoral immunosuppression ex vivo.

In order to assess relevance of combining anti-PD-1 and anti-galectin-9 mAbs, s.c. melanoma B16 model was treated with single agent anti-PD-1 and anti-galectin-9 as well as the combination. Intra-tumoral presence effector T cells were enhanced in the combination arm.

Significant increases in the level of cytotoxic T cells (CD8) are observed in treatments with anti-galectin-9 mIgG1 200μg+anti-PD-1 (p<0.001) compared to that of anti-galectin-9 mIgG1 200 μg, and between anti-galectin-9 IgG1 200 μg+anti-PD-1 compared to anti-PD-1 alone (p<0.01). Such results suggest that anti-Gal9 antibody and anti-PD-1 antibody in combination would be expected to achieve superior therapeutic effects.

Table 39 below summarizes results from in vivo pharmacology studies.

TABLE 39
In Vivo Primary Pharmacodynamics

| Study Title | Test System | Key Results |
|---|---|---|
| Efficacy study assessing tumor | Orthotopic | Efficacy observed with single agent IgG1 |
| volume and flow cytometry of intra- | KPC model | mouse galectin-9 mAb, p = 0.05. Flow |
| tumoral immune cells in mice | | cytometry: CD8 T cells: Increase in CD8+ T |
| treated with IgG1 mouse anti- | | cell TNF alpha (p = 0.027), increase in |
| galectin-9 mAb at 150 μg/dose i.p. | | CD8+ T cell CD44 (p = 0.0008) and |
| | | reduction in CD8+ T cell IL10 (p = 0.0026). |
| | | Increase in CD4+ T Cell TNF alpha (p = |
| | | 0.0007). |
| Efficacy study assessing tumor | Orthotopic | Efficacy observed at 200 μg (p = 0.0005) and |
| volume and flow cytometry of intra- | KPC model | 400 μg (p = 0.01) dose levels of single agent |
| tumoral immune cells in mice | | anti-galectin-9 mIgG1 mAb. Flow |
| treated with IgG1 mouse anti- | | cytometry: CD8+ T cells: increase of CD44 |
| galectin-9 mAb at 200 and 400 | | (for dose levels 200 μg and 400 μg p = |
| μg/dose i.p. | | 0.002). CD4+ T cells: Increase in CD44 (for |
| | | dose level 200 μg, p = 0.015 and for dose |
| | | level 400 μg p = 0.0003). |
| Efficacy study assessing tumor | orthotopic | Efficacy observed at both dose levels (p < |
| volume and flow cytometry of intra- | KPC model | 0.01). Flow cytometry: CD4+ T cells: |
| tumoral immune cells in mice | | increase in CD44 (p < 0.0001), PD-1 (for |
| treated with IgG1 mouse anti- | | dose level 100 μg p = 0.005 and for dose |
| galectin-9 mAb at 100 and 200 | | level 200 μg p = 0.001); CD8+ T cells: |
| μg/dose i.p. | | increase in CD44 (p < 0.0001). |
| Efficacy study assessing tumor | Orthotopic | Efficacy observed at 50 μg (p < 0.05) and |
| volume in mice treated with IgG1 | KPC model | 100 μg (p < 0.0001) dose levels and no |
| mouse anti-galectin-9 mAb at 20, | | significant efficacy at 20 μg/dose. No |
| 50 and 100 μg/dose i.p. + 100 | | significant TV synergy effect with |
| μg/dose IgG1 mouse anti-galectin-9 | | combination of 100 μg anti-galectin-9 mAb |
| mAb with anti-PD-1 | | and anti-PD-1 |
| Efficacy study assessing tumor | Sub cutaneous | Highest efficacy observed at 200 μg (p < |
| volume and flow cytometry in mice | B16F10 | 0.005) single agent mouse anti-galectin-9 |
| treated with IgG1 mouse anti- | model | mAb, superior to anti-PD-1 mAb. No |
| galectin-9 mAb at 200 and 400 | | significant TV synergy effect with |
| μg/dose i.v. + anti-PD-1 mAb | | combination of 200 μg anti-galectin-9 mAb |
| | | and anti-PD-1 on tumor growth. However, |
| | | significant increase in cytotoxic CD8 T cell |
| | | levels were observed in mouse anti-galectin- |
| | | 9 mAb + anti-PD-1 mAb (p < 0.01). |
| Efficacy study assessing tumor | Sub cutaneous | Efficacy not superior to anti-PD-1 mAb in |
| volume in mice treated with IgG1 | MC38 model | this model. Combination with anti-PD-1 is |
| mouse anti-galectin-9 mAb at 200 | | equivalent to anti-PD-1 alone. Please refer to |
| and 400 μg/dose i.v. + anti-PD-1 | | CFCH001 for flow cytometry data |
| mAb | | explaining low expression of galectin-9 on |
| | | MC38 cells. |

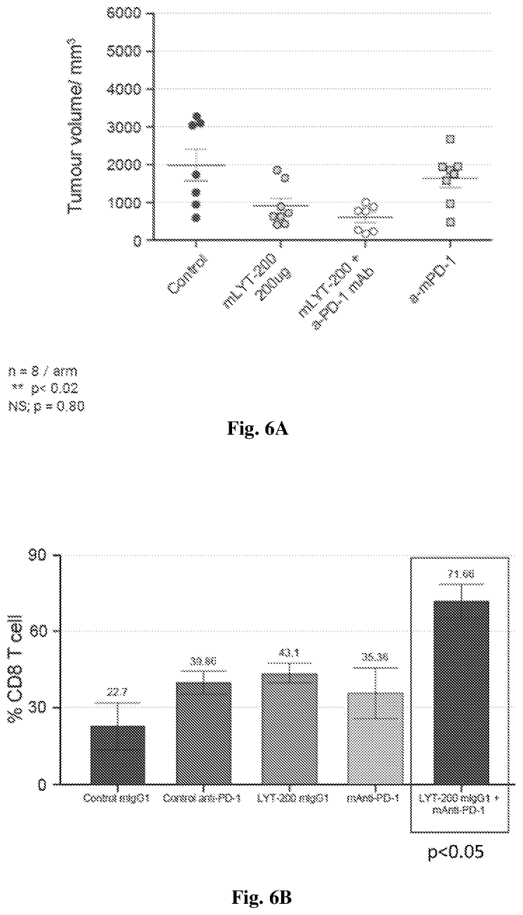

Further, tumor immune responses to treatment with G9.2-17 IgG1 mouse mAb (aka G9.2-17 mIgG), anti-PD1 antibody, or a combination of the G9.2-17 IgG1 mouse mAb and anti-PD1 antibody were investigated in the B16F10 subcutaneous syngeneic model described herein (results in FIGS. 6A-6B).

### Example 13. A non-GLP Single-Dose, Range-Finding Intravenous Toxicity Study in Male Sprague Dawley Rats with 1- and 3-Week Postdose Observation Periods

This study evaluated the anatomical endpoints of G9.2-17 IgG4 following a single intravenous bolus administration to Sprague Dawley rats followed by 1-week (terminal) and 3-week (recovery) necropsies on Days 8 and 22. All animals survived to the scheduled necropsies. There were no test article-related macroscopic findings, organ weight changes, or microscopic findings in either the terminal or recovery necropsy animals on this study.

The objective of this non-GLP exploratory, single-dose, range finding, intravenous toxicity study was to identify and characterize the acute toxicity G9.2-17 IgG4 following intravenous bolus administration over 2 minutes to Sprague Dawley rats followed by 1-week (terminal) and 3-week (recovery) postdose observation periods.

This non-GLP single dose toxicity study was conducted in 24 Sprague Dawley male rats to determine the toxicokinetics and potential toxicity of G9.2-17 IgG4. Animals were administered either vehicle or 10 mg/kg, 30 mg/kg or 70 mg/kg G9.2-17 IgG4 by slow bolus intravenous injection for at least 2 minutes on Day 1 followed by either a 1-week (terminal, Day 8) or 3-week (recovery, Day 22) period after the dose. Study endpoints included mortality, clinical observations, body weights, and food consumption, clinical pathology (hematology, coagulation, clinical chemistry and urinalysis), toxicokinetic parameters, ADA evaluation and anatomic pathology (gross necropsy, organ weights, and histopathology). Summaries of the experimental design is provided in Table 40 below.

TABLE 40
Experimental Design

| Group | | Dosage Level | Number of |
|---|---|---|---|
| Number | Treatment | (mg/kg) | Males ^{a} |
| 1 | Vehicle ^{b} | 0 | 6 |
| 2 | G9.2-17 IgG4 | 10 | 6 |
| 3 | G9.2-17 IgG4 | 30 | 6 |
| 4 | G9.2-17 IgG4 | 70 | 6 |

^{a} 3 animals/sex/group were euthanized at the Day 8 terminal necropsy; the remaining 3 animals/sex/group were euthanized at the Day 22 recovery necropsy.
^{b} The vehicle was Formulation Buffer (20 mM Tris, 150 mM NaCl, pH 8.0 ± 0.05).

All surviving animals were submitted for necropsy on Day 8 or Day 22. Complete postmortem examinations were performed and organ weights were collected. The organs were weighed from all animals at the terminal and recovery. Tissues required for microscopic evaluation were trimmed, processed routinely, embedded in paraffin, and stained with hematoxylin and eosin.

There were no unscheduled deaths during the course of this study. All animals survived to the terminal or recovery necropsies. Histological changes noted were considered to be incidental findings or related to some aspect of experimental manipulation other than administration of the test article. There was no test article related alteration in the prevalence, severity, or histologic character of those incidental tissue alterations. No G9.2-17 IgG4-related findings were noted in clinical observations, body weights, food consumption, clinical pathology or anatomic pathology. In conclusion, the single intravenous administration of 10, 30, and 70 mg/kg G9.2-17 IgG4 to Sprague Dawley rats was tolerated with no adverse findings. Therefore, under the conditions of this study the NOEL was 70 mg/kg.

### Example 14. A non-GLP Single-Dose, Range-Finding Intravenous Infusion Toxicity Study of G9.2-17 IgG4 in Cynomolgus Monkeys with a 3-Week Post-Dose Observation Period

This non-GLP single-dose toxicity study was conducted in 8 cynomolgus monkeys to identify and characterize the acute toxicities of G9.2-17 IgG4. Animals (1 male [M]/1 female [F]/group) were administered either vehicle or 30 mg/kg, 100 mg/kg, or 200 mg/kg G9.2-17 IgG4 by 30-minute intravenous (IV) infusion followed by a 3 week post-dose observation period. Study endpoints included: mortality, clinical observations, body weights, and qualitative food consumption; clinical pathology (hematology, coagulation, clinical chemistry, immunophenotyping and galectin 9 expression on leukocyte subsets, and cytokine analysis); toxicokinetic parameters; serum collection for possible anti-drug antibody evaluation (ADA); and soluble galectin-9 analyses; and anatomic pathology (gross necropsy, organ weights, and histopathology).

No G9.2-17 IgG4-related findings were noted in clinical observations, body weights, food consumption, clinical pathology (hematology, clinical chemistry, coagulation, or cytokine analysis), immunophenotyping, galectin-9 expression on leukocyte subsets, soluble galectin-9 or anatomic pathology.

In conclusion, the single intravenous infusion administration of 30, 100, and 200 mg/kg G9.2-17 IgG4 to cynomolgus monkeys was tolerated with no adverse findings. Therefore, under the conditions of this study the No-observed-Adverse-Effect-Level (NOAEL) was 200 mg/kg, the highest dose level evaluated. The study design is shown in Table 41.

TABLE 41
Experimental Design

| | | | | Adjusted Dose | | Dose Volume | | Animal |
|---|---|---|---|---|---|---|---|---|
| | | | | Concentration | | (mL/kg) | | No. |
| Group | Treat- | Dose Level | | (mg/mL) | | Necropsy | | Necropsy |
| No. | ment | (mg/kg) | | | Males | Day | Females | Day |
| 1 | Vehicle | 0 | 0 | 20 | 1001 | 22 | 1501 | 22 |
| 2 | G9.2-17 | 30 | 1.5 | 20 | 2001 | 22 | 2501 | 22 |
| | IgG4 | | | | | | | |
| 3 | G9.2-17 | 100 | 5 | 20 | 3001 | 22 | 3501 | 22 |
| | IgG4 | | | | | | | |
| 4ª | G9.2-17 | 200 | 10 | 20 | 4001 | 22 | 4501 | 22 |
| | IgG4 | | | | | | | |

ªGroup 4 was administered 1 week after administration of Groups 1 through 3.

The vehicle and test article were administered once via IV infusion for 30 minutes during the study via a catheter percutaneously placed in the saphenous vein. The dose levels were 30, 100, and 200 mg/kg and administered at a dose volume of 20 mL/kg. The control group received the vehicle in the same manner as the treated groups.

The animals were placed in sling restraints during dosing. The vehicle or test article were based on the most recent body weights and administered using an infusion pump and sterile disposable syringes. The dosing syringes were filled with the appropriate volume of vehicle or test article (20 mL/kg with 2 mL extra). At the completion of dosing, the animals were removed from the infusion system. The weight of each dosing syringe was recorded prior to the start and end of each infusion to determine dose accountability.

### Detailed Clinical Observations

The animals were removed from the cage, and a detailed clinical examination of each animal was performed at 1 and 4.5 hours post-start of infusion (SOI) on Day 1 and once daily thereafter during the study. The animals were removed from the cage, and a detailed clinical examination of each animal was performed at 1 and 4.5 hours post-start of infusion (SOI) on Day 1 and once daily thereafter during the study. Body weights for all animals were measured and recorded at transfer, prior to randomization, on Day-1, and weekly during the study.

Clinical pathology evaluations (hematology, coagulation, and clinical chemistry) were conducted on all animals pretest and on Days 1 (prior to dosing), 3, 8, and 21. Additional samples for the determination of hematology parameters and peripheral blood lymphocyte and cytokine analysis samples were collected at 30 minutes (immediately after the end of infusion) and 4.5, 8.5, 24.5, and 72.5 hours post-SOI (relative to Day 1). Bone marrow smears were collected and preserved.

Blood samples (approximately 0.5 mL) were collected from all animals via the femoral vein for determination of the serum concentrations of the test article (see Table 42) (for a deviation, see Appendix 1). The animals were not fasted prior to blood collection, with the exception of the intervals that coincided with fasting for clinical pathology collections.

TABLE 42
Bioanalysis Sample Collection Schedule

| | Sample Collection Time Points | | | | | | | | | | | | |
|---|---|---|---|---|---|---|---|---|---|---|---|---|---|
| | (Time Post-SOI) relative to Day 1 | | | | | | | | | | | | |
| | | | | | | | 24.5 | 48.5 | 72.5 | 120.5 | 168.5 | 360.5 | 504.5 |
| | | | | | | | hr | hr | hr | hr | hr | hr | hr |
| Group | Pre | 0.583 | 1 | 2.5 | 4.5 | 8.5 | (Day | (Day | (Day | (Day | (Day | (Day | (Day |
| No. | dose | hr^{a} | hr | hr | hr | hr | 2) | 3) | 4) | 6) | 8) | 16) | 22) |
| 1-4 | X | X | X | X | X | X | X | X | X | X | X | X | X |

X = Sample was collected.
^{a}Only the 0.583 hr post-SOI timepoint from Group 1 animals was analyzed for test article content.
Additional timepoints may be analyzed at the discretion of the Study Director.

For processing, blood samples were collected in non-additive barrier free microtubes and centrifuged at controlled room temperature within 1 hour of collection. The resulting serum was divided into 2 approximately equal aliquots in pre labeled cryovials. All aliquots were stored frozen at −60° C. to −90° C. within 2 hours of collection.

Postmortem study evaluations were performed on all animals euthanized at the scheduled necropsy.

Necropsy examinations were performed under procedures approved by a veterinary pathologist. The animals were examined carefully for external abnormalities including palpable masses. The skin was reflected from a ventral midline incision and any subcutaneous masses were identified and correlated with antemortem findings. The abdominal, thoracic, and cranial cavities were examined for abnormalities. The organs were removed, examined, and, where required, placed in fixative. All designated tissues were fixed in neutral buffered formalin (NBF), except for the eyes (including the optic nerve) and testes. The eyes (including the optic nerve) and testes were placed in a modified Davidson's fixative, and then transferred to 70% ethanol for up to three days prior to final placement in NBF. Formalin was infused into the lung via the trachea. A full complement of tissues and organs was collected from all animals.

Body weights and protocol-designated organ weights were recorded for all animals at the scheduled necropsy and appropriate organ weight ratios were calculated (relative to body and brain weights). Paired organs were weighed together. A combined weight for the thyroid and parathyroid glands was collected.

### Results

All animals survived to the scheduled necropsy on Day 22. No test article-related clinical or veterinary observations were noted in treated animals. No test article-related effects on body weight were observed in treated animals during the treatment or recovery period. There were no G9.2-17 IgG4-related effects on hematology endpoints in either sex at any dose level at any interval.

There were no G9.2-17 IgG4-related effects on coagulation times (i.e., activated partial thromboplastin times [APTT] and prothrombin times) or fibrinogen concentrations in either sex at any dose level at any interval. All fluctuations among individual coagulation values were considered sporadic, consistent with biologic and procedure-related variation, and/or negligible in magnitude, and not related to G9.2-17 IgG4 administration.

There were no G9.2-17 IgG4-related effects on clinical chemistry endpoints in either sex at any dose level at any interval. All fluctuations among individual clinical chemistry values were considered sporadic, consistent with biologic and procedure-related variation, and/or negligible in magnitude, and not related to G9.2-17 IgG4 administration.

There were no G9.2-17 IgG4-related effects on cytokine endpoints in either sex at any dose level at any interval. All fluctuations among individual cytokine values were considered sporadic, consistent with biologic and procedure-related variation, and/or negligible in magnitude, and not related to G9.2-17 IgG4 administration.

Review of the gross necropsy observations revealed no findings that were considered to be test article related. There were no organ weight alterations that were considered to be test article-related. There were no test article-related changes.

In conclusion, the single intravenous infusion administration of 30, 100, and 200 mg/kg G9.2-17 IgG4 to cynomolgus monkeys was tolerated with no adverse findings. Therefore, under the conditions of this study the No-observed-Adverse-Effect-Level (NOAEL) was 200 mg/kg, the highest dose level evaluated.

The animals were removed from the cage, and a detailed clinical examination of each animal was performed at 1 and 4.5 hours post-start of infusion (SOI) on Day 1 and once daily thereafter during the study.

### Example 15: Intravenous Infusion Study of G9.2-17 in Cynomolgus Monkeys

The objective of this study was to further characterize the toxicity and toxicokinetics of the test article, G9.2-17 (a hIgG4 Monoclonal Antibody which binds to Galectin-9), following once weekly 30-minute intravenous (IV) infusion for 5 weeks in cynomolgus monkeys, and to evaluate the reversibility, progression, or delayed appearance of any observed changes following a 3-week recovery period.

### Experimental Design

Table 43 summarizes the study design.

TABLE 43
Experimental Design

| | | | | Dose | | | | |
|---|---|---|---|---|---|---|---|---|
| | | | Dose | Concen- | Main Study | | Recovery Study | |
| Group | Test | Dose Level | Volume^{a} | tration | No. of | No. of | | No. of |
| No. | Material | (mg/kg/dose) | (mL/kg) | (mg/mL) | Males | Females | Males | Females |
| 1 | Vehicle | 0 | 10 | 0 | 3 | 3 | 2 | 2 |
| 2 | G9.2-17 | 100 | 10 | 10 | 3 | 3 | 2 | 2 |
| 3 | G9.2-17 | 300 | 10 | 30 | 3 | 3 | 2 | 2 |

^{a}Based on the most recent practical body weight measurement.

Animals (cynomolgus monkeys) used in the study were assigned to study groups by a standard, by weight, randomization procedure designed to achieve similar group mean body weights. Males and females were randomized separately. Animals assigned to study had body weights within +20% of the mean body weight for each sex.

The formulations lacking G9.2-17 (“vehicle”) or encompassing G9.2-17 (“test article”) were administered to the animals once weekly for 5 weeks (Days 1, 8, 15, 22, and 29) during the study via 30-minute IV infusion. The dose levels were 0, 100 and 300 mg/kg/dose and administered at a dose volume of 10 mL/kg. The control animals group received the vehicle in the same manner as the treated groups. Doses were administered via the saphenous vein via a percutaneously placed catheter and a new sterile disposable syringe was used for each dose. Dose accountability was measured and recorded prior to dosing and at the end of dosing on toxicokinetic sample collection days (Days 1, 15, and 29) to ensure a ±10% target dose was administered. Individual doses were based on the most recent body weights. The last dose site was marked for collection at the terminal and recovery necropsies. All doses were administered within 8 hours of test article preparation.

In-life procedures, observations, and measurements were performed on the animals as exemplified below.

Electrocardiographic examinations were performed on all animals. Insofar as possible, care was taken to avoid causing undue excitement of the animals before the recording of electrocardiograms (ECGs) in order to minimize extreme fluctuations or artifacts in these measurements. Standard ECGs (10 Lead) were recorded at 50 mm/sec. Using an appropriate lead, the RR, PR, and QT intervals, and QRS duration were measured and heart rate was determined. Corrected QT (QTc) interval was calculated using a procedure based on the method described by Bazett (1920). All tracings were evaluated and reported by a consulting veterinary cardiologist.

To aid in continuity and reliability, functional observational battery (FOB) evaluations were conducted by two independent raters for all occasions and consisted of a detailed home cage and open area neurobehavioral evaluation (Gauvin and Baird, 2008). Each technician scored the monkey independently (without sharing the results with each other) for each home cage and out of cage observational score, and then the individual scores were assessed for agreement with their partner's score after the completion of the testing. FOB evaluations were conducted on each animal predose (on Day-9 or Day 8) to establish baseline differences and at 2 to 4 hours from the start of infusion on Days 1 and 15, and prior to the terminal and recovery necropsies. The observations included, but were not limited to, evaluation of activity level, posture, lacrimation, salivation, tremors, convulsions, fasciculations, stereotypic behavior, facial muscle movement, palpebral closure, pupil response, response to stimuli (visual, auditory, and food), body temperature, Chaddock and Babinski reflexes, proprioception, paresis, ataxia, dysmetria, and slope assessment, movement, and gait.

Blood pressure of each animal was measured and recorded and consisted of systolic, diastolic, and mean arterial pressure. Blood pressure measurements are reported using three readings that have the Mean Arterial Pressure (MAP) within 20 mmHg.

Respiratory rates of each animal were measured and recorded 3 times per animal/collection interval by visual assessment per Testing Facility SOP. The average of the 3 collections is the reported value.

Clinical pathology evaluations (e.g., immunophenotyping and cytokine evaluations) were conducted on all animals at predetermined intervals. Bone marrow smears were collected and preserved. Blood samples (approximately 0.5 mL) were collected from all animals via the femoral vein for determination of the serum concentrations of the test article. The animals were not fasted prior to blood collection, with the exception of the intervals that coincided with fasting for clinical pathology collections. At the conclusion of the study (day 36 or day 50), animals were euthanatized and tissues for histology processing and microscopic evaluation were collected.

Soluble galectin-9 was evaluated as follows. Blood samples (approximately 1 mL) were collected from all animals via the femoral vein for determination of the serum for soluble galectin 9 predose and 24 hours from the start of infusion on Days 1, 8, 15, and 29, and prior to the terminal and/or recovery necropsies. The animals were not fasted prior to blood collection, with the exception of the intervals that coincided with fasting for clinical pathology collections.

Soluble galectin-9 samples were processed as follows. Blood samples were collected in non-additive, barrier free tubes, allowed to clot at ambient temperature, and centrifuged at ambient temperature. The resulting serum was divided into 2 aliquots (100 μL in Aliquot 1 and remaining in Aliquot 2) in pre labeled cryovials. All aliquots were flash frozen on dry ice within 2 hours of collection and stored frozen at-60° C. to 90° C.

All results presented in the tables of the report were calculated using non-rounded values as per the raw data rounding procedure and may not be exactly reproduced from the individual data presented.

### Results

### Mortality

All animals survived to the scheduled terminal necropsy on Day 36 and recovery necropsy on Day 50.

### Detailed Clinical and Veterinary Observations

No test article-related clinical or veterinary observations were noted in treated animals during the treatment or recovery periods.

### Functional Observational Battery

No test article-related FOB observations were noted in treated animals during the treatment or recovery periods.

### Body Weight and Body Weight Gains

No test article-related effects in body weight and body weight gain were noted in treated animals during the treatment or recovery periods.

### Ophthalmology Examinations

No test article-related effects in ophthalmology examinations were noted in treated animals during the treatment or recovery periods.

### Blood Pressure Values

No test article-related effects in blood pressure values were noted in treated animals during the treatment or recovery periods.

### Respiratory Rate Values

No test article-related effects in respiratory rate values were noted in treated animals during the treatment or recovery periods.

### Electrocardiology

No test article-related effects in electrocardiograms were noted in treated animals during the treatment or recovery periods.

### Hematology

There were no G9.2-17-related effects among hematology parameters in either sex at any dose level at any timepoint.

### Coagulation

There were no G9.2-17-related effects among coagulation parameters in either sex at any dose level at any timepoint.

### Clinical Chemistry

There were no G9.2-17-related effects among clinical chemistry parameters in either sex at any dose level at any timepoint.

Urinalysis No G9.2-17-related alterations were observed among urinalysis parameters in either sex at any dose level at the 13-week interim.

### Cytokine

No definitive G9.2-17-related effects on cytokines were seen at any dose level or timepoint.

### Peripheral Blood Leukocyte Analysis (PBLA)

There were no G9.2-17-related effects on PBLA endpoints in either sex at any dose level at any timepoint.

### Bioanalysis, Galectin-9, and Toxicokinetic Evaluation

G9.2-17 was quantifiable in all cynomolgus monkey samples from all G9.2-17-dosed animals after dose administration. No measurable amount of G9.2-17 was detected in control cynomolgus monkey samples. Soluble galectin-9 was quantifiable in all cynomolgus monkey samples from all animals. G9.2-17 serum concentrations were below the bioanalytical limit of quantitation (LLOQ<0.04 μg/mL) in all serum samples obtained predose from most G9.2-17 treated animals on Day 1 and from control animals on Days 1 and 29.

### Gross Pathology and Organ Weight

There were no definitive test article-related macroscopic observations in main study or recovery animals. There were also no test article-related organ weight changes for main study or recovery animals.

### Histopathology

There were no definitive test article-related microscopic observations.

In conclusion, once weekly intravenous infusion administration of 100 and 300 mg/kg of G9.2-17 for 5-weeks to cynomolgus monkeys was tolerated with no adverse findings.

### Example 16: Intravenous Infusion Study of G9.2-17 in Sprague Dawley Rats

The objective of this study was to evaluate potential toxicity of G9.2-17, an IgG4 human monoclonal antibody directed against galectin-9, when administered by intravenous injection to Sprague Dawley Rats once weekly for 4 consecutive weeks followed by a 3-week post dose recovery period. In addition, the toxicokinetic characteristics of G9.2-17 were determined.

### Experimental Design

Table 44 summarizes the study design.

TABLE 44
Study Design

| | | | Dose | | | | | | | |
|---|---|---|---|---|---|---|---|---|---|---|
| | | Dose | Concen- | Dose | | | | | TK/Gal- | |
| | Test | Level | tration | Volume^{a} | Terminal | | Recovery | | 9/Cyto | |
| Group | Material | (mg/kg) | (mg/mL) | (mL/kg) | M | F | M | F | M | F |
| 1 | Control | 0 | 0 | 10 | 10 | 10 | 5 | 5 | 12 | 12 |
| 2 | G9.2-17 | 100 | 10 | 10 | 10 | 10 | 5 | 5 | 12 + | 12 + |
| | | | | | | | | | 6^{b} | 6^{b} |
| 3 | G9.2-17 | 300 | 30 | 10 | 10 | 10 | 5 | 5 | 12 + | 12 + |
| | | | | | | | | | 6^{b} | 6^{b} |

^{a}Individual dose volumes were calculated based on the most recent body weight.
^{b}SSD animals: 3 animals/sex/group for TK collections only following a single dose administration on Day 1.

One hundred eighty-six animals (Sprague Dawley rats) were assigned to treatment groups randomly by body weight. Control Article/Vehicle, Formulation Buffer for Test Article, and test article, G9.2-17, were administered via a single IV injection in a tail vein at dose levels of 0, 100, and 300 mg/kg once on Days 1, 8, 15, 22, and 29. Test article was administered at dose levels of 100 and 300 mg/kg once on Day 1 to animals assigned to the SSD subgroup.

Clinical observations were performed once daily prior to room cleaning in the morning, beginning on the second day of acclimation. A mortality check was conducted twice daily to assess general animal health and wellness. Food consumption was estimated by weighing the supplied and remaining amount of food in containers once weekly. The average gram (g)/animal/day was calculated from the weekly food consumption. Body weights were taken prior to randomization, on Day-1, then once weekly throughout the study, and on the day of each necropsy. Functional Observation Battery (FOB) observations were recorded for SSB animals approximately 24 hours post dose administrations on Days 1, 35 and 49. Urine was collected overnight using metabolic cages. Samples were obtained on Days 36 and 50.

Animals were fasted overnight prior to each series of collections that included specimens for serum chemistry. In these instances associated clinical pathology evaluations were from fasted animals. Blood was collected from a jugular vein of restrained, conscious animals or from the vena cava of anesthetized animals at termination.

Parameters assessed during the In-life examinations of the study included clinical observations, food consumption, body weights, functional observational battery. Blood samples were collected at selected time points for clinical pathology (hematology, coagulation, and serum chemistry) analyses. Urine samples were collected for urinalysis. Blood samples were also collected at selected time points for toxicokinetic (TK), immunogenicity (e.g., anti-drug antibody or ADA), and cytokine analyses. Animals were necropsied on Days 36 and 50. At each necropsy, gross observations and organ weights were recorded, and tissues were collected for microscopic examination.

### Results

### In-Life Examinations

Mortality: There were no abnormal clinical observations or body weight changes noted for this animal during the study.

Clinical Observations: There were no G9.2-17-related clinical observations noted during the study.

Food Consumption/Body Weights: There were no G9.2-17-related changes in food consumption, body weights or body weight gain noted during the study.

Clinical Pathology: There were no G9.2-17-related changes noted in clinical pathology parameters.

Cytokine Analysis: There were no G9.2-17-related changed in serum concentrations of IL-2, IL-4, IFN-y, IL-5, IL-6, IL-10, and/or TNF-a, MCP-1 and MIP-1b.

Gross Pathology: There were no G9.2-17-related gross observations. Further, were no G9.2-17-related changes in absolute or relative organ weights.

Histopathology: There were no G9.2-17-related histologic findings.

In conclusion, intravenous G9.2-17 administration to Sprague Dawley rats once weekly for a total of 5 doses was generally well tolerated. There were no G9.2-17-related changes in clinical observations, food consumption, body weights, FOB parameters, clinical pathology, cytokine, gross observations, or organ weights.

### Example 17: A Study of the Safety, Pharmacokinetics, and Anti-tumor Activity of G9.2-17(IgG4) Alone and in Combination with Chemotherapy in Subjects with Ocular Melanoma

Galectin-9 is a molecule overexpressed by many solid tumors, including those in pancreatic cancer, colorectal cancer, and hepatocellular carcinoma. Moreover, Galectin-9 is expressed on tumor-associated macrophages, as well as intra-tumoral immunosuppressive gamma delta T cells, thereby acting as a potent mediator of cancer-associated immunosuppression. As described herein, monoclonal antibodies targeting Galectin-9 (e.g., G9.2-17) have been developed. Data have demonstrated that G9.2-17 halts pancreatic tumor growth by 50% in orthotopic KPC models and extended the survival of KPC animals by more than double. In addition, the anti-Galectin-9 antibody shows signals of therapeutic synergy with chemotherapeutics in animal studies.

The purpose of this study is to determine the safety, tolerability, maximum tolerated dose (MTD), and objective tumor response after 12 to 24 months of treatment in subjects having an ocular melanoma such as metastatic ocular melanoma. The study also examines progression-free survival (PFS), the duration of response (by RESIST), disease stabilization, the proportion of subjects alive, as well as pharmacokinetic (PK) and pharmacodynamics (PD) parameters.

Subjects undergo pre- and post-treatment biopsies, as well as PET-CT imaging pre-study and once every 8 weeks for the duration of the study. In addition, immunological endpoints, such as peripheral and intra-tumoral T cell ratios, T cell activation, macrophage phenotyping, cytokine profiling in serum, tumor immunohistochemistry, and Galectin-9 serum levels are examined. The study is performed under a master study protocol, and the study lasts for 12-24 months. Subject, disease, and all clinical and safety data are presented descriptively as means, medians, or proportions, with appropriate measures of variance (e.g., 95% confidence interval range). Waterfall and Swimmers plots are be used to graphically present the ORR and duration of responses for subjects for each study arm, within each disease site, as described below. Exploratory correlations analysis are also be undertaken to identify potential biomarkers that may be associated with ORR. All statistical analyses are performed using SAS, version 9.2 (SAS, Cary, NC).

### (A) Study Design

The lowest anticipated pharmacologically active dose (PAD) is currently estimated to be 2 mg/kg, based on the mouse model KPC004 data, in which 50 mcg/mouse (2 mg/kg; human equivalent dose HED=0.16 mg/kg) was established as an active dose. Alternate models were active at the 200 or 400 mcg/mouse dose range (8-16 mg/kg; HED=0.65-1.3 mg/kg).

Table 45 below shows proposed clinical starting dose levels dependent upon the outcome of the repeat-dose GLP-compliant toxicity study at the proposed dose levels of 100 and 200 mg/kg G9.2-17. The estimated starting doses use either 1/10 of the no observed adverse effect level (NOAEL) or 1/6 of the highest non-severely toxic dose (HNSTD) as a starting point and then convert that dose in mg/kg to the HED in mg/kg.

TABLE 45
Proposed Clinical Starting Doses

| Dose | | HED of | | | 3-Fold Safety | | | |
|---|---|---|---|---|---|---|---|---|
| Level | | the GLP | | | factor added | | | |
| in GLP | | toxicity | 1/10 of | | to 1/10 | | ⅙ of | |
| Toxicity | | study | NOAEL | | NOAEL | | HNSTD | |
| Study | | dose | (HED) | | (HED) | | (HED) | |
| mg/kg | Species | mg/kg | mg/kg | | mg/kg | | mg/kg | |
| 300 | Rat | 48 | 30 | (5) | 10 | (1.6) | 50 | (8) |
| 300 | NHP | 97 | 30 | (10) | 10 | (3.2) | 50 | (16) |

Proposed clinical starting dose with additional 3-fold safety factor
based on the outcome of the GLP repeat-dose toxicity study in NHP

| | | | | |
|---|---|---|---|---|
| 300 | NHP | 6 | 2 | 3.6 |

This study includes both monotherapy of G9.2-17 (IgG4) and combination therapy including G9.2-17 and gemcitabine/Abraxane ((paclitaxel protein-bound particles for injectable suspension; albumin-bound). The study is split into 2 parts: Part 1 (Phase la) and Part 2 (Phase 1b)

### Part 1

Part 1 of the study is a dose-finding study using a continuous reassessment method (CRM) (O′Quigley et al., 1990), a model-based design that informs how the dosage of G9.2-17 should be adapted for the next patient cohort based on past trial data. Two patients at a time are dosed with G9.2-17 alone, with a maximum available sample size of 24. Patients receive 5 dose levels every 2 weeks until progression of disease, unacceptable toxicity, or withdrawal from the study development of dose-limiting toxicity (DLT). The dose levels are:
Dose level 1=2 mg/kg;
Dose level 2=4 mg/kg;
Dose level 3=8 mg/kg;
Dose level 4=12 mg/kg; and
Dose level 5=16 mg/kg.

Alternatively, the dose levels are:
Dose Escalation Cohort 1=0.2 mg/kg
Dose Escalation Cohort 2=0.63 mg/kg
Dose Escalation Cohort 3=2 mg/kg
Dose Escalation Cohort 4=6.3 mg/kg
Dose Escalation Cohort 5=10 mg/kg
Dose Escalation Cohort 6=16 mg/kg

The dosing regimen is once every two weeks (Q2W) by intravenous (IV) administration. Dose reduction of up to 25% may be adopted when needed.

As a safety precaution, at each dose escalation, new patients are entered and treated only after the first patient of each cohort has been treated with G9.2-17 and after a minimum 7 days post-treatment have elapsed. Part I is complete after six consecutive patients have received the same dose and that dose is identified as the optimal biological dose (OBD).

### Part 2

Part 2 of the study is a Simon's two-stage optimal design. The study investigates the use of the G9.2-17 alone (single agent arms of the study) and in conjunction with gemcitabine/Abraxane. The dose of the anti-Galectin-9 antibody used is below the level found to exhibit toxicity in Part 1.

The optimal two-stage design is used to test the null hypothesis that the ORR≤5% versus the alternative that the ORR≥15% within the single agent arms. After testing the drug on 23 patients in the first stage, the respective trial arm is terminated if ≤1 patients respond. If the trial goes on to the second part of Simon's optimal design, a total of 56 patients are enrolled into each of the single agent arms. If the total number responding patients is ≤5, the investigational drug within that arm is rejected. If >6 patients have an ORR at 3 months, the expansion cohort for that arm is activated. The above approach is applied to the single agent arms of the study.

Combination Treatment with G9.2-17 and Gemcitabine Abraxane

Combination treatment with G9.2-17 and gemcitabine/Abraxane is evaluated in patients with ocular melanoma (e.g., metastatic). The primary objective of this study is progression free survival (PFS) at 6 months. Secondary objectives include improvements in objective response rate (ORR), disease control rate (DCR) at 6 and 12 months, patient survival at 6 and 12 months, time to response, duration and depth of response by RECIST 1.1 criteria, safety and tolerability. In the case of the combination arms, the starting dose of G9.2-17 is administered at one dose lower than the OBD identified in Part I (e.g., the RP2D dose level identified in Part 1). Doses of gemcitabine/Abraxane follow those on FDA-approved label and may be adjusted in light of regimen specific side effects, if any (e.g., 2 weeks on one week off). If 3 or more patients develop a DLT, the dose of G9.2-17 is reduced in a stepwise manner not to exceed dose 3 amounts unless low doses continue to provide a clinical benefit.

In the patient cohort consisting of ocular melanoma (e.g., metastatic) patients, the primary efficacy endpoint is PFS at 6 months. In the 1st line metastatic setting using gemcitabine/Abraxane, the 6 months PFS was reported to be 50% (Von Hoff et al., 2013). After testing the G9.2-17/chemotherapy combination on the first 11 patients in the first stage, the trial is terminated if 6 or fewer patients exhibit PFS>6 months. In the second stage of the trial, a total of 25 patients are studied. If the total number of responding patients with PFS of >6 months is ≤16, the study arm is rejected.

Expansion of cohorts is implemented where an early efficacy signal has been detected. Once a promising efficacy signal is identified within one of the five trial arms that is attributable to the tumor type, an expansion cohort is launched to confirm the finding. The sample size for each of the expansion arms is determined based on the point estimates determined in Part 2, in combination with a predetermined level of precision for the 95% confidence interval (95% CI) around the ORR/patient survival.

### Part 3

Part 3 includes expansion of cohorts where early efficacy signal has been detected. If a promising efficacy signal is identified within one of the trial arms that is attributable to the tumor type, an expansion cohort is launched to confirm the finding. The sample size for each of the expansion arms is determined based on the point estimates determined in Part 2, in combination with predetermined level of precision for the 95% confidence interval (95% CI) around the ORR. The study duration is 12-24 months.

### (B) Patient Population

Patients with relapsed/refractory metastatic cancers, irrespective of tumor type, are eligible for the dose-finding study using the continual reassessment method (CRM) as described by O'Quigley (1990). Expansion is envisaged in ocular melanoma where mode of action and/or an early efficacy signal are captured in Part 1. Expansion is also considered in ocular melanoma.

Patient inclusion and exclusion criteria are the same for both Part 1 and part 2.

### Patient Inclusion Criteria:

1. Written informed consent
2. Age≥18 years, male or non-pregnant female
3. Histologically confirmed unresectable metastatic cancer (adenocarcinomas and squamous cell carcinomas)
4. Able to comply with the study protocol
5. Life expectancy>3 months
6. Recent archival tumor sample (obtained within 5 years) available for biomarker analyses.
7. Patient able and willing to undergo pre- and on/post-treatment biopsies.
8. Measurable disease, according to RECIST v1.1. Note that lesions biopsied should not be target lesions.
9. Expected survival>3 months
10. For Part 1: No available standard of care options, or patient has declined available and indicated standard of care therapy, or is not eligible for available and indicated standard of care therapy. For Part 2: expansion cohort—received at least one line of systemic therapy in the metastatic cancer setting and for patients who are either gemcitabine-containing regimen naïve or at least 6 months out of having been treated using a gemcitabine-containing regimen. CCR and CCA expansion cohorts—received at least one prior line of therapy in the metastatic setting.
11. Vaccination for COVID-19 is allowed before or during the study period. Information on timing and type of vaccine must be recorded.
12. Eastern Cooperative Oncology Group (ECOG) performance status 0-1/Karnofsky score>70.
13. High microsatellite instability (MSI-H) and microsatellite stability (MSS) patients are allowed for Part 1 of the study.
14. Adequate hematologic and end organ function, defined as neutrophil count≥1×10⁹/L, platelet count≥100×10⁹/L, for HCC in Part 1≥50×10⁹/L; hemoglobin≥8.5 g/dL without transfusion in the previous week, Creatinine≤1.5×ULN, AST (SGOT)≤3×ULN (≤5×ULN when HCC or hepatic metastases are present), ALT (SGPT)≤3×ULN (≤5×ULN when HCC or hepatic metastases present), Bilirubin≤1.5×ULN (patients with known Gilbert's disease may have a bilirubin≤3.0×ULN), Albumin≥3.0 g/dL, INR and PTT≤1.5×ULN; amylase and lipase≤1.5×ULN
15. No evidence of active infection or infections requiring parenteral antibiotics, and no serious infection within 4 weeks before study start.
16. Women of child-bearing potential must have a negative pregnancy test prior to study entry.
17. Women of child-bearing potential and men who have partners of child-bearing potential must be willing to practice highly effective contraception for the duration of the trial and for three months after the completion of treatment
18. Four (4) weeks or 5 half-lives (whichever is shorter) since the last dose of anti- cancer therapy before the first G9.2-17 administration
19. Continuation of bisphosphonate treatment (zoledronic acid) or denosumab for bone metastases which have been stable for at least 6 months before C1D1 is allowed.
20. For CCR and CCA expansion cohorts, at least one prior line of therapy in the metastatic setting is required.
21. For Part 1: Hepatocellular carcinoma that progressed while receiving at least one previous line of systemic therapy, including sorafenib, lenvatinib, nivolumab, atezolizumab and bevacizumab, or who are intolerant to or refused sorafenib treatment following progression on standard therapy including surgical and/or local regional therapies, or standard therapy considered ineffective, intolerable, or inappropriate or for which no effective standard therapy is available.
22. Biliary or gastric outlet obstruction allowed, provided it is effectively drained by endoscopic, operative, or interventional means.
23. Pancreatic, biliary, or enteric fistulae allowed, provided they are controlled with an appropriate non-infected and patent drain (if any drains or stents are in situ, patency needs to be confirmed before study start).

### Patient Exclusion Criteria:

1. Patient diagnosed with metastatic cancer of an unknown primary.

2. Patient unwilling or unable to follow protocol requirements
3. Prior or current illicit drug addiction (medical and recreational marijuana/CBD/THC is not considered “illicit”)
4. Clinically significant, active uncontrolled bleeding, and any patients with a bleeding diathesis (e.g., active peptic ulcer disease). Prophylactic or therapeutic use of anticoagulants is allowed.
5. Lactating females
6. Receiving any other investigational agents or participating in any other clinical trial involving another investigational agent for treatment of solid tumors within 4 weeks or 5 half-lives of the administered drug (whichever is shorter) prior to study dosing.
7. Radiation therapy within 4 weeks of the first dose of study drug, except for palliative radiotherapy to a limited field, such as for the treatment of bone pain or a focally painful tumor mass.
8. Patients with fungating tumor masses
9. Patients with locally advanced ocular melanoma
10. ≥CTCAE grade 3 toxicity (except alopecia and vitiligo) due to prior cancer therapy. Grade 4 immune-mediated toxicities with a prior checkpoint inhibitor. Grade 2 or Grade 3 pneumonitis or any other Grade 3 checkpoint inhibitor-related toxicity that led to immunotherapy treatment discontinuation. Low-grade (<grade 3) toxicities, such as neuropathy from prior treatments, manageable electrolyte abnormalities and lymphopenia, are allowed.
11. History of second malignancy, except those treated with curative intent more than five years previously without relapse or low likelihood of recurrence (for example, non-melanotic skin cancer, cervical carcinoma in situ, prostate cancer, or superficial bladder cancer)
12. Evidence of severe or uncontrolled systemic diseases, congestive cardiac failure>New York Heart Association (NYHA) class 2, myocardial infarction (MI) within 6 months, or laboratory finding that in the view of the Investigator makes it undesirable for the patient to participate in the trial
13. Any medical condition that the Investigator considers significant to compromise the safety of the patient or that impairs the interpretation of G9.2-17 toxicity assessment
14. Serious non-healing wound, active ulcer, or untreated bone fracture
15. Uncontrolled pleural effusion, pericardial effusion, or ascites requiring recurrent drainage procedures
16. History of severe allergic, anaphylactic, or other hypersensitivity reactions to chimeric or humanized antibodies or fusion proteins
17. Significant vascular disease (e.g., aortic aneurysm requiring surgical repair or recent arterial thrombosis) within 6 months of Cycle 1, Day 1
18. History of pulmonary embolism, stroke or transient ischemic attack within 3 months prior to Cycle 1, Day 1
19. History of abdominal fistula or gastrointestinal perforation within 6 months prior to Cycle 1, Day 1
20. Active auto-immune disorder (except type I diabetes, hypothyroidism requiring only hormone replacement, vitiligo, psoriasis, or alopecia)
21. Requires systemic immunosuppressive treatment including, but not limited to cyclophosphamide, azathioprine, methotrexate, thalidomide, and anti-tumor necrosis factor [anti-TNF] agents. Patients who have received or are receiving acute, low dose systemic immunosuppressant medications (e.g., dexamethasone 4 mg) may be enrolled. Replacement therapy (e.g., thyroxine, insulin, physiologic corticosteroid replacement therapy [e.g., ≤10 mg/day of prednisone equivalent] for adrenal or pituitary insufficiency) is not considered a form of systemic treatment. The use of inhaled corticosteroids and mineralocorticoids (e.g., fludrocortisone), topical steroids, intranasal steroids, intra-articular, and ophthalmic steroids is allowed
23. Tumor-related pain (>grade 3) unresponsive to broad analgesic interventions (oral and/or patches).
24. Uncontrolled hypercalcemia, despite use of bisphosphonates
25. Any other diseases, metabolic dysfunction, physical examination finding, or clinical laboratory finding giving reasonable suspicion of a disease or condition that contraindicates the use of an investigational drug or that may affect the interpretation of the results or render the patient at high risk of treatment complications
26. Received organ transplant(s)
27. Undergoing dialysis
28. For part 1, hormonal androgen deprivation therapy allowed to continue for subjects with metastatic castrate resistant pancreatic cancer.

A patient shall discontinue the treatment if one or more of the following occur:
Pregnancy
Unmanageable toxicity
Symptomatic deterioration attributed to disease progression as determined by the investigator after integrated assessment of radiographic data, biopsy results, and clinical status.
Intolerable toxicity related to G9.2-17, including development of an irAE determined by the investigator to be unacceptable given the individual patient's potential response to therapy and severity of the event
Any medical condition that may jeopardize the patient's safety if he or she continues on study treatment
Use of another non-protocol anti-cancer therapy

### (C) Objectives

### Part 1 (Phase 1a)

Primary Objective(s): Safety, tolerability, optimal biological dose (OBD) or maximum administered dose (MAD), recommended Phase 2 dose (RP2D)

Secondary Objective(s): Pharmacokinetic (PK), pharmacodynamic (PD) parameters, immunogenicity

Exploratory Objective(s): Exploratory end points for Part 1, in addition to exploratory end points listed below: Objective Response Rate (ORR), disease control rate (DCR), progression free survival (PFS), patient survival at 3 months (for Part 1), 6 and 12 months (for Parts 1 and 2).

### Part 2

Primary Objective(s): Objective Response Rate (ORR) or Progression free survival (PFS) at 6 months

Secondary Objective(s): Objective Response Rate (ORR), Progression free survival (PFS), disease control rate (DCR) (e.g., at 6 and 12 months), duration and depth of response by RECIST 1.1, patient survival at 6 and 12 months, time to response, safety and tolerability

### Exploratory Endpoints for All Study Parts:

iRECIST criteria, immunophenotyping from blood and tumors, cytokine profile (serum), soluble galectin-9 levels in blood (serum or plasma), galectin-9 tumor tissue expression levels and pattern of expression by immunohistochemistry (tumor, stroma, immune cells), tumor mutational burden (TMB), PDL-1 expression by immunohistochemistry, mismatch repair status, tumor markers relevant for the disease, ctDNA, and correlation of these parameters with response. Time to response (TTR). Quality of life and symptom control.

### (D) Study Procedures

### (i) Schedule of Assessments

The schedule of assessments is divided into 4-week cycles after the pre-dose 1 cycle 1 screening, which may take place up to 4 weeks prior to commencement of treatment. Table 33 lists the pre-dose screening assessments and tests, as well as indicating those to be conducted during the treatment cycles. Optional visits are allowed during each cycle, if medically indicated, during which any of the study assessments may be performed.

### (ii) Screening and Assessment Procedures

The following procedures (outlined in Table 33. Schedule of Assessments) must be conducted within 4 weeks of initiating treatment:
Written informed consent
Verify inclusion and exclusion criteria
Record of prior COVID-19 infection and latest RT PCR and/or SARS COV2 IgG/IgM test results, if performed
Record patient's intention to receive a COVID-19 vaccine if and when available
Record vaccination status for seasonal flu
Tumor imaging assessment CT with or without contrast is preferred, MRI with or without contrast if required based on investigator's judgement, PET-CT (diagnostic CT) if required based on investigator's judgement)
Tumor biopsy (pre dose 1 and repeat biopsy)-scheduled depending upon scan(s)
For the pre-screen assessment of galectin-9 expression by immunohistochemistry, archival tumor tissue may be used, if available, provided it was acquired within a 5-year time frame and details of treatment (s) administered post tissue acquisition are known and documented. This is not a pre-requisite for enrollment and investigators will endeavor to provide archival specimens whenever possible.
Relevant tumor marker per tumor type-e.g., CA15-3, CA-125, CEA, CA19-9, S100, alpha fetoprotein, etc., will be assessed every cycle pre-dose (which may be decreased to every 3 cycles after 6 months of treatment, following the same schedule as restaging scans), as appropriate.
Patient demographics
Personal medical history, including prior treatments/surgeries, record of any implants in situ or past implants, prior and/or current use of medical devices, concomitant medications (name, indication, dose, route, start and end dates dose modifications if any and reason), pre-existing symptoms, and adverse events), hereditary diseases at risk of based on family history and complete family history to the best knowledge of the patient
Any and all additional test results previously acquired (next generation and/or whole exome sequencing results, circulating tumor DNA testing, germline sequencing results, DPD test results, G6PD test results, Oncotype Dx and/or Endopredict test results, , consensus molecular subtypes (CMS) classification, DXA scans, if available. These are not a pre-requisite for enrollment.
Record of any dental surgery/root canal or ophthalmology surgery performed in the past 12 months
History of mandibular or maxillary necrosis
History of any prior port-a-cath infections requiring intravenous antibiotics and/or anti-fungals, port-a-cath replacements.
If the patient is deemed dehydrated according to investigator's assessment, oral and/or i.v. rehydration is allowed and advised prior to dosing at any cycle. Investigator may decide to order a BUN test on the day of dosing to guide decision making.
Physical examination and visual sign recording
ECHO, Ejection fraction (EF)
12-lead ECG
Record of the site and status/dimensions of any keloid scars
In patients with stable and pre-treated brain metastases, perform a neurological exam
Record of any dietary requirements or preferences (for example, practice of a particular diet regimen: intermittent fasting, keto diet etc.)
Use of supplements (current and with the past 12 months), type duration of use, dose and frequency
ECOG and/or Karnofsky status
Vital signs (temperature, HR, BP, RR, including weight) post-supine for 5 minutes
   Pregnancy test, if female of childbearing age. If prior history of bilateral salpingo-oophorectomy and/or hysterectomy then not needed but record these surgical procedures.
   Allergies past and/or present (allergen, severity)
   Complete blood count (CBC), differential, platelets, hemoglobin
   Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4, lipase, amylase, PTH, FSH, LH, CRP, and/or troponin:
   Blood coagulation (PT, PTT, APTT)
   Urinalysis
   PD blood—biomarker analysis
   PD tumor—biomarker analysis on the pre-treatment biopsy
      (iii) On-Study Procedures

Ensure that PD blood biomarker analyses are done at each blood draw; and tumor biomarker analyses are done on pre- and on/post-study biopsies. Screening process must involve and document a neurological exam. Any >grade 2 irAEs will be referred to the relevant specialist and documented accordingly. Management of irAEs will be conducted according to: Management of Immunotherapy-Related Toxicities, NCCN Guidelines Version 1.2020. Study-related procedures and assessments performed during on-study treatment are detailed as follows and in Table 46, Schedule of Assessments.

For COVID-19 infection diagnosed while on treatment, investigators and the Sponsor will follow FDA guidelines and local policies, and the investigator should contact the medical monitor to discuss best course of action.

### Cycle 1 Procedures

### (a) Cycle 1, Day 1 Procedures

The following procedures will be performed on Day 1 after all the previous screening and baseline procedures have been completed.
12-lead ECG
Physical examination
ECOG
Vital signs (temperature, HR, BP, RR, including weight and/or BSA) post-supine for 5 minutes
Concomitant medications (name, indication, dose, route, start and end dates, any and all dose modifications, timing thereof and reason)
Adverse events
Complete blood count (CBC), differential, platelets, hemoglobin
All within 7 days of C1D1: Blood chemistry (glucose, Hgb Alc (if history of DM1 or DM2), total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK)—Fasting glucose will be taken pre-dose, only if clinically indicated. TSH, fT4, lipase, amylase, PTH, FSH, LH, CRP, troponin
Relevant tumor marker—e.g., CA15-3, CA-125, CEA, CA19-9, S100, alpha fetoprotein, etc., will be assessed every cycle pre-dose (which may be decreased to every 3 cycles after 6 months of treatment, following the same schedule as restaging scans), as appropriate
Blood coagulation (PT, PTT, APTT)
Urinalysis (including protein and culture/antibiogram)
PD blood—biomarker analysis (soluble galectin-9, tissue IHC for galectin-9 from pre-treatment biopsy and immunophenotyping)
PK blood samples at time points as annotated in Table 46.

### (b) Cycle 1, Day 2 Procedures

The following procedures will be performed on Day 2 of Cycle 1.
Concomitant medications (name, indication, dose, route, start and end dates)
Adverse events
PD blood—biomarker analysis
PK blood samples

### (c) Cycle 1, Day 4 Procedures

The following procedures will be performed on Day 4 of Cycle 1.
Concomitant medications (name, indication, dose, route, start and end dates)
Adverse events
PD blood—biomarker analysis
PK blood samples

### (d) Cycle 1, Day 7 Procedures

The following procedures are performed on Day 7 of Cycle 1.
Vital signs (temperature, HR, BP, RR, including weight) post-supine for 5 minutes
Concomitant medications
Adverse events
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK)
PD tumor—biomarker analysis
PK blood samples

### (e) Cycle 1, Day 15 Procedures

The following procedures are performed on Day 15 of Cycle 1.
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK)ECOG
Vital signs (temperature, HR, BP, RR, including weight) post-supine for 5 minutes
Adverse events
PD blood—biomarker analysis
PK blood samples

### Cycle 2 Procedures

### (a) Cycle 2, Day 1 Procedures

The following procedures are performed on Day 1 of Cycle 2.
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4
Relevant tumor marker—e.g., CA15-3, CA-125, CEA, CA19-9, S100, alpha fetoprotein, etc., will be assessed every cycle pre-dose (which may be decreased to every 3 cycles after 6 months of treatment, following the same schedule as restaging scans), as appropriate.
Physical examination
Adverse events
ECOG
Vital signs (temperature, HR, BP, RR, including weight) post-supine for 5 minutes
PK blood samples
PD blood—biomarker analysis
Concomitant medications (name, indication, dose, route, start and end dates, any and all dose modifications, timing thereof and reason)

### (b) Cycle 2, Day 7 Procedures

The following procedures will be performed on Day 7 of Cycle 2.
12-lead ECG
Vital signs (temperature, HR, BP, RR, including weight) post-supine for 5 minutes
Concomitant medications (name, indication, dose, route, start and end dates)
Adverse events
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK)
PD blood—biomarker analysis
PK blood samples
Pregnancy test, if female of childbearing age and ovaries and uterus in situ

### (c) Cycle 2, Day 15 Procedures

The following procedures are performed on Day 15 of Cycle 2.
Restaging scan (CT with contrast, MRI, PET-CT or X-ray)—may be done 6-8 weeks from onset of study drug administration, scheduled as an additional separate visit
Tumor biopsy −3/+12 days if feasible and scheduled as a separate visit/can coincide with the scan as imaging guidance may be required to facilitate obtaining the tissue sample (target lesion should not be biopsied)
PD tumor—biomarker analyses
ECOG
Vital signs (temperature, HR, BP, RR, including weight) post-supine for 5 minutes
Adverse events
PK blood samples
PD blood—biomarker analysis

### Cycle 3 Procedures

### (a) Cycle 3, Day 1 Procedures

The following procedures are performed on Day 1 of Cycle 3.
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4
Relevant tumor marker—e.g., CA15-3, CA-125, CEA, CA19-9, S100, alpha fetoprotein, etc., will be assessed every cycle pre-dose (which may be decreased to every 3 cycles after 6 months of treatment, following the same schedule as restaging scans), as appropriate.
Physical examination
ECOG
Vital signs (temperature, HR, BP, RR, include weight) post-supine for 5 minutes
Concomitant medications (name, indication, dose, route, start and end dates)
Adverse events
PD blood—biomarker analysis
PK blood samples
Pregnancy test, if female of childbearing age

### (b) Cycle 3, Day 7 Procedures

The following procedures are performed on Day 7 of Cycle 3.
ECHO
Vital signs (temperature, HR, BP, RR, including weight) post-supine for 5 minutes
Concomitant medications (name, indication, dose, route, start and end dates)
Adverse events
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK)
PD blood—biomarker analysis
PK blood samples

### (c) Cycle 3, Day 15 Procedures

The following procedures are performed on Day 15 of Cycle 3.
ECOG
Vital signs (temperature, HR, BP, RR, including weight) post-supine for 5 minutes
Adverse events
PK blood samples
PD blood—biomarker analysis

### Cycle 4, and Beyond, Procedures

### (a) Cycle 4, Day 1 Procedures

The following procedures are performed on Day 1 of Cycle 4 and subsequent cycles.
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4, lipase, amylase, PTH,, troponin, FSH, LH, CRP
Restaging scan (CT with contrast, MRI, PET-CT or X-ray)—may be done 6-8 weeks from onset of study drug administration
Relevant tumor marker—e.g., CA15-3, CA-125, CEA, CA19-9, S100, alpha fetoprotein, etc., will be assessed every cycle pre-dose (which may be decreased to every 3 cycles after 6 months of treatment, following the same schedule as restaging scans), as appropriate
Physical examination
ECOG
Adverse events
PK blood samples
PD blood—biomarker analysis
Pregnancy test, if female of childbearing age

### (b) Cycle 4, Day 7 Procedures (Same as C3 D7)

The following procedures will be performed on Day 7 of Cycle 4

### (c) Cycle 4, Day 15 Procedures (Same as C3D15)

The following procedures will be performed on Day 15 of Cycle 4

### (iv) End of Study or Early Termination Procedures

The following procedures are done on Day 59 or thirty days after the last dose, including patients who have discontinued treatment early.
Restaging scan (CT with or without contrast id preferred, MRI with or without contrast, PET-CT if required by investigator)-repeat if end of study is >6 to 8 weeks after last cycle and in shorter intervals, at investigator's discretion
Relevant tumor marker—e.g., Ca15-3, CA-125, CEA, CA19-9, S100, alpha fetoprotein, etc., will be assessed every cycle pre-dose (which may be decreased to every 3 cycles after 6 months of treatment, following the same schedule as restaging scans), as appropriate
12-lead ECG
Physical examination
ECOG
Vital signs (temperature, HR, BP, RR, including weight) post-supine for 5 minutes
Concomitant medications (name, indication, dose, route, start and end dates)
Adverse events
Pregnancy test, if female and ovaries and uterus in situ
Complete blood count (CBC), differential, platelets, hemoglobin
Blood chemistry (glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK), TSH, fT4, PTH, Estradiol. prolactin, testosterone, FSH, LH
Blood coagulation (PT, PTT)
Urinalysis
PD blood—biomarker analysis
PK blood samples

### (v) Long-Term Follow-Up

Once a patient has completed the treatment period, overall survival follow-up will be performed every 3 months for up to 2 years. Radiological assessment will continue, where possible, for patients withdrawing due to clinical progression.

Survival data as well as information on any new anticancer therapy initiated after disease progression will be collected approximately every 3 months. Follow-up may be performed by telephone interview or chart review and will be reported on the case report form. During the Follow-Up Period, deaths, regardless of causality, and serious adverse events thought to be related to study treatment will be collected and reported within 24 hours of discovery or notification of the event.

### (vi) Study Assessments

### (a) Physical Examination

Medical and physical examinations must be performed by a qualified physician, nurse practitioner, or physician assistant, and should include a thorough review of all body systems at Screening, during treatment, and at End of Study. Physical examinations include a breast examination, if clinically indicated, as well as vital signs-temperature, heart rate (HR), blood pressure (BP), respiratory rate (RR)-measured after resting in a supine position for 5 minutes. Patient weight will also be measured and recorded.

### (b) Medical History

The medical history includes oncology history, radiation therapy history, surgical history, current and past medication.
Personal medical history, including prior treatments/surgeries, record of any implants in situ or past implants, prior and/or current use of medical devices, concomitant medications (name, indication, dose, route, start and end dates dose modifications if any and reason), pre-existing symptoms, and adverse events), hereditary diseases at risk of based on family history and complete family history to the best knowledge of the patient
Any and all additional test results previously acquired (next generation and/or whole exome sequencing results, circulating tumor free DNA testing, germline sequencing results, DPD test results, G6PD test results, Oncotype Dx and/or Endopredict test results,)
Record of any dental work performed in the past 12 months
Record of the site and status/dimensions of any keloid scars
For patients with previously resected pancreatic adenocarcinoma, record whether the primary tumor was localized to the head of pancreas, pancreatic body or the pancreatic tail.
Bowel habits/typical frequency and consistency

### (c) Clinical Laboratory Evaluations

Patients have blood samples collected for routine clinical laboratory testing, according to the Schedule of Assessments. The clinical laboratory parameters will be analyzed at the site's local laboratory. Laboratory assessments to be completed will include hematology and serum chemistry and will be defined as following:
Serum Chemistry: To include glucose, total protein, albumin, electrolytes [sodium, potassium, chloride, total CO2], calcium, phosphorus, magnesium, uric acid, bilirubin (total, direct), SGPT (ALT) or SGOT (AST), alkaline phosphatase, bilirubin, lactate dehydrogenase (LDH), creatinine, HgbAlc, blood urea nitrogen, CPK, TSH, fT4, lipase, amylase, PTH, testosterone, estradiol, prolactin, FSH, LH, and CRP. Fasting glucose is taken pre-dose on C1D1, C2D1, C3D1, C4D1 and on additional days, only if clinically indicated.
Hematology: To include complete blood count, differential, platelets, and hemoglobin
Coagulation: To include prothrombin time (PT) and partial thromboplastin time (PTT), activated partial thromboplastin time (APTT)
Biomarker Analysis (PD Blood): To include galectin-9 levels in patient serum/plasma, peripheral blood immunophenotyping, cytokine measurement.
Pharmacokinetic (PK) Blood Sampling: If the Investigator determines that the dose of study drug should be interrupted, additional PK, and safety assessments will be collected pre-dose (within 2 hours of dosing) & 4 hours+/−30 minutes post study drug. Centers that are not able to hold patients more than 2 hours post dose due to COVID-19 restrictions, will contribute samples at 2 hrs post dose only.

If administration is interrupted for any reason and then resumed, additional PK assessments may be performed during the interruption at the discretion of the Investigator. If the dose of study drug is reduced upon resuming administration, additional PK assessments will be collected pre-resumption of administration and at 2 hours+/−15 minutes post dosing completion. Additional PK and other blood assessments may be taken if clinically indicated at the discretion of the Investigator.

Blood for additional PK or PD assessments may be obtained approximately every 7 to 14 days, when possible, for up to 4 weeks after last study drug administration in patients who discontinue the study. Blood for PK assessment will be collected pre-dose, at 2 hours+/−15 minutes post completion of dosing) and 4, (+/−15 minutes) post-study drug administration.

### (d) Urinalysis

Patients will have urine samples collected for routine urinalysis. The urinalysis will include color, appearance, and dipstick for specific gravity, protein, white blood cell-esterase, glucose, ketones, urobilinogen, nitrite, WBC, RBC, and pH, and urine culture at screening.

### (e) Electrocardiogram (ECG)

The following parameters from 12-lead electrocardiograms will be evaluated: heart rate, PR interval, QRS duration, QT interval, and QTcF interval.

### (f) Tumor Imaging Assessment

CT with contrast is the preferred modality (MRI, PET-CT and/or other imaging modalities instead of or in addition to the CT scan if CT is not feasible or appropriate, given location of the disease). Assessment should include the neck/chest/abdomen/pelvis at a minimum and should include other anatomic regions as indicated, based on the patient's tumor type and disease history. Imaging scans must be de-identified and archived in their native DICOM format as part of the patient study file. While the type of scan obtained is at the discretion of the Investigator as appropriate for the disease, the same method should be used for the duration of the study. Assessments are done every 6 to 8 weeks+/−1 week and at the End of Treatment if not assessed within the last 4 to 6 weeks.

### (g) Tumor Biopsies

Pre and on/post-treatment biopsies are collected. Pre-treatment to be collected before administration of Dose 1. On treatment may be collected on any treatment day after Cycle 1 where a biopsy is feasible. Preferred next biopsy would be before the first on-study scan. In instances where the procedure cannot be performed within the protocol-specified timeframe, alternatives may be permitted but must be discussed with the Study Director/Medical Monitor. It is recognized that a variety of clinical factors may make it difficult to obtain adequate specimens. Decisions not to complete biopsy on-treatment should be discussed with the Medical Monitor.

### (h) Tumor Markers

Exploratory markers, e.g., CA15-3, CA-125, CEA, CA19-9, S100, alpha fetoprotein, etc., will be assessed every cycle pre-dose (which may be decreased to every 3 cycles after 6 months of treatment, following the same schedule as restaging scans), as appropriate.

### (i) Adverse Events

Adverse events (AEs) starting or worsening after study drug administration will be recorded. AEs should be followed until resolved to baseline, stabilized, or deemed irreversible. All serious AEs (SAEs) must be collected from the date of patient's written consent until 30 days post-discontinuation of dosing or patient's participation in the study, if the last scheduled visit occurs a later time.

TABLE 46
Schedule of Assessments

| | | | | | | | | | | | | | | | | | | | | Post- |
|---|---|---|---|---|---|---|---|---|---|---|---|---|---|---|---|---|---|---|---|---|
| | | | | | | | | | | | | | | | | | | | | Dose |
| | | | | | | | | | | | | | | | | | | | | End of |
| | | | | | | | | | | | | | | | | | | | | Study |
| | | | | | | | | | | | | | | | | | | | | or |
| | | | | | | | | | | | | | | | | | | | | Early |
| | | Treatment Phase | | | | | | | | | | | | | | | | | | Ter- |
| | Pre-Dose | | | | | | | | | | | | | | | Cycle 4 (28 Days) and beyond | | | | mina- |
| | Screening | Cycle 1 (28 Days) | | | | | | Cycle 2 (28 Days) | | | | Cycle 3 (28 Days) | | | | (every 14 Days) | | | | tion |
| | Study Day | | | | | | | | | | | | | | | | | | | |
| | | | | | | | | | | | | | | | | | | | | 30 |
| | | | | | | | | | | | | | | | | | | | | days |
| | | | | | | | | | | | | | | | | | | | | after |
| | | | | | | | Optional | | | | Optional | | | | Optional | | | | Optional | last |
| | −28 to −1 | 1 | 2** | 4** | 7** | 15 | Visit^{@} | 1 | 7** | 15 | Visit^{@} | 1 | 7** | 15 | Visit^{@} | 1 | 7** | 15 | Visit^{@} | dose |
| | Name of Cycle Day | | | | | | | | | | | | | | | | | | | |
| | | C1D1 | C1D2 | C1D4 | C1D7 | C1D15 | C1DXX | C2D1 | C2D7 | C2D15 | C2DXX | C3D1 | C3D7 | C3D15 | C3DXX | CXD1 | CXD7 | CXD15 | CX4DXX | |
| | +/− days allowable | | | | | | | | | | | | | | | | | | | |
| | | 1 | 1 | 1 | 1 | | na | 2 | 2 | | na | 2 | 2 | | na | 2 | 2 | | na | 3 |
| Study Drug administered | | X | | | | X | | X | | X | | X | | X | | X | | X | | |

Disease Assessment

| | | | | | | | | | | |
|---|---|---|---|---|---|---|---|---|---|---|
| Restaging Scan (CT with or | X | | | X +/− | | | | X +/− | | X |
| without contrast is preferred, MRI | | | | 3 days | | | | 3 days | | re- |
| with or without contrast, PET-CT | | | | | | | | | | peat |
| if requested by the investigator)^{B} | | | | | | | | | | if |
| may be done 6-8 weeks from onset | | | | | | | | | | end |
| of study drug administration | | | | | | | | | | of |
| | | | | | | | | | | study |
| | | | | | | | | | | is >6 |
| | | | | | | | | | | to 8 |
| | | | | | | | | | | weeks |
| | | | | | | | | | | after |
| | | | | | | | | | | last |
| | | | | | | | | | | cycle |
| Tumor Biopsy^{C} | X^{&} | | | | | | | X& | | |
| schedule will depend on the scan | | | | | | | | | | |
| Relevant Tumor Markers^{D} | X | X | X^{D} | | X | X^{D} | X | X^{D} | X | X |

Study Procedures & Examinations

| | | | | | | | | | | | | | | | | | |
|---|---|---|---|---|---|---|---|---|---|---|---|---|---|---|---|---|---|
| Eligibility Assessment & Informed | | | | | | | | | | | | | | | | | |
| Consent | | | | | | | | | | | | | | | | | |
| Demographics^{E} | X | | | | | | | | | | | | | | | | |
| Medical History^{F} | X | | | | | | | | | | | | | | | | |
| ECHO^{G} | X | | | | | | | | | | | | | X | | | |
| 12-lead ECG (QTcF) | X | X | | | | | X | x | | | X | | | X | | | X |
| Physical Exam^{H} | X | X | | | | | X | | X | | X | | X | X | | X | X |
| ECOG | X | X | | | | X | X | | X | | X | | X | X | | X | X |
| Vital Signs (Temp, HR, BP, RR- | X | X | | | x | X | X | x | X | | X | | X | X | | X | X |
| include weight) post-supine × 5 | | | | | | | | | | | | | | | | | |
| minutes | | | | | | | | | | | | | | | | | |
| Concomitant Medications^{I} | X | X | X | X | X | X | X | X | X | X | X | X | X | X | X | X | X |
| Adverse Events^{J} | | X | X | X | X | X | X | X | X | X | X | X | X | X | X | X | X |

Labs

| | | | | | | | | | | | | |
|---|---|---|---|---|---|---|---|---|---|---|---|---|
| Pregnancy Test^{K} | X | | | | | | | | | | | X |
| Blood Hematology^{L} | X | X | x | X | X | x | X | X | X | X | X | X |
| Chemistry^{M} | X | X | x | X | X | x | X | X | X | X | X | X |
| Coagulation, Glucose & | X | X | | X | X | | X | X | X | X | X | X |
| Urinalysis^{N} | | | | | | | | | | | | |

Research Studies (PD/PK)

| | | | | | | | | | | | | | | | | |
|---|---|---|---|---|---|---|---|---|---|---|---|---|---|---|---|---|
| PD Blood - Biomarker Analysis^{O} | X | X | x | x | x | X | X | x | X | X | | X | X | | X | X ^{J} |
| Exploratory Biomarkers (Soluble | X | X | | | | X | X | | X | X | | X | X | | X | X |
| Galectin-9 and | | | | | | | | | | | | | | | | |
| Immunophenotyping) | | | | | | | | | | | | | | | | |
| PK Blood Samples ^{P} | X | X | X | X | X | X | X | X | X | X | X | X | X | X | X | X ^{J} |

^{A}Study Drug Administration: treatment will be administered, and assessments performed as an outpatient for 4 hours on Day 1 of Cycle 1 and days xxx.
^{B}Restaging Scans (CT, MRI, PET-CT or x-ray): CT with contrast is the preferred modality (MRI if CT is not feasible or appropriate given location of the disease). Assessment should include the neck/chest/abdomen/pelvis at a minimum and should include other anatomic regions as indicated based on the patient's tumor type and/or disease history. Imaging scans must be de-identified and archived in their native DICOM format as part of the patient study file. While the type of scan obtained is at the discretion of the Investigator as appropriate for the disease, the same method should be used for the duration of the study. Assessments are done every 6 to 8 weeks +/− 1 week and at the End of Treatment if not assessed within the last 4 to 6 weeks.
^{C}Tumor Biopsies: pre and on/post-treatment biopsies are collected. Pre-treatment to be collected before administration of Dose 1. On-treatment may be collected on any treatment day after Cycle 1 where a biopsy is feasible. Preferred next biopsy would be before first on-study scan. In instances where the procedure cannot be performed within the protocol-specified timeframe, alternatives may be permitted but must be discussed with the Study Director/Medical Monitor. It is recognized that a variety of clinical factors may make it difficult to obtain adequate specimens. Decisions not to complete biopsy on-treatment should be discussed with the Medical Monitor.
^{D}Relevant Tumor Markers: Exploratory Markers, e.g., Ca15-3, CA-125, CEA, CA19-9, S100, alpha fetoprotein etc. will be assessed every cycle pre-dose (which may be decreased to every 3 cycles after 6 months of treatment, following the same schedule as restaging scans), as appropriate.
^{E}Demographics: includes date of birth, sex, height, race, ethnicity.
^{F}Medical History: includes oncology history, radiation therapy history, surgical history, current and past medication
^{G}MUGA/ECHO: repeat test will be collected, only if clinically indicated while on study.
^{H}Physical Exam: includes breast exam if clinically indicated
^{I}Concomitant Medications: name, indication, dose, route, start and end dates will be collected.
^{J} Adverse Events: starting or worsening after study drug administration will be recorded. AEs should be followed until resolved to baseline, stabilized or deemed irreversible. All SAEs must be collected from the date of patient's written consent until 30 days post discontinuation of dosing or patient's participation in the study, if the last scheduled visit occurs at a later time.
^{K}Pregnancy Test: Must have HCG sensitivity ≤ IU/L or equivalent units of HCG and within 24 hours of first treatment cycle)
^{L}Blood Hematology: complete blood count, differential, platelets, hemoglobin
^{M}Biochemistry: glucose, total protein, albumin, electrolytes [sodium, potassium, chloride, total CO₂], calcium, phosphorus, magnesium, uric acid, bilirubin (total, direct), SGPT (ALT) or SGOT (AST), alkaline phosphatase, bilirubin, lactate dehydrogenase (LDH), creatinine, blood urea nitrogen, CPK
^{N}Coagulation, Glucose and Urinalysis: PT, PTT, Glucose and UA are collected. Collections at *Cycle 3 and beyond will be done only if clinically indicated (e.g. signs of bleeding, especially GI bleeding). ** Fasting Glucose will be taken pre-dose on CID1, C3D1 and on additional days, only if clinically indicated
^{O}PD Blood - biomarker analysis: Gene expression, metabolites, oxygen consumption rate (OCR), other biomarker analysis and PDX development. Additional cycles to be performed on the same schedule as restaging scans.
^{P} PK Blood samples: If the Investigator determines that the dose of study drug should be interrupted, additional PK, and safety assessments will be collected pre-dose (within 2 hours of dosing) and 4 hours +/− 30 minutes post study drug administration upon resumption of dosing; additional PK assessments may be performed during the interruption at the discretion of the Investigator. If the dose of study drug is reduced, additional PK assessments will be collected pre-dose (within 2 hours of dosing) & after starting the reduced study drug dose. Additional PK, and other blood assessments may be taken if clinically indicated at the discretion of the Investigator. PK Blood samples Blood for additional PK and/or PD assessments may be obtained ~every 7 to 14 days, when possible, for up to 4 weeks after last study drug administration in patients who discontinue the study. In addition to the time points indicated in the Schedule of Assessments, blood for additional PK assessments may be obtained at the discretion of the Investigator.
^{@}Optional visits are allowed during each cycle, if medically indicated, during which time any of the study assessments may be performed.
§ LYT-200 will be administered every two weeks
Dosing: Dose will be administered to patient on Cycle 1 Day 1 and Cycle 1 Day 15; and will follow this schedule thereafter.
1 collected pre-dose (within 2 hours of dosing) and 4 hours (+/−30 minutes) post-study drug administration
2 collected pre-dose (within 2 hours of dosing) and 2, 4, 6, 8 and 12 hours (+/−15 mins) post-study drug administration
3 collected pre-dose (within 2 hours of dosing)

### (E) Safety Assessments

All observed or volunteered adverse events regardless of treatment group or causal relationship to study drug will be recorded on the adverse event page(s) of the case report form (CRF). Adverse events will be coded using the MedDRA coding system and all AEs will be graded according to the National Cancer Institute Common Terminology Criteria for Adverse Events, version 5.0 (NCI-CTCAE) [NCI, 2017].

### Adverse Events

An adverse event is defined in the International Conference on Harmonisation (ICH) Guideline for Good Clinical Practice as “any untoward medical occurrence in a patient or clinical investigation subject administered a pharmaceutical product and that does not necessarily have a causal relationship with this treatment.”

This definition of adverse events is broadened in this study to include any such occurrence (e.g., sign, symptom, or diagnosis) or worsening of a pre-existing medical condition from the time that a subject has signed informed consent to the time of initiation of the investigational drug. Worsening indicates that the pre-existing medical condition (e.g., diabetes, migraine headaches, gout, hypertension, etc.) has increased in severity, frequency, or duration of the condition or an association with significantly worse outcomes.

For all adverse events, the investigator must pursue and obtain information adequate to both determine the outcome of the adverse event and to assess whether it meets the criteria for classification as a serious adverse event requiring immediate notification to the sponsor or its designated representative. For all adverse events, sufficient information should be obtained by the investigator to determine the causality of the adverse event. The investigator is required to assess causality. For adverse events with a causal relationship to the investigational product, follow-up by the investigator is required until the event resolves or stabilizes at a level acceptable to the investigator and the sponsor clinical monitor or his/her designated representative.

### Serious Adverse Events

A serious adverse event (SAE) is defined as an adverse event that:
Results in death;
Is life threatening (places the subject at immediate risk of death);
Requires in-patient hospitalization or prolongation of existing hospitalization;
Results in persistent or significant disability/incapacity; or
Is a congenital anomaly/birth defect

Important medical events that may not result in death, be life threatening, or require hospitalization may be considered an SAE when, based upon appropriate medical judgment, they may jeopardize the patient and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization. A hospitalization meeting the definition for “serious” is any inpatient hospital admission that includes a minimum of an overnight stay in a health care facility.

Inpatient admission does not include: rehabilitation facilities, hospice facilities, skilled nursing facilities, nursing homes, routine emergency room admissions, same day surgeries (as outpatient/same day/ambulatory procedures), or social admission (e.g., subject has no place to sleep).

Safety will be assessed throughout the study by a qualified physician, physician assistant, or nursing staff. Measurements used to evaluate safety will include history, physical examination, vital signs, clinical laboratory tests, urinalysis, 12-lead ECG, and monitoring for AEs.

Laboratory measurements that deviate clinically significantly from previous measurements (as determined by the investigator) may be repeated. If warranted, additional or more frequent testing than is specified in the protocol should be done to provide adequate documentation of AEs and the resolution of AEs.

For all adverse events, enough information should be obtained by the investigator to determine the causality of the adverse event (e.g., study drug or other illness). The relationship of the adverse event to the study treatment is assessed following the definitions below:

Unrelated: any event that does not follow a reasonable temporal sequence from administration of study drug ANI) that is likely to have been produced by the patient's clinical state or other modes of therapy administered to the patient.

Unlikely: any event that does not follow a reasonable temporal sequence from administration of study drug OR that is likely to have been produced by the patient's clinical state or other modes of therapy administered to the patient.

Possibly: any reaction that follows a reasonable temporal sequence from administration of study drug OR that follows a known response pattern to the suspected drug AND) that could not be reasonably explained by the known characteristics of the patient's clinical state or other modes of therapy administered to the patient.

Related: any reaction that follows a reasonable temporal sequence from administration of study drug AND that follows a known response pattern to the suspected drug AND that recurs with re-challenge, AND OR is improved by stopping the drug or reducing the dose.

### (a) Dose-Reduction Procedure for Adverse Event Management

In the event where dose-reduction is used for AE management, two dose reductions are allowed. By 30% of the baseline dose at each dose reduction. Dose reductions are pursued when clinical benefit is expected and may continue to be derived.

### (b) Criteria for Discontinuation of Study Treatment

Patients should ordinarily be maintained on study treatment until confirmed radiographic progression. If the patient has radiographic progression but no unequivocal clinical progression and alternate treatment is not initiated, the patient may continue on study treatment, at the investigator's discretion. However, if patients have unequivocal clinical progression without radiographic progression, study treatment should be stopped and patients advised regarding available treatment options.

### (c) Ongoing Safety Review

G9.2-17 should be withheld in the event of a serious or life-threatening immune related adverse reaction (IMAR) or one that prompts initiation of systemic steroids, although specific exceptions (e.g., for certain endocrinopathies in clinically stable patients) may be allowed. Provide a detailed monitoring plan intended to limit the severity and duration of IMARs that occur during combination drug development.

Abraxane is given at 125 mg/m2 intravenously over 30-40 minutes on Days 1, 8, and 15 of each 28-day cycle. Gemcitabine is administered on Days 1, 8 and 15 of each 28-day cycle immediately after Abraxane. One or more of the following may be performed based on development of potential adverse event in a patient:
No adjustment is necessary for patients with mild hepatic impairment.
Withhold Abraxane if AST>10×ULN or bilirubin>5×ULN.
Reduce starting dose in patients with moderate to severe hepatic impairment.
Dose reductions or discontinuation may be needed based on severe hematologic, neurologic, cutaneous, or gastrointestinal toxicities.

### Contraindications

Neutrophil counts of <1,500 cells/mm³
Severe hypersensitivity reaction to Abraxane. Patients with a known hypersensitivity to gemcitabine.

Table 47 below provides exemplary guidance with respect to recommended doses and reduced doses of Abraxane and gemcitabine. See also Abraxane monograph: Abraxis BioScience, LLC. Highlights of Prescribing Information [Internet]. Summit (NJ): Celgene Corporation; 2019 December [cited 2020 May 7].

TABLE 47
Recommendations for Abraxane Starting Dose
in Patients with Hepatic Impairment

| Degree of | SGOT | | | |
|---|---|---|---|---|
| Hepatic | (AST) | | Bilirubin | |
| Impairment | Levels | | Levels | Abraxane Dose |
| Mild | <10 × ULN | AND | >ULN to # | 125 mg/m² |
| | | | 1.25 × ULN | |
| Moderate | <10 × ULN | AND | 1.26 to 2 × ULN | not recommended |
| Severe | <10 × ULN | AND | 2.01 to 5 × ULN | not recommended |
| | >10 × ULN | OR | >5 × ULN | not recommended |

^{a} Patients with bilirubin levels above the upper limit of normal were excluded from clinical trials for pancreatic or lung cancer.

Given the possibility of extravasation, it is advisable to closely monitor the infusion site for possible infiltration during drug administration. Limiting the infusion of Abraxane to 30 minutes, as directed, reduces the likelihood of infusion-related reactions

### (d) Identification of Potential Safety Issues

Dose Limiting Toxicity (DLT) period: One (1) cycle.

One cycle encompasses CID1 (cycle one day one) and C1D15 (cycle one day fifteen).

### Monitoring Plans

In Part 1, the dose-escalation phase, dose escalation to the next cohort will proceed following review of Cycle 1 of each cohort. Safety and available PK data will be used to assess for DLTs in all patients of each cohort by the SMC. As a safety precaution, during dose escalation, new patients will be entered and treated only after the first patient of each cohort has been treated with G9.2-17 and at a minimum 7-14 days post-treatment has elapsed. Select DLT safety analysis for each patient will be performed following completion of Cycle 1. During the expansion phase, toxicities will be monitored by the SMC, which will convene to review aggregate toxicity rate prior to each dose escalation. The frequency of SMC meetings will increase as warranted by an increased toxicity rate. The SMC has the right to recommend to terminate or alter the study design of this clinical study at any time, including but not limited to testing of intermediate dose levels or initiation of the intermittent dose schedule.

### (e) Dose Limiting Toxicity Criteria

Dose-limiting toxicity (DLT) is defined as a clinically significant non-hematologic adverse event or abnormal laboratory value assessed as unrelated to metastatic tumor disease progression, intercurrent illness, or concomitant medications and is related to the study drug and occurring during the first cycle on study that meets any of the following criteria:
All Grade 4 non-hematologic toxicities of any duration
All Grade 3 non-hematologic toxicities. Exceptions are:
   Grade 3 nausea, vomiting and diarrhea that does not require hospitalization or TPN support and can be managed with supportive care to ≤Grade 2 within 48 hours.
   Grade 3 electrolyte abnormalities that are corrected to ≤Grade 2 within 24 hours.
   Other grade 3 asymptomatic laboratory abnormalities

DLT period includes one (1) cycle, i.e., four (4) weeks. One cycle encompasses the administration of G9.2-17 on days 1 and 15 (C1D1 and C1D15; Cycle 1 Day 1 and Cycle 1 Day 15, respectively).

### (f) Dose Delays and Reductions

Any AE>Grade 3 possible, probably, or definitely related to one or more study drugs will be discussed with the Medical Monitor before continuing with dosing, with the following exceptions, for which no discussion with the Medical Monitor will be required:
Local injection site reactions lasting <72 hours including pain, redness, swelling, induration, or pruritus
Systemic injection reactions lasting <72 hours of fever, myalgia, headache, or fatigue

Where judged appropriate by the Investigator (after discussion with the Medical Monitor) a dose delay may be necessary for >Grade 3 adverse events until resolution of the toxicity (to Grade 1 or less).

In Part 2 of the protocol, if 3 more than 3 patients develop a DLT, the dose of G9.2-17 will be reduced to 1 dose below the recommended Phase 2 dose (RP2D)

### (F) RECIST Criteria for Tumor Assessment

At the baseline tumor assessment, tumor lesions/lymph nodes will be categorized as measurable or non-measurable with measurable tumor lesions recorded according to the longest diameter in the plane of measurement (except for pathological lymph nodes, which are measured in the shortest axis). When more than one measurable lesion is present at baseline all lesions up to a maximum of five lesions total (and a maximum of two lesions per organ) representative of all involved organs should be identified as target lesions. Target lesions should be selected on the basis of their size (lesions with the longest diameter). A sum of the diameters for all target lesions will be calculated and reported as the baseline sum diameters.

All other lesions (or sites of disease) including pathological lymph nodes should be identified as non-target lesions and should also be recorded at baseline. Measurements are not required and these lesions should be followed as ‘present’, ‘absent’, or ‘unequivocal progression’.

Disease response (complete response (CR), partial response (PR), stable disease (SD), and progressive disease (PD)) will be assessed as outlined in Appendix 4.

The disease response measures will allow for the calculation of the overall disease control rate (DCR), which includes CR, PR, and SD, the objective response rate (ORR), which includes CR and PR, progression-free survival (PFS), and time to progression (TTP).

### (G) Patient Completion or Withdrawal

### Patient Completion

### Part 1—Dose Finding:

Patients will receive study drug at one of 5 dose levels every 2 weeks until progression of disease, unacceptable toxicity, or withdrawal from the study development of dose-limiting toxicity (DLT).

Two patients will be dosed, with a maximum available sample size of 24. Dose escalations will only be initiated when approval from the SMC has been received. At each dose escalation, new patients will only be entered and treated after the two patients in the previous cohort has been treated with G9.2-17 and at a minimum 7 days post-treatment has elapsed.

Part 1 will be completed when six consecutive patients have received the same dose and that dose will be identified as the OBD.

### Part 2—Tumor-Type Specific Treatment:

An expansion cohort for patients with ocular melanoma (e.g., metastatic) will entail combination treatment of G9.2-17 and gemcitabine/Abraxane. Completion of study will be dependent upon patient response at 3 months and responding patient survival at 12 months.

### Part 2—Expansion:

If a promising efficacy signal is identified within one of the five trial arms attributable to the tumor type, an expansion cohort will be launched to confirm the finding. Completion will be as described for Part 2.

Discontinuation from Study Treatment

A patient may be discontinued prior to completion of the study treatment for any of the following reasons:
Dose-limiting toxicity-defined as a clinically significant non-hematologic adverse event or abnormal laboratory value assessed as unrelated to metastatic tumor disease progression, intercurrent illness, or concomitant medications and is related to the study drug and occurring during the first cycle on study that meets any of the following criteria:
   All Grade 4 non-hematologic toxicities of any duration
   All Grade 3 non-hematologic toxicities. Exceptions are as follow:
      Grade 3 nausea, vomiting and diarrhea that does not require hospitalization or TPN support and can be managed with supportive care to ≤grade 2 within 48 hours.
      Grade 3 electrolyte abnormalities that are corrected to ≤grade 2 within 24 hours.
Progressive disease according to RESIST criteria or significant clinical progression at an earlier time point, if judged by the Investigator to be in the patient's best interests

Intercurrent illness that prevents further administration of treatment

Equivalents

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several inventive embodiments have been described and illustrated herein, those of ordinary skill in the art are readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art are readily appreciate that all parameters, dimensions, materials, and configurations described herein are meant to be exemplary and that the actual parameters, dimensions, materials, and/or configurations are depend upon the specific application or applications for which the inventive teachings is/are used. Those skilled in the art are recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” are refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for treating a solid tumor that expresses galectin-9 in a human subject, comprising administering to the human subject in need thereof an effective amount of a pharmaceutical composition comprising an antibody that binds galectin-9 (anti-galectin-9 antibody),
wherein the antibody comprises (a) a heavy chain variable region (V_{H}), which comprises a heavy chain complementary determining region 1 (CDR1) set forth as SEQ ID NO: 4, a heavy chain complementary determining region 2 (CDR2) set forth as SEQ ID NO: 5, and a heavy chain complementary determining region 3 (CDR3) set forth as SEQ ID NO: 6 and (b) a light chain variable region (V_{L}), which comprises a light chain CDR1 set forth as SEQ ID NO: 1, a light chain CDR2 set forth as SEQ ID NO: 2, and a light chain CDR3 set forth as SEQ ID NO: 3, and
wherein the solid tumor is an ocular melanoma.

2-3. (canceled)

4. The method of claim 1, wherein the antibody is an IgG1 or an IgG4 antibody.

5. The method of claim 1, wherein the antibody comprises the V_{H} set forth as SEQ ID NO: 7 and/or the V_{L} set forth as SEQ ID NO: 8.

6. The method of claim 4, wherein the antibody comprises a heavy chain constant region set forth as SEQ ID NO: 14, and/or a light chain constant region set forth as SEQ ID NO: 11.

7. The method of claim 1, wherein the anti-galectin-9 antibody comprises a heavy chain comprising the amino acid sequence of SEQ ID NO: 19 and a light chain comprising the amino acid sequence of SEQ ID NO: 15.

8. (canceled)

9. The method of claim 1, wherein the ocular melanoma is uveal melanoma.

10. The method of claim 1, wherein the ocular melanoma is metastatic.

11. The method of claim 1, wherein the anti-galectin-9 antibody is administered to the human subject at a dose of about 0.2 mg/kg to about 32 mg/kg.

12. The method of claim 11, wherein the anti-galectin-9 antibody is administered to the human subject at a dose of about 0.2 mg/kg to about 16 mg/kg.

13. The method of claim 12, wherein the anti-galectin-9 antibody is administered to the human subject at a dose of about 0.2 mg/kg, about 0.63 mg/kg, about 2 mg/kg, about 6.3 mg/kg, about 10 mg/kg, or about 16 mg/kg.

14. The method of claim 11, wherein the anti-galectin-9 antibody is administered to the subject at a dose of about 0.5 mg/kg to about 32 mg/kg, or about 2 mg/kg to about 16 mg/kg.

15. The method of any one of claims 1-14, wherein the anti-galectin-9 antibody is administered to the human subject once every two weeks or once every week.

16. The method of claim 1, wherein the anti-galectin-9 antibody is administered to the subject by intravenous infusion.

17. The method of claim 1, wherein the human subject is free of other anti-cancer therapy concurrently with the anti-galectin-9 antibody.

18. The method of claim 1, wherein the method further comprises administering to the human subject an effective amount of a checkpoint inhibitor; wherein the checkpoint inhibitor is an anti-PD-1 or anti-PD-L1 antibody, and wherein the antibody is selected from the group consisting of nivolumab, prembrolizumab, tislelizumab, or cemiplimab, durvalumab, avelumab, and atezolizumab.

19-20. (canceled)

21. The method of claim 18, wherein the antibody that binds PD-1 is nivolumab, which is administered to the subject at a dose of about 240 mg once every two weeks; or wherein the antibody that binds PD-1 is tislelizumab, which is administered to the subject at a dose of about 200 mg once every 3 weeks or about 400 mg once every six weeks.

22. The method of claim 18, wherein the immune checkpoint inhibitor is administered by intravenous infusion or subcutaneous injection. 23 (Previously presented) The method of claim 1, wherein the method further comprises administering a chemotherapy. 24 (Previously presented) The method of claim 23, wherein the chemotherapy comprises an antimetabolite, a microtubule inhibitor, or a combination thereof.

25. The method of claim 24, wherein the antimetabolite is gemcitabine and/or the microtubule inhibitor is paclitaxel.

26. The method of claim 25, wherein the paclitaxel is a protein-bound paclitaxel.

27. The method of claim 25, wherein the paclitaxel is administered to the subject at 125 mg/m² intravenously; and/or wherein the gemcitabine is administered to the subject at 1000 mg/m².

28. (canceled)

29. The method of claim 25, wherein the method comprises one or more cycles, each having 28 days, and wherein in each cycle the anti-galectin-9 antibody is administered to the human subject on day 1 and day 15 and the gemcitabine and paclitaxel are administered to the human subject on day 1, day 8, and day 15.

30. The method of claim 1, wherein the human subject has undergone one or more prior anti-cancer therapies; and/or wherein the human patient having an elevated level of galectin-9 relative to a control value.

31. (canceled)

32. The method of claim 30, wherein the human subject has progressed disease through the one or more prior anti-cancer therapies, or is resistant to the one or more prior therapies.

33-36. (canceled)

37. The method of claim 1, further comprising monitoring occurrence of one or more adverse effects in the human subject; wherein the one or more adverse effects comprise hepatic impairment, hematologic toxicity, neurologic toxicity, cutaneous toxicity, gastrointestinal toxicity, or a combination thereof.

38. (canceled)

39. The method of claim 37, further comprising reducing the dose of the anti-galectin-9 antibody, the dose of the checkpoint inhibitor, the dose of the chemotherapy, or a combination thereof, when an adverse effect is observed in the human subject.

40. The method of claim 1, wherein the human subject is examined for one or more of the following features before, during, and/or after the treatment:
(a) one or more tumor markers in tumor biopsy samples from the subject, optionally wherein the one or more tumor markers comprise CA15-3, CA-125, CEA, CA19-9, S100, alpha fetoprotein, neuron-specific enolase (NSE), and/or cytokeratin-fragment-21 (CYFRA-21);
(b) cytokine profile, which optionally comprises interferon gamma (IFN-γ), IL-10, IL-12p70, IL-13, IL-1B, IL-2, IL-4, IL-6, IL-8, TNF-α, MIP-1b, monocyte chemoattractant protein 1 (MCP-1), MIP-1a, IL-17a, IL-5, and/or TGF-β; and
(c) galectin 9 levels, optionally in blood and/or in tumor tissues;
(d) PD-L1 level, optionally in tissues;
(e) Mismatch repair status, optionally in tissues, and
(f) Tumor mutation burden (TMB).

41. The method of claim 1, wherein the ocular melanoma is uveal melanoma, which is located at choroid, ciliary body, or iris.

42. A method for identifying an ocular melanoma patient and optionally treating the patient, the method comprising:
(i) providing a biological sample of a subject suspected of having an ocular melanoma,
(ii) measuring the level of galectin-9 in the biological sample, and
(iii) identifying the subject as having the ocular melanoma or being at risk for the ocular melanoma or based on the level of galectin-9 in the biological sample, wherein an elevated level of galectin-9 in the biological sample of the subject relative to a predetermined reference level indicates that the subject has the ocular melanoma or is at risk for the ocular melanoma.

43. A method for determining tumor burden or metastatic status in a patient having ocular melanoma, and optionally treating the patient, the method comprising:
(i) providing a biological sample of a patient having an ocular melanoma,
(ii) measuring the level of galectin-9 in the biological sample, and
(iii) determining tumor burden of the patient based on the level of galectin-9 in the blood sample, wherein an elevated level of galectin-9 in the biological sample of the subject relative to a predetermined reference level indicates that the subject has a high tumor burden.

44. (canceled)

45. A method for assessing progression of ocular melanoma in a subject and optionally treating the subject, the method comprising:
(i) providing a first biological sample of a subject having ocular melanoma at a first time point;
(ii) providing a second biological sample of the subject at a second time point, wherein the second time point is after the first time point;
(iii) measuring the levels of galectin-9 in the first and second biological samples; and
(iv) determining the progression of ocular melanoma in the subject based on the levels of Gal-9 in the first and second biological samples, wherein an increased level of Gal-9 in the second biological sample relative to the level of Gal-9 in the first biological sample indicates progression of ocular melanoma in the subject.

46-47. (canceled)

48. A method for determining a response to a treatment in a subject having an ocular melanoma, and optionally modifying the treatment, the method comprising:
(i) providing multiple biological samples of a ocular melanoma patient who is under a treatment comprising an anti-cancer therapy, wherein the multiple biological samples comprise at least a first biological sample collected before the first dose of the anti-cancer therapy and a second biological sample collected after the first dose of the anti-cancer therapy;
(ii) measuring the level of galectin-9 in the biological samples; and
(iii) determining the patient's response to the anti-cancer therapy based on the difference between the level of Gal-9 in the first biological sample and the level of Gal-9 in the second biological sample; wherein a reduced level of Gal-9 in the second biological sample relative to the first biological sample indicates a response to the anti-cancer therapy in the subject.

49-54. (canceled)
